SEQUENCE LISTING (22 sequences)

SEQ ID NO: 1 - DNA, 401 bp, Homo sapiens
gccattctattacctttttttttttcagagatgggagtctcactctgtcacctggggtgcagtggcacgatccaatcttggttcactgcaacctccgcctcccaggttcaagcaattcttgtgcctcagccaccaagtagctggaaccacaagtgcatcaccacatatggctccttttttgtagagacagggttttgtcaygttgctcaggctggtcttgaactcctgaactcaagcgatccgcctgcctcggcctcccaaagtgctggggattacaggcatgagccactgcactccaccccattctatcactggaaataaaaaaatttcattacaggctgggcacagtggctcatgcctataatcccaacattttgagaggccaaggccggaggactgca

SEQ ID NO: 2 - DNA, 401 bp, Homo sapiens
caggagttcaagattacagtgagctatgattgtgccatagcactacagcctgggtgaaagagcttaaaaaaaaaaaaaaaaaaaaaacagaggctgggcacggtggctcacgcctgtaatcccagcactttgggaggccgaggcgggtggatcgcgaggtcaggagatcgagaccatcgtggctaacatggtgaaaccccrtctctactaaaaatacaaaaattagccgggcgtggtggcaggctcctgtagtcccagctactcgggaggctgaggcaggagaatggcgtgaacctgggaggtggagcttgcagtgagccaagatatcgcaccactgcactccagcctgggcgacaaagctagactccgtctcaaaaaaggaaaaaaaaaaaaaaaaaaag

SEQ ID NO: 3 - DNA, 401 bp, Homo sapiens
agctgggactacaggcacatgccaccacgcctagctaattttttgtattttagtagagacagggtttcaccatgttgcccaggctggtcttgaactcctgtgcttaagcaatccgcccacctcggcctcccaaagtgctaggattacaggcgtgagccaccgcacccagcctcaaaatgcttcttaaattcaatttaattwttttttttttttgagacggagtctcactctgctgcccaggttggagtgcagtgtggcaatctcggctcactacaacctccgtctcctgggttcaagtgattctcctgcctcagcctgctgagtagctgggattacaggcacctgccaccatgcccagataatttttgtatttttagtagaggcggggtttcaccatattg

SEQ ID NO: 4 - DNA, 401 bp, Homo sapiens
tgtttattctataagctgtataaccaaaaagaaaaattaaagatactttaggaataaaataatttgaaaaataaaagaacttatttgcatgatgtatcctgaaaaattagggcagccagtattactgaaataatggaggagtccgggagtggttgtggtatttttgtatttaaagcttacacaatagagtagaaacgttgyggtacatatttacttacaagaatatgttttgaataagtttactttcctaaatgtcatctgttttaatgacctgctacttggattctttctcacagtgttaggttgctgactatgtgaaatttcctacttcttattattgctatcagaataggaaatttaaaattaaatcagaaatgtaaaagtcttaaaacttcaccttt

SEQ ID NO: 5 - DNA, 401 bp, Homo sapiens
taggtactgttcaaagctctttgcatgtactaactcatttaatcctcacaaaaaacataaggcagaaactattattaacttcattttagagatgagaaaacataaatgaggaagttaagcaatttctctagcatttcacaagaagtagcagagtaggaatttaaacccaagaaatctatccccacagtctgtactatttartctctcaaactataatgggaaataaccatttttcatccactgaatcagcatcctactaggcaaggctttgggaaaacaggcactttaactggtgggaattcaaactgatattacaggggctgggcatggtggctcacgcctgtaatctcagcactttgggaggcctaggtaggtggatcacttgaggcaagcagttcaag

SEQ ID NO: 6 - DNA, 401 bp, Homo sapiens
cgggctagaacagttctcacatttttaaatgggaggaaaaacatcaaaagaagattttatgaaacctgaaaatcacatgaaattcacatgtcaggctcataaataagttttaatgcaacagagtcatgtctgttcacttatgtattatctatggctgctctccctatgatggcagagttgagcagctgtaacagagaccgkgcggcctaaagatacttactgtcgggctggttacagaaaatgttttccaaacagaagctgtattccaaattctttcctaatgtggcatacaaagcctttaggatctggctcctgtgtgcctctcctgccgctgctgctgctgctgctgctcctgtttccgcttctgccatggccaccgccactgccgccgcctcctcctt

SEQ ID NO: 7 - DNA, 401 bp, Homo sapiens
tttaaaacacaaatatatcatacctggtgccagaaagttttttgttttccttaactatattggaaatcacctatttcccaacttgaagactttaataaatattattctcaaagaattttcaaatatagtcattccttggtgtcatgggaaattggttccagaacctccctcagataccaaaatccacagatgctcaagtcmctgatgctggtactatatttgcatatgacccacgcacatccccctgtatgcttcaagtcatctttagattacttataatacctaatacaatgtaagtgctatataaatagttgttaacactgtattgtttgggaatcatgacaagaaaaaagtctagacatgttcggtagagacgcaaccatccttttttttttcacaga

SEQ ID NO: 8 - DNA, 401 bp, Homo sapiens
ttcactgcttgggtaagtgtataactctttctgggaacatatattaagaaatttatatatttacatcctctgatttcatatgtaggaatctaactcaaggaaatttttgtatgttgatgtgtatatataaatgtcttttggtggtagaaattatgagggttttcagcttctcattcttttctatttcttccacacttcttyaatgatcatctcttactttcttacttttctttctacttacctcccctctttttctctgttcctcccagtacttcattgcttgatttctctgagtattaaaatggtaggccaggcgtggtggttcatgcctgtaatcccagcactctgagagccaaagccagaggatcacttgaggccaggagtttgacaccagcctggtc

SEQ ID NO: 9 - DNA, 401 bp, Homo sapiens
agggtaaccccaattcataaaaataaaaatcatattatcatcacatacaaaaaggcttatcttcctggtcttgatagcaagggagtttgaaggaagttcatcaaaggggtaaaatctttaagatacaaagttaaaaagaaagaagatatctcccgattatgtttttttaaaatcttcaagaaacctaagaaatacccaatrgaaaaatggaggctgggcacggtggctcacacttgtaattcaagcactttgggaggctgaggcgagtgatcatttgaggtcaggagttagagatcagcctggccaacatggtgaaaccccatttctactaaaaatacaaaaatcagccgggcgtggtggcaggtgcctgtaatcccagctactcaggaggttgaagcagg

SEQ ID NO: 10 - DNA, 8166 bp, Homo sapiens
gctattgggcgctgggagtcgagggggcgggaggcgggaattcgtctcgggttgtgtggttgaggggtctggtgggtcgaggaaaggtaacggcggccccagtcctgcacacaaggccggggaagtagcagcacccccaggaagagggaggaggaagggctcgtgccctttcttctcttccagggctccgctttatttgctctcagaagtcggtttcctttccttttcttcagtgaatcggagctcagagcgttgcttcggtttccctccagacagttaggaatctgaaataaacaggaaagcactatgtcttcaatgtggtctgaatatacaattggtggggtgaagatttactttccttataaagcttacccgtcacagcttgctatgatgaattctattctcagaggattaaacagcaagcaacattgtttgttggagagtcccacaggaagtggaaaaagcttagccttactttgttctgctttagcatggcaacaatctcttagtgggaaaccagcagatgagggcgtaagtgaaaaagctgaagtacaattgtcatgttgttgtgcatgccattcaaaggattttacaaacaatgacatgaaccaaggaacttcacgtcatttcaactatccaagcacaccaccttctgaaagaaatggcacttcatcaacttgtcaagactcccctgaaaaaaccactctggctgcaaagttatctgctaagaaacaggcatccatatacagagatgaaaatgatgattttcaagtagagaagaaaagaattcgacccttagaaactacacagcagattagaaaacgtcattgctttggaacagaagtacacaatttggatgcaaaagttgattcaggaaagactgtaaaactcaactctccactggaaaagataaactccttttcgccacagaaaccccctggccactgttctaggtgctgttgttctactaaacaaggaaacagtcaagagtcatcgaataccattaagaaggatcatacagggaaatccaagatacccaaaatatattttgggacacgcacacacaagcagattgctcagattactagagagctccggaggacggcatattcaggggttccaatgactattctttccagcagggatcatacttgtgtccatcctgaggtagtcggtaacttcaacagaaatgagaagtgcatggaattgctagatgggaaaaacggaaaatcctgctatttttatcatggagttcataaaattagtgatcagcacacattacagactttccaagggatgtgcaaagcctgggatatagaagaacttgtcagcctggggaagaaactaaaggcctgtccatattacacagcccgagaactaatacaagatgctgacatcatattttgtccctacaactatcttctagatgcacaaataagggaaagtatggatttaaatctgaaagaacaggttgtcattttagatgaagctcataacatcgaggactgtgctcgggaatcagcaagttacagtgtaacagaagttcagcttcggtttgctcgggatgaactagatagtatggtcaacaataatataaggaagaaagatcatgaacccctacgagctgtgtgctgtagcctcattaattggttagaagcaaacgctgaatatcttgtagaaagagattatgaatcagcttgtaaaatatggagtggaaatgaaatgctcttaactttacacaaaatgggtatcaccactgctacttttcccattttgcagggacatttttctgctgttcttcaaaaagaggaaaaaatctcaccaatttatggtaaagaggaggcaagagaagtacctgttattagtgcatcaactcaaataatgcttaaaggactttttatggtacttgactatctttttaggcaaaatagcagatttgcagatgattataaaattgcgattcaacagacttactcctggacaaatcagattgatatttcagacaaaaatgggttgttggttctaccaaaaaataagaaacgttcacgacagaaaactgcagttcatgtgctaaacttttggtgcttaaatccagctgtggccttttcagatattaatggcaaagttcagaccattgttttgacatctggtacattatcaccaatgaaatccttttcgtcagaacttggtgttacatttactatccagctggaggctaatcatatcattaaaaattcacaggtttgggttggtaccattgggtcaggccccaagggtcggaatctctgtgctaccttccagaatactgaaacatttgagttccaagatgaagtgggagcacttttgttatctgtgtgccagactgtgagccaaggaattttgtgtttcttgccatcttacaagttattagaaaaattaaaagaacgttggctctctactggtttatggcataatctggagttggtgaagacagtcattgtagaaccacagggaggagaaaaaacaaattttgatgaattactgcaggtgtactatgacgcaatcaaatacaaaggagagaaagatggagctctcctggtagcagtttgtcgtggtaaagtgagtgagggtctggatttctcagatgacaatgcccgtgctgtcataacaataggaattccttttccaaatgtgaaagatctacaggttgaactaaaacgacaatacaatgaccaccattcaaaattgagaggtcttctacctggccgtcagtggtatgaaattcaagcatacagggccttaaaccaggcccttggtagatgtattagacacagaaatgattggggagctcttattctagtggatgatcgctttaggaataacccaagtcgctatatatctggactttctaaatgggtacggcagcagattcagcaccattcaacctttgaaagtgcactggaatccttggctgaattttccaaaaagcatcaaaaagttcttaatgtatccataaaggacagaaccaatatacaggacaatgagtctacacttgaagtgacctctttaaagtacagtacctcaccttatttactggaagcagcaagtcatctatcaccagaaaattttgtggaagatgaagcaaagatatgtgtccaggaactacagtgtcctaaaattattaccaaaaattcacctctaccaagtagcattatctccagaaaggagaaaaatgatccagtattcctggaagaagcagggaaagcagaaaaaattgtgatttccagatccacaagcccaactttcaacaaacaaacaaagagagttagctggtcaagctttaattctttgggacagtattttactggtaaaataccgaaggcaacacctgagctcgggtcatcagagaatagtgcctctagtcctccccgtttcaaaacagagaagatggaaagtaaaactgttttgcccttcactgataaatgtgaatcctcaaatctgacagtaaacacatcgtttggatcatgccctcaatcagaaaccattatttcatcattaaagattgatgccacccttactagaaaaaatcattctgaacatccgctctgttctgaagaagccctggatccagacattgaattgtctctagtaagtgaagaagataaacagtccacttcaaatagagattttgaaacagaagcagaagatgaatctatctattttacacctgaactttatgatcctgaagatacagatgaagaaaaaaatgacctagctgaaactgatagaggaaatagattggctaacaattcagattgcattttagctaaagacctttttgaaattagaactataaaagaagtagattcagccagagaagtgaaagctgaggattgcatagatacaaagttgaatggaattctgcatattgaagaaagtaaaattgatgacattgatggtaatgtaaaaacaacttggataaatgaactggaactgggaaaaactcatgaaatagaaataaagaactttaaaccatctccttccaaaaataaaggcatgtttcctggttttaagtaataatacttaactctcaagctaagtaaaaatatgtcatcatgcttatgttaaactctgttgtaagtaataatttgtaaattgaataagtggcatactttttaaaaaactattttatgttcagaaatgtaaatgttattattcttgagtttttgggtttttttttttgagacagagtcttggtctgttgcccaggctggaatgcagtggtgtgctctgggctcactgcaaccttcacctccaggttcaagtgattctcctgcctcagccttctgagtagctgggactacaggtgtgcaccaccatgcccagctagtttttgtatttttagtagagatggggtttcaccatgttggccaggctggtctcgaactcctggcctcgtgatctgcccttctctgcctccctaagttgctgggattacaggtgtgagccacagtgcctggcccattcttgagttttgataaagtaattcatacaaagtactgtcctcaaataagtcttccttagctaaatgcaatttaaaattattcaaagatcctagggcacttctagtttcacgtaaatattcatattaggtggttctcttcatccatttgttttcacactgatacataaaaattaacagcagtctaatctagtgacacctcagtcatatttcgctatagattttacctcaaatcagtccaagactttttcagagatcaccatttgtcttgaaaggtttatttcgttattaaactgcctacttataagtaattaagagaaattaagaaagtagtatgcatttttaattgaaattgttttacattctttgtataataaacctaaaaccaaacatgtcataaacaaattgacgtaaagatataaaatgccaaatgaagtattccaaattttctattctaattatttagcttcaccatcattgtggaaaaaaatactagatcctgcttagtattatatatttttcctagtggatcagtgagtaataagtaccaaacactagactagaaggtaatttctacattgtttagaaagggtgaaacaatttatcccctctggtattgttctagcataagctttagttatacaatgattaagatagaaaacttcatatataaatttgataagcaaacccacatttatagctgcagctaaaatatgtttccttagggcacagtaatcctttctgtgaattttgaccttgtttgtgtttttgtgaatgaagctatatgtctaatcaaaaatgattataaaagaggctcatctctgacatcattccaaaaatacattcattgatctctttttaagaaacatctgttattcactgggcattgggactttttgtgagtaatttgaattgaaattttatgagctatccaagaattctgtatggtctattattttcaagtcaaaatttccagtaaggatttactttacatttcatttggataaatgaatcattatataggtatgtctttgcttccattttgagacatttagatttttacagcctgtttctatagcatttgatgttacaactctaagcgtagttcaaagacatttaaattgacaagttaccagttaaagaatttagaatatattagatcccatctagtattatatattttttctagttgatcattgagcagtaaataccaaatactcgattagaaggtaatttttacattgttttgaaagggtgaaacaatttatctcctctggtattattcttaaaccacagatagggatagtagggtagtgaaacgaataaatacctggtagaagacaagagacttgggctctacacctggctctgccactgatttgctaagtcatattggcaatcaccacacccttcagggaattagtttcatctgtaaaatgcagcggttagtactataaaatcatacaaatttctttgtgctttgagaatctataaaggaatgtctgttgatattctgagtcgattttcatttgcttttgttccagaacggttaaaataaagcatattatttcatttaaaaagtaaagtggctcttactcatttacatttagggaacgatggtgacgtggtttggctatgtccccagccagatctcatcttgaattgtagttcccataatcccgacgtgttgtgggaaggacccagtgggaggtaattgaatcgtgggggcggttaccctcatgcggttctcgtgatactgagttcttatgagacctgatgattttataaggagcttttccctgcttcactctcattcttctctcctgctgccctgtgaaaagttgccttccgccatgattgtaagtttcttgaggcctcctcagtcatgcagaactgtgagtcaattaaacctctttcctttataaattacccagtcttgggtatgactttattagcagcatgagaacagactgacacagatggtatatgtgcataataacttggaaagctagatatttattttcgcaatgctacaattaaaacattttgaggacttttaaaattacctttagggccaggcgcggtggctcacgcctgtaatcccagcactttgggaggccgaggcgggcggatcacgaggtcaggagatcgagaccatcctggttaacacggtgaaaccccgtctctactaaaaatacaaaaaattagccaggcgtggtggcgggcgcctgtagtcccagctactcaggaggctgaggcaggagaatggcgtgaacccgggaggtggagcttgcagtgagccgagattgcgccactgcactccagcctgggcgacagagcgggactccgtctcaaaaaaaaaaaaaaaaagaaattacctttagaattggtaaactacatgagaaaattaagcatattattcatatcttagtgttattacccaacttcatctccaatactttcccctttccctagctagatatgtttttgtttgtttgttttagacagtcttgctctgtcgctcaggctggagtgcaaaggcacgatcttggctcactgcaacctctgcctcgcaggttaaagcgattctcatgtctcagcctcctgcgtagctgggattacaggtgtacaccactgcacccagctaatttttttttttttttttttttaagtatcgacagggtttcaccatgttggccaggctggtctcaaactcctgacctcaagtgatctgcccacctcggcctcccaaagtgttgggattacaggcgtgagccaccatgcctggcctagatattttttattctttccaaaatttaattctccctgaagttaaaaattttcattactgagaatgtacatagagatgtgtcacaaccctttagtaattccaaaaggtgtttcaaaaattttatacaatgataatcactgttgaaacaggtgtatattctcctcagatcatgactgaccaagatgatattccaagaagtaaactactggcctttatagagtaggatgtaggccattttcattactgataacatacttttaaaagaaatgtttacagatttaaataagttaaaacatctaaatgcttttaaaagagcacctggcacattgcaagttattcattattaattagtagatgaatcatatatctggagtgcacccttgctctatatgaaagcttccctaactatagatatataggtgatagactgacaataaagatttgaggaaagaaaaattatttggctggcttattttttaagcttttgagatgtataaggtagagattgtttatcaaataatttatgtgacacattcaatgcatatgaaataattatctaatactgaatttattcaaggactgaaagtatatagagcacacaggctgcaaaccaggaaaactaccagttagagacagggctttattcttggaataaaggtacaatgtaaagagtagatgtttcatgacttgattaaattatttaaaactgtctagaattgtgttataaaactattactatgtttctatgcacttagttattacatgggtttaaatttggcactgtttctaagtttctataaggctttgttgttaagatctttctattcaaaacattaattttaacaaaaagcttttccccgttatttttctgcaa

SEQ ID NO: 11 - DNA, 117 bp, Homo sapiens
cttgtaagatggcaagaaacacaaaattccttggctcacagtctggcacacagataacaaaagtgctcccacttcatcttggaactcaaatgtttcagtattctggaaggtagcaca

SEQ ID NO: 12 - DNA, 118 bp, Homo sapiens
  variant 59: aa(insertion,ins)or -(deletion, del)
cttgtaagatggcaagaaacacaaaattccttggctcacagtctggcacacagataacnaaaagtgctcccacttcatcttggaactcaaatgtttcagtattctggaaggtagcaca

SEQ ID NO: 13 - protein, 1249 aa, Homo sapiens
MSSMWSEYTIGGVKIYFPYKAYPSQLAMMNSILRGLNSKQHCLLESPTGSGKSLALLCSALAWQQSLSGKPADEGVSEKAEVQLSCCCACHSKDFTNNDMNQGTSRHFNYPSTPPSERNGTSSTCQDSPEKTTLAAKLSAKKQASIYRDENDDFQVEKKRIRPLETTQQIRKRHCFGTEVHNLDAKVDSGKTVKLNSPLEKINSFSPQKPPGHCSRCCCSTKQGNSQESSNTIKKDHTGKSKIPKIYFGTRTHKQIAQITRELRRTAYSGVPMTILSSRDHTCVHPEVVGNFNRNEKCMELLDGKNGKSCYFYHGVHKISDQHTLQTFQGMCKAWDIEELVSLGKKLKACPYYTARELIQDADIIFCPYNYLLDAQIRESMDLNLKEQVVILDEAHNIEDCARESASYSVTEVQLRFARDELDSMVNNNIRKKDHEPLRAVCCSLINWLEANAEYLVERDYESACKIWSGNEMLLTLHKMGITTATFPILQGHFSAVLQKEEKISPIYGKEEAREVPVISASTQIMLKGLFMVLDYLFRQNSRFADDYKIAIQQTYSWTNQIDISDKNGLLVLPKNKKRSRQKTAVHVLNFWCLNPAVAFSDINGKVQTIVLTSGTLSPMKSFSSELGVTFTIQLEANHIIKNSQVWVGTIGSGPKGRNLCATFQNTETFEFQDEVGALLLSVCQTVSQGILCFLPSYKLLEKLKERWLSTGLWHNLELVKTVIVEPQGGEKTNFDELLQVYYDAIKYKGEKDGALLVAVCRGKVSEGLDFSDDNARAVITIGIPFPNVKDLQVELKRQYNDHHSKLRGLLPGRQWYEIQAYRALNQALGRCIRHRNDWGALILVDDRFRNNPSRYISGLSKWVRQQIQHHSTFESALESLAEFSKKHQKVLNVSIKDRTNIQDNESTLEVTSLKYSTPPYLLEAASHLSPENFVEDEAKICVQELQCPKIITKNSPLPSSIISRKEKNDPVFLEEAGKAEKIVISRSTSPTFNKQTKRVSWSSFNSLGQYFTGKIPKATPELGSSENSASSPPRFKTEKMESKTVLPFTDKCESSNLTVNTSFGSCPQSETIISSLKIDATLTRKNHSEHPLCSEEALDPDIELSLVSEEDKQSTSNRDFETEAEDESIYFTPELYDPEDTDEEKNDLAETDRGNRLANNSDCILAKDLFEIRTIKEVDSAREVKAEDCIDTKLNGILHIEESKIDDIDGNVKTTWINELELGKTHEIEIKNFKPSPSKNKGMFPGFK

SEQ ID NO: 14 - protein, 27 aa, Homo sapiens
CATFQNTETFEFQDEVGALFCYLCARL

SEQ ID NO: 15 - DNA, 182771 bp, Homo sapiens
tctaatcgagtatttggtatttactgctcaatgatcaactagaaaaaatatataatactagatgggatctaatatattctaaattctttaactggtaacttgtcaatttaaatgtctttgaactacgcttagagttgtaacatcaaatgctatagaaacaggctgtaaaaatctaaatgtctcaaaatggaagcaaagacatacctatataatgattcatttatccaaatgaaatgtaaagtaaatccttactggaaattttgacttgaaaataatagaccatacagaattcttggatagctcataaaatttcaattcaaattactcacaaaaagtcccaatgcccagtgaataacagatgtttcttaaaaagagatcaatgaatgtatttttggaatgatgtcagagatgagcctcttttataatcatttttgattagacatatagcttcattcacaaaaacacaaacaaggtcaaaattcacagaaaggattactgtgccctaaggaaacatattttagctgcagctataaatgtgggtttgcttatcaaatttatatatgaagttttctatcttaatcattgtataactaaagcttatgctagaacaataccagaggggataaattgtttcaccctttctaaacaatgtagaaattaccttctagtctagtgtttggtacttattactcactgatccactaggaaaaatatataatactaagcaggatctagtatttttttccacaatgatggtgaagctaaataattagaatagaaaatttggaatacttcatttggcattttatatctttacgtcaatttgtttatgacatgtttggttttaggtttattatacaaagaatgtaaaacaatttcaattaaaaatgcatactactttcttaatttctcttaattacttataagtaggcagtttaataacgaaataaacctttcaagacaaatggtgatctctgaaaaagtcttggactgatttgaggtaaaatctatagcgaaatatgactgaggtgtcactagattagactgctgttaatttttatgtatcagtgtgaaaacaaatggatgaagagaaccacctaatatgaatatttacgtgaaactagaagtgccctaggatctttgaataattttaaattgcatttagctaaggaagacttatttgaggacagtactttgtatgaattactttatcaaaactcaagaatgggccaggcactgtggctcacacctgtaatcccagcaacttagggaggcagagaagggcagatcacgaggccaggagttcgagaccagcctggccaacatggtgaaaccccatctctactaaaaatacaaaaactagctgggcatggtggtgcacacctgtagtcccagctactcagaaggctgaggcaggagaatcacttgaacctggaggtgaaggttgcagtgagcccagagcacaccactgcattccagcctgggcaacagaccaagactctgtctcaaaaaaaaaaacccaaaaactcaagaataataacatttacatttctgaacataaaatagttttttaaaaagtatgccacttattcaatttacaaattattacttacaacagagtttaacataagcatgatgacatatttttacttagcttgagagttaagtattattacttaaaaccaggaaacatgcctttatttttggaaggagatggtttaaagttctttatttctatttcatgagtttttcccagttccagttcatttatccaagttgtttttacattaccatcaatgtcatcaattttactttcttcaatatgcagaattccattcaactttgtatctatgcaatcctcagctttcacttctctggctgaatctacttcttttatagttctaatttcaaaaaggtctttagctaaaatgcaatctgaattgttagccaatctatttcctctatcagtttcagctaggtcatttttttcttcatctgtatcttcaggatcataaagttcaggtgtaaaatagatagattcatcttctgcttctgtttcaaaatctctatttgaagtggactgtttatcttcttcacttactagagacaattcaatgtctggatccagggcttcttcagaacagagcggatgttcagaatgattttttctagtaagggtggcatcaatctttaatgatgaaataatggtttctgattgagggcatgatccaaacgatgtgtttactgtcagatttgaggattcacatttatcagtgaagggcaaaacagttttactttccatcttctctgttttgaaacggggaggactagaggcactattctctgatgacccgagctcaggtgttgccttcggtattttaccagtaaaatactgtcccaaagaattaaagcttgaccagctaactctctttgtttgtttgttgaaagttgggcttgtggatctggaaatcacaattttttctgctttccctgcttcttccaggaatactggatcatctaagaatacaagaatttaagagatttaactttctgctcctagctaacataattgctaggttaaaataattatttattagaaatacctaaataactgtataggatacataacttagagcccccaacgaatactagtggattttcatcttggaacagaatattaactctgaaagaaatcttaggtctctaatgtttcctagcccaagttataatgctgtctggtaaactatcaaattccacagcagggagctcaagaatgaatgaggaaagaaggggtaaaagggaagaggaagggagctgaggaaaggagggaacaggctgtaaacatttgccttgaaaatccactaaccagtgactgagaaaaacttacctagactaattcttttatttaataaataaggaaagtgacataaggctaagttatttgctcaaagttataaaactggttagtaagaagaccaagactagaaactaggtttcctgattaccaagttaatcggcttgaattcactatttatagtgaattttgtattactattttgtagtatttatatacaaaattgtattactattttgtagtacttatatactacaaaatacatgtttaaaaattgatgtttttcttttttttagtccgagttttgcttttgttgcccaggctggagtgcaatggcgcaatcctggctcactgcaacctccacctcccagttcaagcaattctcctgcctcagcctcctgagtagctgggattacagacagctgcctccacacccagctaatttttgtatttttttagtagagacggtgtttcaccacactggtcaggctggtctcggactcctgacctcggatgatccacccgccttggatatatgaattaaaacaactgaaattatatccaatggaatagaccagagtttggcaaaccttttctgtcataggccagatagtaatatttaggctttgtggagcataatgtatctgttgcgactactcaattatgctgttctagtgcaatacacacaaatactcaactctgctgttgtttctaaaatgtaaataaatgggcatgactacatttcattaaactttatttacaaaaacaggcagcaggccaaatttggcccatgggccacagtttgccaatctgatacagatgattatttcaaaatattttaaaatattatttttcatcacttaaaataaatgtctctcacctcctaaaaataagttcataagaactatctatataggaaaagtagttcatatagaagtatctacataaaaattaggccaaatttttatatagcctttagaataacatctaccacttacatggtactttacaatgctgtgatatttataattttgacctttacaacccaacctgaagggatagctaggcaggcattttttttttcctattacgactcaaaaaggacaaatgacttgctcatgatcacaaagctagtaagtggcaaagccagttagtggaaaaccagaaaatatgctctggtctcctgacaaaagtttgttcccattacagtacaccatactgtttcactattttatgataacaccttatattacaaaccaccatatttaaggaattaatctatacccaaataaatatcatttcactaaatataatgaaagacttctctatcaaaggtaaatgggaagaacttttcatacttttctcctttctggagataatgctacttggtagaggtgaatttttggtaataattttaggacactgtagttcctggacacatatctttgcttcatcttccacaaaattttctggtgatagatgacttgctgcttccagtaaataaggtgaggtactgtactttaaagaggtcacttcaagtgtagactcattgtcctgtatattggttctgtcctttatggatacattaagaactttttgatgctttttggaaaattcagccaaggattccagtgcactttcaaaggttgaatggtgctgaatctgctgccgtacccatttagaaagtcctaaagaaaaaggtaaacccagggaaaatttggttacttagttattaaaatattacatgctaaggtaatacacttgctttttctagtgaagtaacctaatttgtattttgaatatacagaatggttctccatatgttttttctgcaattcagcaattttactacatatgtatcagtaacatatgagtaacattctctttatatacagtaattgtcaaatggagagagcaaagaggcagtagaagtagggcagagatgggaaaacatattctctggttaagaataactggtgaacatggcaaatttaatttcatcataattccccacaaaaattgccttttttttttgtagggaaactagaagttgtaaacaatacaactttccattctaatattctaaattttactccttttgcaaaaataatttcaagattctagtctcaagtttttttgttcccttcctccaccaattccaaatatatcttaaattaaaattattctggaggtgaaataaataacactgtcttatgggtaaatcacatcccttctgaatttttatgatataattatttttaatatataatcaagttataaaggatatgatttgacctgtgacataaaaactgcacaaaaatgaaacctaagagtttaaggtgttttctagtatggtgtgcttccaaatgaaatgcaaaatcaggtatttggacttttacaaaataaatattccctgctaacactactaaaactagcatcatcttttggaggaattagttttaaaagtcactatagggtaaaaatttaagttctagattttaaaactaaaaaaattttaaataagccatttcccttgaagttttttattttgttcatttggggaatatgattagaccataaaaatatgtgaaattctctaaaattgaaaatttttaggctgagtgtggtggctcacatgtataatcccagcatttggggaggctgaggtgggaagattgcttgaggccaggagttcaaggtttcagtgaactatgatcacgtcactgcactccagcctgggcaacagagtaagacctgtctctaaaaaagaaataagtaaataaaaataagaattttagtactatatttacctcttcatttcaacctttttataatcaaagaaagccttttcaatgtaaaatatcattgtctgcttttgaaaacttgaaagaatctgagctccacctgctgactagacaccagcacttagcatttttgcctttagttactgtttgacctatgctactgcacactttacgtggaaagggaagataaaacaaataaagcaatattttctgctttatgttttaatatcaaaccaaatacagtcattaagctctgaaattgaaaaggtagagtctacacccacacatggctaatctaaaacaaaatttaacatctctttattataataaaatgtattttttttttagctaaaaggtaataataaagatacacatgtatcccatagctctttagtaatcactcctggtattgactctgaaaactgtttactgttggatgaaaccatcgagtgagaaaaaaatgatactactgcctgcatcaaagcggtaggaaaatttgtggcaccctcttgtcatgatccccacttcagaatatacctggctgcttcttcctggggagaaaagagaaaactggaccttatgtctttttctgggaggctgccccagggactggcttctatcttacctgtactgaagagctgacatgagctggcataccctacatgcccaggggccactaagaacaaaagcgctgtgcagcaagtttctgctccacaggaaccacagtgcaacagaggcccatatagtttgatggccactccctcagggggacagaaaagcctggagtgtgcatccaacatcttggcttttcaaggagctgtctagggactcgtttctgtcttgcttgtcatagagtactgactggacctagcatactctgtatgcctggaatctactaagaacaaaagagagtcaggcgtgatggctcatgcctgtaatcccagcacttagggaggccgaggcaggcagatgacttgagcccagagcccaggagtttgagaccagcgtggacaacatggtgaaaccccatctctacaaaaaatacaagaattaactgggtatagtggtgtacacctgtggtcccagttacttgggagactgaggtgggagaatcccttgaacccaggaagccgaggctgcaatgagctgagattgtaccactgcactccaacatgggcgacagagtgagaccttgaccaaaacaaaaaaaaaccaagcaaatatttctaataaggaatttacagagaagtccaaagaagacagatccacggaaaaggtttgagaggctcccagaatgcctaccagggctgatgagtgaaggtctttccctgtacaaaaccagttagtaaacactgtgagaggtggctgttttttcaaatgtgtggataccatacaaggttacaagaaacataaagaaacaggaaaaaataaccctgtcaaaggaacaaaataagtctccagaaaccaacccttaaaaaaaaaaaaaagctatatgaattacctggcaaggaatttaaaataactgtcaacatgatgctcacaagttcaagaaaataatatagaaaaaaaggagaatttcaacaatgataaagaatacaaaaaagaactaaagagattttggagctaaaaaatgcaataactggactgaaaaattcactacaggggttcatcagcagacttgatcaagcagaaggaagaatcaggaaatttattttattttactttattttatatattttatattctgttatgttatgttatgttatgttatgttatttttttgagacagtctcgctctgtcgcccaggctggagtgcagtggcgccatctcggctcactgaaacctccgcctccctgattcaagcgattctcctgcctcggcctctcaagtagctgggactacaggcatgtaccaccacacccggctaattgtcgtatttttagtagaggtggggtttcaccacgttggccaggctggtcttgaactcctgacctcaagtgatccaccttcctcagcctcccaaagtgctaagaatcaggaaatttgaaggcaagtcgtttgaaatcatccagtcagggaagcaaaaagtaaaaaacaaaatggaaaaaaaaaagtgaaggcttatggcttcatttatgacttgacttatggggcaccgtcaagctgaccaatatacacattctgggagtcctagaaagagaaagagagaaaggagcagaaagcttatttatagaaataatgaccgaaaacttaataaacctggggggaggaaaatgacatccaaatacatgaagcccagtgaacctgaacaggatgaacccaaaagtctgtaccaaaacactttataatcaaagtgtcaaaagtcaaagaaaacaatagaaatttgaaaacagcaagataaaagcaacttgtcatatacaaatgaagtcccataagacagtcactaggccgggcatgaaggctcatgcctataatcccaacactttgggaggcagaggtaggagaattgcttgagccgaggagtttgagattagcctgggcaacatagtgagaccccatctctacaaaaaaaattaaaaattagccaggtctggtggtgcatgcctgtagtcccagctgaggctgaggtgggaggatcacttgagcctgggaggctgaggctgcagtgagtcatgatcatgccactggactcctgcctgggtggcagagtaacatcctgtctccaaaaagagaaagatttctcaggcgacatcttgcatgctgtaaggaagtgggatgacatgtttgaagtgctgaaagaaaaaaacctgccagtgaaggatattatatctggtaaagccatctatcaaaatgaataggaggtaaaaacttttccagataagcaaaagctgagggacttcaccactaaacgtgccttacaagatatgctaaaggaagcctgctaaacagcaacacaaaagcataaagtttgctcgtgaagttaaaagtgtacacaaatacagaatactttaatataacaacagtgatacataaataatgtttaattctatacagaagttaaaagacaaagtatgaaaataactataatagtatgttagtggatacactatataaaagggtataatttgtgactttaataataaaatgtgatgggaaaagtaaaagtgtagagtttatacaactgaaatgaatttgttagctgaaaatattgttataactatgtttcatgtaagacccatgtaaccacaaaggaaatatctatagaaggtatacaaaagaaaaatgagaaaggaatcaaagcatttttcttcaaaaaattaaaaaatgaaaagcttttaagaccagcaacaaagcaaggatgttcattctcactacttctatttagcttagtactaagaatcctagccagactattaggcaagaaaatgaaataaaaggaatccaaattgaaaaagaataaagtgaaatgatctctctttgcagatgacatgatcttttatgtggaaaaccctaaagattgaaaaaaaacctgttaggactaataaatccagtaaagttgcagaatacaaaaatcaatgtacaacaatcacttgcaattctatacactaaaaacaaccaaaagaggaatgtaagaaaacaatcccatttataatagtatgaatgaagctggaaaccatcattctcagcacactaccgcaaggacagaaaaccaaacatggcatgttctcactcataggtgagaactgaacaatgagaacacttggacacaggaagggggacatcacacaccggggcctgttgtggggtggggggagaggggggagggatagcattaggagatatacctaatgtaaacgacaagttaatgggtgcagcacactaacatgggacatgtatacatatgtaacaaacctgcacgttgtgcacatgtaccctagaacttaaagtataataataaaaataaaaattaaaataaaaaaactcaaaccagggaaaaaaaatagcatcaaaaataataaaatacctaggaataaacaactaaggaggcaaaaaacctgtacactggaaattataaaacattgctgaaaaacagtaaagataacacaaataaatggaaagatatcccatgttcacagattggaacacctaatattgtcagaatgtccatactactgaaagcaatctttttttgagacagaatcttgctctgttgcccaggctagagtgcagtggcgcggtcttggctcactgcaacctccgcctcccaggttcaagccattctcctgcctcagcctccagaatagttgagattacaggcacccgccaccgcacccggctaatttttgtatttttaatagagatggggtttcaccatgttggccaggctggtctcaaactcctgacctcgtgatccacctgccttggcctcccaaaacactgggattacaggcgtgagccaccatgcccggctgcaatctaaattcatatggaaccacaaaggaccacaaataggcaaaagaaccttgagaaaaatgataaaagctgcaggcatcatacctcccgattataaaatacattacacagctgtagtagtcaaaaaagtatggtactggcataaagagaaccatataagccaatggaacataatagagagctcagaagcagatccacacatatatggtcagcggatcttcaacagggtgtcattggggattgatatgctttggttatttgtttcctccaaagctcatgttgaaatgtgttcccccatgtcataggtggggcctagtgggaggtatgtgggtcatggggacagaaccctcatgaaagccttggtgctatgattgtagactttctgaggtcctcattagaagcagatgctgatgccatgattcttgtgcagcctgcagaaccataagccaaataaatctcttatctttataaattatccagtcttggtattggcaatgattgtttggatatgacaccaaaaacacaggcaataaaagcaaaattagacaaggaagaccatgttaaactaaaaaacttctgtgcagcaaagtaaataatcagcagagtcaaaaggcagcctatagaatgggagaaaataatttgcaacgcatatatctgataaagagttaatccccaaaatatataagaaactcctatatctcaatagaaaaaacaaacaaaaaaacacaccaaaaaccaaaccaaaaaaacagatatgcaaagaaatccaaataacccaattaaaaaatgagcaaatgaaaagatgtttctccaaagaagacatatgatttggccaacaggtatagaaaagatgcttaacatccctattcatcagagaaatgcaaatcaaatccacactgtgctatcacattatacctgctaggagagccattatgaaattttaaaaagaaaaagaagaaaaaaggaaataagcattagggaggatatggagaaactggaacccttgtgtgctattggtgggaatgttaaatggtgcagttgctatgaagaacagtatcaaatcaaaaaattaaacttagaactatgatgattcgccaaatcctacgcttggtatttgttcaaaaaaattgaaatcagcatcttgaagagatatctgcattcccatgtccattgcagcattgttcccaatatccatgagggggaaacaacctaaatgtctactgacagatgaatggataaagaaaatatggtataatgtatacataaaataaaatattaagcagtcattgaaaagaaggaaatcctgcaacatagatgaaccttgaggacattatgctaactgaaataagccagttacagaaggacaaatactgcataattcaatttatatgaggtatccaaaacagtcaaactaatagaagcagaaagtatagcggtggttgtcagtggacaggggggagagggacatggggcattgctgttcagttggtgttaagtttcagttatgtaagatttaaaagttctagatatctgctgtgaaatactgtgcttatagttaacaatattgtactgtgcacttaataagatagtagagctcatgttatgtgtttttcaccacaataaaaatatgaagattgttactagtttttactctaagcccagctgagatcttaccagatatatagcgacttgggttattcctaaagcgatcatccactagaataagagctccccaatcatttctgtgtctaatacatctagaaaaaatagggaaaaagtcaaataattataacatcggaaataaatccagttttccagtgggacagacagaaaattggaaaaaaatcaattttatagattcctttaaacaatttgttattatcagaaaagttacagaagctatccaacacaatgtaacaaactagatgtattaaaaattccctactttttccaaatacagataacttgcatccaaaatgttgtattttattttgcactcaagaaaattatagaaaatatattctaagatctttcttagccacatagactgttttcttttttttaccttaagtattataaataattgtaaaggtcctttttactagtttgatgagagttttaaatcatgaataaacatttaattttgtcaaaaatattctaaaattcaattgacataacatgtagtttttcttcttttgactgttaatatggtagattacactggttcattttcaaacactgaatcatccttgcattcctagtcatggtgtattattctttttatagattgttggattcaatttgctagtttaaaaaaacacattaaaaataaaaacatactttttaataattattaaacaattttaaagtgcacaatgcaatggtttgtggtatatttacaaggctgtataacctataccacttatctatcccagaacattttcaccaccccaaaaagaaaccctgtgcccactaattgttactccaaattatcccaattattccccattacctctggcaaccactaatctttctgtccttagagagttgccaattctggacatttcatttaacgggaatcagacaatatgtagcattttgtatctggcatatttcactgaacatgatgttttcaagattcactcatattgtatcatttctcagtacttcatttataagttaatgtttccactttttggctagtatgaataatgctgctattaaacatttgggtacaagtttttatatgaatacatgttttttattctcttgggcatacacctagaagtggaattggggagtcatatagtaattctatgtttaactttttcaaacactgccatactgttttccaaagcagctgcacattttacattcccaccacctacgtacaagggttccaaaatctccacatcctcaccatcatttgttattgtatacccttctaaaaatatatctaccctagaaagtgtgaaataatatttcattatggctttgatttgcatttccctaacaaccaatgatgttgaacatcattttttacgtgcttactggccatttgtgtatccttttttggagaagtctgttcaaattattggtccatttttaatggcccattttgtctttttattattaagttgtaagagttctttatatattctggatactagatcctgctacacatgatttacaaatattttctccatatgtgagttgtcttttccatttcttgatactgtcctttgaagaataaaaatttgaactgtaacaaagtccaatttatctgctttttctttggttgcttgtgcttctgatagcagatctaagaaactattgcctaatacaaggtcaagaaaatttatacctctgtttttgcctttttctgtctttgatccactttgagttaacttttgtatatggtgtgaggtaggagtctaatttcattatttgcacagatatccagttgtcccaacaccatctgttgaaaagactattctttctttgagttgtcttggcactcttgtcaaaaaccagttcactgtagatgtatgaatttgtttatggaccctcaattctacccattgatctatacatctgtctttatgccagtacaacatagtcttgattattgtagctttgtagtaagtttgaaatagaaaactgtttgttctcctagtatgttcttctttttcaagattgttttgactattctgggttccttgcttttccataataattttagaatcagcttctcaattcctgccagaaagtcagctgggattttgattagtattgtatttaacctctatatcaatataggggagtgttgatatcctaatgataagtcttctaatccatgaacgtgggattctttccatttatttagaattttctttcaataatgtcttgcagttttcagtatacagatctttcatctccttagttaagtatatttctaagtattttcttctttctgatgttattttaaatggtattttttaagttcatttttagatttttggctgctagtgaataaaaatgtcattgattttttaatattggtcttgtaacctgaaaccttgctgaacctatcagttctgttattttaaaaatggatttcttagaattttcttagaatgtaatatatcatttgcaaataaaaatagttctgcttcctcctttccaatctgtatacctttctttcttcttcttgtctcactgccctggttagaacctccaatacaatattgaatagaagtggtaagagtgaaaatcactgtcgtgatcttgaacttaggaaaaacatttcagtcttttaagattaagtatgatgttagctgtgagcttttaatagatgacctcattaaagctaagtaagtaaccttatattcccactttgttgagtattttcatcatgaaaatgtgttgaatactgttttatgctttttttttttttttgcatcttgagaagatcgtgtggttttagtcttcttttaatatggtgtattacattaattgattttataaagcatgttgaaccagtcttggattgctgggatcaatcttacttggtcatggtgtataatccttttagtatgttgctgattctgtcaatggcttttatttctatagtcatatgaaatattgctgtacaatcttcttttctcgtaatgtattttacttgttttagtatcaggtaatactggccacatagaatgagttggtaaatatttctctcccattttttgctagagttaatgaaagattaatgcaaattcttctgtaaatttttggtagaattcaccagtgaagccatttgatcatgaggctgaggcagaagaatcacttgaacctgggaggtagaggttgcagtgagctgagatcacgccactgcattccagcctgagtgacagagtgagactttgtctcacaaaaaaaaaaaaaaaaaggccaggcgtggtggctcatgcctgtaatcccagcactttgggaggccaaggtgggcagatcacgagatcaggagatcgataccatcctggctaacacggtgaaaccctgtctctactaaatatacaaaaaattagccgggtgtggtggcgggcacctgtagtccccagctactcgggaggctgaggcaggagaatggcgtgaacctgggaggtggagcttgcagtaagctgagattgcgccgctgtactccagcctgggcaacagatagagactctgtctcaaaaaaaaaaaaaaaaaagggggggggaagtgttttgatgacaaattctatctttacttgttatacatctattcagatattctatttcagctggtcgcagtggctcacacctgtaatcccagcagtttgggaggtggatcacgaggtcaggagttcaagatcagcctggccaacatagtgaaaccccgtctcttctaaaaatgcagccgggcatggtggcaggcacctgtaatcctagctactcaggaggctgagacaggagaattgcttgaacctgggaggcagaggttgcagtgagccgagatcatgccattgcactccagcccaggcaacagtgtgagactctgtctcaaaaaaaaaaaaaaaaaaaaaaaaaaaaaaaaaaagatattctatttcttcttgatatattctcagtattttgtgtctatctatgaatttgtccatttaatctaagttatttgtcatcacacaatttttgaaggtattctcttataatcctttgtatttctgtaaggtagtaacatcccctctttcatttctaattttagtgaattagaacttctctgtattttctttggtcagtcttggtacaggtctgtcaattttgttgatcttttcaacaaatcaacttttgttaccttggttattctctactgttctttgaattctctatttcatttatttcctttttcatttctagtatttcctttaattgcctcgctttgggtttagtttgctcttttttttttcctaattccttaaggaggaaggttagattattgattccagatgtttattcttttaaaatacagatgtttacagtattttttttttttagacagagtctcgctctgtcagccaggatggagtgcagtggcatgatctcagctcactgaaacctccgccccctgggctcaagcagttctcctgcctcagcctcccaagtagctgggattacaggcatgtactaccacacctggctaatttttgtatttttagtagagacggggtttcaccatgttggccaggctggtctcgaactcctgacctcaggtaatctgcccgtgttggcctcccaaagtgctaggattacacacgtgagccaccgtgcctggcctagatgtttacagcttttttaagtttccctgggagtagtactttcactgtgtccccaaaattttgggacattgtttccattttcatttatcacaatgtatttttcaatttttcttctgatttcttctttgacccactggttgtttaaaagtatatcattgaatttccatgtatttgtgagttttccatttttcctcccattattgatttctggtttatttcattgtagtcagaaataatacttgtaatgatttcaacctcttaacatttatggaaccttatattttatggatgaacggatggcctatcctgaagaacatgccatgttcacttgagcagaatgcgtattttcctgttgttgggtgaagtgtgctacagatgtctgttaggtttgtttgtagttgttggtttgtagttttgttcaaggcttttattttctgttgattttccttctagttgttctacccattattgagcagggtatggaagtctccaagtattactaaattattgatttctgatgctttttgctagatgtgttttggggcctttttgttaactttgtatgtttataattgcatatttagacttgttatttttttttattttttatttttatttttttttgagacagggtcttactccagttgcccaggctggagtgaagtagtgaggtcatggcttactgcagcctcaacttcctgggctcaggtgattctcccacatcagtctcctgaatagctgggattataggcacatgccactacacctggctaatttttttgtacttttagtagagacagggtttcatcatgttacctaggctagtcttgaactcctaagctcaagcgatctgcccacctcagcctcccaaagtgctgggattgcaggcagaatggcacttttatcattataaaatgttaattgtttctattaacttttttttgtcttaagggtctattttatctgatattagtataaccaaaccagctctcttttgtttactatttgcatggtatgtcttttcccatcattttattttcaacctatttgtgttttttaaactagtgtttctcttgcagacagcatatatcacattgttttcaatccattcagccaaactttgcctttagattggagtatttaatccatttacatttaatgtaattactgataaggtaggatttactcagctattttgtaaaatactcactttgctatttgctttctatatgccttacgtattttgttcttctgtctttccattactttccattttctccaatagacactttccagtatataattttaattctcttgtcatttacattactatattttcttatttttattagtggttgaagagtataataaacatatcaaccagtaataaactagtcaggaaaattgccaacttaatttcaatagtacaaaaaaatggtccttctgttccctcatctgtcacctttgggttgttattgtcatataaattatatctttaaacattgtatgctggtgcagaagaattaacattgtaggttggattgtttacccctagaaaggtctgtttaaaggtgttcctgggctgatgaatgaaaacttagattcaggagggtacctgtgattgactgataagagtgtctcactgtgcctgaactgtttgagtaaacaatatattaatgctgaatactcattctccttttgagagtctagtcttctggaaactgctaggcagaggctgcctatatgaccagccccagtaaaatccctgggtgctaatgagttcctctgattggaaatatttgacacatgttgccacaactcattgctcggggaagtgtgtcctgtgtaactgcactgggagaggacacttggaagcttaagactggctccctagactttgtcccatgcacatgttttgttctctttgctgactgtgcttggtgtcgtttcactgtaataaatcctagccatgtatacagttatatcctgaggctatctaactgttccaagtgaattgctgaacctgaaggtggtcttggcaacccacaagacaatcaccacagatttacaattattgttttatgcagttgctcttttaaaaaatcaagagtaaaagaattataagcaagaagtacctttatattaacttttacatttacgtatgtcgttaactttgtgggtattctttatttcatcgtgtgcatctgacttacttcctaatatcctttcatttcagcctgaaagactccttttagtatttctcgtaggacaagtttgctacagagtaattctcttggtttttgtttatttgggaatgtcttaatttttaccttatttctgaaggacagttttgttagatatagtattcttggttgacagtcattttctttcagcactttgaacatgtcaattaactgcttcctggtctggaggatgttttgtatatgatgagttatttctctcttgccgctttctagattctgtcttctggctgtttgatttcaaccatctagataggaatctccctgagttcatcctacttggagttcgcttatcttcttggatgtataaattaacgtttttcatcaaattcggaacgttttcagccataatttctttaaatattctttctattcttttttctctcctctccttctgggactccaaatatgcatatgttggtacgcttgatgaggttttacaggtctctgagattatgctagttgttcttcattctttttctttctgttcttcagagtaaataatctcaattgatgtttaagtaaatcaattctttcttctgccttcttaaatctgctgttgggcccctgtaatgaatatttcattttagctattatactttccaactccagaatttccattaggtttttgttaagcaaattttatctctttattgatattccctatttagtgaggtgagacatcattctcacagtttttgacacagtttgcttagttattgaatgtatttgaactagctttctctcctaaggcatagctttgtctactaagacaaatgtctgggtttcctcagggagagtttctattatttttttctcttagttatactttctttgttatgtgtcatgatttttgttgaaaacagtacattttaaataatataatgtggcaaatatagaaatcggattattctccctcttcaaggttttttgttcttgtaatttttctaaaataatcttgtaaagtctgtattgtcatgtgtggtcattgaagactgctcgattaacttagtagtcagctaataattagacagagatttccttaaatgtctagaaccatcatgtctcccattctttgccaagagtctctgcatatattggggcataatttcaatcctcagcactgtaggtaataactctgatcttgttttcacttcctgcttcagcagagcaccaaggtgatagcttagggccttttcaggtcttttcttagcatgtccacatcactggtcacatgcactctcctacacatatgcatggcccactaaaatcccaggaatatgtcagaactttaaaagctctctatggacattgcattcctcagcttttctctttacattttttggctagcctctcacttgccccaaatgtcattcattgtctcaggcaactgtgaagttaaacagctggctgggtgagctctgagtcaggtcaaataaagatagccttgtgagtggggtcttccaggaaactcccaggtaggtcacataattctctggaaatgtactccagacctgttttgctccctccaatgactgtcaggttgttggctttcctcacgatttcaggctattggtttttaaggctattgtgaacttggagagaggtgagaacagagcaagttaaaataccacaaagcttactgttcttactgcaattcagctatttttaaggagtaaattcttcctagaatgctgtaacttttggttaatacccagagttttgataaagttgcttctaacttatttttgcctattttctcattgcttttagaaatttcagaggtctttgttctgccatttttgaggaccttacccaatttgctggcactctgttgaggacatcttcatctatgtttcagagggatattgatctatagttgttgttgttgttgttgttgttttaacttttattttagtttcaggaatacatgtgcaggtttgttatagaggcagattgcatgtcatggtgatttgttgtgacaaattaggaccgattattttgtcacccaggtaaaaagcagcatagtatccaacaggtagttttccttgaatggtagctcaactcttagttcaaacagcttaattttaagctctccaccctcaagtaggccctggtgtctgttgttcccttctctgtccacatatactcaatgtttagctcccgtgtataagtgagaacatgtggtatttggttttctgttcctccgttagttcatttagaataatggcttccagctccatccatgttgctgcaaaggacatgatcttactcttttttatggttacatagtattccattgtgtatatgtaccacattttcttaatccagtctactatcaataggcatttaggttgattccacgtctttgctactgtgaatagtgctgtgataaacatacagatacatatgtctttatgggagaatgactcacattcctttgggtatacacccaataatggaattgttgggttgaacagtacttctgctttaacttctttcagaaatcaacaaactgctttccacagtggctgaactaatttacattcccaccagcagtacatatgtggtcctttttctctgtaaccttgcgagcatctgttctttttttttttttttttttgactttttaacgatagccattctgactagtgcgagatgttatctcattgtgtatgtatgtgtttttttgtttttgtttgtttgtttgttttgagacagagtctcactctattgcccaggctgaagtgcagtggcacaatcccaactcactgcaaactccacctcccgggttcaaacgattctcctgcttagcctcccaagtagctggaattacaggtgtgcgccacaacacccacctaatttttgtatttttagtagagatggggttttgccatgttgggcagagtggtcttcaactcctgacctcaagtgacctgcccgccttgacctcccagtgctgagattataggcgtgagccaccacgcccagccacattgtggttttgatttgcatttctctaatgatagtgtgttgaacattttttcatatgcttcttggccacatgcatgtctccttttgagaaatgtctgttcatgtcctttggccactttttaatggggttgttttttgcttgtcaatttaagttccttatggattctggatattagacgtttgtaagatgcatagtttgcaaacattttctcgcattctgtggtttgtctttactctgctgatagtttcttttgctgtgcagaagctcttaagtttaattaagtcccattcgtcgattttgtttttgttgcaattgcttttggtgtcttcatgatgaaatctttgctagagcctatgtccagaatgatatttcctaggttttcttcaagggtttttagtttaaggttttacatttaagtctttaattcaccctgaggtggttgttgtatatagtgtaaggaagggggtccagtttcaatcttctgcttacggctagccagttaggtcccacttgtcaatttttgttttgttgcaattgcttttgaggacttagtcacaaattctttgccaaatctgatgtttagaacagtatttcctaggttttcttctaggatttttatagtttgaggtcttacacttaaatctttaatccatcttgagttaattttaatccatcttgagttgatttttgtatatggtaaaaggtagggggtgcagtttcattcttttgcatacagcaagccagttactccagcaccatttattgaacaaggagtcctttccccattgtttatttttgctaattttgtcaaagatcagatggttgtaggtgtgcagttttatttttaggctcttatagttttcttttttcccccttttccttttgtttttgtagtcttcatctggtttggttgctggttaattctggtctcataaaataaattgggaaggattgcatcttctgttttttagaagagattatgtaaaatttgtgttatttcttctttaaatgtttggtaatatttgctagtgaacacagctgggtgaattttttagaaggcttttaactattaattctatttctttgctagatagctatgcgtgttatctgtttcatcttggcctcataaaataaattgagaaggattacatcttctattttttagaagagatcttgtaaaatttatgttatttcttctttaaatgtttggtaatatttgttaatgaacacatctgggtggagaattttttagaaggcttttaactatgaattctatttatttgctagatagctattcatgttatctatttcatcttgggtgaattttggtattttgtgggttttgtggaattaatctacttcatctaagttatcaaatgtatatgtatagagtttttcgtagtattctcatattattcttttaatgcctgtggagtctgtagttatactattaatacctcttttattcctgatattagtaactgtggcttctcttttttttctttgttaattttgctagaggtttatcaattttattgatcttttcaaagagtctatttttttctgtttttcaacttttattttattttagattccaggagtacatgtgcaggtttgttacaaaggtatattgcatgatgttgagtttggagtacaattgaacctgttacccagaaagcaagcatagaacacaataggtagtttttcaacccttgcttccctccttctctgcctccacttacattcattccctagtgtctatgttcccatctttaagtccatgtgtacccattgtttatctcccatttataagtgggaacatatggtatttggttttctgtttctgtgttagtttgcttaggataatggcctccagttgcatccatgttgcggcaaggacatgatttcattcttttttaatggctgtgcagtattccacggtgtatatgtaccacattttctttatctagtccaccgttgataggtatttgagttgcttctatgcctttgcttttgtgaattgtgctgtgatgaacacacagctgcatgtgtctttttggcagaatgatttattttcctttgggcatataaccagtaatgggattgctgagttgaatggtagctcaactcttagttcaagcagcttaattttcatttcactgatttcattatttttgttacaattcatcaatttatacttgtttttattactcctttttttcctgtttgcctcacatttatttagctcttctttttctagcttcataaggtagaaactaagatcaatgatttgaggatgttttttcctaaaataagcatttaatgacataaatatccatctgagcactgctttagctgcagtccttacattttgatatgctgtattttcatttttgatcagttcaaaatattttctaattttacttgaaacttgctgttttgcctgtggattatttagaaatatgctgcttaatttccaagtatttggacatttttctgttattgatttctagtttaaactagattactagtttaattccattacagtcagagaacatactggacttgagttggctaataatgttgctcaagtcatctatatctttattagtttgtctactagttctgttattgagaggaattttgaagtttccaagtataattgtgtatttttctatttttctcttcagatctgtcagtttttgcttcatgtattttgaaaccctgttttggggtgcatacacatttatgattcttatgtcttcttggtgaattcacccttttatcattatgtaatgttctcttttatccctaattttgtttgcttggaattctactttatctggtattatacagttacttgagctagatcattggtattttgaattctggtctcctttcttaatctacctgctagtgttgacttttcagagttctcaaacagctactccacatattctgtctaggttttttagactactaaatatgcttacttcatcttttctaggactgaaacgcccaaactattttttaaagttaattttagctttgtaaattttaacttttatcaaagaaacacgaacacatagttttatgttttcaatgaaaaacataactgtcctttccctacccacctcctgtttacaattcccattcctcagaaacaattactttcaatattttaggtagatcttctggttatttatctccatatttctaaggaacattgatcctactactacttcttggtttttgaactttagaacttacgtactgattctctcctgtggaagatgtttatttctcttataccactgatacttcaccatcccttcctgggaatattaacaactgagtcacatagtgcactataattagatttcctttattgtacaagactttttgccttccctggagttaataactgcctcttctttttgtggattaatttttaatgttttctgtcattaattaatccccaaaccatgccatagctgtaaaaattcttccaagtatggtcaaaccttctatcctctgaactcaatttagactacttgctgcactgttatcattccagagttttccttcctggttgttttaggaattatctttgcctctattctggattggaaccctagttccctgcatccttgatattcctttttaaattttttttttttattttgatggagcacatcttccagtaacatcctgagaaagtgtccatttgaagcaaatgttttaggtccttgtatgttgaaggctttattttgtcttatagtttattgatagtttggctcatgtagaacttaagttacaaatcaaaaatttccctaaaatatttgaagacaatttcttctatttttttggtttcagtgttgcttttcagaagtctgttatgattctgattctctatcttttgtaagatagagacctgtctttttgtctttggaagattttaagactttttctttatccccggtacttggaaagggcacagtgaagtgctttgtagcttttcctcctttacctgtgcctgagtgagccccttcaaccaagaaatcgtgttactcaattctgagaaattttcttgagttatttctttgagatgtttctttcctttattttttctgttctcatgatctgagccttctatgtatcagaagttggttattgcatactgatcctctaattttctagttcttctttcctgctttctctttttgatctggaaaatttccattttatcttccaagccctattatgcatttaaaaattgttggtatcatgttaatttctaagcactttttcttattcttttgttcttgtttcatggaggcaatatggtacatctctatggatattaagatttttttttgaagatttctcctatctgggggttggcaaactatagcctatagtgcctacaaaaatagcccatgtcttgtttttgtacagcctccaatgtgtaggaatggtcttcacatttttttttttttttgagaaggagtctcattctgtcgcccaggctgaagtgcagtggtgcaatctcggctcactgcaacctccgcctcccaggttcaagtgattctcctgcctcagcctcctgagtagctgggattacaggcgtgcgccaccatacctggctaatttttgtatttttagtagagatggggtttcaccatgttggccaggctggtctcaaactgctgacctcaagtgatcatttttaagaggattaaggaaaaaacaaaaaatatgtgacagagactgtatgtagcctgcaaagcctaaaatatacacaatagtcccccacagccacaatcttgcctatggtcaaagttactcacggtcaaatgtagtccaaaaatattaaatggaaaattccagaaataattcatacatttaaaaatgcatgcccttctgagttgcatgataaaaatctcatgctgtcccactcaggatgttaatcatccctttgtccaccatgtccatgctgtatactctttttgcccattagtcacttagtagccatcttggttatcagatagaaaaaacatagtatacgtagagtttggtactatccatggtttcaggcatttagtggaggttgtggaatctatcttctgtggataaggagagactactgtactgtctggccctttatagaaaacactggccaatccttgttctagtctgttgtgttgtttctattttctcagagttcttctgctgtgtttttgtttgctttgttggacttgttttgttgttgttgttttggtctgtttttcatgttacagactttctttgacaatctggtgaagactctgcacttacttaagagtaaggcactaaaacactgattggcagctctgaatgtatgggtagagcttataggctgggaagtttgctatagggtaatttggcaggaactgggccaatttgtagatattttctcatggggtggtcagtttccctaaaaaggaattctacacgcttggtagatgtaagactgattgtcagcctactcagagctgaaaagaaaaggggggtggagttctcacattcagtatgtaaaatccccgtatataatattaacctccatctttaatacgtcctcagctgtgcctggtgttgaagaatacagagtctctgttgttcaacctttccagaagtaaacctgggtgagggaggaggctaattgtttcttatactaatttgcaaccaagccacctgtttttaaaccttactttcattgacattttagattgctccaaatcctaggctttttcatgcataaatctgaaagtgcttgttgactatcatcaatgtagatttaaatttcagctttctcttgcctgctaagtcattcagtcattcggctgttttccagtgttcaaaatgtgttgatatcgcttgtctgtcactgtctcctctctctctttgtaatattgtaggtttgacattttcatttctttattatcataggattttgagggggtgaagagtaacataaatgttcaatttttctacaaagacaaaaaaaatctaaattttatagggcattattttttgatattattttaaagtaacctataggctccaaggcaacagaccataacactgggaaggataggaagaagattaatattgtcaatactacagaagagtattgaaagtcaatggaaagtcagtgtgccactgtattttggttccagtttgatttatgtttaaagcattctatggtatggtagaaaggtatttctgttaatgaaacaattcaaagaaaaaagatttcaccttttctttttaccattgttttcaaactgccttgcttaaatgaattatgccggcttgagacaaagattatactttcagtaaagcaacaaagactagttagaaaactcatagtgcagttgtaacctggttgcaaaaccatcttgtatgtttccaaaggctgttcttgtatttcatagacttatattacttcaaatactctaacagtcccatgtgtgcttaaaaaaattaacattaaaaggaatccattggtttctgcttactttagatcttctgtatcccttacaagaagagtgcctcagagtataagcaccttttctgtcaatcaatatacattatgagttttattaaataacataaaacatttgcttggtctttatttttttaatttaaaaaattttacttattgattggttttaaagggtaagttcctttgtaacaaattattaattattgacattattccctgaaaatcagtaaaatgatagctgggctctccatttaaaaaaattaataaaattctaaaagtacagtattatatatcaatactataaatacatgatttttatgatattccaggaaagcatgttttgaagctctaaaagctctctgggcaatgttgtatggcaggaaaaagcactgaacctcacatttttcactaattcaacaagcatttgagacttagttggttgctgagaatgtaacaacgtaacacatgttcaaggaagaaaacagataggctattacaatacaatgagatatgtgctataatacagatagcaaccaaaatgctataggtttactaaggtataaataactctagttggagaagttttaaaggtcctcataaaggacatgactttcaatgaggttttgattgctaaatttgccaaacgaattgtatgataacttataagccaaggcaaagcataatgaaagaatatagcatgtttgatagtttaatactgacatacttatgtttaatagtgaaaaattcggtggctagaggtagaggataaggatgaaaaagcaggtattacagttttagaaatggatgggaacaagatgtaaagggccatttagtagccatgtggccttagaaaaataatttaatttctcacaaccagtttaaaaacaattcaggctgaggcaggtggatcatctgaggtcaggagtttgagaccagcctgtccatcatggtgaaaccccgtctctactaaaaatataaaaattagctgggcatggtagtgcgtgcctgtaatctcagctactcaagaggttgaggcaggagaatcgcttgaacttgggtggaggaagatgcagtgagccaagactgcaccactgcactgggcgacagagcgagactccatctcaaaaaaaaaaaaaaaacaaacccaaaaaaacccaaacaaacaaacaaaaacaaagcaaatcaaataaaacatcaagtagcaacatgcattgctactggaagtgattggtagaagaatcaaatgagataatatatgtggagaaggtttgtaaacgagaaaaccacatacaaatatgacttctgtttcacatggtttcttgaaattcaaggcattgcttatactaaaaggaagaaaaaaatcaactgagctaagaatacttattttccgcaaattgagtataatgactaaaagtcaacaatttcaaaaatatattcaagcgcccctagaaactattagctgaatattaaaattcaaaaaaaaaatgtcaatcagcagggagacacaaagtaagcagatgtgatgcttcaataaccttcagtgcaacttgattccaagtaaaatgggatttctagggaaaaaaggggagcaggatctctgtagcttaaccatagacctgaatcactcagtttgaagaaaaacacatgcagaataccattctaaaatgatggggagtggctgggtgtggtggctcacgcctgtaatcttaacactttgggagcctgaagcaggaggatcacttgaggccaggagttcaagaccagcctggccaacatagcgaaacccgtctctactaaaaatacaaaaattagccaggcgtggtggcatgtgcctgtaatcccagctactcaggaggttgaggtaggagaatcgcttgaacctgggaggtagaggttgcagtgaactgagatcgcaccactgctctccagcctgggaaacagagcaagactctctcaaaaaaaaaataataataataataaaaacaaaaataaaaaataaaatgatggggagtacagtgggtgacatgttctagggcttaaatctagttgttgtagtcttatgtctaagctatacacaaatacacatagcttatgtatttgtatatatatacatatagctatgtctaagcgatacacaaatctgaacccacatgccatctggtttacaaatgggctaatgtaaatacacaagacaccagaaaaataaaaattttttatttgactggttaagattatgattagttgtgggacttctggttgaagtgcaaatatgtcaactgatcatgttttcatgattttactcttaaccaatttcttttttttagatggagtctccctctgttgcccaggctggagtgcagtggcgcaatcttagctcaacgcaacctccacctaccaggttcaagcaattctcctgcctcagcctccccagtagctgggactacagacacacaccaccatgcccagctaatttttgtatttttagtagagacggggtttcaccacgttgaccaggctgatctcaaactcctgacctcaggtgatccacccgcctccacctaccaaagggctgggattacaggcatgagccacctcacctggcccaacctgtttctatgaattatccaaatgccttacagcttcactaggaaataaataattacatggtgaagttaattaaaatttcatagagaatataaaatattaatttgtgggccgaatgtggtggctcacgcctgtaattccagcactttgggaggccaaggcaggcggatcacaaggtcaagaaatcgagaccatcctggccaacatggtgaaaccccatctctactaagaatacaaaaattagccaggcgtggtggtgcgagcctgtagtgccagctacttgggaggctgaggcaggagaattgcttgaacgcaggaggcagaggttgcagtgagctgagattgtgccactgcactccagcctggctacagagcgagacttcatctcaaaaaaaaaaaaaaaaatttaatttgcacaaaacctaattatagcatttacaaattagatgttgatgccctacaatatgaaattaagggatagtaatgcaccacataatgatgtttcagtcagtgtgaagtgcatatatgatagtggtcccataagattataatggagctgaaaaattcctatcacctagtgatgtcaccattgtaacattatagcacaatgcattacaagtgcttgtggcgatgctggtgtaaacaaacctactgtgctgctagtcttataaaagtatagcacatacaattatgtatggtatataatacttgataatgataataactgtgttactggtttgtgtatttactatactgtacttcttatcactacagtgtactcctttcacttacattaaaaaaaaagttaactataaaacagcttcaggcagatccctcaggaagaattccagaagaaggcattgttatcacaggagatgacagctccatgcatgttatttattggtcctgaagaccttccagtgattcaagacatggaggtagaagacagggatattaatgctcctgatcctgtgtaggcctaggatactacatgagtgtttatgtcttcatttttttttttttttgagatggagtctcgctctgtcgcccaggctggagtgcagtggggtgatctcggctcactgcagcctccacctcccgggttcaagtaattctcctgccccagcctcccaagtagctgggattataggcacctgccaccacatccagctaatttttgtagttttagtagagatggggtttcaccacgttggccaggttgctatcgaactcctgaccttaagtgatctgcccaccttggcctcccaaagtgctggattacaggcttcagctattgcgctccccatgtcttcatttttaacagaaaagtttaaaaagtaaaaaaataaaaaataaaaatttcaaaaatagaaaaagcttatagaataaggatataaaaattatttttgtacagctgtataatttgtgttttaagctaagtgttgtaaaagagtcaaaaaggtaaaaaaaaagataaaaagataaaaagtttataaagtaaaaaagttacagcaagctgggcacagtggcgtgtgcctggagttccagctacttgggaggctaaggtgagaggactgcttgaacccaggtgttcgagaccagcctgggcaacatagccatacctcatctctcaaaaagaaaaagtcagaataagctcagggtaatttattattgaaaaaataaaaataaataaatttagtatagcctaagtgtacagtgtttataaagcctacagttagtgtacagtaatgtcctaggccttcacattcactcactacttgatcgactcactcagagcaacttatagtcctgtaagctctattcgtggtaagtgccatatacatttgtaccatttttaatatactgtatttctataccatatctttactgtactttttctacacttatatactcaattacttatccttgtgttacaattgtctatagtacagtaacatgctgtataggtttgtgatctaggaacaataggctatatcatatagcctatatgtgtagtagactatactatctagatttgtgtaagtacattttataatgttcccacaacaatacaattgcctaacaactcacttctcagaatatatccgttattaagtgatgcatgactgtataaaataatgatttaaaaatgagtctcacaaagcacaaatcttaatgggcaagaatatttagaagtcggagtatcatgattaaatgtttagttggtgttaattctttaggatcacgattatagtttgttttaacccttcttcaatgatttgctaatttttttttttttttttttgagacatggtctggctctgttgcccaggctggaatgcagtggcatgattttggctcactgcaacccccacctcccaggctcaagcgatcctcctacctcagcctcccgagtagctcacatctgtaatcccagcactttgggaagctgacgtgagtggatcacttgaggccaggagttccagaccagcctggccaacatagtgaaaccccatctctactaaaaatacaaaaattagctgggcatggtggcgcacgcctgtaatcccagctactcaggaggctgaggcaggagaattgcttgaacctgggcggtggaggatgcagtgagccgatatcacgccactgcactccagcctgggcaacacagtgaggctgtctaaaacaataataataataataataattaattattttattttttctttaattattaaatatccttcagtttcaatgttatccttcaacattatcactatacataaaaggcacaagaaaatatagattgtaatcacattgttatctaaagcttacaaataacattaaaatgaaaatgatttttttttctgtcagatgtcttctttacatagaagcagaatgaaaaattgtgcaactacagaagctcactgggcagtaaaatagattttgtttctatttgatgatattttaacagatacacagattccgtactgaaagtacactttcaaatagaatatctcctcacactggctggtatatcaattttcttccctgacattttgggggaggtgaggagagaggtctgtttatctataaaattgttatctgagctttgatgttcacaagacaaaacttttttgatttgataagctttctttaatatttactgagcatctttgtgtgctattcattgttagatcaatcacaaaatttctttaaactttagcaaattgtgagttttgttttgggaataacttattcttcttactcttttcaaattctacttgtgacagctatcattgaatacataccagttcctatggttccagttaaataaaattttaccaagtttattataaagagtaaagtagcaagactagatttatatatatagccctgtcacagataatattatattaaatttcactccacttacctaccaagggcctggtttaaggccctgtatgcttgaatttcataccactgacggccaggtagaagacctctcaattttgaatggtggtcattgtattgtcgttttagttcaacctaataattttaaaatatatttaaaaaattagtagataattaaagctcattttaaacatcatattaacttctaacagtttgaatatacatatttttagggtagagattttattttgtttttcagtactgatttttttcgtttcatttaaaatagttttatttagcttcacattccttaaattacctaatgcatataactaatcagctgtgtgatctcaagcagttttattgaacttacattcattaaatccacacaattttcagtgtacaagtaggtgagttttgacatatatagtaatgtaaccaccatcataatatagaacattttcatcacccccaaaagttccctcttatccctttatagtaaatttctttcttccactgctggccctggcaaccactgatctgctttctgttttgccctttatagaatttcatataaatattatcatatactatgtaatgacttgtgacatttttcatttaatatacttttagaattcattcatgttgaaaggtttatgagtagttcattcttttacgtttatccagtctggtaatctctgctttttaattgcaatgtttagaccatttacatttaatgtagttattgttatgattgggtttaagactaccaccttgcttcccccctacccccccaatctgtaccattttgtttttatcttttggatcaatcaagttcccccctccccccatattatttccactactggaaataatatgcttccatattatttccactactggattattagctatgtctttgtgtgtgtgtgactcctctagggtttatgatatacatccttattagtctaccttaaaataatattttaccacttcacatatgatgtaaaaacaataccacaatatacttccatttccttttattgtcctttatgctattgctgccatacattttagttttatacatgctataaacccataaaacattttgattatgctttaaacagtacattatcttttaattttaaataagtttataattttgaaataagtttagatttacagaaaagttccaaagatagtacagagattccttataccctttacccaatttcccctaatgttaacatcttatattactatagctcatgtcaaattaagaaattaacattagtacattagtattaaataatctatgtattttattcagatttctgttttgtcccaaaagattatcttttaaaatataccgtatttacccacagatttaccatttctggcactcttcattttctcacgtagatttaaatttccatctggtatcattttccttctacctgaagaactttctttaatattagtagtgcacgtttgctagcaatgagttctttcaacttttctttttctgaaaagacttcactatcttcatttttaagagattgtcccacttgatcaagaattctaaattgacaggttttttcttttgttaaagacggcttttcattgttttctgatttgcatagttctgaagaaaaaatatgctattgtcctatctctgtaatgttcccctttcttttagcgactttggattggattacagtgtgctgttgtgtgtattttgcttgcgtttttattaagcttcttggacctgtgggcttatagtttctatcaaacttgaaaaattttcagccattatttctctaaatatatatattattctgcctcgaaactcttcttcttagactccaattacaactatattagactactttatattactccacatgtcactgaccctcttttttttcactctacacttcattttggataatttttatttataagatttcaagttcaccaaattttccttctgcagtgtctaatccatccagtgtattttttgtttcatatagagagatttcacataggtatttaaaaaatagcttctgtttctcttttcagtatgcttatgttttctgctatcttctttgaacttacagagcataaaacataagatattttaaaaatccttgtctgctagccagttctaacatctctgtcatttctggttttgtttctattgattgattttcctctgtattatgggttaaattctcttctttgcatgcctgctaatttttgattggatactagacattgtgggcattaagtcagtggttactggattttgctgtgaatgcaattagggtacttggaaccagttcattctttggcagctttgttagggaaagtcaagagcagcctttagtctagggctaatttatcccccatacaaaggtgaaactctactgatgattgtttcaagtaccccatgtgttatgaagtttttctcttctggctgctgggaactcaaactattcccagactttgtaagcactaggaagttttctgcctactcatttccctggccttatgtagcttcctctcatgtatatgccaatcagtacttgaccaaaaacttaaggggacgcctatgcagatacctggagctttctctctatgtagctccatcctctctagtactctaccttgcaaattctagctgccacggactttccctaaactttatctctttaactcagtgtgaccactgagctctgtttgagtttattctccctgaactgtggcctagtaattgtctccaggtaataatctggtgcaattgtagtattccaggacaagtatatcaaatggtacagttgtcccttggtatccatgggtgactggtttcagaacttccctcagataccaaaatccatggatgctcaagtacttgatacaaaatgacttagtatttgcataacctatgcatatcctcctttatactttaaataatctctggattactttaatagctaatatgatgtaaagctatgcaaatagttgttacattgtagtgtttagggaataatgacaagaaaaaaatctgtacatgttcagtacagaagcaattttttttcaaatatttttgattcacacttaggtgaatccacagatgcagaacccataaatacagagggccagttgtgtatttttgtagtagaagcctaaataaagatatgtggagttactttcttgaaggaaatcattatattgaagagacattgccccccccccatgtttattgcagcactattctcaataaccaagatatggaatcaacctaggtatccaacaacagataagtggataaagaaaatgtggtatatatacacaatggaatactaaacagtcataaaaatgaatgaaatcctgttattcacggcaacatggatggaactgaggacactaccttaagtgaaataaataaaaaacataaagttaaacaccacatgttcttgctcatatgtggaagcttaaaaatgttaatctcggccaggcacggtggctcaggcctgtaatcccagcactttgggaagctgaggcgggtggatcacaaggtcaggagatcaagaccatcctggctaacacggtgaaatcccgtctttactaaaaatacaaaaaaattagccaggtgtggtggcaggcgcctgtagtcccagctactcgggaggctgaggcagaagaatggcatgaacccaggaggcggaggttgcagtgagccaagatcgcaccactgcactccagcctgggtgacagagtgagactctgtctcggaaaaaaaaaaaaaggttaatctcataaaaataaaaagtagaacagaggatactagcagatgggaaggggagaaggaagggagggatggggagatatttgttttttgtcttgtttttttgagagacagggcctcagtctgtcccccagactggacggcagtggtgcgatcatggcttacttcagccttgacctcctgggctcaagtgattctcccacctcagcctcccaagtaggttggactacaggtgtgtgctgccatgcctggccaatttttttcttttttaatttttatagagatggattcttgctatgttgcctatgatggtctccaactcataagctcaagaaaacctcccaccttgacctcccaaagtgctaggaatacaggcatgagccactgcgtctggctgagatttgttaaaggatataaagttacagctagatagaaggaataagttctaatcttttataccacagtaggatgactatagttaacaatattacatcatttcaaataactagaaggaggagattacatgttccatacacaaagaaatgataaatgtttgagatgacagatacgctaattaccctgatctaatcaataaacatcatatgtatcaaaacatcactatgtaccctatgaatatgtacaataattatctgtcaactaaaaaataaaataaagagtaaacttcttggaaacattttaggctttcaagatatattaacaaaacttatacagcagttccctcttatctgcagtggtttaagttaactgcggtcaactgtggtccaaaaatattaaataaaaaattccagaaataatttatgttttaaattgcccactgttctgagtagcatgatgacatctcatgctatatcctgctccatcccactcagatgtgaatcatccctttgtccagtatatccatgctgtacatactacccaccagcagttagtagttacttagtagctgtctaggtctaatcgagtgtcatggtattgcagtgcttaggttcaagtgacctctattttacttaataatactttactaaattgtaaacctactcaagaaaaaacaaagtattttccgtgtactctataacactgttaactttttcttttttttttgagacagagtctcgctccattgcccaggctggagtgcagtgacatgatcttggctcactgcaacctccgcctcccaggttcaagcagttctcctctcagcctcccaagtagctgggactacaggcggccgccaccatgcctagctaatttttgtatttttagtagagttgggatttcacattgttggtcaggctggtctcaaacgcctgacctcaggtgatccacctgcattggcctcccaaaatgctgggattacaggcgtgagccactgtgcccagccactattaacctttttaaataaaagaacatgctttaggaaagaaaacagaacagtacacttctgaaaagcagctttcaaattgggaggtcaaaaaattttatcttactgtaaaaataaaatctaagctttttttttttttttttttttaacacggagtcttgctctgtcaccaggccggagtgcagtggcgcgatcttggctcactgcaacctctgcctcccaagttcaagagattctcctgcctcagcctcccgagtagctgagactacaggcgtgtgccaccacgcccagctaattttttgtatttttagtagagacaaggtttcaccatgttgaccaggatggtctcgatctcttgacctcgtgatccacccgcctcggcctcccaaagtgctgggattacgggcgtgagccactgtgcctggccaaaatctaaaaacccaaaaccccaccctttttattttacattccctcaaaagaatctgtcaaatttctaaaatgaaactttacaactagaatataaaggacatataaaggacatagactttgccttttttttttttttttttttttttggacagagtaccgctcagtcacccaggctgaagtgcagtggtgcgatctcagctcactgcaacctccacctcctgggttgaagtgattctcctgcttcagcctcccacgtagctgggattacaggtgcacgccaccatgcccagctaattttttgtgtatatatatattttttgtttgtttgtttgttttttagtaaaaacagggtttcaccatgttggctaggctggtctcgaactcctgatctcaggtgatctgcccacctcaacctcccaaagtgctgggattacaggtgtgagccaccacgcctagccaactttgctttttttttttttctttttttttgagacggagtcttgctcaatcacccaggctggagtacggtggcatgatctcggctcactgcaacttctgcctcttgggttcaagtgattctcctgcctcagcctcctgagtagctgggactacaggtgcatgccaccatgcccagctattcttttgtatttttagtagagaggggggtttcactgtgttagccaggtgatcttgatctccagacctggtgatccctctgcctcggcttcccaaagggctgagattacaggcatggagccactgcccccagcccaactttgctttttagggcaacataagtagtaaatgataacataacaccatagtaaatattagatgatatgttaataccctccctggaaattcagcaataaagcacatgataattaagactataagaaaaagtacttttaaaattctgtccagctgggaaaaatatatgtatttcctacctatctctcaaaaatgtaactaaagcagcttctcttctttaacaatattttcctcaaagaaagttgacatctactttgtcagacataatagcttatgtgttttcaaaatttcctacaccatggacagactgatacattatagtggtcactgttccgaagacaacagttgctctaggacgtattagttataaatggaatatttagagtgaatgtagcaactattagattaaataaaattatgactccttggtcttctatttttcattaacataattaaaattttctgtatggctacattgttttaatttattgcaaattttcaaacaccttttacactccaaatggcctttaaaatactgccaatattttacccaggggaatttattttagataaaaagtttaaaaacctacaaaagaacaaaaaactcccaaacaaaaaacaaaaccctgaaatgaactgtatataaaggttaactttgaaatagttcttattacataaacagatttttaaaattaatcaataataaaaattactttgtgagatcacagttaattggaaaaagcaaaactaagggttaagaaaaggtggaaagtgagtacacctcattttaatgacatcagaatctatttcattgtacacttagggattatcaagcatgaaaatcaatgaccaatttaaaatgacatacagcatatgtgaaaaagccagtttctcctgccagcaaatagcctaaatgttaagtaaaccaaaggctggaagggattagttgaaatgtcaaaagacaaccttttacttttacctgctcatccttataaacacattcttatctctcaggcatgctttccaaacaaatagtttcaatgtgcaccaccagcaaaaggcccaaggcaagctctgccatcttgtaactgtcaaagagattagtctttgatctatttgggaggtcatgcccacattcacattaccttataatctaacaacattatacctgttgaccaatgcttccttaatgcctaatgagacagacattttacattatggaagttaactgtgaatgttttttctaagttttgacagaaagtaagcaccataaactgagctcaagtgtggtttgggaagatgatcttttagatcagttaacattacttttcaaatacattttacaagcctttcccaaaatagaaagctatacttaattgtactttttggaaacagttaaactacttgcttcccatccccaataaattagtgtatgtgtaattttggaggggtgtgaagagcaggaaaaagacaaactattttattaacagttgcaaactgaaggcccatagttgtgttttgtgtggcccttactgtggcttaaaataattaaaatttatttgctaccatctaaaactttggctatctcaccactgtcctcaactaacccactcatttaaataacctgtttaattcatgtagctgcttgagtttgcaacccattggttcagatgctgcatagattcacaaaaatttatcatttcataattgaaggtcaagtttgtaccaaatgtaaagctcccttactatgatagtttttagacaaataaatatatatctgcatcaaaatctaacctaactgacataagtagaaataagatcctcaatatcaaaggcattgataattaagactgaggcggtttagaattgggtaaaaatacccaatagacagatggcattcaagcagaagttatatgattttggattctgatttttaccattatatcataatgtacttgagattgaacagaaagtttcttaagttctccatgccttggttttattggaaacagtataagtagcagagcacttatagcagcaatagacaatatgaaataatcaaaaggattatactgcatatgtcaacgagaaattaaggtcacaattattaacattagactttttcaactttcagtcaaactctttgcacaaaagtagcctttttaaagtttatcagtagcttctgaaaaaataaatggtgtgataaatatgagctagaactataaatgcatctcttcattttactgctgcttcacatgaaagatggtggattttatcccaactcataccaatacatattatgatttcagtaataaatgccttcctgaaagcaatcaatagaagaatataatttttttcttgtatgtaagcttgaagacaaaagttcaagcctccaaatgagactaaggacaaataatcagctaaactacataattataaaaatattaagaaaactctgcttcagaaaaataaacacatttgtctagcaaacacttgagtagaacagttttatctttagccaaggtgcagggcctaaagcctaaacatttacagcaaaccaaatctggaagacagagactgtgcattagagacatttgctgctccgtatcatcatagtgtaaacacttctatcagggtatcacaatataaattccaatggttagaattataaactcagcataagatcttgtatgtgatataatttagctagattaaaaagaatatagctggttttatttcatctggcatattttcaagatttctgacacagttaaccaaatagtgtgtgtgtgtgtgtgtgtgtgtatatacactttttttaaatggggaaaatgagttagtttctgccaagagttaagaagccaggctcccttttaaataagaataataatgataaaagcacattagaaatggtttttagttgttacttactttccctgttacgttacaacttgaccacaataatctatcaagacatagtaacagcaatatatttcacatatgctttaagactgttttggataaggtgctttttttggtagagaaatctatattaaacttagtatcgctaattgtcagtgttgactaaaataacctggatatttcaaatttaagaaatagaacaaaatatatccctctgaaatcaggtatctcctcttctcaacatattcagcctcaactggttataaaaatataaccagtttttcctattaaaaagttaatactctaagacatgcaagaaatcttagcatgcttattctgtaaagggctaagttaaagttttcatttttattttttagagacaaggtcttgctctgtcacccagaatggagtgcagtggcatggtcataggtcactgcaacctccaactcctgggttcaagcaatcattctgcttcagcctcctcagtagctaggacttacagatacgtgccccaacaccaggctaattaaaaaaaaaaattattttagagacgcagtctcactacattgcccaggctggtcttgaactcctggcctcaaacaatcctcctgtcttggcctcccaaagctgtgggattaaggtgttatccaccatgcctggcctaaaaaattctttttaagagtaatgacctttgtgtcaatttaggtaggttatccatgttaaaatattactcaaacaaacctgcaaatgtttaaattatcatctctagaacatgccaccattcgtgttatagtttattttgaggaagaaggtataatggaaaaaaagaaaaagcacacggcttggaatcaaggccttgagacttaggttgtttgaccttggacaagtcacagtagccctttataatttcctttctcaatttcttcgtctgtaagaaggggcttagaattagaagtctcttagctttattgaagttggagactgtgtgccaaccttaccttagagaattggttgaagaaataaatgacaatatgcatgaaacagtaaagcatcagatactagaggtaagtggacgagaaagtcattacttaccccatatttctgaaacccagtgtaggtttcagtggactttaaatgctgtcgtaatatattgctgcatggtatgtgaaataagccataaggctgtggaggccatatagtcattaccttagtcagttcaccccaaatcattttaccacctggaccgattccttctgaaagagctctgactacattattcaatgtttggcacataactttaggtgacttaaaaagtaagtttcttgggttcttggagtggctactttttcatcaagtgtcaatgaaaatatatacattggcagaatacatacagaacagtgttcaaacagttttgaaacaaatattttaaaaaataacttctacaattgatcttaaattttctaagaaattttagctaacattagcactctttaatatcctccataatactttttggtcacttttcaacttatgaagtgtttttgaaagcataactatataaaatatgctagtggtgtaatctatgagaatgagtgcatgattggtaccatattagtctaaatattattttattcaaatatcttacaatctagagtttttcttgttaaagtcacatatgagagcccgataactgtgaataaacttttgctagcaataacaactaacaaaataaaatattatcttatgtaagaaagcagtaaatagaatcagaggttcttaaaggaatgagtcaatattaagaggatttaataaggggactcagtaatggtgcattcagcaggtgacagagacctacttctaccttatataacactggaaaaaggcaagattgctcatggaagaggcttttggatgccagaagatgtaatatgacgcataatggcactcagggaattatataaaggttcctggcaatagtagcataaaaagtaaaagttaatcccagtgagaggaggctcagactcagctttcatatgattaaaccctaacatctaaaaacaaacctaagcaagagcgagcatgtacataagaaaaaagattaaatgagtactggaaaatgctgaaaatacacttaaaactttattaccaccacagtccaaaagatgaagtataacacttttgctatctagatttttattattacatccaaattatatttcagggccaggtgtggtggctcacgtttataatcccagcactttgggaggatgaggcaggaggatcacttgaggccaggagtttaggaccagccagggcaacatagcaagaccctgtctctacaaaataaaaattagctgggcatggtggtaggagtctgtagtcccagctacttaggaggctaaggcaggaggatcatttgagcccaggagtttgaggctgcagtgagccatgatctcaccactgcatttcattgtgggcaacagagactctgtctgtctgtctgtctgtctctctctctctctctctctatatatatatatataatctttcaaacatgagcaaaggtttgattttttttttttttgagacacagtcttgctgtgttgtccaggctagagtacagtggcatgatcttggccactgcaacctctgctccctcagttcaagcaattcttgtgcctcagcctccacagtagctgggattacaggtgtgtgctatgacgcccggctaatttttgtatttttagtagacacggggtttcgccatgttggtcaggctggtctcaaactcctgacctcaagtgatccacccgcctcagcctctgaaagtgctgggattacaggcgtgagccaccgcgcctggccacaaaggtttgatttttaaaatatatgatcactttctacctttgaaatattattgccaagaatatttgattcagagcttttcaaatgtgaatgtgagttttggaataagacatatggaatcatagcactttgataactcatgctttatcttagaaagtaatgcagacttctgatttcaataagtcaaaaaaaaaaaaaaaaccccagacatttctttttccctcacctgtcagagaaactgcctttaaattaaaagacaaataggaaacagaaacacagtctccaacttcagtaaagctaagagacatctgattccaagccccaatatatgaacataaaaggtagatggacaaatgttaaatgactaagcagaaaagagaaagttcaaaccaacgtatccacagagctactgctgggacataagccagtttgacctgcaggagttcagaaagtctcaggaaatggaggtataaggaaactgttgggaggatgtgtaccgcaaagatgaaacagtaagcaagaaacaagaggaagatctgagacccaggaaactggcaatccatcacagaagacagacaaatcaaattcctaggatgatagcagttattggatgatagcaggatattggcagttatctgggcttgatgagcaatttagccctgaccgaagtagaagatcaggaggctccagaaatgatatctccaggagaaaaaaggaactggcctgttatctgacaggtctgatagaaaaactgtattgagaggcatggcacacaactattggaaggtgacacatttatttagataaagatcttttaaaaagagaaaaaggaagtggaagagaggagggttctatcaaccctaaagttcaaaacagaaaggaggacgggcggctgtaaccccagcactttgggaggctgaggcgggcggatcttctgaggtcgggagttcgagaccagcctgaccaacatagagacaccccatctctactaaaaatacaaattagctgggcgtggtggcgcatgcctgtaatcccagctactcgggaggctgaggcaggaggatcgcttgagcctgggaggtggaggttgtggtgagccaagatcacgccattgcactccagcctgggaaacaaaagcaaactccgtctcaaaaacaaaacaaaacaaaacaaacaaacaaacaaaaaagaaaggaaatcatttgattacagtgaacagtattttcatagtcatgataatgtacataataactatagatttaattaaaaattgtaatgtaactatcttgggaggctataagaaggaaaaagcagagtaagtgaggtaaaatcctgtctcaaataggaatttaatagttcatgtctaaaactgattaaataaagataaaatatggaggtaaataccagataaaatagctaaagaagtgaaagtcactgcctgtcagaagcaggggctagaagtggggtgcaggccaaaagactgttataagaaaaagctatctactgtatttagtgtatattagactcttggtaagtgctttattggacgtttttcaacttgataaatcataatttaaaaacaaatgcagattattaattttaatttattgtatacatttgtatgcctcactatgtaaaacagtggtgctcaaccgagagcaattttgccaaccagaagataatggcaatgtctagagatattttgggttatcacaactggtggcagggtgaccgtggggggtgtctagatcccactaccggcatctagtgggtaaatgctagagatgctgttaaacatcctggaatgcacaggacactcctcggaacaaagaattacccagtccaaaatgtcaatagtgccaagtttgagaaacacagcttgaaagtaatttcaagtcacaaatctggtatacttgatgcagtaggaaaatacactgagttatttgtatagcttactatagtcactgacgcactggggtgatgtgtcctacagttacaaagtataaactgaactcattcccaagctttcaagcctttaatcctagggaaaaacaggaaagagtgatgttgtaaacatggaacagcaatatctactacagtttatgttatttgagaccttaaagctttacagttagaaaaaaaagtgacatagtcacctgaagcccctctacatgtctacaatctgtaggcattgagagaatttgactgaataaactgattaaaattcacaactaaaattctcctcaaaagaaaaaaattagttattttcagagaagctaaaaaacaatcccaccactgcccttaaggtagagctgctgattatgctccatgccaacataagtaggtaagtgtcaatcaaccttacaagtcctcagggtgataatttcacttttaaatcctccaacctagtttgagagcaaaattaatatataaaggtcaaagttccttttctcaccaaaaataagatgaacacaagaaaataacttcagaattgttcaaaaatcaaaaccaaaagtttttatttattaattatccagagaaacttccagcaatattttcaggcatggctttatgtatttatttatttattttttgctaaggaaatacaatgacatgagtattaggagaaaaaggttatcaaccgaacttgcttaaaattttgggattttaaatatttccatccttgatcccaaaatatcttttaaaaatatggagaatttaaaaattaacctcagcaattcatccatttgttttgtgtttttttgtttgttcatttgtttttcagaagggatggaaacagaggggtttcattaactcattttttgctggccaaggtaaatatacaatttattaacctagacagcttacttagatttaaactaggttttaattttggaatttcaagatatacttagaaactgctttcaaaatacttttggccaaactatcttaaaaaaaaaaaaaagctggggaaaaaaatactctgaggaggaaaaaaaaggtttaagtaattgaggatatactcactctagacttttagaggtgacaaggttttttcttctatatctgtgatggaacctaagtatttccaaaaattccaagagcaagttgctgaacttgtaaatgttaaagagttaagtattacagcactttaagtttatattcacatccatttcccctgggtaaccatgtattattatcagagctatatccaggtgggtctctgtatcagatgcaacactataaccagaagtccactccactctgacctcaggctgtattcctggatctggtttcccataacttagaatgtcccaaatcctgaaaatggcatacagagtcctaagtgatttggctcctaaatacctctcccagcttctattcacatgccattctctcagactcacaacttcagcttctttcagttcttggaaactgctatcctctttcaaccttagaaccttaacacatgctgttctctctgttgagaatactcttatccattctgtacacattcataactcctagttttctttccagtgtcaggttaaatatcacttcctcaggaagcctttcttgatccccaaagacctgctataacattcccagggttcctgtgactttttcttatagcctttatcacaactgtgattgtttcttcattacattaatgtcaatattccttgctagactataagcaacatgagacaaggggctataaactgtgttgtgtatcactctgtcccctgagtctattataatacttggtgctctagccagatattaaaaaaaaaaaaggttgctttattattctgaatagttgctttttttcttttctttctttctttctttcttttttttttttttttttttttgagacagagtctctgtcacccaggctggagtgcagtggagcaatctcggctcactgcaacctccccctcccaagttcaagcaactctagtgtctagtgtctcagcctcccaaatagctgggattgtgccatcacacccagctaatttgtgtatttttagtagagagagagagtttcattatgttggccaggttgttctccaactcctgacctcaagtgatccacccacctcggcctccaagtgatggaattacaggtgtgagccactgcacccggcctattctgaatagttttataatctgaatagttttgttgtaatctaacctctgatgaggccattccactttatgaaatgtttgtatatttgaatctcactgtagagaagattcaaatctcatctctctggctcctccatcccttttaggcaagttagtcaaagtaaattggtatctgcaagggaggcgaaaggtgttggtcaacttcagctttcctaagcatctgagcacctggtttctattccaccaaagcatcttgttatatgttcctgttcttccataaattattgacattactagtgtagagacagcatcttgttcatttttacacctactgcattttgtacaaattaagctcttaaaaaaagtttgttaaatgaatgaataagtgcatgacaaaacattcaatatcattaacatttttttttttttgagacagtgtcttgctctgtcgcccagactggagtgcagtggcgcaatttcagctcactgcaacctttgccccttaggtacaagtgattctcctgcctcagactcccaagtagctggaattacagacgtgtgccaccacaccggggctaattttttgtgtttttagtagagactgggtttcaccatgttggccaggctggtctcgaactcctgacctcaaatgatctgcccaccttgggctcccaaagtgctgggataacaggcataagccactgcacacggccgtcattaacatagttttgatagaagagtaattcaaaagggatttataaatctattcaaaaggaacagatgaatctacctaaaaggtttctgaagtcaagttcctcttgatatactacagtcctttgcattcctgccctcattcattttctcgtgaatattcatattgccgtcaaagcaaaaggaataacaatggaacaatatcattctccttaaaaagaaatctgttagatttaagaattctaatacagccaggtgcaattggcgcgagtctgtaatcccagctgctcgggagctgaggcaggaggatcatcacttgagcccaggaccagcctgggcaatatagtgagactctgtctcaaaaaaaatttttttttaattccaatactaccatcatctgataatattttgacagttgaaagcactggtttaacatggtttaatgtgctttattactttattaatgctgttatcctttagggaaagcaccctttttctgtggtttataaaatgccaacaatgcatggaagcctatcaatttttttgtgtgtgtttgttataaagtcgataagtaactaatataactaaaatagatgtctgaatttttcaatcataaatgtaattttaaaaaattgattaaaataatatatacccaaatagtttggtacattgagagctaagataagtttcctaaacaaaaaaaggaactcataatggttattgtatgctaatagtaagatagacaattcagatcactatgcgaattttaaatagtttccaaataaaatctagtttggtaactcaacatactatattaccaaaaaaacccacaaatataaaatttattagcatgataaattattttaatcattgtaagggcacttctgacaatgaaaagtaccatcaaattttattaaaccaattataaaaatctaaacacaccacttcaacattttaaaaatataaacatttttagccatatgccatggttttatacatttttcttcagagaagtaaaggaacaatacctacgaaggagaacttttctatatactattatactatactatgctaggcaatacaaataaaatgcaattttaaaacctaaatatatttgtaccttatcatatctcacaaactatgtccatgtcaccaattggagaggtggatgcctcaattataaagctgaatactcctgtggttgttagatcacagtaattggccagaggatttgctttagatatccattaaaaaggcagccttgttatactgtgatttaattacatgaaaaagaaaacaacttttattttagttatacttttccatcgacatctagtcatataaaagccttggttcattctcatgcccctcaaatgctttggtttagtgcacagaaagttttctttttaactattatacgattaagtatatccttatgaatgatcagaagcatctgggaaaggtggaagtgaaaaaaatcctcactgggaaagttagcttaatgcttaccttcagagtagcccattatcaacatttcatagtgggtttctttattttcaggtccctcaggtatgtgcaataaattatgacttgaactaaacaagtcacacttggctccaaaatgaaattttcctacctctactttctagtccctaaggtgggaagaggaaatcctggtcacagccaaaggagtcatatttttggctctaacatacattttccttcttctcaaaaagccaaaccctgctattgagactgcaaagtcttatgttagtattttaaactgtattttaacttttacttccataatctttctcaaatttaagaaaaaattttagtatcataaaattaatgtcactgccttctttttcccactcaaatcacttttctgactcaatgatcttaacctttttaaatatgaagtcaaatgaaatagacaaaataatgcagaaataggttgtttcgtttctataaggtagaacatttaaatttttatttactaaataacttctacttattagactattaatgtaaaagaaaactataaagttctaatttaaggtcctataatttgaagcatagttttagttttggaagagccatctatgtaggtcatttagatcatcatctcttttcatagggaataggagtatggcagacacaccctaaggtgatatacagagagtcacaactttgtaatgctctccccttaagtgagggcagaaccagtgatttgcttctagccaacagaatatggaaagggttctgggatagtcactcttaattaggttatattatatagcaaaaatgatgggaagttattcccatgattttttaattttatttttttcctcacaattttattaagttgtgtaagactctgtattggctgactggggctagagagcttcctattggcctctggaagtatactgtcatgatacgagggggcttgtgaaaaggccacatggcaagaaactgtgaatagcttcttgaagccaagagtacaccttagggctaggcacggtggctcacacttgtaatcccagcactttgggaggctgacgcgggtggattacttgagctcaggagttcgagaccagcctgggcaacatagcaagatcctgcctctacaaaaaaaaaaaaaccacgtttaaaaattagctgggtgtggtggcgtgtggctgtagtgccagctacccaggaggctgaggtgggaggatcacctgagcccgagaggccaaggctgcagtgacccatgaccgcaccactgcactctagcctgggcaatggagtgagaccctgttttcaaaaaaatttaaaaaagactgcctcttggatggattaggaagaacctcagtcctacaaccacaaacggaccccaagtgccagataacaacacaacctggccaacactctgattgcagcagttttgtggaaccctgacccagctaagccatgcccacacttgtgaccaatggaaactgagataataaatgtgtgttaagccactatgtgataatttgttacacagcaataaaaaactaatacaggctgggcatgatggctcacacctataatcccagcactttgggaggctgaggtctgagcatcccttaaggccaggagttcaaggccagcttgtacaacatagcaagacctcgcctctccaaaaaaaaaaaaaagtttagccaggtgtggtggcacatgcctacagtcctagctacccaggagactgaggcaggaggatcaattgagcccatgagctcaaggctgcagtgagctatgattgtgccactgcactccaacctgggcgacagaacaagaccttgtctataaaataaaataaaaatatttaaataaataaataataaaagagggatagctaatatagtcaatataagatactccctccacttattatgttctactatgtattaaataaagtgtaagatgctgttgaggattgagcattcccttctacatatcctctaatgatttactactataactaccctgtattgcctccttatgagtcatatcactgtttactaccatcctgttcattattatctaatgattactatcatttccatccttaaagtctccaggaatggagagtggttgctgttgtttttaaaaacaaataacaaagtcaaatacaattttaacaacttctactaaaaccccattcaatcattaactaaaacatttattaaacactgtgtttttaggtaatatcttagttgtagagacacaaagatgctgaagaaattatccctatcctcaaggtacaccatgaaacagattataaaacatctttataaacactaaatatgtgctttaggaaataataaaggacactggtaaaggtaactgcaggtgaatactacaatatgcttttgaattgcaattcctctgttttccctatattatttaaaaggcaaatgcataaaaacaataattaaaaactaacagccacacaaagtataaagatggaatctgtgacaataacaatataaaaagggaggaacagagattcagatgatagttataatccccaaggtaaatactaaaaaccaaaaaatacatataaaaggaaagaagggaatcaaaatagtatactacaaatcaactaaatacaaaaaaggaaatgatgaaggattgaggaacaaaaagcacatgacacaagaaaacaaatagctacatagcaaaagtaagtcctttcctatcagtaattactttaaatgtaaatggtttaaactctccaattgaaaggcagagattggccaaatggatttttaaaaacctgatccaactatacgctgtaaataaaagattcacttagatataaggaaattggacctcaccatatatcacatagaaaaatttactcaagaggaatcaaatatctaaattaaagagttacaactataaaactcttcaaggaaaacattaggggcaaatatgcatgatgttggatttgccagtattttcctaaatatgacaccaaagcacaggcaacaaacaaacaaaaaaaacagataaactggactgcattagaattttaaacttaacttttatattccatagaggcatacaaatattttcaaatacttgtttaaccatttataaaaatgttaagaaatcacccataatgatgctctgacatatttcaagtattttaggtattgactgctatattctgggcatttccaaataataaaaagccttcttaatcattataaaaactgcatgcgtatactcttaaagcttgtaacacaccagtcttataaatggataactaacaaataccttttaattatgaaaaaatgcgatagttgaggacttctttttgaagtcatgattctaagtcctttgccatgtgcggtagaaattatcccttttctccccttccttgactttgagattgtgcatggacgtgttttgggcaacaggaggttagcagatgtgatgtaaataggtatttgacttaactgcttacagttaagcctgccctcttgtactactgccattgcaataataacatgccttggctagtctgttggtccacaaagaatgaaatacaggtggaacagagctgactcagttgacttcaacacagatcagccaactagggcagagtagctgagcccagctaaaatcgagagagccaaccaaccaagcccaagcctagatcatctgtcacccaatcactccagagatgtatgacctgtaataaatgattgctgttttaagtcactaagttttggagttgttacagaagaagaactgatccagacatatggcaataagaaatacacttcccagtacatctagtagtgctggtaaataatttcactttttctatcccttgtccctgcagcttgaactattcttgtgtccactctacttgtaccttttgggaaccactagggataaccctttagaaccaccttcctcccctctacccagaacccattgatgcttctgttattttgctgttttatgcacataagcagaactatgacaaataggatcatgactgaggaatgtcagagtaggccatcaggtgtcttttttgcatgacagactgagatattgtctaggaagaatttatcctgaggaaaaaaagcattaagaaccccagattggtataataacaatgttcaaaatcatagctgttgaggcaaaggtggagaaaagagaaatggaattttctcacaaatgtatatttttaattttaagaatttttggaaaataatgaagaataacataataaatacctgcattctcatattttcttgcagcttattttcctttactaaaaattatggaaaaagttaaaattcttttgataactactctgaatatcattcccttctcttctcctcatatgtaatcagaattataatcttgatgatcctttcaggccttgttactttgtacgtataatatttagtacaacctagtattagccataaagtttttcttcttttccaggtctcaagaactcatatgtagggtccaaggaagagagaggacataggcaaaaatggaaataattagttttagggaacaaaattgtattttattaagatattggaagaagatgtggtctaaagaagggaaaaggattctttccactttaacaaagcagccttagtgcaataatgtagagctagagtaaaatgataagacattaaaaggagacaataccagtaccaacagcagtgccatcagtagcactctacttcagtggaatgcagcaccaatgctgttgatggtgacagtagatttcttgggtagatgtcaactgtttcatcagtagctgtagtcaaggcagcatcagccattttagaataggagacccagctttgggcatagccattagtgccctcagcagtagcactggttaactggagaaaggtagtagaaaccaccaaagttactggaggctgaaaaagtattagactctgaatcatcaagtgagataccccctgaggactttcagaaattttgaagaggtccagaggttacggaagagtaagggctaccaaaagaaatactgcttattactgttaatgttacctaaagactgctacggactagaaaaataaaagaaaaattatctaattcacagtacctagtaaagtattaacaattaaaaagataactattctaatgaaagaactcctgctttggtatgagaccctaactgactgtatcctagggggagggatataggtcataatcaataagtcagctcagtggtcagaaaatctcaaattttctcagattgccagcactttcaaatgcctgtaggaagtaaaagaaaatcctccttgatgaacaataagttcaacttaggctcaaattattactatagtttttcaaatatattgtttaggaaacagtcagtgataatgagatacaactgtttctggccaagattgagtaactggtttaagaatatttcttccattgtaagtaatcgcataaccgggcaaaatggagcaattgttttcaggtagtagacaccaaacagcacaagacagtaattctggagctaagtggaattcattaggtgaactccattatcccagttctctgcttgggtataatttcctgaccacagcacagagcattaaagtacaagcagagcgcagtggtcctgttgagctgaggtggaagagatcagaatatggagtgactaaatcacaagatctgcaaggcaggacactggaaagaagcgagccagatggacaaaagaccccaggattccccggaggaacccatgagtctgtggttaaaagactgataaaacacaaattgctgggcttcacttccacagtttctgatttggcaggtctggatgagcctgaaagtctttatttctattaagttcccaggtgatacgaatgcacctggcccaggaccagactttgaaaataactcctgttgaccaaagacagtataagttgaaaataaaataaaatctgtatctactaaatttttctcactcattcagattcaccaaagatttaaaaatgtctaaggatacaggaaaacatatacaggcatccctgagagacattgcaagtttggttccagaccaccccaataaggagaatatcataacaaagtaagtcacatgaattttttggtttcccaacacatataaaagctatgtttacactatactgcagtctattaagtgggcaacagcattatgtctcaaaagccaatgtatataccttaacttgaaaatattttattgctaaaaaaggtgaatgatcatctgagccttcagtgagtcactctctttgctggtagagggtcttgcctcaatgatgactgctgactgatcagggtggtggttgctaacgttggagtggctgtggcaatttcttaaaataagacgaccatgaagtttgctctatcatgtgactcttcctttcacaatagatttctctgtggcataagatgctctttgatagcattttacccaacataaaacttctttcaaaattggggtcaagcctctcaaactctgccgctgcttcatgcactaagtttatttaatattctaactcctttgttgtcatttcaacaatgttcacagcatctttactaagaatagttcctatctcaagaaaccactttctttgctcatccataaaatgcaactcctcatctgttcaagtgtcctcatgagattgcagcaatttagtcacgtcttaagctccacttctaatttagttctcttattatttccaccacagctacagttacttcctccactgaactcttaactccctcagagtcatccaatgagggatggaatcaatgacttccaaactcctgtaagtgttgatattctgacctcctcccatgaatcatgaatgttcttaatggcatctagaacggtgaatcctttccagaaggtttttaatcgactgtgtccagatgcatcagaggactcgctatctatagcagttatagcctcgtgaaatgtacttcttaagtaatgagacttggaagtagaaattactcctttattcattggctgcagaatggatgttttgttagcaagcatgaaaacaacattaatctccttgtacatctccatagagttcttgagtgaccacatgtgtggttaatgagcagtaatattttgaaaggaactgatttttctgaggagtagatctcaataatggacttaaaatattcagtaaaccatgctgtaaacagatgtgctgtcgtgcaggctttgttgttccatctatacagcacaggcagagtagatttagtataattcttaaaggctctaggatttttaaaatgttaaatgagcattggcttcaacttaaagtcaccagctgcactggcccatcacaagagagtcagcctgttctctgaagctttgaggtcaggcattgacttctcctctctagctatgaatgtcctaggtggcatcttctttcaatagaaggctgattcttctacactgaaaatctgttgttttgcgtagccaccttcatcaattatcttggccagatcttctggataacttgctgtagcttctccatcagcacctgctgcattaacttgcacttttaagttatggagatagcttctttccttaaacctcatgaaacaacctctgctagcttcaaacttttctgcagcctcctcacctctctcagctttcacagaattgaagagagttagggccttgctctgaattaagttttggcttaagagaatgttacggacagtttgatcttctttccagacactaaacctttctctatatcagtaataagactgtttgcttattatttgtacattcactggagtaacacttttaattcctttgaagaacttttcctttgcattcacaatatggctaattggttggtgcaagaggcctagcttttgtcttgtctcagctttcaatatgccttgctcactaagcttaatcatgtctcgcctttgatttaaagtaggaagcatgcaacacttcctttcacttgaacactctggggccattgtagggttattaactggcctaatttcagtattgttgtgtctcagggaatggagagtcccaaggagagagaaatgggggaagggtagtcagtggagcagttagaatacatacatcacttatgaattacgtttgctctcttatgtgggtacagtatgagatgccccaaaacaattacaatagtaacatcaaagatcacttatcacagattaccctaacagtataataataatgaaaaggtttgaaatatcgtgagaattaacccaaatgtgaaacagaaacaagtgaccacatgctgttggaaaaatggtgctgaaagacttgcacaatgcagggttaccataaaccttcaatttgtaaaaaagcactataaaagcaaagcgcaataaaatgagggatccctgcaattaactttatacaaaaccaatgactcctgtaataataaaacttaaggttttgatggcctacctgtagatctttcacatttggaaaaggaattcctattgttatgacagcacgggcattgtcatctgagaaatccagaccctcactcactttaccacgacaaactgctaccaggagagctccatcttaaacaacagaaaaaagcatatccaaaattctcagaaattgcttattcttgtcattttgaaaatacctgatttataattatagtaattacagtagcaaatttaaaataatcaaaataaaacactattatgagataaagttttaaaagttcaatgtctttaagtaaataaggggaacaaaattacatttatatgcaaaagggtagcaaaccaatttacaaactctgcatcttccagataacttatacagtaaaaacatgtcagaatagcaacttgcatatgaactgtgtgattataaacaggtttatttaataaaaattgaagttgcattattacctttaccacctgaaagtaaaattctcagaataattgaaataattgtaattgataatacatatcatatggaatcaatatatttaaaagtattgaaaacagacacagagaacaccataatatatacaccataaaaagatactgatatgttatattacctctttcttgcattgttcataaactactactatcccttcacatcaaacttttttattttattttattattttttgtgtgtgagacagggtctcactctgtcagccaggctggagtgcagtggcacaatcacagctcactgcagccttgacctcctgggctcaggtaatcctcccacctcggcctcctaagtagttggaactataggcacatgcaccacaccctgctaatgtttgtactttttgtagagacaggatttcaccatgttgcccaggctggtctcgaactcctgggctcaagtgatccacccgcctcagcctcccaaagcgctaggattacaggcaagagccaccacgcccggcctcacatcaaactcttcatggatatgctctaacttggaattgagttttcagttatgttttgaaaattcaagtttaccttatgtaatagaatacaagcaccaagtgtactaaatgcatcagaatataggtctcatttggctagtaaggaatatatttatatatatcctaactgcctttttgcacaaactatttcaggtagtgcactgaagactgtgagatgatggcatcgtttttcttaaagttgaatcagcatactcaagtgaaaatattcataggagaacaagtacaattaaaagaatttctttacccaatttattttcttttcactcaggattataatttttctcttaagtgtaattcatctaaaaatataaaattataattgtaccttcttactttgtaataaaaaatattttttcaccgaccatgaaataatttccagttacctttctctcctttgtatttgattgcgtcatagtacacctgcagtaattcatcaaaatttgttttttctcctccctgtggttctacaatgactgtcttcaccaactccagattatgccataaaccagtagagagccaacgttcttttaatttttctaataactaaagaggggaaagaaaaaaatgattttttgtgtgtctagctaaacaaacttaacttcatttgtttaagccaatgtgactacggcaagtatattaatctctctgtgtcttttctcatttataaaatgaggaaaacaatatatctcatagagctgttatggggttaaatttattaacagatttagagagatctgaatggtgctggcacagttagctgctgctgctgctactactactattatcttggcaatttctaccactactactgctactactactattactactactacttctactattatcttggcaatttctaccaccaccaccactactactactactactactatcttggcaatttcaaaagcaaaagcaaaaaacaaaaaacctctgtcaaagttaacaaactttaagttctctctttaaacctaaggtgaagtttcttaatattcttaaatagtcttcaaatgaagatgatttaagatcattttactaagtgtataggcagataagctcattatttgaatatttgatcatcattaataacctgcattgaaaaaagatagaaataaagactcagccaagaagtagaaaatatatgggcacaaatggaacatttacagccatgggctagattacaaaaggacattcaacaaattttaagcaatccttatcatattgatcacatatttcaacagttatgccatggtagattaaaagtcaataatataaagtcatggtagattaaaagtcaagcccaaaacaaaaactcatcttgattgattgattgattttagagacagggtctctgtcacccaggctggagtacagtggcaagatcatagctcagtgcagcctcgaactactaggctcaggcaatccctccaccttagcctcctgagtagctgggactacaggtgcatgccactacacccagctaatttttatattttgtgtagagatgagatcctgctttgttgcccaggctagtctcaaacttctggtctcaagggatcctccagactcagccttccacagtgctgaaattacaggtgtgaaccaacacacgtggctgtaaaactcatattcttaaaaaccatatgagttaaatagaaaaaacacagtataactcatgaaatctttagttatgaataagaaaagcactcacaattcgaaacatgtggcatacagcaatggcaatacatggaaatacatttatagctttaaatatatttatttttaaaatgtcaaaataaaagaatcaagtattctttccaagaagctagaaaatgaaatatagcaaatgcaaagaaagaagttaataataaagctgaataaatgaaaaagacaaaagctaatattagagattattatttaaattgaactgttttatccacttgttatatactatgagttctacacaatttggacaagttattataagaaaccacccaaaattctctggaaaacagcaactgatcaatttttataagatagtttatttcctcaattttacctttaactatttttagaaataaacttcttaataagactataaaacaaaacattagtaaaatagttaactgctgtacaatatgctttaattaaataggaagaactactttaattccataaacacagctaagaagttattatattactttttattctccagaaagacatcaagggaggaatagaggaacaaaaaagctgtagttagacagaaaacaattattgaaatgataataatgtctttccctatcggtcattactttaaatgtaaatggattaaattttctaatcaaaagatataaagtagctggatagataaaaacgctgtctgcaagtgactcactttaaatttaaggatacccataggctgaaagtgaagggatagaaaaagatattacttataaatggtaaccaaaagacagcagaggtggcaattctaagatactaaatagattctaagtcaaaaattgtcacaagagacaaaaaaaaaggacattatataatgataaatgggtcaattcactgggaatatttaacaactataaatatacacacaaccaacatcagagcacccaaatatagcaagcaaatagtgattgtactgaagagagaaataaacagcaatataagaggagtaggaaagtcatatatctaaccttcagtaatgaataaaacatccaaacagaagatcaacaaggaaacggaacttgaacaatactagagaccagatggacctgacatatatagaacattttacccaacagcagcaaaatacatattcttcacaactgtacatggaacattctctaggacagatcatgtgttaggtaacaaagcaagtcttaacaaatgtaagataaaaattaataccaggtatcttttgcaaccacaacagaatcaaactaaaattcagtatcagaaagaaaaccagcaaatttacaaaaatatgaaaattaacacacttttgaacaaccaatgaatgggtcaaaaaagaaatcaaaaggaaagttatgaaattatcttgagaccagtggaaaaaaaaccacaacataccacaagtaagaaatacaccaaaagcagtactaagaggaaagtttatagcggtaatgccaacattaaaaaagaaagatctcaagtaacccaattctacacttcagggaactagaaaaagaacaaagtaaatacaaagtttgcagaaggaaagaaatattaagattagagcagaaataaaatagggaacagaaaaacaatttaaaaagttggtttttgaaaagatcaacagaattgacaagtctttacctagattggttaaggaaaaaaaaaagagaaaatcagaaatgaaagaagacacactgatgccaccgatacaataaaagactactctgaaaaattatatgccaataaattggataaaatagatgaaatggacaaattcctagtaatatacaacctactaagactgaataatgaagaaacagaaaatctgaacagactagtaaggagactgaatcagtaacaaaaagtctcccatcaaagaaaagctaaggacctaaatggcctcaataccgattctacaaaacatttttatttatttaattttattaattttttattttcatttctttgctaattaattaatttatttttgagatacactcttgctctgtcacccaggctggagtgtagtaatgtgatcatagttcactgtaacctcaaactcctgggctcaagcaatcttcctacttcagcctcccaagtagctaggactagaggcatgagccaccatgccctggtaattttttttttttttttgtaggtatggagtctcactatattgcccaggttggtcttgaactcctggcctcaagtgctcctcctgccttggcctcccaaagtgctgggattacaggtgtgagccactatgcacagtcctctatgaaacatttaaagcaggggtccccaacccccaggccacagaatactggtagaatccaggctgcacagcaggaggtgagtggcaggtgagtgagcattactgcctgagctcagtctcctgtcagatcagcagcagcattatattctcataggggtacaaacactattgtgaactgtgcttgcaagggatctaggttatatgctccttatgagaatctaatgcctgatgatctgaggtggaacagtttcatcccgaaaccatcacccccacccccaacccaggtctgtggaaaagctgtcttccacaaaaccagtccctggtgctaagaaggttggggaccattgattttaaagactaatacaaatgtagattcaatgtaactcctatcaaaatcccaatgacattttttacagaaacagaaaaaaatacaaaaattaatatgcaaccacaaaggacttggaataatcaaaacaatcttgagaaagaacaacaaagctagaggcatcgtgcttcatgacacttacatcttacaaacccaccataatcaaaatagtaattgcattgccataaagacagacatatagaccattggaagagaatagagagcctggaaataaatccacacatgtatgatcaactgatcttcagcaagggtgccaaaaatacataatggggaaaggacagtctcttcaacaaatagttcagggcaaacaatatccacatgcaaaagaataaaactggacctttatcttacacaatacacaaaagttcactcaaaatggattagacttaaatgtaagatttcaaactgttggctgggcgcagtggctcatgcctgtaatcccagcactttgggaggccaaggcgggcagatcacgaggtcaggagattgagaccatcctggctaacacggtgaaaccccatctctactaaaaatacaaaaaattagccgggcatggtggcgcacgcctgtagtcccagctactcgggaggctgaggcaagagaatcgcttgaacctgggaggcggaggttgcagtgagccaagatcacgccactacactccagcctggcgacagagcgagactctgtctaaaaaaaaaaaagacttgaaactgcaaaactcttagaaaaaaacacaggagaaaatttttgtaacattggatttaacaatgattttatgcatatgacataaaagcacaggcaataaaagtaaaacctacgattgggaccacaacaaactaaaaggtttctgcaaagtaaagggaaaaaaaagagtggaaaggcaacctaaaggatgagaaaaaatatctgcaaaccatatatctgataaggggttaatatccaaaatatattaagaaacccaatattaaaaaaaaattgaaaaacaggcaaaggacatgaatagacagttctccaaagaagacatacaaatggttaacaggtatatggaaagatgctcaacatcagtaattatcagggaaatgcaaatcaaaaccacgatgagatattggttcacagctgttcggaaggccattatttaaaaaagtaaataaaataactattagtgaagatgttgtgatattggaatccttgtgtattgctggtgggaatataaaacagtatagccactgtggaaaacagaaaaggaaataaaaatcaaaataaaacagtatatatagaaatgttttatgggtttcaatatacaagtcttttacctccttaagtttattcctaagtgttctatttgttttgatactactgcaaatggaaatgttttcctaatttgcttttcagacagtttgttgtcagtgtatagaaatacaactcatttttgtatgttaattttgcatattgcaactttactcaatttattagtcctaacaggttttttaaaaataagatctttaggaaagatcacgtctgtaaataaggacaattttactttttcctttcccatttggaagacttttattcctttttcttgcctaactgctctggctaggaattctagtactacgttatacagaaatggcaagagtgggcatccttgccctgtttctgatcttagaggaaaacctttcagtttgcaccactgagtatgctacgagcttcttacatatgacctttattatattaaatttccttctattcctaccttgttgagaatttgtatcatgaaagtgtgctgcattttgtcaaatgcttttttgcattattgaaacaatgcatgaagattcttttcagcagatggtgttgagaaaactggatcaccacatgtaaaaagaatgaagtaacaccttgcctacataataaaaattaactcaacattggtcaaagacattactgtaagagctaaaaaatataaaactctaaaagaaaaaaatagaaggctttatgaccttggatttggcaatgatttgttggctgtggcatcaaaagcataagtaactaaagaaaaatacagataaactcggatttcataaaaaattataaaacttttttggatactgtcaagaaagttaaaatgatagtacacagaatgcaaagcatatggctgataaaagattaatatccagaagatataaagaactacaactcaacaacgaaaaaaccaaacagattcaaatataggcaaaggactttaatagacatttcttgaatgattaaaaaaaaaaaaaaggcccaaaggacatgaaaagatgctcaacatccttagtcattaggaaactgcaagtcaaaaccacaatgaagtcccacttcacttagtataactgctgtccaaataagtaagtgttgcaaggacacgcagaaactgtaactgtcacacattgctagtggcaatgtaaaatggtacaactgctgaggaaaacagtctggcagttcctcaaaagttcataggcatagaattaccatatgatccagcaattccactcctaagtatataaccccaaatatctgaaagtactaggatccaaacaaacagctgtatgctgatgtttatagaagcattattcccaataaccaaatagtggaaacaactcaagtgtcaatcaacagatgaataaacaaaatgtatgacctacgtgcaatggaaaattattcaactataaaggaatgaaattctgacaaatgcttgaatatggataaacccatagtaagtgaaaaaagccactgtatttttgtacacaaagtacaaatattatatgatttcacttatatgaggtacctagaatagacaaattcttagagatgggaagtagaacagaggttaccagggcctgagggaaggggctaaacaggagtcattgttccatcagtacagtttctgactgggatgattgaaaagttttggaaagggatactggtgatggttgtacaacatcatgaatgtacttgatggcactaagttgtacacttaaaaatgcttaaaaggtaaattttatgtcatgtgtattttaccacaattttaaaaattgaaaagaacacacaaatgaatactatttagtaacataaataaatgaactagtttctcacatattaaccaggatgaatcataaacttctagatgttgagtaaaaaatgcaaatacagatagataacaaatatgatttcatttatataaagttcaaaaacaatatgttctttgagaaaaatatatataactttaaaaaactcatgaaactaatacctttagctttagggggatacaattgggaaggaacaaatggaggatttttttttttgagacagagtctcgctctgtcacccaggctggagtacagtggtgcgatctcggctcactgaaatctccgcctcctgagttcaagcaattctcctgtcccagcctcctgagtagctgggactacaggcggacaccaccacacctgactaatttttgtatttttagtagagacggggtttcaccatattggtcaggctggtctcgaactcctgacctcaggtgatccacccctcagcctcccaaagtgccgggatcacaggcatgacccactgcgcctggccagatggaagcttctaatgtagaaataatgttctatttcttaagctgggtgatggatactcagatctccattttattattatttaaaatgtacatattagttttgtagacttatatacacacatatttcacaaatataaaatataaaaattaaacaacattagtctgattattaatatgatgagttagaggaggggttataacaggtacatttaggctactaatatagtaatccagagatggtggtagcttgaattagaaagagttaagaaaaaaaatgcaaaccttgagaataagataaaaaagaaacagtacaaactttccatttcaaatggaggcaaaggcccactagaagattatctagggaaatacactttgttgaactcactgttcactattaattaaggttccagacttagcaaaattatatttcaacttagatatttttgtctaatacaaatacaaatcatttctgttcaggagaaaagataaccctaaaagttactatttattgccctgtagtacatcaaccagttttcttatagtctattcctttattaaattgcctacaagtacgtgaatccttaaatgctctctgaataggccaatcatcatactttttcttcattaatcagtgagtttaataaaatctgtgataggtgttaactttatatgtgagctatgactttatctttttgaaaattaactgtagagaaagaaaagtgaacagattataaaaatacttaaaattaaaatgagtaagatatggtgttggatgggacatgacagggaagggagaaaaaggttcacaggtgctctgtaggtatatgattagcccaaatatgattacagtacctgctatgttttgaacatttgtcctctccaaaatgttgaaatttaatctctaatgtggcagcattgagaggtgggggattttaagaggtgacagggtaacaagagtgctgccctcatgaatggatttgtccagtcatgtattaatggcttatcatgggagtgggactagtggctttatacaaaaaggaatagagactcgagcttgtatactcagcctccttgccctgtgatactctgagccaccctgggactcagcagagagttcccaccagcaagaaggcccgcaccagatgcagcctctcagtcttgaacttctcagcctctgtaactgcaagaaataaattccttttaaaaaataaatcacccagtttcaggtagtctgttacaagcaacagaaaatgaactaagacagtaccattcattcatacaggcaacactggcaaaggaccaagactggaaggaaagattaaaagtttgttgcctttaagatatccaaatggaaatgttgatatggcagttgggtatatgggcatagtacttaaggaagaagtcagaatttaattatatattaggaaagcatgacaaaagataaaactaaaaaagaatgtatagagatgagattgcctagtgggagagtacagatttaaaaggggggaccagcaagaaaatcttatttcctctccatttttttttttttttagagatgggatctcactctgttgctcaggctgaagtgcagtggtgtgatcacagctcattgtagcctcacattcctgggctcaagcgatcctcccacctcaacctctcaagtagctgggactacaggtatgcactgccatgtctggcttgaaaagaacaacaacaacaacaacaacaaaaaaccagagagggtcttgctgtgttgccaaggctggtctcaaactcctggactcaagcactcctccaccttggcttcccaaagggattatatgcatgagccaccacacccagcccctcatctctactactgctcatccaattcatcagcaaatcctgccagcactacttttaaatttatctagactctaaacatttctcactatctttactgctaccacagtggtccaagccaccaccatctatcatctagattacagcagactccttaacagtcacccttgcttcagcctatgctctctacagtccattttccatacagcagccaacataatccttttaaaactaagtttaaaccctccaatggcttcctactgcatttagaacaaaatctaaattcttactgtggtgtgattttacccagctatttctttggccgtatctccttcaattccccatacttcagtcctactctgctacaatggcttccttttgctcaaactaggtactctcctgtctcaagattcccactatctgaacattcttccccctaaatcagaatggtttgcttcctctcttcctttacacttttactcactgtcaccttctcagataggccttcccaaaccatccaattaaattctaaatgagcaccctccagcccccacccaaaaaaattccaaccccaggatcctcttttccccctttccttgctttagttttctacatagcacttatcaccatctgtattagtcccctcaggctaccacacaaaaatatcagactgaatggcttaaacaaaagaaatttatttcttacagttattagtttaagccacagattgagtggcttaaacaagagaaatttatttcttacttcttggctatgaagtccaagataaaggtgcctgttaacttgatttctggttagggctctcttcttggcttgcagacagccaccttctgtgtcctcacatggcctttcctctctattcagagaggaacagggagagagagcaatgaagttttctggtgtctcttcttataagaacactaatcctatcagataaggaaggcccttacagcctcatttaaccgcaattacttccttagaggccccatctccaaatataaccacactgggggttagggcttcaacataaaaaatttggtggtatacaaacattcagtccataatatcatctaacatattagtagctttctgtgtctctcatttattttctgtctctcaccctggaatacaaaaggacaatgatttttgtctgttttacttactgctgattgcccagtgcatagaacagtaccttacatatagaaagtggtcagtaaatactttttgaatgaattttataaatgaatgaaaaatgaaggtccaaaacaatggaaggtcagggaggagagtagggaggggtaaaacataactgtaaaaccagatgcccaagtccttaataaaagaagagttggctgggtgcagtggcccacacctgtaatcccagcactttgagatgctgaggctggaggatcacttgagcccaggagttcaagaccagcctgggcaacttggcaaaaccctgtatctacaaaagtacaaaaattagctgggtgtggtggtgtgtgcctgtgatcccagttactcaggaggctgaggtgggaggactgcttgagccctggaggttgaggctgcactgagctgtgatcatgccaccgtactccagcctgggtgatagagtgagaccctatctgaaaaaacaaataaaaataaaatattaaaaataagagtatccagatgctttgtaagatgacaaagatgagggggaaagaagggtatgactaaaagacatgagcaaacagactagctagactagactagttttctcttggtgagactttgttcattcattccttcctcccttcctttcttctcccctcccttccggaagaaaacagacaggtaaagatttagaaagaacaatggagagtaaataggatactaagtctgagatactgcaagttacgagaggaaaggatggtcacaatttggaaggtctgacgaagtggtatgtcctctgggtctatccggattttagttcgggcaaagagctatgaggaacacttcataaaagggagaatttggtgactatgacacatttccacagaacacaatgtgttttattttaatttcttcagtggagattttttttaaagtaccttaaaaccttttgttaaggattacacaggggctacatgcaaatttggagtacaattctatagcaaaaacatactggttattttagtgaaaatggcctacagcaaattttcagtttcagataacaaaatatttaggtctacagcttttatattctcattgcaggtgacacttgaattacacatttagacagcttaactatttctgtcttgccagagaaatcacataaataaacacaaaaataactcaattgttaaattctcttaggtatcctaactaggcacctaaaaactaacttaaaggaaagtacgatgacacagagctgtctctcaggtcttatggcttacatatatgaaaaatgtgaggtatctttgttgaaaatatcctacttaatagctcataaagggtaaatactattcatatagggagaccagccaacatatagtgatagagatgatttaatttactaattggtgttcacatcacttcaatttgttaacctcagaggcaattttttttaaagtcagatctcacaaactaaaagacactttagaatgcccatgtcttatctggatattgtcattttactttgtcaaacttctttaaaactagtatttcgctagggagggaataagttagaatgcaacctagtggggaaatcctgcatttgctcaatgtccatagcttttacacagttccagatgttcagtacattaataatacactaactcaaagcctctttttgaaactttagaaagtgcaaattgcattaagacctctttgccctcattagtggtattgttacaatcccagtcctctggaaaatgttttttggcatgtgtttaactttataatattgccatttactcactttaatagccagtactcaagtcttaatgcttttaataggcctgcctaaacagactaccatcttttgtaatcttactcacataatttatttcataagtagaacctggtatatttttccatctaaagatgattcttcattattcatttcatgaggtactcatatgttttaaatgtttactttcttttattcagttgatatgtacccatatgtggcatatgttagaaataaaaaagagttccttttaaatactaataaaaatgaaaaagtagtagtgttttttaaataacttttttagttacagtaaaaatgaatgtccttaatttttgctaaatacaaactgaaaatataagaaaaccaagaatatgaatttctattgaaacagcttcctgtttggtgatatttgactcagcactttcacctcaagaggctgggttatatttcagaatcaaactttcaatgaacacaggtgcagttggaggtgcacttggaggaataatatagacaaatactgccctaaagtgaaagtaagacctctgttggcaatctaactggaaatgaagatcctacagccctctctgaaaaggaacactctctctcataaattagttaaaatactgaggactctgaaaatgtcagagctgcactactagtgcagtaatgcactagtgacacctgatttacagttacttcttgatacattctgataaaaatatacaatgatatttacaatgcaaaaaacttcaaaaaatgacatttgtaaaacaactaacatatttacacatgaaattctactttcctgaaatcatgtgaaaagaaaacaataaaagccagacgcggtggctcacacctgtaatcccagcactttgggaggccaaggcagacagatctcttgagctcaggatttcaagaccagcctgggcaacatggctaaaccccatctctacaaaaaaaataactgggcatggtggtatgttcctgtagtcccagttacccaggaggctgaagtgggaggatcacctgagccagggaggtaaagactacagtgagccagtgattgtaccaccacattctagcctgggcaacagaatgagacactgtcttgaaaaaaaaaaagagaaaaaagaaaagaaaacaataagaactcaatactatgttatgtatggaacaaattcttaactctttgaaacataaattctgaattactgttgtttcatcaacagttaattcatcaatgtctaaagtaatcatttagttaaaatgtatataatctgacttgttttgctttttaaaaatgaagaactgttgactgagttaataatgttggctaaaatctgaatgtataaacttattcacacaaattcaactatatgtttcattcttttcctgaatagattaactgtaggcgaaaaaggcaactcgttttctatacttctgtaaatgggctaataattaaactataatgaagtcagtgctcttgcactattagaattcaggctgagggaagcagatgaataagaagcaaccactataatgttacctaaaagttactactttcaagtattgccatctgaatctgctgtaaatttgttgctctaaaacttgaaagaaattttttggagaaagcatcgcttcacaatctagtgaataagatccaaaagaaaaatatttaaattacagtgcagtgcataacaaacatagaaaggcctcaaggcataccactacaggaaaccatcaaatcacaaaggaagacagcaagagaaataaagaaacaaagtacctacacaacaactagaaaataattcacaatgctgggtgggcgtggtcactcatgcctgtaattccagcactttaggaggctgaggcagaaggattgcttgaggccaaaggattgcttgaagccaggagtttaagaccagtgtgggcaaaacagcaagaccctatctctaaaaaaaaaaaagtttttaaaaaatgtatctgggtgtggtggcacatgcctgtagtaccagctactcaggaggctgaggttgatcccttcaacccaggagttcaaggttatagtgacctatgatcacacccctgcactctgacctgggtgacagagcaagaccctgtcataaataaataaataaataaataaataaataaataaatggcagtaataactctttacctttcaatacctaccttgaatgtaaatgaattacactctagcaaaaagacatagagtcttaattcccagagcaatttagaaagagtaagaaaagaagatccaactatatgctgcctacaagagatttgcttttaaggatgcacagaggctgaaagtgagcagctgaaaaaagctattcaacaaaatggaaaccaaaagagagtggggttatctatacttacataagacaaaatagactaagtcaaatactgtaaaaagaaacaacgaagaacattatttaacaaaaaaaggtcaattcatcaagaggatataacaattgtaaatacacatgcacctaacatcaaagtacctaaatatataaagcaagtattaaaggatctgaaaagagagatagattctaatacaataatagtaggagacattgataccccactttcaacaatgaacaaatcatccagacagaaaatatatttaaaaacattggacttgaataaatttagaccaaatggacctaacaggcatttacaggcataccttggagatattgtaggttcagtttcagaccaccacaataaagcaaatatcacaataaagtgagtcatacatttttgttacccatcatatataaacggttaatgtttatactatactgcagtctattatgtgtacaagtggcattatgtctaaaaaaatacataccttaattttaaaatactttattgctaaaaaaatgctgacacagagacatgaagtgagtacatagtattggaaaaatggtgacaatagacttgcttgacacagggttgccacaaacctttaatttatgaaaaaaaaaaaacccagaatatttgcaaagtgcgataaagcaaagcacaataaaataaggtattcctgtacagaatatcccattcaagagcaacagaatacacattcttctcaaataccataaaatagtctccaggatagattatatattaggccatgaaacaagttttaacaaatttaagtttgttatcatatcaagtatctttgctggccacaatggtataaaactagaaatgagtaacaggaaaatttgggaaaattcacaaatatatggaaattaaacaacatgttcctgaacaaccaaacaaccaagggggcaatgaagaaattaaaagggaaatttaaaaaaaatatattgagacaaacaaaaatggaaacacaacataccaaaacttaagggatgcagcaaacgtagtcctaagaggaaagtttacaataacaaatgcctacatcaaaaaagaagatcccaaataaataacattactcctcaaggatctagaaaaagaagaactaagcccaaaattaacagaaggaaagaagtaataaagatcagagaataaataaataaagtagagactaggaaaacaacacaaagatcaacaaaactatgaattggtttttgaaaagataaatgaaatcaacaaacctttagctagattaacaagaaaaaaagagaatattcaaataaaatgagaaacaaatgaggagatattataactgataccacagaaatacaaaggatcaaagtgactattacaattatatgccaacaaaataaactggatacctggaagaaatggataaattcctagacacatacaacctaccaagactgcatcataaagaaatagaaaatttaaacagaccaataatgagttctttccaatcaaagaaaagcccaagacctgttggctcactgctgaattctacctaacagttaaagaactaataccaatcattctcaaatgctttcaaagaagttgaaaaggaggatatacttctaaattctttttacatgaccagcattatcaccctgataccaaggccagataaggacaatacaagaaaagaaaactgtaggccaatatccttgataaatagaaaagcaaaaatcctcaacaaaatactagcaaaccaaattcaacaacacattaaaagaacaattcaacatgatcaaatgggatttatcactggatggaaagatagttcaacatatgtaaatcaataaatgtatacagcacattaatagaatgaaggacaaaaaccatatgatcatctcattagatgtagaaaaatcatttgacaaaattcaacatcctttcctgagaaaaacttttactaaattaggtatagaagaaatgtactgcatatttggccaataaaggccaaatatgataagaccacacttaacattatattcaatggtgaaaaaattgaaagccttacctctaagatctggaacaagacaaggatgcccactttcactatgtccacttaacacagtactgaagtccttgccagaaaaatttagaaagagaaagaaataaaaagcatccaaatagaaaagaaaaaagtgaaactgcccctgtttgctgaagacataatcttatatatagaaacccctaaagactacaacaaaaaactgttagaactaataaatgaataaatacagtaaacttgcaggatacaaaatcaacacacaaaaaatcagtagcatttctatatactaataacagactatccaaaaaagaaattaagagaactccttttacaatagctaccaaaaagagagagaaagaaagaaagaaatttaaccaaggtgaagaaaggcttgtacgctgaaatctataaaatgttgatgagagaaattgtagaagatacaagtaaatagcatgatattctttgctcatagactggaagaatattgttaaaatgttcatattacccaaagtgacatattgattcaatgtaatctctatcaaaattccaatgtcatttttcatagaaatagaaaaaagaatcctaaatttcatatagagccacaagaagctttgaatagccatggcagtcatgagtaaaaagaacaaaaatggaggcatcacacgacctgattccaaaccacactgcaaagctatagtaattaaaacagcatggtattggcataaaaaacagccacagtgaccaacgaaacaaagagcacagaaagaaacccatgtatgtgctcaactgattttcaacaaaggtgccaagtatacacaatgggaaaaaaagactctctaaaaatggtgctaagaaaattagatatccacatgcagaagaatgaaaatggacccttatctcacaatatttaaaaaaaaactcaaaatgaattaaagatttaaacccaagaactgaaactgtaaaactactagaagaaaacagaggggggaaaactacataacattggtttaggcaatgattttttggatttgatcccaaaagcacaggcaacaaaagcaaaaataggccaatgcaattacaacaaagagaaaacaataaacaaagtggcaacctacagattgggagaaaatatttacaagctatatatccaataaggagttaatgtccaaaatatataagaaactcaaacaactcattagcaagaacacaaataactcaatttaaaaatgggcaagggatttgaatagacatttctaaaagaagacaacaaatgaccaacagatacatgaaaaaatgctcaaaatcactaatcattagggaaatacaaattaaaaccatgatgaaacattaccttatgcctgtaacaatggctactgtcaaaaagatgaaagataacaaagtgttggtgaggatgtggagaaaagggaagccttggacactgttggtaagaatgtaaattagtacagccattataaaaaactgaatggacattactcaaaacactcaaaacagaattaccttatgatctagcaatctcacttctgagtatttacccctaaaatttgaagtcagtttgcagaagaaatatctacactccctcgttcactgtagcattgttcatttggctggaattggagaacattatgctaagtgaaataagccaggcacagaaagataaatgtctcatgcccttactttacgtggcatctaaaacaactgaactcaaagaagtgacagtaaaatggtagttaccagagtctggggcaaccacccttctactttctatctctgaatttgactactctaagtacctcatataagtgtaatagtgtatttgccgtttttatgacgagcttatttcacttagtctaatgtcctccagattcatccatgttgtagcatgaaaagaaatacccatttattatctcacagtctctgtaggtcagaagtccagacacagggcagctggtatgggaatgtggggaaggttgtgcatgtgtcgggtaaaggggtatatgagagctgtctatactgtccactcaattttgctgtgaacctaaaactggtataaaaaataccatgtgacctatggtattctctacattgagctgctatctgtagaatcttttccttgatgacttttttcctttatgatctctcatgttagcatgacttcaagctaacattatctacactaggcaagcaaggtgaatcttatttctaaaaactcctcagaaatagtactttgtacaaaggcaaacaaccaacgcactgatatgagctctaaccattcattcaccatgtcctgtagagactgatgtatcttttgaaagacttttcaaaaatgcaataacccttgagttaactagatatcttaaaatcgaaggctagagattgggagagttcgcttctataatttgcatctgctgtatatttgagaatatataaaatacaagaacccaaatagatgggtgaaactcttaaaatgttaaaatcgtagctagaagttagaaacctgtgtaagccatattaaagagataaagtaagtacagcatttctctatgaacatagatgaaaaaggatggaaagacactagatattcaagatacactacatatacatgaggtatcaaatatccaagatacattaaatatatctgaggcatataaaatactacccaatatccataaaatcttattcagccacccattggtgacacccattctggctccatgtatcctctgcccaagaaaggcaaataaaatacattgtagctcagcaggcaaactgatggactacagctgtggccaagtgcatttgcgctggccttactctaataaagtgtgtcaattgtgcatctgttgggtccccttgcataaacctcatttctagtctctgtaagcacaccatcagcaaaggagagctgatgtggaaaagagaaagacaagacataaacattgcacagcccaggatttaggtagtgtggtccttccacccacttacattgtattcacatgttaacctaccctcaactctctccccttgcctcccactgtgtgttttaactatctttactaaactatatgattcaagcatcccaatgtggtgtcacttttttgtcccatgacctctgggataagtatatttggagactaccactgcatcaagttaacgtccctcctgacaccacacaaatcatctaaattcaaaaggtgccaatatgaaactgtcagccaaaaaggaaaatcaattggttgctattatgaagattaactgggtagatattaaagattgtaaccaagatgaaacaactagaacctccaagtttttctagtccatatcaagctcaaaaattgctttaatatgtaaaatgtacagtaaattaatcataagttatatttaactaatttgtctgtcatttcccaaaagtgcttcctatatttatccttacatccatgaaaaatagtttagaactaagggggtggggggagcacagggagttaggatgaattcagcttctaaatttgctacttgatccctgcctgctatggtttgaatgtcccctcccaaagtcatgttaaaatgtaattgtcattgtaacagtattaacaggtgcagcatttaagagaccattaggccagaaaggttctgtcctcatggatgggtttaatgcctttaaaaaataaaggagctttttctttctgtgtcacttgccctttcacctttcacctcgagatgatggagcaagaaggcccttactagatgctggcaacttgatacttggacttcttagtctccagaattgtgagaaataaatttattttctttctgtccagtctgtggtattctgttatagcatcaaaaaattgaccaagacactgccttctcaaactaagttacctggacatccttgactctagtttaatcttaacttctctgcctttattctccctagatttttcacttaaaaattataatacatatacatacataaacacacacatacacatatacacatacatatgtatgtgtgtgtgtgtgtgtgtatatattatatatatatatatatatatatatatatatatatatatatatattttttttttttttttttttttttttttttgagacagagtttcactcttattgcccaggctggagtgcatggcgtgatcttggctcactgcaacctccgcctcctggattcaagcaattctcctgcctcagcctcccaattagcagggattacaggcatgtgccaccatgtccggttaattttgaatttttagtacagacggagtttcaccatgttggccaggctggtcttgaactcctgacctcaggtgatccaccctccttggcctcccaaagtgctgggattacaggcatgagccagcatgcccagcctaatcaatatatttttataaagcttttttgaaaccagatgtgatacaaatatattcatgttatttatagaaagtacataaatgactatattcatgcacacacacacacacacacacacacacacacagagcaaaaaacaacttggttattaaattgttactttccaactgccctactattttcattcaattgttaaagcagctttttaacatcctctttcagagtagctcttatatctatcttcctaacagtttagctcataatcaaaaacaaatgtgaaaagcaaacataaatataggtataccaatatgtaggtatttttatataagtgcatattgttctaaataaaaataataggtcaaattaatatttatcagatcaactatatttcaaacagtagaatactattacgaggatagcattgaactagaaaatcaggagttctaaggcaaatgctttcactaattaaatgtatgaccttagccatatgaattgtctactcagaatgtagtatgtgcccagtaaatgtttgtcaaatgaataaacaaaggaacaaacaaaggaatactttttgtttaataaccaagagcagaagttctcaagaactacatctaacctgagagatatacaacctctctaccacatcccaaagcctcaatggcataatgattctttctatttaacactgtagaaaaaaatatttcttcaaaaggctcagttgatcacttaaagcaaatttatatctatagaaatttcagccttctacacgaggggaaaaaaaacactttttaatgtagcaatttagtttctcctttaggttaagtgcggtacagctttaaaaatgagaaccaaaacaaactagtagcactgcaaatttcttaacatacaaatttagtgctgaacaatactttaaaaaactacagctagagagtgaacaggaatgtttgcattttattcctcatctgacattcagtgatgaatgtaaaatatgccctcagggaaaatggtttatacatattactcataaagatgtatttttttttaagtcgaagtctgtattgtatgaataaaaagatccaacttgttactttggcaccagagagtctcttttcaaaatgatttaatagaaaagtttctattctctttatatggacagaaacttctctgtccatataaagagtttagaaactcttctacccataaaaatgaatttcagagagttctaaagcaatgctgctactacttaaataacaacagagtcaaaatcaaatttatcttagtggaagaatggcattcctcctagttaccataaactattggtctgaaagaatgtggggaaaaaaactagatgaacaacgttctcactcttacccttaaacaaatgctgcagtttacatcaaaagaatattagtttttactcagtgaaagagtaaagttttgagtaaatcacccattccaattatctagtacaggaagaaaactggccattaaatataagggggaagaaatgaccactaaaatgtatggtttgaaaaactttagacatatatttttcttttttagagatagggtcttgctattggccaagctggagtgcagtggtgtgatcacagctcactgcagcctcaaacttctgggctcaagcgatcctcccatctcagcctcctaagtagctgggactcacaacaccacacctggctgatattttttgttttgtttttttaaacactgggtctctctatgttacccaggctggtcttgaacatctggcctcaagtgatcctcctatctcagcctcccaaattgctgggattataggcttctgcactgggcctagacttctaccatatgtttctttctttctttttgatttttagtagagatgaggtttcaccatgttgcccaacctgatgtcaaactcctgagctcaggcaatccacccaccttggcctcccaaagtgctgggattacaggtgtgagtcaccgcacctggcccttatcctatgtttcttttatgaaactctgcaaccagtctttacattacattttcacacatgccaagctaaactctgtgattatgaaaatattttctgtgcctttgctaatacatgtctccttggtccagaatattatattcctcactcccacatctaccagtagaaatctatccacctttctttaatacccactgaaatatcaaccacctcctctaggaaactcctctgcttcattcaatcacatgtgatctcttcctactcttaattccattatcattcttcttaatctctctaatgattacaaattttgtcaaataatgcaaggatgtgtacacttattttataccacaacccaaactgaaatcttcttgaagagacagtaacttgtttatttttgaatctttaacaggacttagtgcagtcctccgcacaaagtatgtactcaataatgatttcttcaatggattaagtaaataaaaataaatgtgaagtaaagttatgagatttctcaggaagatcttaaatataatataagcaaggctctcaatataaatattttggtattttaaagaaattcaactaaagatatttaaagttctttctcaatacaaaataagtattagtatatggaattaggctgtattgctctacgggaaatcacaggaattttagtttgcagttctaaaatacctctaacttcaaaggcagtaagaaagacttggaacaataagagttagtctccatccattcgtgtgtttattcatcaagcatttatggaaagattattatgtgcagttgtgatgcctttgctacctaaatttcataaatatcatttcacttctattaatattttcatatgccatagtcacaaatctttattctaatggttcttcctaacctttttcttaccctgataaattaacccctgtgataaaaagtttatgatagcccctaagattcctactccctatatacaacctgcataaacttctccccgttgtgagataaaaataagactttacttccatgattagattatattatgtagtaaagatgaggggattttatgaacgtaattaaggtctacctcaattgattctgagttaatcaaaagggagattatcttgggtggacctgacttaatcaggtaaaagtccttaaaagatacaatgcacccctacagaagagacacctttgttggctttgaagaaggaaaaaaaaaagtagtagtagtaataagctgccacgttgttgagagggcgatatgactaggacctgaatgcaacctttaagacctaagagtcatccttagccaacagcccgcaaaagactgggtatctcactcagtcctacgactacaagggataaattctgcaaacaaactgagggaccctggaagctaatctttccccagtcaagcctttagtgagaccacaaccccagttgactctgctgggagccttgtaatacactgagaagaaaaacccagataagccatgcccagactcctgacccacagaaagagtaagatgataaatgtgtactgttttacggtgctaattttgtggtaatttgttatgcagcaacatagaactaatacaacccatctaattatatttcaatcctggttcctcatgtccccctgaaatgaattctaatgtaggctttctgtcttctgcatggtcagtatcaaacccctctgaaatgaaaactagatggcataatattaatacatttaataactgtagtcactgtactatgtgagaattgacaacggtactgacaaaagattataataatgtgctagtttgctcttatgactagattctgaaactcagtagcaaatttatttttgccaattttattggacttctgatttcagctccaatatgtaaagaactggaagtcatcacacctgttttacaacagaaaaaagttaaacaaactaaaaatcaattacttttctgggatccatctgagaactgaggtcacaaggcaaactgccaccctgaaatctggagagacaggtaaatacagagaatcaagatatgcttactgggacagcggccactaaaacataaattgacagaaatagttaaaagataatattgatgaattgctggagataaaatgtgaactagtttataagtgagaaactcctaggggctgcagtcttgccagggggcccacaatttttaagacattacctctaaaaatcccaccaggttcccacagtaaagagccaagaaaaaccctactgtttcgggcaggaggagggaaaaaaacaaccattttgaaatacgttcaacgcattctctaaaacaatggccagtctgccagtaaggaagattttaccagatccttatccaacatgagggaagggatcatttcctaaactgcagctccctgccatctaatcttatgatatgaatgaagagctcagaaacacttgtgaaggtcacaggcaagggacacagtcatgctaaaagactgagacataatcataggattagaagatgcttcctgttccccataccttaccaccacatcaacagggctccagtataacagcagaggattacaacagagagagacagctgcaaggctcagattctatttaagaataagtttttagggacattggagagtaactcaagtccacattaagaattaattaaagagcactggtcaagataactacaaggtagttataaaagacagtataaaatgtgttttaacatagatacaaaagacaatataaaatgtattctgtttttaattttttttatctgatttgaaagtcagttgcaaaaggtaataattgtaaatctgtgttgagagacatacaatgtgtaaagatgtaatttttataataatgacacaaagaaggtaggattaaaaaaacagctacataggattaaagcttctgcattctattgaaaattaaattagtattaatccaaactacataagatgttaaatataattcctaggacaaccactaagaaaataactaaaaaatacacagtaaaagaaaagccaagggaattaaagtgacacattagaaaaatttaaatggcacactagaaaatatctatctattagattcaaaaggcaatgatagagaaatagagggggaaaaggcaccagatatacagaaaacaaacagcaaagtggcatttgtaaacactaccttagcactaattacattaaacgtaaatggagtaaacactcaagttaaaaggcagagataagcagaatgaatttttcaaatgctccaactatatgctgtctataagagacacactttaaatttagacacaaataggttgaaagtaaaaggaatgttattcagccataaaaaggaataaagtattaattcatcctataatgctgaacatggaaacattacactatgtgaaaaagccagacacaaaaagtcacatattgtatgattcaatttagatgaaatgttcagaaaaggcaaatctatagaaatagcagattagtggatactagggaatagcaggtaagagtaggcagtgactgttaatgagttaggagtttctttttggggtcagaaaaatattctggaattagacagtggtgatgattacacaactttgtgaatatactaaaaatgactcaatgcaaaaatgcgcaaagaatttgaataggcatttctctaaagaagatgtctgaattactaataagcacatgaaaatatacttaacatcactagtcatcagggaactgcaaatcaaaaccacaaggagatactacactttatacccatttgaaaggctattatcaaaaaaaacataaaataacaagtattggcaaaaatgtggaaaaattggaatccttgtgcactgctgatgggaatctaaattatgcattgactataaaaaaaacagtttggtggttcttcaaaaagttaaatttaggctgggcgtggtggttcacgcctgtgatcccagcactttgggaggccgaggcaggcagatcacctgaggtcaggagttcaagaccagcctggccaacatggtgaaaccctgtctctactaaaaatacacaaaaattagccagacatgttggcggacacctgtaatcccagctactcgggaggctaaggcaggagaatcactggaacctggaaagcggaggttgcaatgagctgagatgatgccactgcactccagcctgggtgacagagtgagactccatctcaaaaaaagaagttacatttagaattatcacaagatccagaaattacatttctaggtatatatccgaatgaattgaaagcaaggtcacaaatagatatttgtacaccagcgttcatagcagcattattcacaacaggcaaaaggtagaaacaatccaaatacccatcgacagatgaatggataagcaaatgtgagattatatatatatatatatatatatatatatatatatatatatatatatatataaaatgaaatattattctgccataaaaaggattccgatgtatgctacaagatggacgaaccctgaaaacattatggctaatgaaataaatcagaaacaaaggacaaatactgtatgattccacttatatgaagtagctacaatagccaaattcacagagacagaaagaatactagagggtaacaggaaatggggaaaaggaatctggggagttattttgtgggaacagagttcttatttggtatgatgaaaggttctgaaaatagatagtggtcttggttgtctaacattgtgaatgtacttaattgtacacacacaaaaaatgctaaggccgaacgcagtggctcacgcctgtaatcccagcactttgggaggccaaggcgggcagatcatctgaggtcgggagttcaagaccagcctgaccaacctggagaaaccccatctctactaaaaatacaaaattagccgggtgtggtggcacatgactgcaatcccagctactcaggaggctgaggcaggagaatcacttgaacccgggaggcagaggttgcagtgagccaagatcacaccattgcactccagactgggcaacaagggtaaaattccatctcagaaaaaaaaaaaaaaaaaaaaaaacctaaattttgttatgcatgtttcaccacaattagaaatgacaataaatatcagaaaaataaatcaaagacctgcgaagcctctggggatgtgggactgggaagaaaggaggagtagtatagtgaagggagctgttgttttttcattataaccattctaacattatttaattatgtataagaattattttgataaattaagatatttaaagctaaaaaagaaattaaagaatttaatgacaaacaacaaggttttaatgatatgtattataaaggattactatccacactatatatttaaaagcccatacaaatcaattaaaaagacaaatacaaccaagacaagcagacaataaacaaaaattttaaaagaattaaacaaaaacctgcagaacaactaattgacaaacctgacaaaaacaaaacctgacaaaacctgacaaacttgataaaaacaagcaatggggaaagaattccctatttaataagtggtgctgggaaaactggctagccatatgtagaaagctgaaactggatcccttccttacaccttatacaaaaattaattcaagatggattaaagacttaaatcttagacctaaaaccataaaaaccctagaagaaaacctaggcaataccattcagaacataggcatgggcaaggacttcatgactaaaacgccaaaatcaatgacaacaaaagccaaaattgaaaaatgggatctaattaaactaaagagcttcagtacagcaaaagaaactaccatcagagtgaacagcctacagaacattctgtaggctcccatccctacagaatgggagaaaatttttgcgatctacccatctgacaaagggctaatatctagaatctacaaagaacttaaacaaatttacaagaaaaaaacaaacaaccccatcaaaaagtgggcaaaggatatgaacagacacttctcaaaagaagacatttatgcagccaacagacacatgaaaaaatgctcatcatcactggtcatcagagaaatgcaaatcaaaaccacaatgagataccatctcacaccagttagaatggtgatcattaaacagtcaggaaacaacagaagctggagaggatgtagagaaacaggaacgcttttacactgttggtgggagtataaattagttcaaccattgtggaagacagtgtggcgattcctcaaggatctaaaactagaaataccatttgacccagtcatcacattactgggtatatacccaaaggattataaatcatgctactataaagacacatgcatacgtatatttattgcagcctattcacaatagcaaagacttggaaccaacccaaatgtccatcaatgatagactggattaagaaaatgtggcacatatacaccatagaatactatgcagccataaaaaaggatgagttcatgtcctttgcagggacatggatgaagctggaaaccatcattctcagcaaactatcacaaagacagaaaaccaaacaccgcatgttctcactcataggtgggaattgaacaatgagaacatgtggacgcagggtagggaacatcacacactggggcctgttctggggtgggggcctaggggagggatagcattaggagaaatacctaatgtaaatgacgagttgatgggtgcagcaaaccagcatggcacatgtatacgtatgtaacaaacctgcacgttgtgcacatgtaccctagaacttaaagtataataataaaaaaagaaactatgagataataaatgtttactgttttaagccacaaaaaaattaaacaaaaacctgcagaacaattaatttcgcaatcaaagaaataaactcaattggaacaaagtatattcgaaggatttcctgtgtctttatttttaattgaaaagctagaagcagaaaaataatctattcattttgctaatatatatacagtcactaaaaagttatttcaatgttatagtctctagtcacccaattataaatgtctgtaactaaaattcatactgtctgctatatgcattccaagcctagcagaaaaatgtcacccagacatctcctatctccctttttgtgactatgactggaaaatataaaaaggttttagcaatgaaataagattttgctttaatcgcttacataactgtggagtgagtacagtttccataatcccataaataacttttcttccaatttcccggaagatatttgtctccatatttaagaattaaaagtcctaaaaattttattaggtaagaccaactccctacaagttttattgtatgacttagtatcaagcttaattactagttttcctaaatataactgtttggtgtagatgttcacactggaataaatcttgttttccattaagaattttaaaaaatgtttatgtccgttttatttttagatattcttgtaaaaaattatctgattaaatatgtttttgaaaacaaatcctttaatttatctacaagagactaattgctttagtgtatccctagctatcaaagtacatatatataaaaagacagtagaagagtcaaaagtatcaatattgtcattcaaatatattacttccaaatgcttctgtccttagaggtataacttcctgtggaaaaaagagaagggaacgccacttctctacattggatttaggttagattttgatgcaatgtgatgttactttcaggaattataagtggtttcaatgctttgaatgttaggaggaccaaaatatactaaataatttcaaattaactaaaaaaaattattaaaaatcagtgactctaaaaacaacaacaaaattcttctggccagatacatgaagtaaaatgagggcaacgaagaaagtttggcttagattaactttaatgaaaccttcagaaatgtaatttatgaaaataaaaaagaatgctctgtttctttctactctgaaaatatcgacttgtccctaagaataaaaatgtacacataaacacttctcctatataattaacattgcttgaagacttttattctttcagattattttattttattttagagagagtcttattctgttgcccacgctggagtgcaggggtgcaatcatagctcactgtaacctcaacctcctgggctcaagcaatcctcccacctcagcctcgcaggtagctgggactataggtgtgtgccaccacacccagctaatttttgtattttgtagaggcagagtttcaccatgttctccaggctggtctcgaactcctgggctcaagtgatcctcccagctcagcctcccaacctgctgggattacaagcatgagccaccacgtccagccttgctctttcagacttttaagtaaaacagaggtaggacaatcaggcagtatcttaatcaaaaaataagtaaaataatatgtgatgtttagaaaactatagttattaccttgttgcctctaccctaggaagcttactgtggtaattttaaaattagcatgccaatgtttaaaatgtaaatgattatttaaaggcaaaagaaacaataaatattcccttaccttgtaagatggcaagaaacacaaaattccttggctcacagtctggcacacagataacaaaagtgctcccacttcatcttggaactcaaatgtttcagtattctggaaggtagcacagagattccgacccttggggcctgacccaatggtaccaacccaaacctagaatatgaatatgtcattattagagttatgcctgaaaaaggcatggaaattagtatttatttggaagtatgtacataaaagatcaagcaacaagtttaacaatttatttttaaattgtcagggggttttctattaccttcaattaactgtatacagatttaatttataaatgttttgctttaggcagatcactaaattctcttagagtgaaaattctttgaaagcaaaacaggttgtacctgtctctgtttacttaatagctttgcaaaatttaagcatgtaacaaatttttgttaggtgaataaatgaacaagtgaaatgatgaatatataccaatagcagaatcagataattccaaaatactggggatgaaggccagtataatttgccataggcaagtctcaaaaatgacaggaactactctttcaaaaattcattggtgctcaaagatccttagtcatagaaaaaaatcacacccaccacatcttacagttatttgcaattctatctcccttatatgtagagagagggagagagacgaaaattttgcaaaatgttaacaagtgatgaatctaggtgaaggatataaagatattcacaatattaatattatctttccaacttttctgaaatttctaattttttgaaatacagttgaaagaatcaatgaatgaatgatcaaaatgtagcattgagtagaaaaaaacaaactttttcctttgctctcacaccacagcaatcaacacagaaaacttctgtgaccaaatgtgtgggaggttttttccacataccaagcaatcaattctgcaacagacatcaactgggtgtcctctaattcaattcaattctgatactatctactgggagatgataacttcagatcccagaggttgagggctcagtccccaagactgcccctgatttctgtggccagtcacagttgtttttacttgcacttctgactgatcagctagaaatcgggcatcccacaaccctctgcttgggttcaattaatttggtagagctgctcacagaacccagggacacatttacctacacttactgatttattagaaaaaatattacaaaggatacagataaagagatgtatgtgggaaggggctcagagcttcaattcccaggcacatcaccctccaagtgttcaggaacctccaagtgttcagctatctggaagctctctgaaccctgtccttctaggtttttatggaggcttcattatgtaggcatgattgattaaaccactggccattaagataaacttaacctttagcatcatctcccccaagccctccccagaggttgaggaatgggggtgaaaatcccaactctcctatcatgtcttggtcttcccggaggccagccctgatcctgatgctacctaggagccccctaccattagtcatctcattagcatacaaaatgtcactttggagattctaaggattttaggagttgtatgccaggaaatggagtggaagaccaaatatatatattataaatcacaggcatatacacacaagaaacattattcacctttaaagaggaaactctggcacatgctacaacatagatgatgaactttgaagacattatctgcaacatggacaaaccttgaggacattactaagtaaaataagccagtcacaaaaacgacaaatattttatgattccacttatatgaagtatttatagtaatcagattcatagaaacagaaagcagaatggtagttaccaggagctgaggagagggggagaatagagagctgttgtttaatgggaacagagtttcagtttttcaagaggagaaagtcttgagattggttgcacaacaacatatacatacttaataagactgagctgtatactcaaaaatggttaagatggtaaattttatatatattctaccacaattaaaagttttttaaattttaagttgaaggaaaatattacagtgtaacagaatccagctgcacaactacattattataattcagcgacattataggttaaatggacttttgtgcagtaggctgagaacatagagcctgtaataacattcttatgagaaaacgtatgctgaatttaccaacttcccatatttatctgtgtctctaaagaaagcaacttgaaaacgtggaacatgtattcttccttcactgctgttcatagtcacatttctcagcaggaacactctggcatttggaaagcacaattcttagttacataggagtagcattctaggttcccaactaccaaatgccgtagttgctcccaagtaattgtgaggaccaaaaatgttcccacctataaacctgtgtatatagcttaataaagagctagcattgtgttaggcacacaggtgatgttaacttaaccactttagcattaaccacagcttaacatcacctgtgtgcctaaccactttaaaatatttcataaaaagaaaccagaaggcataatgaaaaataacaatctatacatttccacaaaaattaaaataacatttagtagccatcgttataaaacattaaaatcttaaatcataggactcataagagtacttttaactcatttcagatattcaaacatgctgaacacttgctaaataccagctattaatctaggcacagaggaagaacgtggatgtaaaaggaagaatcaggatacactgtagtctaatggaaaagagaagcataaataattatacaagctgggcatggtagtacatgcctgtagtcccagctactcaggaggctgaggcaggtggactgcctgagcctgggagtttgaagccagcctgggcaacatagcaagcgaccttgtctctaaaaagataaagtctggccaaaatggcaaaaccccgtctctactaaaaatacaaaaatgtgccaggcatggtggcatacacccataatcccagctactcagaaggttgaggcaggagaaccgctggaacccaggaggcgaaggctgcagtgagccaagatcgcaccactgcactccagtctgggtgacagagcaaaaccttgtctcaaaaaataaaagtaaaaataaaaatacagagataaaataaataaaatataaataggctgggcacagtggctcacgcctgtaatcccaacactttgggaggctgaggcaagtgcatcatttgagggcaggagttcaagaccagaatggacaacatgctgaaaccccatctctaccaaaaaaataaaaaattagccaggcgtgcggcgcacacctgtaatctcagcaacttgagaggctgaagcaggagaattgcttaaacacgagtgagccaagatcacaccactgcactccagcctgggcaacagagccagactctgtctcaaaaatcaatcaatcaataaataaaacataattatacaatgagaattaaagaaatcaaaattttgttttcattacaaaactaaaacttacttagaagaagaagctgaaatacagccttttgcatcccaagtgactggattatttccttctgttgacacatcattaagtagctgacagattttcttttattgtaaaactggaatgttgaatttcctaccaagatttacttgctggcacttcaggtatcttctaacttgtttacatagttatattgaagtagaaacactgaaggccttccaaaaaaaaaaacaacaactaacctgtgaatttttaatgatatgattagcctccagctggatagtaaatgtaacaccaagttctgacgaaaaggatttcattggtgataatgtaccagatgtcaaaacaatggtctgaactttgccattaatatctgaaaaggcctaaaagaaaacaacattagataaataaaattatctttagaagaggctgggcaaagtggctcacacctgtaatcccagcactttgggaggctgaagcaggaagatcgcttgagtctaggagtctgagaccagcctgggcaatatggtgaaaccccgtctctacaaaaaatacaaaaattagccgggcatggtgacacatgcctatagtcccagctactcgggaagctaaggtgggaggatcgcttgagcctggaaggtcaaggctacagtgagctgagattgcaccactgcactccagcctgggtgaagagcaagaccctgcctcaaaacaacaacaacaacaacaacaaacaactatctttaaaagagtcaaccacatttattaaaatgctggtactgagcaagaagacaaaatttccatttacatgatgagcttaccacagctggatttaagcaccaaaagtttagcacatgaactgcagttttctgtcgtgaacgtttcttattttttggtagaaccaacaacccatttttgtctgaaatatcaatctgatttgtccaggagtaagtctgttgaatcgcaattttataatcatctgcaaatctagatgcaaagaaagtgctaattaagtggcaaaacttttaaaacctatgacccagctacatacaatataaaaactgattacattaaaactcatttgaaagagctggtaccttcccattcaaaaataacattctccttaccattaattaaattcagtaattctgatattaataaagccattaatcatagaatcaactgattctcagagggaatattagaaatcatctaaccatctatcttttatcagctacttcctaacctcattttcaaacattacatatgtcatggcagaagcagaattaaaatccaggcctcctaactcccagtccagagctctctgcatgctctactacatgatgagttacaaaatagaaaacagtattatggttaaagagactaaaaggtgggttatacttttttattaaatgagaaaatagaaggataatttttactgaaagtttttaaagtattagcaacaagttataaaattcttacattcctttgaaacctgagtctcagccttcatctacttttaatgaaggtgctgaaccagatgatatctgtgatcccatccagtgtataactgctttcaataagaatctagaaaatggttaagcttaaaaataataaaatgtggccaggcgcggtggttcacacctgtaatcccagcactttgggaggccgaggcaggtggatcacgaggtcaggagttcgagaccagcctggccaacatggtgaaaccccgtctctactaaaaatacaaaaattagccaggtgtggtggcagtcgcctgtaaccccagctactcaggaggctgaggcaggagaatcacttgaacccgggaggtggaggttgcagtgagctgagatagcgccactgcactgtagcctgggtgacggagcaagactccatctcaaaaaaaaaaaaaaaagtaaaatgtgatattctctaccatgtcttcgctatagtatttccttattcatttcactcctagagagattagatctcaaaattaaagttgggaacttaacaactgtggagagaatcttataaaaatactgggagccaaaaactgtaaaactcttaaaacaaaggggagaccgggtgcgatggctcacgcctgtaatcccagcactttgggaggccgaggtgggcggatcacgaggtcaggagatcgagaccattctggctaacacggtgaaaccccgtctctactagaaaatacaaaaaattagccgggcttagtggcgggcgcctgtagtcccagctactcgggaggctgaggcaggagaatggcatgaacccgggaggctgagcttgcagtgagccgagatcgcgccactgcactccaacctgggtgacagagcgagactcccgtctcagaaaaaaaaaaaaaaaaaggaaaagaaaacataggggaaatcttcatatcagatttggccatgatttactggatgtgacactaaaagtacaggcaacaacaagaaaagagaaagataaattggacttcaacaagattaaaaaccttttctgcatcaaaggacactatgaataaatgaaaaaacaacccacagaatgagagaaaatatttgtaaagtatatatctcttacaggactgacatccagaatatctaaataactacagctcaacaacaacaacaaaaaaacccaataattcaaaagtgggcaaaagacttcaatagatatttctccaaaaaaagacatacaattggccaataagcacatgaaaagattctcaacttctctagtcattaggaaatacaaataaaaaccagatgatatcacttgatacctattaggatggctattatacaaaaaataaaataacgagtgtcagcaaaaacaattctgatatatgctgctacataaactgttggcaaggatgtgtagaaattggaacccttgtgcattgataatgagaatggaaaataatgcagttgctgtgagaaacagtatggtgagtcctcaaaaagttaaatgcagcattaccatatgattctgcaattccactgctaggtacttatatacacccagaagaactgaaagcagaactcagatatttgtacaccaacgttcatagtagcattgtttacaataggcaaaagatgaaaacaacccaaatgtccattaacataagaataaattaacaaattgtggtatatacatacaatggagtattattcagccatagaaaggaatgaaattctgatatatgctactacataaatgaactctgaaaacactgtgccaaatgaattgaaataagccagacacaaaaagacaaacattgtatgatcgctcttatgtgaaatacctagaaaggacttcataagatgaaaatagaatacaggttagcaggtgatgaggggaaagactctaggggttattatttcatgggtacagaattcctatttggaataataaaaaatttctggaaatctctggaaatgagtacaatgtacaacattgcaaatatactgtacttaatggcactgagttgtatacgtaaaatggttaaaatggtgtattttatgtatattttaccacaataaaaaaaaaataccagaactaggtactactataatttaagttacaggatgagagggattggttgtttttcattttccacagttcatacaataaaaaagggactttctgacactgtaggaacataagaaaactttagcaatagaagataatttaatatgaattaattaaatatatttatttaatgatttcaatattcattaatataaaagactaattttggccaggcatgggctcacacttacaatcctagcactttgggaggctgaggtgggaggatcacttgaggccaggagttcaagaccatcctgatcaacatagtgagatctcatctctacaaaaaaaataaaaattaaaaaattagctgggtgtgggtgttgcacacctgtaatcccagctactcaggaaacaggtgggaggattgcttaagcccaggaggtcaaagttgcagtgagctgtgatcatgcaactgcacttcagcctgggtgacagagaagtccctgtctcaaaaaagcatcagactaatttcaattcctctaagtctgtgtcaataagagcaaatatataataatatctataacacttgttttcaattctcctatttactcacccaaaataggtatgtattaaacacatgctagcatccaaattaggctatttttaaaaggaaaatacatactagttatcttcacttacctgctattttgcctaaaaagatagtcaagtaccataaaaagtcctttaagcattatttgagttgatgcactaataacaggtacttctcttgcctcctctttaccataaattggtgagattttttcctctttttgaagaacagcagaaaaatgtccctataagaaattaccatattaagtatagaggggttgggagggaattggaaaaagaaacttctcaaaaatacaatgaaacccaacaaaacatgcattgaaattttaggtgaacagaattgctatagtttgatagtctgacttctccaaatctgatattgaaatttcatccccagtaatggaagtggggcatatcaggaggtttgggaccatgggggtagatacctcatgaatagattaacgccctccctaagtagagagggggtgagttagtgctctgttagttcccacgagagctggttgttagaaagagcctgacacctccctcccttctctctctcgcttcttctcttgccatatgatctgtacaaaccagctccccttcaccttttgccatcagtggaagcaggctaaggtcctcaaaattagatgctggagccatgcttcgtacaagctgcagaactgtgaaccaaataaacctcctttctttgtaagttacccagcctcaggtatttatagtgatacaaacaaagacaattattaacagcacaatgctaacaatatattttagattgccatattagcaaaattctcctcagagtcaaagactctttaataataagttaggtatttagaagattcaatcagggtattatttggtgcctttcagatgtaaggaattaaataaaaatgtagcaaatggcttctccaaaaggctttaaagagttgatatgttgccttaaatccatgagttatttccagaaagtaattctgatgctgagaataatatgactacatcacaagcaaaactaaatgacttaaggaaacaatacatgctaaataacaaaatgtatgttaactggttctttggtctttacatggctcataagttttaatatggatgaaagtgtggacattaaaacaaaattataagaaaacagaagagtatgttccccacattgtaaaagagaaaattctgaatactgaagcaataaaagatattggtgacaactctgatgtctgacaataaaatgtgttcaatgaaaataaaatggggaaataggataaataagaaagatacattttaatattcagatatatcatgaaacaatacaaatctataagagtaaaaatcagccttcactgaacaaaacattacagaatacaaataagtagaaagacttaaaaaacaaaccaacaaagaacaaacaagaaagacaacatgaacttggaaaaatattcagccctgcaatgaaatacaaatatcaaattattctttttgaaaaatgaagtcccctcagcaacaccttatatcaaagcttaatatccttctccaacactggcagaacactggcaatttactctgataaaatatagaaaatataaactactaaaggtgaggcgctgtactaaaaacagaagggctaaatgaaatcctacatattcaattattagagtactccgagctcaattccagttagcttacctacacattggcagctgggttggggggggtagaaaaatagaaaactaagcaatggaaaaataaataaaaagttaattatggttccttaggaaacatacataaagctgtataaaaatataatcaagtaagctacatgcattactatcatgggctataataatacatacacgtgatgatatccacgctgaatatcaatataactatactggcagaatagagagtgaagagagagaagaaagaagggcagaaaagcaggaagcaaaggaagaagagagagcgcatgagtgagcgtgtgtgtgtgtgtgtgacagagagagaagagtgtgtgtgagtgtgtgcgtgtctaaaggagctaaatcatcattatacttaacagaaagtcaacagacaacatctaaaattgaaaactccaaagaagcagaacccacgttatttttttaaatgtggttaaatatcagaagaaaccactacaagagataaaagtgcactccaaagtgagaattgggattgaggagtgggtcagaggaatgcttttctcaacagaactgactctttatattctataggtatactactttggtaaaaattcctaaaaatattgataatcataatgatgaatgtcttagccaactagaattagggcaaatttccttaatccaataaaaataactataaaatatataatataataaaaatataatatatatattatttaacagctctaattaattttttaatttaattaactgaattgaattgaattaaaattaacagctttattgagctataattcacatgcttacaatttacctttttaaagtgagttctgtgggttatttctatattatatattttatatattatatatttatatataatatagtatatattatatatttatatattttatataaatatattttatataatatatttatataaaatatatattcatatatgtattaatatattaatatataaatatattttatatataatgtaaatataaaaatatatatttatatataatatatttatatataatatatttatattatatttatatttataaatttatataaatttataaataattttataaaattataattttatttataaataaatttatataaatttatataaatttataaatataattatatataaatatattgcatgtttatgtattaataaatatattaatatataatatattatatactatataaaatatataaaaatatattatatactatataaaatatataaaaatatattatatactatataaaatatataaaaatatattatatactatataaaatatataaaaatatattatatactatataaaatatataaaaatatattatatactatataaaatatataaaaatatattatatactatataaaatatataaaaatatataaaacattatatattaatatattatatatagttatatactatatatagtatatatatttatataaaatatttatataaaatatataatatatatactatatattatatataaatatataatataaaaataacccacagaactcactttaaaaaggtaaattttaagcatgtgaattatacctcaatagagctgttaattaaaaaagaatttttaatcacagcaaacatctcaataaagggtgcaatactagtaattcctcctttaagcaacaaaaccaagatgtctttttcttttttttattttattttatttcttttttgagacagagtctcgctctgtcgcccaggctggactgcagtggcgcgatttcggctcactgcaagctccgcctcccaggtttgcgccattctcctgcctcagcctcctgagtagcggggactacaggcgcccgccaccacgcccggctaattttttgtatttttagtagagacggggtttcaccgtgttagccaggatggtctcaatctcctgacctcgtgatctgcccgcctcggcctcccaaagtgctgggattacaggcgtgagccactgcgcctggcccaggatgtctttttcattgcctgtgataaacaacgcactggaagtctgagacagcatagtaagaaataaaaacatgaagaaatagaaagaaatgagaaagaaagaaaccggtcattattcagacaatatggttatctacatggaaaatttaagataatctgtgggcaaacctgtaaaataattcaacaaggtggttaataagacaattataagataaatatataaaaatcaaacaccagttttatatagcaataacaattaaaaatgtaattttaaaaatatatcatttgtaatggaaacagaaactttaagctacccatgaataaatctaaccaaagccatctaatcttttaaaaataaaattataaaattttattgaaagataggaaaaaataatgaagaactatatcttgtcatgaatgggaaaacttcataatgtaaatgcatgaattctttccaaattaatctataaatacagtgcaattacaataaaaattctaacagaacttttcctagagattataagctaattctaaaattcaaatggcagagtaaaacaaaggacagctgatacaattccaaaggaaaataataaagtgagggatatgccctactatatataagcacttttcttaaatcattaattaacaaatgtgacaaattagagaaacagaatggtggaacacaatcaagtccagatataaattcacacatatatgaaaattagagggactgggtgtagtggttcactcttgtaatcccagtgcttcaggaggccgaggtgggaggatcgcctgaggccaggagttcaagaccagcctaggcaacatagtgagaccctgtctctacaaaacataaaaaaaattaagccaggcacgggtggctcatgcttgtaatcctagcactttgaaagaccgaggcgggtggatcacttgagctcaggagctggagaccagcctgggcaacatgacaaaagcccgtctctacaaaaaaatacaaaaaatgagccaggtgtggtggcatgcttgtggtcccaggtacttgggggcgctgaggcacaagaattgcctgaacctgggaggcggaggttgcagtgggctgagatcacaccactgcactccagcctggttaacagagtgagattctgtctcaaaataaataaaaaaataagtaattagccaggtgtggtaatgcatgcctgtagtcccagctactgagcaggctatggcaggaggatcccttaagaccagaaatccaaggttacagtgagctatgaatgcaccattgcacttcattccgggcaacacagcaagactctgtctcttaaaaacaaaaaaaaaaagttaaaaaatatttttttaattaaaaaaagaaaaataggtagcagaggtagcattacaaattagagggaaattaatatgcactatttaggaaacagtgctgggacaattctctatccatacaggaaaaaaattgattcataaattcacaacataaacaaaatgaatccaaggctttgtaaaagtatttataaatcccatcctttataaacaataaataccttaaaatttttaggagaaatataacataggataagaaaagactgcttaaagtaaattcaaaaaccacaattcataagggatgttcatatcagaactgtttattatactgggaaactggaaataacctatatttcaacttatcagattaatcaatttagaataagtacactcataaaaatgctacaccgcaattaaaaattatgtgaaggaatctttacttaaataggaaagtatacacaatatattgttaaattaaaagagggtaaaacaaatgtgtcatatcagaaatatatttataataagcaaatatgggcatagaagaaggcataagtagacataaatcacaatgttcacaatgttcatctttgagtttatgagtcatttttctttgtgccattctgtattttccagtttttttcccttgacatacattttatttgtacattttataactgacatgtaacatatacagtagaatgaactaatcttacatgtacacctcactaaatacacacacctccccatgtaactaacacctgaccaggatacaaaacattcctagaatcccagatgttcctttgtgccccttctcagcccaaactgcttcaaacccccacatagttgtaaccagtacttcaacttctgtcaccataattaggtttgtctgttcttcaacttcataaaaatagatttatacagtttatacccttttgaatctggtttccttcacctaacataatatttaagattcatctagagccaggtgcggtggctcacacctgtaatcctagcactttgggaggccatggtgggcggatcacctgaggtcaggagttcgaggccagcctgaccaacatggagaaactccgtctctactaaaaatacaaaaaattagccaggtgtggtggcgcatgcctgtaatcccagctacttgggagtctgaggcaggagaatcacttgaacccaggaggcagaggttgcggtgagctgagatcacgccattgcactccagtctgggcaacaagggcgaaactccatctcaaaaaaatatatatatatatctatatggttgtgttggaagcagttcattttttattgctatgtagtattccactatatgaatatgctacaatttatccttttctttttttttaaacagggtcttgctctgtgccccaggctggagtgtagtggtgcaatcatggttcactgtagcctcaacctcctgggttcaggtgatcctcctgcctcagcctcccaagtagctgggactacagcgcatgccaccacaccagctaattttttgtagacaacagggtctttgttgcccaggctggtcttgaactcctgggctcaagcaatccgccagcctcagcctgtcaaagtgtaggattactggcatgcaccaccaccctcagccccattttactgttatataagtagctttcaaaatgcaaacaatttatgcaaaaaatattatgaaagatgtcacaatatggaaaaattcttacaatagataccacgctgcattttataaatgcagggcaaaaatttaagtatatcctgattaaaatatatgttttaatagtcacatgccccgaatactattgtgatcatgttggcaattattttcatcattttttttgttcaaaaacctctttgagaaataacatcagcaagatggaagaatttgagttttctagccaggcacggtgactcacacctgtgagcccagcactttgggaggccaaggcaggtggatcacttgaggtcaggagtttgacaccagcctggccaacatggtgaaaccctgtctctaccaaaaatacaaatattagccgggcatggtggggcatgcctgtagtcccagcaactcaggaggcttagacatgaggatcacttgaacacgagaggcagaggttgcagtgagccgagatcacgccactgccctccagcctgggcaacagagtgagacttcatctcaaaaaaaaaaaaaaaaaaaaaagaatttgagttttccattgtcatctcccaacaatgaaccaattctgacaaccactcgcacatgagagtacctttgttaagagtctaagagtctagcagagaagttccaggaccccattggagcaaaaaatccaagaacagatgcattgaagagggcaaaaagtatagtttcatattacctacatcacccctgccccatggaagtacagcccagtgccaagagagcctccattagcccacaattcttcccatggggaaaagtgagaatatagtgagtgattaatgaaataaagaaaatgtggcacatatatatataatgaaatattcagtcttaaaaaagaagaaaattttgccatttgcaacaacatggatgaaactggaagtattatgctatctgaaataagcctgatagaaagacaaatactgcatgctatcactcatatgtgtcatctaaaaaagttgtactcgtagaagcagagtcctcagccccaaagttcactggtgaagatgtacaacaacaggaacttttgttcattgctggtgggaatgcaaaatggtacagccactttttgtcaagagacaaagtcttgctatgttgctcaggctggagagcagtggctattcacaagcatgcaatcatagtgcactacagcctcaaattcctgggttcaagcaatcttcctgcctcagcctcccaagtagctgggactacaggcgcataccaccatacccggcagttacagccacttttgaagactgtttagcagtttcttgcaaagctaaacacagtcttaccatataatacaacaattgtgttcctaagtaaatatccaaatgagtagaaagcttgtcactaatagaaatgtttataatagctttattcacaattgccaaaaactggaaaccatcaatgtttctttcagtaggtgaatggataaactgtggtacatccaaataatgaaatatcattcagctataagaagaaatgagctatcaaggcaaaaaaatacatggaggaatcttatatgcatattgctaagacagaagccagtctgaaaagactacatactgcacaatttcaactatatggcattccagaaaaggcaaaacaatggagttagtaaaaaaaatctgtggttgccaagggtttggtgggtgtgggcgtggtgtggtggagggatgaataggtgtaacacagaagatttttaggacagtgaacacactctgtatgatactgtagtggtggattcctaggcaaaacctaattgaactctacaacacaaggggtaaaccttaaggtaacctatggactttaatgaacaataatgtagcaataatggttcatcaattataacaaatatactacactattgtaagaggtcaataatggggaaactatggagttggtgtgtattgtgtatgtgtataggtgtgtagctgggagtgagagtatatgagaattctggaccttctgcttaatttttctctaaacctaaaacaaccattctaaaatacataatctattaatttaaataagtaaatagcaacaaaaaaagaaaatatattcttatcggcaaaaaacattagaaaatataatgaaatttatgcagcctctatttagaaaaatgtacaaaaggatacgctcataaaattttgcatatagcttcataagtttcatagtcttccaaagcctactcatgatctctccatgaatatcaggtttataatttctaaattaagatgaacaaaataggaatttctcctctttttattttctaaaattttaacttttacaattgactaagtaagtctacaacctaaaaatattgactccttttagctttaaacaaagcaattaatccctttaaagactaatttccttcaaactaaataatgctatattttcacccacaatttacccatgccatcacttaaagattatcaaatttagataataaaatttttaaaaaattaaattgctatatttaacaattctgggttactcactagatttaatctggatttagtcacgactaaatcacttctaattcactaaatacgtttcacaggtagaaaaaatatcttacctgcaaaatgggaaaagtagcagtggtgatacccattttgtgtaaagttaagagcatttcatttccactccatattttacaagctgattcataatctctttctacaagatattcagcgtttgcttctaaccaactgaaataaaataaaacaattgtgtcaaccagtatcatccttacacacactatttcagcagaacaagagaatcatcattattgtcatgcgttgatctgtatatcttgacattcttaggacatgaatgtggattaattaagacaccttttaaaaagcattcatgtctcatattgcaaaataaactttatataactgaaaagaaagcttttattcagaagaaacaaacatgcatatgatttcccacttgttttgtggattcacattgctttacttagctaatgtctcttcaactgagagccaggtcttaggtacttatcccaaatatatataaaaggaaatgcaaaacaacctaataaagaaattaaagtgttacatattaacacaaaaaagagaattactagaaaatatataaatacattcattgatatatatacacattcatacattaagtgtttgaatgcctattttattccagacattgttctgggtagtgaaaataacagcagttaaataaaataggcaaggtccttactctcatggaaattacattctactaaagggagacaggaataaacatcaaaacaaatacataaacaagaaaatgtcaggtagtcatgggatactatatggccataaaaaagaacaagaacatgtcctttacgggaacatagatggagctggagatcatatccttagcaaactaatgcaggaacagaaaatactgcatgttctcacttataagtgagagctaaatgatgagaccacatggacaagaggggaacagcacatactggggcctatcggatggtgtagcgttggagacgggaaaagatcaggaaaaataactaatgagttataaggcttaatacctgggtgatgaaataatatgtacaacaaacccccataacatgtttacctgtataacaaacctgcacatgtacccctgaacttaaaagttaaaaaaaaaagttctaataaaatgaaaaaaaaatcaggtaattatatacaaataaattatgaaaataaattagtttaatataacctagttgcaactttaattttaatgtcaagaatgctctccctgaggtgacatttattctaataactgaatgcaaaaagtatccagtcatgggagaatcagaagaaaagaaatatccaagcagaaggaacagctaggagaaaggtcctgaggtggttagcaaattttgcttgtttgaggatctgaaagaaagccaatatgatttgcagcacaatgggcaaaaatgaataatggtatgaacaatgtcagagtgataagcagaaatcagtcatcttgagtcatcataagaaatctgaatttattcaaagggcaatgggaaggcttgggttttaatttttaattttcgtgggtacatagtaggtgtatatatttatggggtacatgttttgatacaggcatgcaatatgtaataatcacatcatggaagatggggtatccatcctctcaagcattaatcctttgtgttacaaataatccaattatattcttttagttatttttaaatgtacagttaaatcattattgactatagtcacactgttgtgctatcaaatactaggccttattcattcattctatttttcttttttttgtactcattaaccaacccctcctcccctgtgatccccatctactacccttgccagcctctggtaaaccatccttctactctctatctcaatggattaaattgttttcatttttagatcccacaaataagtgagaatatgtgatatttgtctttttgtgcctggcttatttcacttaacatagtgacttccaattttatccattttgttgcaaatgacaggatctccttctgtgttatggctgaatggtactccattgtgtatatgtaaccacattttctttatccattcatctgttgatggacatttaggctgctttcaaatcttgactattgtgaatagtgctgcagcaaacatgggagtatagctatacctttgatatactgatttcctttattttgggtatatatctagcagtgggattgctggatcgtttggtagctctattttaagttttttgaggaacttccaaattgttcttcatagtggttgtcctaatttacattcccaccaacagtatacgagggttgtcttttctccacatccttgccagcatttgttattgcctgtcttttgcataaaagtcattttaactggggtgaggtgatatctcattgtagttttgatcagcagagaaatgctgatcaatgatgttgagcacttttcatatgtctgattgccatttgtatgtcttctttagagaaatgtctattcaaatcttttgtccatttttaattagattattagattttttcctacagagttgtttgagctccttatatattctggttattaatcccttgtcagatgggtagtttgcaaatattttctcccattctgtgggttgcctcttcactttgttgattgtttcctttgctgtgcagcagctttttaacttgatgtgatcccatctgtccatttttgctttggttacctgtgcttgtggggtattgctcaagaagttgttccccagaccgatgtcctggagagtttttccaatgttttctggtagtagtttcatagtttgaggttgtagatttaagtctttaatctattttgatttgatttttgttaatggtgaaagataggggtcttgttttattctcctgtagatggatatccagttttcccggcaccatttattgaagagactgtctttccctcagtgcatgttcgtggcacctgtgtcaaaaatgagttcactgtaggtgtgtggatttgtttctgagttctctattctgttccattgatctgtgtgtctgtttttatgccagtactgtgctgttttggtgggaaggttttaaatacagagttttaaataaaagacagactatctattttacatgtttaaaaaatgattctagctaccaagctagaattgattactggagaaagatgagtgaaagtagagacctgttaggaggttgttataatatccttgttgagagaagactgtaggatgatagatatgaacaatcaatgatattgaggaattggggatatactgtgcaggtggcatctataagacttactcattaattggatgaggtgaggcagtaaggatattattaaggatagtatttaacttttttacttgaggaactggtacatactgtttgcttagatgaggaagactggggagaaaacagaacacaggaagaaaagcaattctgttttagatgtaaagttgaaatgtctattttgatatacaaaaagagctataaagtagatagttggatataagaatggcattctagggagaggctgaagctagagacataggttcaggcctcaccagtactgaagattatattctaagctatgatactacaaactatttagggaaaatagaactacagaagaaactaggtcccagtaccaagtaagtcctgaggcaccccaacatttagatatcaaaaagaataagaattatcaaaagaaatgataaaaaaaaatggactgtgagaaggaggaaaatcagagatgtcacagaagctaagacaaaaatgcttttcaaagaggtagtcatcaactacccaacaccactgactggctgagataaaagaaacaataacctctgtatttggcaatatagagatcactagtgatcttgatcaaaacaatttgcgtagaattgtccagagagcaacactgctaagatcgatgagggaagaatgtcaagtgagaaaatagaattagcaactacagacaactctttcaacaagttatgctgtaaaataaaaaccacaaaaatctgcatggcaaaacaccaccagaaaagacacaaatggcaaaatggggtggaaagtatttacaactcttattgcaggcaaagattttaattccctaatatatgaatggggttctataaatcattaagaaaaagaaccacaataagaacaaaaaaaggacagagatgtcaacaggcagttgacacaaaaggaaatataaatggcctttaaagatgcttaatttcatgcatattaaggaaaatgaatattaaaactacattgggataccacttttcacctagcaggctagaagaaaacaatttccatactcttatacattgctggtagaagcatacattagcccccctgtcatccacctattgtgtaaatgttagctatatctattgaaattacaaatatgtctatgctttcacccaacaattccacttctaaaaagattcctatagatatatttgcatacaaatgaaattatgtatgttcaaagttattcattatagcagtttaagagcaaaaaactagaaacaacctcctctattaatcagaggctggttaaataaactattgcatatctatctaatggaattctagatatctattttttttttaaactgagaaagctttctaggtcttaacatggaaaggtctccaaaatacactgggaattgaaaaaagcaagttgcagaaaaatgtatgtatactatgccatcatttggaaaatagtgagggacaagaaaatatatctaatattggttcataaatgagtaatatgcattgaatgataaacttgtttaggagaacacagacattaaacagatcagggatggaactggaaggaggtttttactatacaaatttgtatacattttgtcttcaaaccatgtgactgtgtaacattcaaatttttttaaagtgtaaatgtgaaggaaacagaaaaatgctacaataaatgaaatgggattcagagttgggaagtttttttctatggtaacaagattctagatcaacaacttaatgtctaataaaagaaaaattagtgtaaattgtcataaagctagtactaaatatattattttagagtactgaaatcttgattaattaaaacccatgaaactagaatcctatttaaattggagaataaagcaataaattaaagccatgatccagaattttaaaatatattaattaaaagcacgtttagtttctggttcaatatcctagacacatccttcggggctattatgttatcctgccaaattaggaataagattcacaattattcatttttaaaaatcacttatgttcattatattcttgtgaacaagacaaaaggttggactagccttgtttttaaagcttaactggcaaggaacaattcatttcccaagaagcctagttaaccaaagtttactaactttaaatactctggcataatcaaacatatttttcatataaaggcagcacaaatacactaatagacaaatcttcttacttaatgaggctacagcacacagctcgtaggggttcatgatctttcttccttatattattgttgaccatactatctagttcatcccgagcaaaccgaagctgaacttctgttacactgtaacttgctgattcccgagcacagtcctcgatgttatgagcttcatctaaaatgacaacctgttctttcagatttaaatccatctataagataaaagaattttcttgtaaaacatttggcaaaatagatttaacaacagcaggcaagatatttcattttaaaattcacactataggccaatattgcaaatgcaattacataagcaacagagattctaggtcttaaataaaacttctgaagcactatggccaaccaaatatcagctctacattattgtgtttgccatttttttactcttaaaatactttgcaaacctatcgacagcaacaaaaatatgtcagtgagtctggatcaaggtaccacaggctacacagaaaggtataaaatcccactatgaaagtgtttccaacataattttacaaactaattataagacctttaaaaataaaataacaatataagattaaaagcataagacctttaaaagtaaaataacaatacaagattaaaagcataaaacctttaaaagtaaaataacaatacaagattaaaatgactcttaaaatactaacataatatatatgttttgttttaatctgtcctgcaatccttctccaacatgggagttggagaattgttcttctccaactcccatgtgatgctggcaggactattaacctgtgaatcaaaggacatcaccactttcacaatgggggtaaggcccaggattgtcagttaaatagagtattccatccttctgtggtcacagtgactggttcaggttcaggaacagacatgtgatccacggtaggccaaccagagtaatttggagggactaatatgtggccatgctcttttctccaggtgagctagctgcttatggttatctttgttccataagcaagagtgagaacttgcctgagaataaagccatcacaataaagagaatcaagtattttttcttaagcattttttagggtaggtttgtgtcacttgcaatagaaagagtactgactaatacaaggaactgtcacaaagcaattgtcaatgagttattttgaggaagtaagaatacacagacagctgaaaagttgagaaaaaaccatggaagagtaggaaatatattggggtttaaaagataagtaggatttggatttggacagacaaagagtggacagggcattccaaagaggtgtcatcaaaagcaaagatagagtaaagaacaaggtatcctcaagggagtatgaggtaatttttgaaggattcaaattgggcagatccaggtcagactattgagcatcttgagtgtaagcttaagaaatatgaactttatctcatgagcaaaggaagcaatagatttttaggcagataaaaggatgtaataaaagcaatatttggaaaactgacctggcattgatatgcacagtgtctcagaaggaagagatctagttaagagataaccaaataagaggtaggatagaaggggtagtaaacataatgcctattaatttggatcttaaagaaatagaaagctgagatgaattgtgcagtcagatctgaagacaggaataacaattgactatgccaaattattcactttttgtcttatgctgttaacaattgcatgtctctaaaaagaaccaggcaaacaaacacaaatgccattatctgaagttcacttctcaaagctcacaaggcataattttattcccccacataagttcttcaaaacatagttttttcagagaaatgtttctaaataattctttttgaatattcataaatgcaaagagaaatcctgctgtaatcccacttcagtcactatgttccttaaaactgcttgttattttggaaattaaaatgagtgggttgattttaacccatgttaggatatgttttcagtactctaatatgtttacacaaatttatttacataaattaaagcaaaaaaaattaaaacatctaaaagcttttacattcaacatttacatctccatgagtaggaagaaggttctcatttttacacatatactcacactttcccttatttgtgcatctagaagatagttgtagggacaaaatatgatgtcagcatcttgtattagttctcgggctgtgtaatatggacaggcctttagtttcttccccaggctgacaagttcttctatatcccaggctttgcacatcccttggaaagtctgtaatgtgtgctgatcactaattttatgaactccatgataaaaatagcaggattttccctagaaacaaatatgcataactgaaatgtgaaccaatattagcatagaaggaataaaataagcttatctcagaatttgaggaacatcattaaagccacaaggctaagattttactggctacgccaccacacctggctaattttttgtcttttttgagacaataccatagctacaatcacaacgaagtctggttgactcagacttagatagaagtccaaatcggaagagctccaaataccctaaattaacttgaaaaaaatttcttttaaatatgtttgttatgcctttttttttctgattaaactgatgttaggcatgaaatacttctgaaaggattcacaaaacaatggtatcactggttgcttccaaggatgagaattaagtgactgggcacaggagtgagagatttttctctgtagacccttttgtaccttttcaactttgagccatgtgatagtatttcttattcaaatataaataagaaggaaggaaggtaagtaggtaggttggaatgtcctaaggggaaaaaaagaaagtaagagtattcactacaaaatattcaggagtaccaaaaaaagcctaaattatccataattctattacacaaagaatgcactagaaacattttggtgtatttcatttcatatttcttctatacatgtgtatataaggatatctctacaatgtgacccaatattaaaataacaagaacaagcaaggcacgatgacaggtgtgagcactttgggaggcttaggcaaaaggactgttttaggtgaggagtttgataccagcctgggcaatgtagcaaaaccctgtctctacaaaaaataaaaaattttctaggattggtggcgtacatcaatggtcctagctactcaggaagctgaggcaggaggatcacttcagcccaggagttcgaggatgcagtgagccatgatcaagccactgcactccggcctggatgacaaaacaaagccctatctctaaatagtaataaaaacaagaacaaagcaaatgttccccaggcattgaggattattaacaattcagccacaataaacaaaaatttttataaagcatattccctttgttttcttctgtctagcgtattaaaaatcatacaaggaaaatcttgacatcactaagcatcttttcgttgctgctcatatatgtacattgaattaatgtgccatagctgatgaaccagttttcttattactgggcttatagactgttaccaatttctgctttagacataaggctgcagtgagcattcttatgctggtttctaaatattttcttagtctatatttttagaaatggcattatgggatcagacgctacaaggtatttttaagtttcttgaaaacatatagtgaaattatcctccagaaagtatatattgctttgcattacccttttcccacatccttatcaacactggtgttatcttttgttatctctatcttaaaagtaaaaaatagaatcttgttttgatttgtattttttatagtttctagcaaagtttaacatagtgttgtatgaatagtctaacaaggttcaatagtaatttgtgtttcttcttttatgttatctactaatgccctttgccattctattacctttttttttttcagagatgggagtctcactctgtcacctggggtgcagtggcacgatccaatcttggttcactgcaacctccgcctcccaggttcaagcaattcttgtgcctcagccaccaagtagctggaaccacaagtgcatcaccacatatggctccttttttgtagagacagggttttgtcatgttgctcaggctggtcttgaactcctgaactcaagcgatccgcctgcctcggcctcccaaagtgctggggattacaggcatgagccactgcactccaccccattctatcactggaaataaaaaaatttcattacaggctgggcacagtggctcatgcctataatcccaacattttgagaggccaaggccggaggactgcatgagcctaggagttcaagaccagctgggcagcacagggagactcaatctctacaaaaaaacaaaaaaaattaaaccgcagtgagcagtgaccacgccactgcactccagcctgggcaacagagtgagatcctgtctcaaaaaaaaatactcattatgtgcattaataaaaaaataaaatgtttgataaaacccagccctaacaagtgtctcatatactattaaaaatgtacaaaattagaaaagtatatatatttttagtagaagtataaaattctagtagtagaagtacataggcaatttgtcaaaaaactaccaaaatttaaaattttaccattaatgtgaatagatgtaccctgtactgttaaatagccaaaaaattgaaaattaaccaaatgtctacccataagggacatgttaaataaattatggtaaattaatacaaaaactttagcccttaaaaatgatttagatttgtctatgtgttgattaggctctctatgttaagtgagaaaaacatgttgaataacgtgtatagaatgattaaattcatgtaaattgcttttttaaagaatatgtgcacatacattatagcaaatacattttttaaacattctggtagaatagatatataaaatcttacagtagttaccttaaaggattaaggagatggaggatctagaaaaagcctctcttttcattttctaaccacatacatgtactacttctctttttcaattgtcattggaactaagtggtagaataatgggccacttttattttaattaattctactatttattttaaaacgattttgaaataaaaatatttatttttcctagtttgctatccttaacaaaaaacaaccatacataaaatatataaattatatcagggataaaagactttgaggcagctatatacatatgtgtgtgtgtgtgtgtgtgtgtgtgtgtgtgtgtgtatgtgtgtgtacatacacatacatatatacatatgggttcaagcacttctcatgcctcagcctcccaagtagctgggattacaagcacgcatcaccatgcccagctaattttttgtatttttagtagagacaggttttgccatgttggccaggccagtcttgaactcctgacctcaagtgcccacctcagcctcccagagtgctggcattacaggcatgagcatcatgcccggccaatgtattttttacaaatacataataatttccccatatatatatattcaaaatatttttgtctaataattattaaaattataaaatataaaaataattagtctaacaaggttcagcaataatttgaatttcttcttttaggttatctactaatgtcctttgccattgtattactggaaataaaagatttcattacaggctgagcgcactggctcatgcttataatcctaacactttgagaagccaaggcaggatgaaatgtctctctctttctgtgtgtgtgtgtgtgtgtgtgtgtgtgtgtgtgtgtgtgtgtgtgtaaaattaaattgtatttgtatacgtctgtataataaattaaattgtatatatatacacacacatatatatgcatatatataatctttacatgtatttttatctatgaattatccaaaaggagaatccgtgttttcaaaacatacatacatcaatacatacacaaatgtatctagatattgctagaaacaaagtaaaccataagagattcataacctataaaagaatgtcagcaaatcaaaggacaagacatgaatagctgaggaccagttctcctaaagctttgtattcagccatgttaaaggatgatacctgttagtcctccaaagtattaaaaataaaaattaggaggaaaggactcatctaactatatttatttaggccattttctacattattttcactcactttctctaacctctactgttcttattcttcatactgtgtcaacttctccctacctcaatgccatctttggattttgtttctagtgtaaccaaaatagcaatggatgaggaatcagaaaatttgggttctggtccctccttgctaattacttgtctgactgagaaaactccagggcccatttttctcatgtgactaagcatcaaaagaatatttgaaccagaaggcttgtaagattccttctgactcactgactatatcatctctaaaattccttccagctctaaaattctgtgattcaggtaaaaacgtgtcaatcatctttcatactctcagtttgatacagactgagtttaataataggacattcaccatttcttcattccaagcttcttccatacaattctgaaagcttacctttatacaaaatgatcactaaagtgcctttagaatctatgactatgagctattaagtctcattttaaccaaaaaaagatatatatctaaaataatatttcagaattgtcattcctgactctagttaaaattattttttcttaactgaaacaaaaattaacttttaacagataagtgcatacttttccagtataagggcactttagtaaaagaaacaatgaacaaaaacacatgtgccttggatcaagccaaaaaatcagaaagtttacactttaagggcagttagaatgacacccctctgcaggctgacagcaaaaccagaaaagctcatccattttagcagtgtaacacaaccctgaggaagtctgtcaattgaagtgttatcacactttaaacacttccattcaaactgtcagttatctatttatcatttcgctttggtaattcagtcaaagtaaacacagacacctatggtgactttgtaattgcccttcattacaataccaaattactaacagggcttgagaacaagaaagaggatgttcatgtatgcccgtgaggtaagcattctgaggcagaattaggtcaagacaaaccacaagcagatatcttcaattattcatattactaatattaaaaaataaaatacctgttcactttaagacaaactaaaggccaatgaaaaattcggaacattgaagtctactgaattcctttttatttttacatgattcaaacttatgagtaaagatcaataaaaacgtaaccagcactgtaggattctaggttacacaaaatgaaactgtctgccttcaatacacaatcttacactttatcattgcttttaccaatgtattttaccaatgtatgtttttggttggttggttgtttttgagacaagagccttgctctgttgcccaggctgcagtgcagtggcgcgatcttggctcactgcaacctctgcctcctgggttcaagcgattctcatgcctcagcctcccaagtagctgggattacaagcatgcgccaccacgcccagctaattttttgtatttttagtagagatgtgttttgccatgttggccaggccagtctcgaactcctgacctcaagtgcccgcctctgcctcccaaagtgctgggattacaggcatgaaccatcaggcccagccaatgtattttttacaaatacataattaatttccccaataacaagtaaaacaaaaaaagataaaaatatggtgacaatgccagcaagtcttcttgcataggctatatagccaaaaaataaatttcaagcatttgaggaattcagcagatcacttagtcactatggaaagcagtctacttatgcatgaaattcatggagttttaccataaagagctttattttattatttttgaacacctgacacttaatactttcatctctccttgaagagaagttttttaatatacagagtaaaggaatttcacaattcctcagttagttagaaataactctttggaaataaatatgactgtaggatttcaacaatgactttgattcagtaattctgtattctctcctggctcggaacaagtcactcaagttttgccattggaacaccttactacttatactttataatctcgtttgaatatctgttaaagtatcttttgaccatccctaaataaagtttaaataaattttcatctataaaagagagtattaaggaagaaaagcataaaacaaaataattcacattatatggtagggggtgatatatcaatgaaatgaaactctgggggctatagctcacagttttttgtgtagaaccaactctaataagatactagtgaaaaacaaaacaactctaaaaatatattaaaatatcattggtcacttcatagtaatcaatttatggttcactgatttgggggtataatttcatacttgcaacttcaattttagagtaaactaattatcacaccagaacataggctcccaaactaatactgccaaaagaaattatttgaaagtcatataagtctttagtaagaaagaattctctatttccaccaagcccaaattcatattattctaatttccctggaaaatttcaaaaccaccaatgacatcttttgaaagttaataatttagtataacttttaaaagaggggtttgtatttatcagaattatgaggtgggataatcattatatttctcattcaattaagaaaattcaaactttgcccagacagcctaaataaactatctgcttttgaagtttttaagtatataaagtatattaattagatctaattagactaaaccatttaaacacttctatctcctcatatgatatcatccaagtttgactatttttggtagcaggatttaaaagaagaaactaagatgtctgactacaacagaaattaatttcaaggaattaaatatcccaagctttaagaccaaatcccatactttaaaacttgactaaagattttattacaggaaaattgttttttaaaagatatcaatactagcagttaatttgattttccgaagttgattatcactaaaatgtacatataaaacacatactgagtaatttaaatattttcagccttattttttctctaacacaaaataactttactcacgtttttcccatctagcaattccatgcacttctcatttctgttgaagttaccgactacctcaggatggacacaagtatgatccctgctggaaagaatagtcattggaacccctgaatatgccgtcctccggagctctctagtaatctgagcaatctgcttgtgtgtgcgtgtcccaaaatatattttgggtatcttggatttccctgtatgatccttcttaatggtattcgatgactcttgactgtttccttgtttagtagaacaacagcacctagaacagtggccagggggctgtaagaaaggaaagaaacgataactaatatctaaactaccataaaaaacgttatcaaacctcacatggaatcagaacctgtaagtaatgaatcacaatggtcaaataaagagaaataacaagaaattatagtatctaaaacttgccattaaagaaaaaactacaatttaaaattggtcctcattctttcttttaaaagttataaatgatatttccaaatttgcattctttccaacataccactctaactctccaattccaaatgttttgagtacattttaactctcctctttcataaattataaaaatgataaaacaaagtaggaacaattttaattttttcttaggcttcatacgaacttgtgatattatagtttagagttaatagttttgttcacagcatagttcattcaaatattccttaatacaccaatgcttttgaagcacttttaaatatagtttacaaaagaatagaattatcaagttgaatctaaaggatcacttaacaacttcaaaaagagaaaattcttctttagcaaaggtacgaaaataaatgcaaaatattaatatttaataatgaaagcaggttgcttgtgtggaacaagtatatctgaaattcagttacctgtcatggagtgtaaaccaatcagtaaaattatcatccagggtagtttttcctggacaatttcacttgtaactcagtttcataactgaaatagttatataaatgtctctttaaacaaaccatatggaacatcaggaatatagtctacttaatttacctgatttttctcttctgtttccaaattctcaaatactaaaaaaaaaagtttaatgctatatattagaactcacaggttcttaataactacttccttctaagtctcaattattacaccaaatgacacagaacaaagtctcattttagagaaatagtgtaaaatggttaatttatagaagtggtcattgatggggagggaaaaaaggtaaatagcattgtaacttaaaagactgtgtgctaaagagttttaatttagtagaacaaacactctacttgactgtttttttttattattgtaatcagtaagattagccttcataaacaaatgctaagtcatagcatgttacaccacaatactggggatgaagctgtcttttagcctcaactcagtggaaaagcaagttcaaattactcccagtctcactcaaccttgtccgccattaaatattacaatacatgatagaatggatgtaaatgtgacccttctggagctgtcactcactgggataaaaggttcactgatgtgccaaataaacagtcagtcaaagcagataaagttacagccatgctggaattgttacattctcctctcaggttgccagtaacagcagatttcattacagagtgctgccacattaaatagccagttccaaatatcctgccttctatattgaataattacttattcactaaaaggataaaaagaagagttatgaatggggctcttgtcaacagtgaggcaggaaacagctgacatgtggtttatgttacatatgttgcaatatccaacaatttacggtcacagagaaaatggaatgcaaattatatcatgatatttctgactgaattctttttggcttttgcaatgatcacattaatcaattaacatacaaaaagaattaaggtaactcaattagaattaatttactttttcataccatcaattttttccctcaattatactatttctcttccttatgatacagttaaattcatgtgaatactagaatgctatggcccaatgcactgttatgtactatatagatacattctatctaagcataaaagtaatccaataaagtagctttttctcaaggctgggaaaacttactttttgatgttacacttttcaacatcaattccacttaaagctttaccatctactccctttaatttcaccttttctcttggtcaaaaaaattcaaacaataatacagaaatattttttaaaaatcaactatcccatcacgaataatttagtgtataacttactgaccttcatatatacatatctttcatatctttttttcaaatagaaagggatgctattatacagttttacaactagatactttgttttcaaagtttttaatttttaaaaggggtgaatttggcacaaataatttaatgttctacttatctttaaatgttacactagtagtggtactaacaaaagcaaaagatcaagaactagtctcagaaaacttttctcactagagtgactttagttaggacttgaaatctgttttttcactccataattcagaatattataaaccacagctaactttatattaaatgtttttgtttgtttttttaagttgtaaagtaatatgaacaaattataacatctatgaacttaagtagactcctcaagaagatggttttctttttttgtttttctttttgttttgctttttttgagacagactttcactctttttgcccaggctggagtgcaatggcatgatctcggcttaccacaactccatctcccaggttccagcaagtctcctgcctcagcctcccaagtagctgcgattacaggcgtccaccaccacgcctggctaatgttgtgtttttattagagatggggtttctccatgttggtcaagctggtctcgaactcccgaaatcaggtgatccgcctgcatcggcctcccgaagtgctgggattacaggtgtaagccaccatgcccggccaagaagatggttttcaataggtacttaggcaagtaaggtataaggtggaaattaaaagttaattcttttccctttgcattattaaacaggaattatttcttcagaagcaatcttctattaaagtttccatttaaaagattcggtacaggccgggcatggtggctcacatctgtaatcccagcactctgggaggccgaggcacgtggattgctttgggtcaggagttcaagactagcctggccaacatggtgaaaccctgtctctactgaaaaaacaaaaattagccaggcatggtggcatgtgcctctagtcccagatactcgggaggctgaggcaggtgaatcacttgaaccagggagacggaggttacagtgagccaagaccacgccactgcactccagcctgggtgacagagcaagattccatctcaaaaaataaataaataataaaagatttggtacaatcatatttgaagacagaaaaactgattgtagtaatataacaaaggcttcaaaaagtcacttataacaaaagataattacctgtaccaaaatcagcaactttcaatgctctaagaaaactagaggaaggatcctgatgccagcagtgtcaggaggtcagaatatcccccaatcaaattaccaggctaatactttatggcctctctctgaaaccctttgacccaggggtcttaaaacaattagatgaataaaattaaataattttgtatctcttattgagtatttttcctgctaacaaatgatcatgtttatctttcaaaaagacttttttattttccctttgctgtttaaattgacagagatttccttttctccaggaaactagagaataaattttataatattcactctcatccaataatatatctgaaaatcaatacataagacatggggctttccaaaatctgtaaactctaagacctgtaagtggctacacagtgccagaaccaaactctttaagaaatggcttcttctccaagctagaaactgttcagagtccttttacttcaagaaaaggcaagaaaacccaacccaagtaactgcaaaaatatactatcattaaaaaaaaaaaacaggagatacatgtggatgtgacagctataatatttccatgtgcagtagccaattgtttaaaaatacatttcaaatctacaattatcaaaactgttcttagaacatcagcaaccaaggattttcagaaaatttttcttgtgtttaataaattatattccctctcttgttgtattatatagagacatgtctacattctttcaaagatgaagagttcgaagatttgatcttgaaagaaagatgaattgctgagtccttgccactgttcctttcacaagctataattataagttcaaacggtaacataccacaatgtcaacttctaaagatttataactttctccaaactttccctaccactcacacaaaaaaatactatgcctaaatacctgaaaaatgagcatagcaattaaatatttttagaaatggaaaatgataaagaaataggaactaatttcattgccaaagaagaagttcctttgctaactgtaaatcaggagagattctcaaaggagactacaaattgaatttcaaaagcaggcaggagtacgaaaactaagtatataaagaagtagagagaaaaagagaatgaagagaaacctgtttgagtgaaataaaaggaaaactaggtcaatgaaacagtaaaaaaaaaaaatacaaaaacttgtgttcttccataactttccacatgtgcatgtattgtctttccaaccacactgcaaattccttgaagacagaaactatgtcttacatttctttatatagctcattcatcgagagtggtgttttgcacataactgtaacttagctttttttaaactgatgaattatgactaaatgtccctgaaccaaatactgtacccatgcattacaattaaatctgacagatgcttaaatgatgtcctaatttgaagtgctgcacatatcaaaaagatacttcaaaatccagattagcttttaaaggaaaaagtacaggacaattactatttttcaaggccaaaaagaaaaaccatcttatgctagtattttagtcgtgacaaacatatatcccaatattttcattagtaataatactgccatttttaagcctgtccttgtgctaaacatttttcatctaatatcccatttcattcattcattgactcaagagacattggaatccctatatatcaagccacggagaagacaatgacaaggatcacctcatcttactgacaaagagagcttcattcttaaattgttctacaaagtgtagttcttggagtttatcacaccattttcactcataaaccaaggaattgtttaaagttagttatattattttatgaatgcaattacaaaatattcctaagtatgatcttcataaaaattgttttaacttattagttgacatttaaatcaaatatgttataacaagatccatattatgtcccccaagggaaaaataaaaaacacaaactttcaattaatagactctttagagcaagagtcagcaaaagagccagacagtaaacatttcaagcttcaaaggctatatggtctcttcttccataggaagatatcttttatgatcgaaatgtagagaaggatttcctaaaacaataaaccaaaaacacaaatgagtgaaaacacagcttcattaacatgttaaacttctatttaaagatacatattccttaaagaaggaaagaaaaataggtaaaaagggggcacaggctgacagaagatacttgcagtgcttacagtcaagtgaggatcagtatctacaacatataaagaattccaataaatctatttttaaaagacaaaccctacagaaaaccaagaacaggcaaatcacagaagagaaaagctgaaaggctaataaatatctgaaaagatgtgccatctaatgtacagcttcactagtaattaggaaaatgcaaattaaaaccataattagattccatttcctatccaccagattgggtactggagaggatatgaagaaactcttattcactgctgatcgaagtataaattggcacaactacttgagagcaaattggtgatctagtgaaatcaaagatacatataccctttgacccagcatgacactgttaggtatacatgctagaaaaactcttgcacattaagacagatacaagaggaatattaattgcagcattgttttaatagtgagaaaaacaagctacatgtatcaacagaaggaatggatgagtaaattgtgggctctttccaatcaatggaataatattataatgaaaatatatgaatgacagttgtatgcatcgacatcgataaacatgaacaagcagttgaacaggctgagtatggcttaaacaaaattgtattttccccataaaattctaaataagttgatgatagaaataaaaatataaaacttcaggtagagagtgaggaatcaagactatggaagataatcttaaaagctaacaattgtcatgaaaaacatacctgaaaaatgctagaagtgctggaaaaaaaaaaaaaaacccacagtaaatatactctgttctggtttctcctcagttgtttcatacccagcagaatagctttcatcttaacacctcctcctcaaaagttttcaacctaatttattaggaaatgggttctcaaattagggtccatgggctattacagatcacccagaaactacagacacaattttgtgtgcatttttagaaagtgaacccctaactttaatcaagtgctcaaagcagattgtgactgatttgggttagggctttattccagaatttatatctggttttttaacgaaaatcaaatggaaacaacattttctttataaccaaattagccagacatagttattctgtaaaggaataaacaacagccaagagaatttcataaagtttcctccactgcttctgaactcatttctattttaatgtttgttaattacctaatattaacataaggctaaaatactcagttcactaatgtatatttctaacttcattttacagataaaaaaggaaattcagaagaattcaaattcccacaaaatacacaataaatttgtggagaagttgaaattcataaccttaaatctttaaagtgaagctttaacaggattgttttcatctaccacttaagcaaaaaagcaaaacactgccctttatttcaaaaagtcatgcatatgaagagctagtggtaaaaggccaatgaacatagaattatactacaaactttgagacataaaaaagggttttttaatctttatcaaccatttgctgttgtccaagccatctaaacaggtactgaatgcccgttgtgcaaaatctcacattagcacacactctagtaagatagcaaaagtaacataactataccttttcaactattctttttttaaaaattcaaacagtagtagtaccctactgccaccgtttcatgattagactgcacaaagcagctgtttatccactcacagcaaccagcaggtgaacctgattcaatatttatcacaggctttcatagatcactgctaaccaagcaaacgttttaattaagctggaacttgcttcaaagagctcattcaagatgtcaatcagcaggtaggggatcagatacagtgtcaggtgagagtgtgtaatacaaatccacaactttcttcatcccatcgctgtaaattagcctgctctaaagttcagactctgattgtaacagactgccagtctcctggaatgtgtaactgaagtcactcaaattctattgtgaaggttgtaagcaatatgttaaactgcagtgggctagcacattaagacagatccttttcacggttgttttttgataccataagtgtttgttaccctactctccacttactgtttactttcttgcctagtgtaaatgggaattccttaacacagttgctccagtttctgaaaagtctgtgtttcaatagagtaaatcaataagaattatcagtttaagaacaagcatatcataaagatgactgacctgctataaagcaaaaattatcctggtataatagaactttacatagatgtgattatagcaatgagtctcaaagcagtgatgagtcacaccgatcaacttcatgagaaaaacccagggtgtttataaaaaataaaaaggggaaaaagaagactcaaacgagcaaaatggggaagtgggcgggtaggcggaaaatatcttctttaaataagaatgccagctaagaattgtaaataaaatgatagaattttaaaaatcaccattttgcaactttcctgtactaactgattcaggcaagttacataaatggatgctgaaatctttcagtgaaaagttattagggaacagaatattgacaaagtctcaaaatatcaccccacagattattcattaattacaaaaggaaaatgtacctttacaatgaagagacctggcagtgacactctaaccaagtggtgaaattcagcatcaatggtattgagactaactggcttcatatgcctcctgatataagaaatatactcttgtgcccatcttcaataaaactacatgattattaaatccaaacaatcaagacatgaatcaaaaagaacctgggaatagagaaactgtggtaaaaagacatatatgtattatttagtcggtgaaaattcatccaattgtaatttatgatatgtacacttggttatagattgtatttcaaaaaggcttttaaaaaaaaccctatctcttgagattgcaggtaatttttactttctcctttatggtatttttcaaactttcttcaatgaaccttttaaaatgagaaaacaatgctatcctatcatgtcattttccacatattaaccaaaatgctataaacaaaacatatgacatagagatttgtgcttctatgggctttaaaaattaaatggttattattacatacaagtctctaaagcagcactacccaacaggattttctgtgatgacagcgtatttgatatctgcactattcaatatatttggcattagccgcatgggcacttgaaatgtaattagtacaactaactgaattttaattttaattaatcaaaaaacataaatacatgtggctagtggacagaatacatttaaagtattatcagagattctagaaaaccaatggttattgcaggtgcattgtatgtgtgagaaattctcttcgtaaattcaagttagaaaggaaaaagcaaaagtacactgtaaaaattgtgtgctcataggtaaaaatgggttctgcaggttgcagctcatgaaaactgggttcacaatttttgtagactagtaaactacttttcttaatcttttttgttctaatttttgtctgaattccaatcaaaatcccagcaagatttttttttgggtaatctaaatttaatcttgaaaataaataagtgagaataaccaaaactttttggaaaaataagatggggacaggtgtgatctctcacacctgtaatcccagcactttgggaggccaaggcaggcatatcacttgtgcttgggagttcgagaccagcctgggcaacatggcaagacctcatctctaaaaaggggggaggggggggaaagatgggcaagtagggtatatatttctatagatgtaagaatttcaaatatatgataaagatgctgtattaggcacctctatggacatgtgtccacctacttactctgaaatcatctatatcccctgctgaaaggctgaacctaatatccacattaataacacatatccaaagacactctcaggcatagcaagatcaattagtttcactttcccaagaacttgtatttggggcatttagactgagttgattaattacagaaaataggccacatcatttggtagattttaggcttccactttgagatgtcatggtaaggtcatatgaaagagtgtaagtatctagaaaaaccaatctacaaaaaataatgagaaaactttgctgagaaatgcagatgtcacaccagaggccccaaagagaacagagaagctgcctaaaaacttttcatttccaatgaggggaattatatctcctgcatgatctttcttttagcgggtttctatttcttaaaaagcagatcactcccttaaaaatctgatttaaaaatgggccaaaaatttgaatagacatttctcaaaagatatacaaatggcaaacagacatatgaaaaggtgctcaacatcactgatcatcagagaaatgcaaatccaaactacaatgagatataatttcatcccaattaaaatggcttttattggctgggcacggtggctcacacttgtaatcccagcactttgggaggccgaggggggcagatcacctgaggtcagtagttcgagaccagcctggccaacttggtgaaacccaatctctaccaaaaatacaaaaatcagccaggtgtggtggtgcacgcctgtaatcccagctactcaggaggctgaggcaggagaatcacttgaacccgggaggcagaagttgcagcgagccgagatcatgccactgcactccagcctgggcaacagagtgagaatttgtctcaaaaaaaaaaaaaaaaggcttttatccaaaacacaagcaataacaaatgctggcgaggatgtggagaaaagggaaccctcatacaccgttggtgggaatgtagattagtacaaccactatcgagaacaatttggaaattcctcaaaaaactaaaaattgaattaccatatgatccagcaatcccactgctaggtgtatatccaaaagaaagaaaatcagtatatcgaggagatatctgcactctcatgtttattgcagcattattcacaatagccaagatttggaagcaacctaagtgtccatcaacagatgaatgaatgaagagaatgtggcacatacacaatgaagtactatttagtcataaaaagaatgagatcctgtcattttcaacaacataaatggaactggaggtcattatgttaagtaaaataagccaggcacagaaagacaaactttgcatgttctcactcatttgtgagagctaaaaattaaaacaggtgaactcatggagagagagagtagaagggtggttttcagaggctgggcaaggtagtctagggaggggagagagtggagttgttaatgggaacaaaaaagtagttagaaagaataaataagacttagtatttgctagcacaacagggtgaatatagtaaaaaataatgtaattgtacatttcaaaataactaagagtataattggattatttgtaacacaaaggataaatgcttcaggtgatggatacctcatttaccctaatatgattattacacattgcatgcctgtatcaaaatgtctcatgtacttcataaatatatacacctactatgtacccaccaaaattaaaaattaaaaaaaaaaaaagcaaaccaccctgtttaaatcaggtggcatttcaaatctgtgggggaaattattcaactaatatttagatgaatgactatttggggaaaaaaattagatcctatctcccaccttacatcaaaataatttacagttgaattaaagatcagtatataagaactgaaaccacgagacttccagtctaatatggcagattaggtacacacatttgccctccttcccacctaaaacaccactaaaataaacttacttagtagctgaagttgcaaactggaggtccacaaatcatacctgcttcacaatcatgttttgaatggctacacagagttttataaatttgaactacttaaaaattatgagatgaaaatccatagtgctggtttcttctagaaaaactggttgactggccacattaggcctgtattcttacaggacagcaatccactaacattgactagattgccacatttggatgagacatatactctcaaggtatcacagtccctgccacttaaagaatgtaatcacatgcacttatacttagaccccttctttcattcatgctacctatctggtgctcatttaagttgccaaccctggttacaaaagtgtaaaaagggccaggcacagtggctcacgcctgtaatcccaacactttgggagaccgaggggagtggatcacttgaggtcaggagttcaaaatcagcctagccaacatggtgaaaccctgtctctactaaaaatacaaaaattagccaggcgtggtggcatgcacctgtaatcccaactactcgggaggctggggcaggagaatcacttgagccagggaagcggaggttgcagtgggctgagatcccgctattgcactccagcctgggcaacgaagcgagactctgtcttaaaaaaaaaaaaagaaagaaagaaagaaagtacactccacagagtgggagggggcttgagcaagaggctcaagagcactggttacaaaattttctggagtttaaatactctctagaggtttccattggttattttgttgcacactatgtaaataaacacgtggcctgtgaccagtctgattggttgtgggaggcaaccaatcagaagctgaagggaagctacagttacatcctatgcaaaggaagacatggcccatgaccagtctgattggttgtgagaggggaccaatcagaggctgaagtgaagttacagagttacaccctatgcacatgaagactggttgtgggaaggagaggtactttccattctttaacctgccattcagtagaaaggcagggtgttgcaaacagagtagcctctgatccttttgttacttgggggtggagaggtgagtttttctttttgattcagttttaggaagtcagtgtgaatcggccttaggttccctgcctccagaccctattctcctgcctcaatttcattctctttttttttcttgtattttttacctttttatttttaattttatttttttttacagacagggtctcattctgttgcccaggctggagtacagtgatataatcatagttcactgcaacgttgaactcctaggtttaagcaatcctcctgcctcagcttcccgagtagctgggactataggcacatgctaccacgcctggctactttttgcattttttgtctttttaaaatttttcttaagatacaggatctctctctctctctcgcccatgctggagtttaatggcacaattatgactcacttcattctcaaccctcaacctcctcggctcaagtgatccttccgtctcagcctcccaagtagctggggctacaggtgaatatcaccacatcaggccaccatttcattctcagcagatattgaaggctcaatgaatattactggtagaaataagcaacaaaataaataaatctcacattaactaaaactgcacatgaaaacatttgttaatgactaaaaattgtcagaagaatcagtaatagccaccagtaaaagaactaaatgacagaagacataataaaaacctcgtgtgactggtttttgatgtgaaaaatataaattaatgaatgaaatgtcttccctataagcgttacataaaattagtaaatgacaaaaaataaaagacaataaaagaagaagaaaagctatttgacgaatagactgataatgggattctcatcatttttttctgtaaaatctttgatgttactagaatttttataagcaaatatcacttttataaaaacaattacattttttaaaaacatacacagaaaagaaaacaatatacagggtagagatactaccaaaaggagaagtaactagtttttcagggcttgcagaatcattttactcaatttgataagatttttgtcgtactaatagatccagttatgtgccctctgggaaaaatgcaccaaaaagcgccaaggaagaaagggaactggacagactatccagtaaggccagtcaaatcaatcatacaggttaatgagatgactgaataggaaaacagactgctctcacatctactattgttcagaatattagattaaggaaagagctgagacaaggtttccttgtggaaggacaggagaaaacataacatggttatagctaaggaaaagaatcaatagaaaggaggaaaaataattgacaaacatccagtgatgtgtaaatgtgtagtgggatgtgaatggggaaaagtttgtgtttaaaaaaaaaaaggccctgtatgactaatatggggaccaacaagttttcagtttaaaggtgggacggttccaaatgacaacgaagtcccatgtgtagtggtgctgttgaattaatggctaaattaggtcgtaagtttcaagaaggctgaggaactatggtcaaaatagcagatcatcaacacagatcatgaagtaccccaggatggtgactgcttctcctactccagattcactgagtaacaaggagaatctgacatttttactgccctgatgtgtgcctacttcactaaacctcaaaagcatctttctttaaaagattcatacatgtttccaatgctgttgtcaatatcaattatcttctgaatgaaacgttaaccttgcacaagtaatgcaacatctggcttatagtagttttcacttagaacaatgcatatatttgtttaagttttccaaaaggccaacatttaaagaaacaataaatgtccttaaaaatatgattgtatggccttctgcttttagccatacgaaaaaacagaaatcttatcttgcaccataaactactaacaaattggacaagatatatgaaacagtacttttcagacactgaattgtgatctcagagaaaaggaaacaaagaggtgataactacattactttacttctacagatgaaaacacaataactgaaaagaaaaatccactagtaagatttaacaccaaattagaaatagcagaagagatcaaggagcttgacaaatcggcaatagaaggtagacaaaatgaagcacaaagaagaaaaaatagcttaaaaaatgaacatatagtcagtgatgtataatatcaagctgtctaatatacacataattgaagtctgaaaggaaaaaaatgagtacttaagaaaatatttttaaatggctcaaattgttcataatttgatgaaaactataaaaccatagatccaaaaagctcaatgaccccaagcacacaataaacacacacacacacacacacacacacacacacacacacaccttagttgaattgctgaaagcagagataaaaatagaatttttttttttgagacggagtttcgcagttgttgcccaaagtgatctcagctcactgcaacctccgcctcctgggttcaagtgattctcctgcctcagactcctgagcagcagagattacaggcatgtgccgccgcacctgcctaattttttgtatttttagtagacacagggtgtcaccatgttagccaggctggtctcaaactcctgacctcaggtgatccacccgcctcagcctcccaaagtgctgggattacaggcatgagccaccatgcccggcctaagatagaatttttaaagcagccacagattactctatagaaactatgtaagacaagccaagggcagaatatcttttaagtgctgaaagaaagaaaatgtgaacctaaacttcaatacccagtgaaaatgtgtttcaaaacaaagcgacggttcctatcaataacccaatgaagtttcttgcagaaatagaaaataccatcctaaaattcacatggattctcaaggaaccccgaataaccacaataatcttgaaaaagaacaaaactggaggtctcacattttctgatttctaaacttactacaaagctacagtaagaaaaacagcatggtactggcataaaaacagaaatatagaccaatggaacagaatagaaattccaaaagcaaacccccacatatatggtcaaataattttcagcagcctatcaagaccattcaatgggaaaagaacagttttttcaacaaatggtgcagggaaacctggatatctacacacaaacaatgaagttgcaccattataatatatggaaaaattaactctacatggatcaaaaccttaaatataaaagctaaaaccataaaacttttccaaaagcactggtgataaaaaatggacttcatgagataaattggactcatgaaaattaaaaacgtttatgtatcatagaccaccatcaacagagtaaaaatgcaacacaccaaatgggagaaaatatttacaaatcatgtatccaataaggtattaatatctggaatacataaagaacttccaccaactcaacaacgacaaaagcaatccaattccaaaatggacaaaggcaacatcatactgaatgagcaaaagctagaagcatttcccctgaaaaccagcacaagaaaaggaagcccggccgggtgcagtggctcacgcctgtaatcccaacactttgggaggctgaggcaggcggatcgcaaggtcaggagatcgagaccatcctagctaacatggtgaaatcccgtctctactaaaaatacaaaaaattagctgggcaaggtggcgggcacctgtagtctcagctactagggaggctgaggcaggagaatggcgtgaacctgggaggcggagcttgcagtgacccaagattgtgccactgcactccagcctgggcgacagagcaagactctgtctcaaaacgaaaaaaaaaagaaaagaaaagaaaagaaaaggaagccctctctcaccactcttattaaacatattattggaagtcctggccagggcaatctgacaagagaaagaaataaagggcatccaaataggaagagaggaagtcaaactatccctctttgcagatgacatgatcctatatctagaaaactccacagtctcagcccaaaacctccttaagctgataaacaacttgtcaggatactaaatcaatatgcaaaatttactgacattccaattcaccatcaactgtcaagctgagagccaaatcaggaatgcaatcccattcacaattgccacaaataaataaataaataaataaataaataaataaataaataaataaaataactgggaatacaactaaccaggtaggtgaaagatctctacaaagggaactacaaagcactgcttgaagaaatcagagatttcttacaaacaaatgaaaaaaccttccatgttcatggataggaataatcagtatcattaaaaaggtcatactgcccaaagcaatttacagattcaatgctattcctattaaactaccactgacattcttcagagaactaggaaaaactcttttaaaactcatatggaaccaaaaaagagcccaaaaagccaaggcaaccctaagcaaaaagaacaaagctggaggcatcgcactacccaacttcaaactatactacgtggctacagtaatcaaaacatcatggtactggtacaaaaacagatacatagaccaatgaaacagaatagagagcccagaaataaggtcacacctacaactatctgatcttgaacaaacctaccaaaaacaagcaatggggaaaggattccctattcagtaaatggtgctgggataactcgctaggcatatgtagaagattgaaactggaccccttccttacaccgtattcaaaaattaagatagattaaagacttaaatgtaaaacaaaaactataaaaaccctggaagacaacctaggcaataccattctggacataggaacaggcaaagatttcatgatgaagacaacagaagcaattgcaacaaaagcaaaaattgacaaatgacacccaataaaattaaagagttaatgcacagcaaacgaaactatcaacaaagtaaacagacaacctacagaatgggagaaaattttttgcaaactatgcatctgacaaaggtctaatatccagcttccataaggaacttaaatttacaaaggaaaaacaacctcattaaaaagtgggcaaaggacatgaatggacacttttcaaaagaagatatatatgcagccaacaatcctatggaaaaaagctaacattactgatcattagagaaatgcaaatcaaaaccaccacaatgagataccatctcacaccagtcagaatggctattattaaaaagtaaaaaaaaaaaaaaaaaaaaaaaaaacagatgccggtgaggttgcagagaaaaaggaaaaaggaacacttgcacactactggtaagaatgtaaattagttcaaccattgtggaagacagtagacagtgtggcaattcctcaaagacctaaaaacagaaacaccatttgacccagcaatcccattactgggtatatacccaaagaaatataaattgttctattataaagacacatgcacacgtatgttcattgaagcactatgcacaatagcaaagacatggaatcaacctaaatgctaatcaatggtagactagattttaaaaatgtggtacatatacactgtggaatactatgtagccataaaaaaaatgagactgtgtcctttacagaaacttagatagagctggagaccattatccttagcaaactaatgcaggaacagaaaaccaaatactgcatgttctcacttataagtaggagctaaatcatgagaacatatggaaacataaaggggaaaaacacacaatggggcctatcagagggtagagggtgggagcagggagaggatcagaaaaataactaatgggtactaggcttaatacctgggtgacaaaataatctgtacaacaaaccactaccacacaagtttacctatataacaaacctgagcatgtacccctgaacttaaaataaaaattaaatttaaaaaaaatggggctgggagtggtggcttacacctgtaatctcagtactttgagaggtaaggcaggaggatcacttgagaccagaagttcaaggctagcctggccaacatggtgaaaccccatctctattaaaaatacaaaaaattagccaggcgtgggggcacacacctgtaatcccagctattcgggaggctgaggtgggaggactgcttgatcctgggaggcggacactgcagtcagctgagatagtgccactgcactccggcctgggcgacagagtgaagccctgtctcaaaaaagggcaaaggacttgaatagacatttctccaaagaaaatacacaaatggctaataagcagaagaaaagatgctcaacatcactaatcattagggaaatgcaaatcaaaaacacaagataccacttcacacccttaaaaatggctattctttaaaaaggacaacaaaacagataatgaggttggcaaagatgtggagaaactggaacccctgtgcattgctgatggaactgtaaaatggtacagccactgtgaaaaacagcttggcagttcctcaaaaacttaaatatagaattaccatatggtccagaaattccactcttaggtgtatacccaaacgaatgaaaacagagactcaaacagatacgtacaccaatgttcacagcagcattattcaaaatagcaaaaagatggaaacaacccagatgtccactggcagatgaacggacaaacaaaatgtggtatatccatacaatggaatattattcagccataaaaagaaataaaattctgacacatactacatggtggatgaccttgaaaacattatgctgagtaaaataagccagacacaaaaggacaaatattgcatgattccacttacatgaggtatctagagtagtcaaattcatagagacggaaaggagaacggcatttttcctggggctggacaatagggaaatacagagatattgtttcatgggtatggtgtttcagtttggaaagatgaaaaggttctggcactagataatagtatataaatatgcataataccactgaatttacacttaaaaacagataaatggtaaattttatgttatatatattttaccattaaaaacatgaaggtaagaaaaaaagctgaaaaaatatttgcaatgcaaatgttaaataaagctctttaagctaatagaaaagttacacaatacagaaacttggacctacaaaatgaagggcaccagaagtgaaaaactggagggttaagaaagaaattattttcctcatttgtaaatttgtacatagctgatgatttaaactaaaaataacaaaacactgtgagggctatgacaaatgtagaagtaaaatatttgacaaaaatggcacaaaacatgggagagtagacacggaagtatattggcagaaggttcttacacattatttgtgagttgaaatattatttgaaggtacactgtgataagtttaagatgcaaagtatgaagcaactcacaaaaataaaactaagaggtatacttaagaagacagtggtgaaggtaaagtagaatcctaaaacatgtttaattaattcaaagaagacagaaaaggggaataaagaaaagttggaacaaacagaaaacaaatagtaaccacagtgataattacactgaatgtaaatagtataagtgttccaattaaaaagaagaggttgtcagattggataaataaagaagacaattatttataagaaacttatcttaaatctaaagacacaaataggttaaaaataaaaggatgaaaaatatatactatggaaacattaatcctaagaaagctggagtggctatatcaatgttagccaaaatagatctcagaacaaagacatttcataatgacagatgggttaatacatcaagaagacataataatcttaaatgtgtatacagctaataacagaccatgaaaacccatgaagcaaaaactggtagaattgaaagaaaaggcaaatccacaattataggacccttacagtattctctaattaatttataagtagaccaaaaaaaaccagcaaggatacagaagatcttagcaagataatcaacaaacttgacctaactgatttttataaatgaatacccatactttccaaaaactcacagagcatttgccaagacagatcgtcaaatttaaaagaactgaaatcatatagaatctattctctaaccacacaaaaataaattagaactcagtaagagaaaaatatctaggaaaaaacacctagttagtgtttatttagacttctaaataaatcttgggtcagagaagaaatcacaagggaaatcagaaaaaaatgtaacaacatcaaaatttgtgggacacagataaagaagtgcttagagggaaatgtatagctttcattggtgttttatttaggaaaaaaaaaggtccaaaatcagttatcaactatttaagtttcaatctttttttttttctttttttttgagacggagtttcgctcttgttgcccaggcttgagtgcaatggcacgatctcagctcactgcaggctccgcctcctgggttcaagcaattctcctgcctcagcctcccgagtagctgggattacaggcgcccaccaccatgcctggctaattgtttgtatttataatagagacagggttttgccacgttggccagcctggtctcaaactcctggcctcaggtgatacgctcgcctcagcctcccaaagtgctgggattacaggggtaagccaccatgcctggccaagtttcaatcttatgaagctagcagaagagaaaattacatccaaagaaagtaaaacaatggtaattaaaaaaaaaaaaagcagaaaccaacaaaatagaaaatggaaaaacagaaagtcaacaaaaactttttaaaagatcaataaaatggataaacctctactacctattacagattgagcaaaatgaattctagattaagcaaaaaaaaggagatagacacaaattcacaaattatgaatatcagaaatgaaagagggcatatcactacaaatcctacagacacttaaagaaataagagaatattttgaacattatgccaataaattcaaaaacttgaatggacaaatttattgaaagatactaattactaaaactgactcagaaacagaaaatgtgaatactcctatactcatttttaaaattaaatttaaaagtaaaagtcttcccaaaaataacacataagggcaagatggcttccctggtgaattctatcaaaaatttaaggaagaagtaatatcaatacttcccaactcattttataaagccaacattaccttgatatcaaagccagataaagacattacagaaaactatagaccaatattcttatgaacatagatggaaagaatccttaacaaaatttagcaagtcaaatctaacaattatataaaatgggtcctataccatgatcaagtgggttttattccactaatttacagtcaggccaacattcaaaaactaatcaatgtaatttaccatatcaacaggatataggagaaaaaacatataatcctaccaaggaatgcagtaaaactttaaatataaaatccaacatccactcattataaaaatgttcagccaactagcgatagaaggaaacttctttagcctgacaaagggcatagacagaacacctagcacaaatgtcatacagaacagtgagggaccagaatgttttccaccacaaggttgtgaacaaggcaagggtatccactctcatcactctaattcagccttgtactgcatgaaaggtaaacactgtaaaggaagaagtaaaacagtatttactagagaatgaaacatgcaactttgggtacttagaaagtcctaaagaaactccaaaaatatcaactagaaccaataagtgatataacaagttgcaggttacagggtaaatatacaaaaatgaattctacttctataatagtaatgaataactggaaggaaattataagtggaatcatacaatatctgtccttttatgactggcttattttacttcacataatgtcttcaagtttcatctatgttgtagtacatgtcagaatttccttccttttaaaggctcaataatattccattgtatgtatacaccacattttgtttattcgtttatttatcaatggacattttggttgtttccaccttttggctattgtgaataacactcctatgaacactggtgtacaagtatctgagtacctgctttcaattcttttggatatttgcctagaaatgttatttctggaccatatggtaattctgtttttaactttcagaggaatcaggaacaaggtttttcagttaaacatacagaaaggaagaaaattagaatgggccctctggtactggaatggaactggatgtattggtataaacactcataattttcagtgtacagacagacagatacagaaaaaatatagacataaatgtatatgtctatgagtctgggtttgtgtacacaatacattgtgtatattctctagctgtctgctgaaagggcctggagcagcaactccaacagcaatgagcaaaactaactcccagaacacagcgttcaaatgttatttttcactaaatggaaccagggttccttggagaaatgtctgaatccgagactagggcagggaaaatataaggtgagcctggaacactatcttttgccaaaaaataaggaagcgttccaagaatgattgatatatgtcagaaagacacaagcaacttaactggtttcccacttgagtaccaaaacaaatcatataataattataactcaccgaataaaatggaaaatcacaagtccataaagatataaataaaatgagtaaactgaatttttgtaaggacatgttatttatataatttcagagttcttccccataaagtcctcttttataaaggcaaaaagataactgtaaagttatattttttacctagcagatatcatattaatcaagttatcaatcattaatgagaaaaaatgaaactgcgtgccaccttataagatgcaatgagcagaaggcatcacttctgtgacattcctgccaaagataaataacctcagtctaatgagaaaacccaaattaagagacattctagataagctgtaatattctgaagtgtcaaaatcatgacagtcaaggaaagactgaagaagtgttccagactgaagaagttactaaagtgataactaaatgcaatgcatggttctgactggattcttttgcttttataaaagcgttattaagacaactgctaaaatttaaaccggttctgagtattagatggcaacaatgaatcaattttaacttcctgattcttatggttatattgtgattatgtataagaatgtctttgcttataagacatagacactaaagtatttcagggtgagggaacatcagggcaacaacatactcttaaatggctcaataaaaaaagatatttggattgtacttccaatttctctataattttgtgacagccaaacttttaaaaagctaaaataaccgaaagataaatttactgggcagacagttggtcaattaacgaagtagttttcaaaggtaaagttatctttccacacccagtatccatgaatgaaaacgttctacatgacaattgcagatactatcaccactgaatagctacactcatgtcggatgggttgctatacatatttatttcattaatgtatctacacatcaagaaaacttagaggagtggctgaattttgtcaactttctctgaaatattcaccaagttcactcaatatgataaagaagatttgaaacttttgagaattaaacatttccaccatagagaatactatgttttctccaagctctcaaaaaattataattactgggccaggcacagtggttcactcctgtaatcccagcactttgggaggccaaggcaagcggatcatttgaggttgggagttcgaaacatgcctggccaacaaggcgaaaccctgtctctactaaaaataaaagaaattagccaggcatggtggcaggcacctgtaatcccagctactcgagcggctgaggcaggagaattgcttgaacccaggaggtggaggttgcagtgagccaaaactgcaccactgcactccagcctgggtgacaaagcaagactccacctcaaaaaaaaaaaaaaaagccataattattggagctgaaggacatactcaagcaattagtcatccattctatcatcctacccctaggcaaatttgcagacaaggcaagagattcacactaactccactaacctctggcctaacttaaccaagattacaatagtatagaacgggctggcaaactttttctctaaaaggccagagattttcctgctggctgaacatgaaaagaaagaaaagaaaaatacataaatagacagagtatatattttaggttttgtgagccatatggtctctgtcacaactactcaactttactgttataacgcaaaagttccacagacaatatgtataaacaaatgtaaaatgcgttcaaaaaaactttatttataaaagcaggcagcaggctgtattctgcccatgggtcatagtttgccaaccactggcataggaaaaaagattacgacaaaaaagtatccatcaaaatagacctgctaaattttactatgaccgtgatatattaggtgaaaaataaaagctatatactataatcctaattttgtgtgtggggagggagtatatatgtaatatatgtgtaatatatgtcatatatactacataacaaatatatgtaatatatactacagtatcaacccctttacaggggttgagctctgtacaacacctgattgcaggagtgagccaccacgctcggcccaataattacgatttttttgagagcctggagaaaacatagtagtgttctctatggtggaaatgtttacatatatactcagttgatactgggtatatatgtaatatatactacgtaacaaaaatggcaacaaattctacttatctttgtatataagctccctttcaaggttactttgtagctcttcctaccaagatgtgagctttaccccttgagtctggatttggtcatgtgatttgcttttggtgactggaacattagcaaacctgacacaaacagactcaaaaggcatttgtacactggggcttgttctcttgctgctcttggaaccctgaggccaccatatacataaaccagaattagcctgctagatgatgagagagacccacctcatggtcccagctgactaccaatcaactccctgaagcagggctgccttgctgacttacagctgacctgcagttgaccacagacacatgagtaggccacctgagatcagcagaagaaatgcccagctgagcccaccctaaattgctgacccccacaacagtaagctaaataaatggttggtttgttacagcaacagataacattgcatgtgtgtgtgcacgcacatgtgcgcacttaagaatagacagcaggcagaatatcaggggttgatattgaggagacagtttaattaaggattaatgttaataatttaagtacacattttggtggttcccaaattttctcttagatgatgcttttatattcagaaaaaagttatttagaaatataaaatttaattcaatataaaagagtatttttcattatttccactaaatattgaaagtctaaaaatatggacatcttgacctgatgtatttaaatgaactgatagattttactttctaacagagagtttggagataaatagttacaggtacataggaaactaagcaaaggacaagacaactactagcaccaaaaaaaataaatactggtttggggaaagaactgtaataagtttaaatatttatagaattataacaatacaaataccaaatgctggcaggctgaagggtagaagtaatgagaaaggagaaaagaataggaggaagacaacagaaagtatctaaaaccgaaaactcaagagaaaagcaaaatatgcatgctacttcaaaaactatcagaatacaaaggataaataattgaaaattatttacttcagatagtgggaattggtgaataatagaattagggcaggggtctactgcttttctgtttgaagccaagtggaattctttgacatattttttcacacatactgttaaaaaaaaaaaattaaagagaaaaaaaagtccaggaaccaatccaagtacatataacaatttagtacatgataacagcaaaacttcaaaactggaaaaatgattaagtgacactgggacaactgatcaactatttgggaaaatatgcaattagagcctgacctcatacatgcatgaaaataaatcccataaacaagagagttaaatgcacaaacaaaactgtaaaagaactattaaaaatatccttataaatttatagaaacggatctagaaagatcctcatgaaagtgttcatggtgtttccttctggaaagcagaaagggagatggggttaacataagggagaacattttaatttttattctgtattggtttttataatgttaaaggtttttcaaaaaatgttattactcttttttttttttttttttttttgagacaggttttgctctgtcacccaagctggaatgcagtggcatgatcacagctcactttaacctctacctcccgggctcaagcaatcctcctgcctcagcctcctggtagctgggaccacaggcatgagccaccacaccaagctaattttgtttgtttgttttttgtagagacagggtcttgctatgttgcccaggctgatctcaaactcctgggctcaagtgatccacctacctcagcctccccaagtgctgggattacaggcataagccatcatgcctggccaaaaactatttttaaatctgcattaagtcacctgtatttaacaatactgtcataccctcttaggtataaggatctgagtaaaaattgtaagctacaagaaacagtggtgaatcatacatctacttaactatggccatttttttcattcatttattcaacaaataatacaagtagctactaggtgcaggctctgtgccagatgctggttataaaaaactgaaaaaaagagacattacacttgtccttatggtcagtctggtaatgagacaaacaaaacaaaaaacagttaaacctacaaataaataaataattataaatttgggtaagcattgtaaatgcttagatttggtgttgagacactaaaaaagggacgaatcttgaataaataacctgcatggtaatgcatataatatgacctgtaacagtataaaaatgaactgtcatcaaaaagtcatctggaagagtttattcagagacagaatagtgaggtggtttagaggatcaattccagagccagactgcctgggttttaatccagcttctactcaccaggtttgtgacatgaacaagttattcaaatactctgtggctcagtttcctcagctataattgcggaaaatattaataccttcctgatacggttgttataaagattgagttaaattcttttattttgaaaactgtcaaacccaaagaaaagttaaaagaacatgacaataagcatcaactcacatgaacctgtatttgccaggtgaattaatatttctaaagtgcttagcagagtgtctggcacataacacagtaagcaccagtatcagcatatatatgcagttatgttaaatagctattttaaataaaagtaaattaaaaccataaagtaaaagaaagaagggtaggaaggaaggaaataaaaatggggtatgggaggaagaaaaaggaagaaaaatagcctaaggatcgccccagtgttaacactttgtaagaaggtcaacagcgcactctgctggacccaagtgtcttccacacaccaggcccaagttatgaattaaaataatatgctactgctacattaaatcaaatataattactttaattaaagtatataaagaatgttagcatttatttaatgaataaataaaaagcaggtataagcataagaatatatctgaagacaaccctggctattgtgaggatttactaaaaaaagaacagaatataaataaatctagagaaaatgattatgaatgatagaacaaagtaatatcaaaacgttaacatactattctaaaaaacaatgtttttcttaactatattttgaccatgtggagacagatgcttcactagaactttgacatatgaattgtcagattaagaaaataaatctgaactgtaaggaaagggatcaagcaggggacaccagaaagttaattatataagaaaaggtactgacactatcttccgacttgaaaagagagttaggagactagaaatgatagaaaaataaagagacaacaatctacttctccaatccactaggtggaaactaaatgattttcataaaaactaagaaaatttgaagaacctaaaaagtttaatgtcaaattcaggggaaaacagaaatcatcaaactaatttaccatttaatttatggaagaaggtttgtcaaaagtaatgcaactgcagatttgaaaaacgtgaagactaagacacacctttgcagggatgagaattaaaccattaatttatgaatccaaataaatatttctatttaaaaaatactgtactccctagtatcatataataaaaagcaagataggaaaagcgtccttagaatttaaaggtgttagaattcaattagatataacgtatctcctgacctcgtgatccgcccgcctcagcctcccaaagtgctgggattataggagtgagccaccatcctggctaacacggtgaaaccctgtctctactaaaatataaaaaattagctgggtgtggtgacgggcgcctgtagtcccagctacttgggaggctgaggcaggagaacggtgtgaacccaggaggcggaacttgcagtgagccgatcgcgccactgcactccagcctgggcgacagagcgagactccctctcaaaaaataaataaataaataaataaataaataaataaataaataaataaagcatcattaaatctagtctcccattctttttccttccatatgaaggaactggttttcacaaaaaagttacttacccaagtctatcaatttaccttattcaagattattacaaggtctagattaaaatacaacagataaaatgtgaggcattttggtctgaagaaaaaagcaaaaataaaaaaatataaaaaaaagtgaggcatatgatctgtcagcataagaaaagttctaatatatggactacttttttttcaattactttatccaaaagttttggcaataaactcatttcaaaagctgctgtataataaatattaaaacttacgacattaatacacatgaaactatttttcttctacttttcatgaaaccacctatcccaaaatctacagaaaatacttttatgatttataataagtcactttgcaaagactaagaaatgttacagcagaaaccaaagtaatttcctacaaggaaaaatatttactcccctatgacacccaaaaatcccattttttaaataaattacctctactcatatccccattattttttcttatattacattcttggaaataattagtccttgttctctgaaaacctagatgtattatttaatgctagaaatccatgagaaatttactaaaaatttacttaacatacaaaaaggagaaacattcatgctcactaaaactatacattttataataattatatttctattataagtacttatcctatttttatgttaggaagaaaaaaataaagttgaaaatacaaatcattttacctatgtatttttgggtttttttttcccataacctttcagaaagcacataaaattactttatacgtcatgtttttcatcatagactaccatgaaaagaaacaaatattagagctcaaccatatgttctttgcattgcaaaagtgctttgatgtctgttaaatttactttatgcatgtttaattagtatgacatatatctcttacatcttcttgcaaacaaaaattcagtgtcacactagtgaaatgttatcatttgagggactagatttaattgagagatttaacttgttcaagaagttttcagacttatgagaaattgtcagaggcctatatcttaatgttatgagtaataaatcacccacttatgaaaaattattccatttctagagatatgtagagttataccacctaaagccaaggaagaaaatatgcaatttctagcttaaaaataaaacagatataaagatacagatatacaattatagatacagctatattttaccaatgagcagaccataatcaatgatcaacatatatcagcatactacataaccaaaaggagaaatcaatcaaagcattaggcttaaaattaaggtactcaaccaagggcacagtagatgtgcttatggtcaagcggtttagactcaaaagatttcagccataaacatcttcaacagatatcaaaagaatttttggccaggttccgtggctcacgcctgtaatcccagcactttgggaggccgaggcaaacagatcacccgaggtcaggcgttcaaggccagcctggccaacatggtgaagcctcatctttactaaaaatacaaaaatgagctgggcgtggtgtcaggtgctacttgagaagctactcaagaggctgatttaggagaatcacttgaacccaggaggcagaagttgcagtgagccgagatcgcccactgcactccatcctgggtgacaggggagactccgtctcaaaaaaaaaaaaaaaaaaaagaaaaggaattttctactaaactctaaacctaactcctcaaaatttataatgttgatcagtattatcaacaataacattaataatagctttcagcagcatttaaaaataagaactacaaggccaggtgcagtagctcacacctataatcctggcattttgggaggccaaggcaggtggatcgcttaagcccaggagtttgagagcagcttaggcaacatagcgaaaccttgtctctaaaaaagttaaccaaataaataaaatgaaaataagaactacatattactccaagcaacctacagattcaatgtaatccctatcaaaataccaataacattctttacaggaatagaaagaacaatcctaagattcatatggaaccacaaagaccctgaatagccaaagtaatagtatgcaaaaagaaaactgtaggcaccacgctatgtactacacaaagctatagtaaccaaaacagaatggtattagtataaaaatagacacatagaccaatggaacagaatagagaaccgagaagtaaatccaggtatttacagttgactgattttcaacaaagatgccaagaacacatatcggggaaaggactccctcttcaataaatggtgctgagaacactggatgtccatatgcaaaaggaaaaaactagatccctctcaccatctacaaaaaccaactcaaaatagattaaagacttaaacataagacccaaaactataagactactagaagaaaacatagggcaaaagcttcaggacactgatctaggcagattttatgggtaagaattcaaaagtacaggcaacaaaaacaaaaatagacaaataggacagtatcaaactaaaaagcttctgcacagcaaaggaaacaacagagtatagagacaacctgtagaatgggagaaaatatttccaaactattcatctgagaagggactactatccagaatatacaaggaactcaacagcaaaaaaaaaaaaatctgattttaaaataggcaaagggtgtgaataaacatttctcaaaagatacataaagccaacaagaacatgaaaaaatgctgaacatcactaatcatcagggaaatataaatcaaaagcacaataagatatcatctcactcagttagaatggcttactatcgaaaagacaaaaaaaaataacaaatgttagtgacgatgtggagaagaaactcatatactattagtggaaatgtaaattagtacagacattacggaaaacagcaatggaggcttctcaaaaacctaaaaatattactaccatataatcatataatccagcaatcctactactatttatccaaaggaaagaaaatctagagtacagtagcatgatcatatctcaccacagtctgaaactcctagagtcaagtgatcgcccatgcctcagcctcccaagtagctaggactgcaggcacgtaccaccacgcctggctaatttttttaaaaattttgtacagatggggttctcgctatgttgccggggctggtctcaaactcctggcctgagtgattctcctacctgagccttccaaagtgttgggattactggtgtgagccaccacacccagccaacaccatgtttattgcagcgctattcataatagacaaggagttggcatcaacctaaatgtccatcaacagatgaatggataaagaaaatgtgacacatatacacaatggaatattattcagccatagaaaaatattgaaaccttgttatttgcagcaacatgggtaagcctggaagatattaagtgaaataagtcaggcataggcaggtaagtactgtatgttcttactcatatgtaggagctaaaaaaaatgagctcatagaagtagagtaaattgtggttatcagaggttaggaaggggtgaggggaggggaggataaggagaggtgggtttacaaattcataactacagctagattggagaaacaggaggaataagttctggtattctatagcactctagggtaaatatggttaacaataatttcttacatattttcaaaaagctgtaagagagaattctgaatattcgcaacacaaagaaatgacaaatgtttgaggcgatggatatgttaattactctgatttgatcattacacatcatatacatgtatgaaaattatcacactgaatcccataagtatgtataattattatgtgtcaacaaaaataaacaaaaaataaagatcagaactacaaatatgtaatattattagtcggtattatatagaatcttttatcagcagcagctaaaggtaagaactacaaatatgtatccatctgctgctgttactactactacagctgctgctacttctactaccacttggcgcttaaactctaaggtatgccagatactctcagcactgtatagatatcaacttgtttaatcctcctaacggccccgtgagataggtactattactaaccccatttaacaaatagctaaacataataccaagagtttaaaaaatcaaggttatacagttaataaagagtagagctatgattaaaatccaagcagtctcaagatatgaaaataacctaagtgtctgtcaatggatgaataaagaaaatgtggtgtgtatgtgtatgtcaatggaatattagccctaaaaaagatcctgctatttgtgacaacatggatgaacctggagggaggacattatgcaaagtgaaatatgtcagatgtagaaggaaaaatactacatgatctcacttatacgtggaatctaaaaaagtcaaatacatagaaacagaaatcagaacaatggttactggggcagggaggtgggggatttgaggagatgttgatcaaagagtaaaaagttgaatctacataggatgaatcaagtctagagatctaatgaacagcatcatgacaacagttaataatactatattctaggctggatatttcgatattggctttaaaattttcaggtgttctcaacacacacacacacacaaaaggtaactatgtaaggagatgaatatattaattagcttgtgatatgatttggaagtgtgtcccctccaactctcatgctgaaatgtgatcccccaagttggaggtggggcctagtgtaaagtgcttgggtcatcaatggctggtgccctcctcacagtaatgagttgagttcttgctctgaggtcacacgagatctggttatttaaaacagtgtggaacctcctctgctctctcctctgtcctctctttccatgtgacaagtttgctctctctttgtcttctggcatgcttgtaagcttcttgaggccctcaccagaagcagatgctgacaccatgcttcctgcacagcctacagaactgtgagtgaaaataaacctcttttctttataaattgcccagcctctggtattgctttataacaattcaaacagactatcacagcttggctgtagtaataatttcactatgtatatatatattaaaacatcatgttttacacctaaaatatatataatttttattttatgtatttattttttatttttttttagagacagggtctcgctctgtcacccagactggagtacagtggttcaatcatagttcaatacagcctcaatcttctggctcaagggatccccctccctcagcctcctgagtagctaggactacaagtacacaccaccacacacagctattttttttcttctattctttataaagacagggtctcgttatgttgcccaggttggtcttgaactcctgacctcaagcaatcctctcaccttggcctcccaaagtgctgagattataggcatgagccatgatgcctggcccaatctttatttaaaaattaagatcaggcagtatgtgtccaaagtccattctcttaaccaataaaaacaaaacaagccaggtgcagtgattcatgcctataatcccaaaactctgggaggcccaggcaagaggatcactggaggccatgagtttgagaccaacctgggcaacacagtgagacctcatctctacaaaaaataaaaataaaaaattagctgggcatggtgttgcacagctgtagtccttgctattcaggaggctggggttggaggatcacttgagcctgggattttgaggttacagtgggctatgatcacaccactgcgttacaacatgggcaacacattgagaccctgtgtctacaaataaagcaaacaaaaacaattcccaagaaaaaaaaatcttaatcacaggtgactctatacttgatttgtttatctgtgagcttctacatatttctttccttatcttggtgttagaaccctatttaggtggctccaggactgcaaagaggcattttagagagaatataaaataaagtgattagggcatttgccagaacatgagaatgcctaggcaggatttagctctactatttactagctttgtgatcttaggccagtttcttccttctcagagtttcctcatctgtacgatgggaataccaatatacctgcttcataatcattgtaagaactcagtaacattcaacaaataaatatttattagacaactaatatgcaccatatgctattctaggcatgagggacacacatgaaaaagactgacattacatcgtaataggacagacaaataataaacaagtaatcaagtaatttcagataatagtaagcccatgaaggaaaggaaagaaaatgaatgagaaaacactgtggcagagctcaacatatagcttacattataatgatgttgatgattattgcaatttaacagaagaagcttctacataatttctccaactattaatataaagcttcagtgaagatcaccaacaaaacacaaattaaatgtacagtaactgaaaacaaatctgtggttgcctgggaatggggggacagggcagaaaaaaggtccatcagtctatcttgcacttaggatggaccttttctaggcatcattttgcaatatcatgcactggtcatttaaataatatttagttcgctgagtttaagttcattgagttttacagatcttacaaatgttgacacatttcattatataaatataaaaagtcatattctgtaatatcaccaatttcatcagaaaagtctgtagttatcacaaagctgccaagctcagagtatatacaaattttttaaaattctgattttcacttgaaagctcaaattttatctggctcattttatcataaagaattaccatggttagtatatcattcactctttttcaagcaaaaatgatgttccatgacaaaaagtggctagtcagctcacaatccaaactatcctacaggcgcttctccccaggacatccatcctgcttctgtatgcaaaagtgttttatgagttctattcatttcttcagacagaatgttaaaaacctaaacggctgagattaaataaaattagtaatttttactgcttcatcaaagacattcttaaatgaaactggttttttttcaaaactgtgatctaatgatgctggaaaactattattaacataggttgggttgaggccaaggtgggcggatcacgaggtcagaagatcgagaccatcctggctaacgtggtgaaaccccgtctctactaaaaatacaaaaaattagccaggcgtggtggcaggcgcctgtagtcccaactactcgggaggttgaggcaggagaatggcatgaacccaggaggcggagcttgcagtgggcggagatcacgccaccgcacttccagcctgggtgacagagcgatactccgtctcaaaaaaaaattaaaataaataaatttaaaaaattatatacatatagagagagagagagagaaaaagagagagagagagaaagagagagagagagaggttggagccaataccttgatttctgctaaggcaccaacagttttacccattcttgcttttgcgccaccagtacaaatgtcaagacaatccaaaaggcaataatgtattttttcctttttttttttgagacggggtctcactctgtcatccaggctggagtgcagtgtggtgcaatcttggctcagtgcacctttgcctcctgggctcaagccatcctcccacctcagcctcccaagtagctgggactacaagtgtgcaccacaacacccggctaatttttgtattttttgtagaatcagggtttcgccatgttgcccaggctggtttcaaactcgtgaactcaagtgatccacccgccttggcctcccaaagtgctgggattacaggcgtgagccaccacgcctggcctacaacatcttaatattattacaaaaataattttcacctcacagatcactccccaaaaaaatctcaatatctagaactccttggactactccttgagaactgttgccctaagaatcctgaaagaactgtaaggagttggagaatttacaaacattaaaatacagaacagaaaaaaatgactttcagatctctctctagtccaattctcttattttataaataaggaaacgaggcatagagagaataggctttgccatctcacacattagtaacagagatgtgacagcattgaggacttctgagtgggttgctactgtcctttgtattatactattgttcctcttttgtcaaaaattggtttagaaaattccatatcttccttctttaaaactgaacaatggcattaatacatactttctgtggcgaaaaggagtttatcttttccagtggagagttgagttttacagtctttcctgaatcaacttttgcatccaaattgtgtacttctgttccaaagcaatgacgttttctaatctgtaaacacagaaccaaaatgaagtttaaggtgaactagaagtttaactggctagttgttctcaaaggccaaaacagctctatgtatttttttcctgcatccaaaaaaagactatttctaacaggcaataataaaagaaattttaaaaatgcaaataagcacctcattacaacacttaaaggaaatagaacatagtagtgactcatgaatcttagccaaactatctttaattagtgatgtatcaaaattagagtattgtaaaatgaacttctcataaaatagtcccatcatatggacatcaaatatagcaatattggaaagtccaatcttatatctaattggaaaataaaagtaaatcaaaatttaagcataattctacattgaaaagaactcagaataaaacttttaatgtaaacagtcatgatttcctacaggatggaataaattactcgattactattatcactgttacttactagatatttgataaaccaagttgcctttgattaaatttcttttataaataccactaagttttctttccaaactgtatccatgcttttcctatttgtgaaaaatctattcactatataattggggagaaaagcatttctattgataatctcattcccgccaaaaatgaaatgagaaccagtgaagtgagggaataaatgttttatgttgagccctacctgatgacatgcaaatctatgaatacattttgtcggggggaaatctctataggttcacacctcatttgtcattggtactttgataaaataaactagttcattctatttctaaaaacaaggctgaattatagaaatttataaagactgtaagtggaacagttaaactacataagacttcaagaaccttgagtatactcaattaaaagtacccagttacttacctacaaataacatcctaattaataatttttttattttatgtttttatttatttattttttagagacagggtcttgctctgtcaccaaggttgaagcataattgcatgatcttagctcaccacagccttcaactcctaggcttgagcaatcctcctatctcagcctcccaagtagctggaaagtagtctgtagggctacatgtctgatgagcctgtagggctcatgccaccacacccggtctttttttttacttttttgtggagacaggagccttgctatgtcacccaggctggtctcaaactgctggactcaagagatcctcccacctcagcctcccagagtgctgggattacagtcgtgagccactgtattcagcctaataattatcttttttatttgtaactaaatctattaaaatcccagccttcctatctcactgccatgaattctgagaaaatattgtgttcatctgcatccaaatttcatttaaaatatctggtaattcaagccttttagtaatttttagaaataaatatacatattttatcatgtatcaatcttaaaaactcagtaagaatacatcttgtcattacattaaaactatcaattccaaaatatccataacatgaataatcatatcaaaggtattttagccaaaaggaaatgctattttaagataaaaacaatggaggagttaaaaagtacaaagtagtatatcactaagatatgccttaagtaaatattttactatcaaattcctcatatataagaaacaccatgcagtttcacttgaacgacatgttactgtgaataatctgctataataaactcctttgtaacaagtaactctacaaaatgaaattacaacaaattattaatttatggctgtcacatgacccaactaatctccacaagtgcattaaaaacatgatacttgactaccatgttcagctgtaactaactgggttatttactgccaataaactctgtttacctgctgtgtagtttctaagggtcgaattcttttcttctctacttgaaaatcatcattttcatctctgtatatggatgcctgtttcttagcagataactttgcagccagagtggttttttcaggggagtcttatataagtaatttaaaaaaaacagcataaataacttacaggtaggcaatttttctagaagaaaactggaaccaggatgtaaggcttatttctagaaatttcataccataatctaaaacatgaaacttaaacatgtttaaaaagtacttctctaccattcagtttactttaccaaagattctgagaaacatgctttcaataggaaaagtccaatttgattttttaaatcgccttatgtacttgtagactactcccaataacatgatttaaaatgtataactgcaaggatctctgaagaaaatatatttcatgtctgtaaaaatctttctttacagtatcaacgtttcttccctactaagtttctccacgttctttagagcaaaaccattgattatagaatcaagctcatataatctgtcttttaatcccataatcctgaaagctccttctcaaggtcatccaggacctcttaatcaccaaataacattaattatcccaatcttacaaatgtgaacactaggtttttaaaagtctagtagcttgcccagtatcatacagttattaaatggtagagcctagacatgactgaatcctgctgtgtgactccaacatccatacatgtaacctcttaggcatacagccaacactgtcttcttggaacacagcaaacacattttcttcttccttaatgtctgtaacactgctcttaactattctcatacattggtttctcccttttcctaaatataaatattcttaaaggtaccctccttcctctttgcttctctctaagcattctctccactcccatggcttgaattaatttaacaatcttttttaaccatctttttttttttttttgagatggagttttcctcttgttgcccaggctgaagtgcaatggtgtgatctcagctcaccacaacctctacctcccaggttcaagcgattctcctgcctcagcctcctgagtagctgggattacaggcatgtgccaccacgcctggctaattttgtatttttaatagagacggggtttctccatgttggtcaggctggtctcgaactcctgacctcaggtgatccacccgcctagcctcccaaagtgctgggattacaggcgtgagccaccgtgcccagccaacaatcatttattacactgagtctaacccatggccagacaccatggaaaaacagtgaaaaagagaatccttggccctaaaggatttacagcagagaaagacctacatgagcaggtccaatactgttttcaaaacacaaatttgaggccggccccagtggctcacgcctttaatcccagcactttggaaggccgaggcgagtggatcacttgagcccaggagttcaagacctgcctgggcaacatggcgaaatcccatctctattaaaaatacgaaaattagctgtgcatgttggtgcaggcctataatcccagctactcgggaggttgaggcatgagaatctcttgaacctgggaggcagaggttgcagtgagctgagatcgcgccatgcactccagcctgggcaacagaacaagactctatgtctccaaaaaaaaaagaaagcttgagattacataaactcagaaatgaacctatgccacatagcactgattttcaagttttttctaaagtgtaggaatattttcttcttaaagaaatgttgcagctgggcacggtggctcacacctgtaatcccagcactttgggaggccgaggcaggtggatcacctgaggccaggagtttgagaccagcctggccaacgtgacgaaagcccatctctactaaaaatacaaaaattagctgggcatggtggtgcatgcctgtaatcccagctacttgggaggctgaggcaggagaatcacttgaacccgggaggcagaggttgcagtgagccaagatcgcaccactgcactccagcctaggtgacagaacgagactccatctcaaaaaaagaaaaaaaaagaaatgttgcttaaaatttccaaaatagggatgaaagacccagaacttccctgatccactttcccccttcccttatgagtcatgtttctttctttataaggttcttagaaacacagtttgaaaaccactatactctaaaaatcctaatagttttgcctatcagaagtaggtctttaatctaccttgaatgggcttttgtatatggtataaagtagggctccaatttcaattttttccatatggacatctaacaggcctagcaccacccaagttcttcctttcccctacaagctgtaggggcacttctataaaaatatcaagtttctggatacatgtgggaccaaattagggttctctatacttttatatgagtctatcactgtgctaacctcagtctgtcttaattactctatatttacacacaataaatccgtatatttggtggatcaaatctctcaccttgacctgcttcttcaaaagcatcctggctatccttagtcctttgcatttccatatatattttagataagataggtaattttgagctctgcatgtccaggcaaccagtaaaataaccacaaaataaacctagatcagtgattctcaaccaggccaattttaacaccaccacttctcatcccaggcatactgggcaatgtctggagacatttttcgttgtcacaaaaagagcagaggtggaaatactactagcatccagtgcataaaggccagaagtattgctaaacatctcacaagggagagctcctacaacaaagagttatcccactcaaaatgttaatagtgccactgtggagaaactctgtcccaaataaatacggggcaaactgacatgggcctattaagagggtagggaatgtccacccaaaatgatcatttagttttagaaaactgaactgccatgtgccaaataagggtaaccccaattcataaaaataaaaatcatattatcatcacatacaaaaaggcttatcttcctggtcttgatagcaagggagtttgaaggaagttcatcaaaggggtaaaatctttaagatacaaagttaaaaagaaagaagatatctcccgattatgtttttttaaaatcttcaagaaacctaagaaatacccaatagaaaaatggaggctgggcacggtggctcacacttgtaattcaagcactttgggaggctgaggcgagtgatcatttgaggtcaggagttagagatcagcctggccaacatggtgaaaccccatttctactaaaaatacaaaaatcagccgggcgtggtggcaggtgcctgtaatcccagctactcaggaggttgaagcaggagaattgcttgagcctgggagctggccagtgcagtgagttgagatcgcaccactgcactctagcctgagcgacagagtgagaccccacctcaaataaaaaacaaaagaaaaatggcaaacaacttgattaggcaattcactaaagagggtatccaaatgaccaataaacatatgaagtcaccagagaaatactaactaaaacccaatgagaataccacacatacacaagaatgactaactttttttttaaactataaatatcaagtactaacaaaaatggtgaacaactttcatatactgctgcaggggttggagggatctgaccggggacgcataaattgattcaaccactttagaaaatgttgtcagtatctactatagctgaacatatgtatatactatgaccaggtatgtgtctacagacaatacttacctatttgcaccctttgaacaagaatgttcataacagcactattaaacaatactgaaaacaacccaatgcctaacaactgtagaacagattaataaactgtggtatattcatacaaggaaataatacacagaaacaagaagtactactatatgcaacagcatgaatgaatctcacaaatacaatattaagcagaagacagacacaagattacatatataattccattttttttttttgagatggagtttcactctatttctgtaaaaaggcaaaactcatcaatggtcacaggaggacaagaattacctatggaaagaagggacagtgactgaaaaagaagatgaggagggcctctggggtgttggtaatgtctctctcttttttttttaatctatgtagtagttatgacagtgaaaattcatcaagttatatacttaggatttgcatttttctatatttaggttacagattaaaggatttacttacaaatataaagagcgtatcagaatacaaccttgatttttcccatttagactattctaaataacactgactcacagtaatgtcaaaatcaagttgtgtacatatggataactcattgttgataaagacacaaaggcaatgtagtggataaaggatagccttttcgatatatccaggtgctgaacaactggatatccacatgcaaaaaaatgaacttgaatccataccttacaccatatacacatattaactcaaaatggatcttagacctaaatgtaaacctaaaattctatctgatcaaagacttgtattcaaaatgtttaaagaagtctcaaaatttaacagtaagaacacaaactaataaaaaataggcaattattcgaacagacatatcatcaaacaagatacatggatagctaataagaacatgaaaatatgggaaataaaaattaaaaccacaatgagatactgccacatgcctattagagtagctaaaattaaggccaggtatggtggctcatgcctataatcccagtgctttgggaggccgaagaaggcagatcacttgagcccaggggttcaagacaagcctggggaacacagtaaaaccaacaaaaactataaaaattagctaggtgtggtggtgcatgccagtagtctcaactactagggagtgtgaagtgtgaggatcacttggttccaggaggcaggggttgcaatgagccaagtgagccaagatcacagtactgtacccagcctgggcaacagaatgaaaccctatctccaggaaaaaagaatggctaaaattaaaaggactgaccataccaagggttcacaaggatgtgaagaaactggaactctcataactgctgatgggaatataaaatggtatgaccacttcatgaaacagtttagaaatttcttaaaaagttggccaggcacggtggctcacgcctgtaatcctaacactttgtgaggcacagatgggtggatcacttgacgtcaggagctacagagcagcctgaccaacgtggtgaaaccccatctctactaaaaatacaaaaattagccaggtgtggtggcacattcctgtaatcctagctacttgggaggctgaggcaggagaatcacttgaacccgagaggcagaggttgcagtgagctgagattgcgccactgcactccagcctgggcaagagtgactccgtctcaaaaaaaaaaaaaaaaaaaaaagttaaaacatacacacctacaaagcaatctggtcattctgctcctagtattcacccaagagaaatgaaaacatacatccatacaaagatgtagacacaaatgttcacagtatctgtctttgtaatagcccagaactagcaataacccaaatatacataaacagccaataaccaatttgtggtatatccatgcacaattactcagaaataaaaagaaacaaactattaatacatgcaacaacatagaggaatctcaaaataattgtcataagtgaaagaagccagacaaaataagtatacatcctgtatgaatccatttatataaactatagaaaatgcaaactaatctatagtaacaaaaatcagacaggtggttggttggggaggtgagtcatgtaggggtgggaaagagggataacaaaagtccatgagaaaactttttgggaaagtgagcatgtttaaaaagttttggataaagtgaatatggggttggtgtgtatgttcactgtcttgattctggtgaaagtttcatggaggtacatatgtcaaaatgtatcaaagtgtaccatttaggctgggcgtggtcgctcacgcctataatcccaggactctgggaggccgaggcaggtgagtaacttgaggtcaggagttcgagaccagcctggccaacatggtaaaatcctgtccctactaaaaatacaaaaaattagctgggtgtggtggcaggcgcctgtcatcccaattacttgggaggctgaggcaggataattgctcgaacccaggaggtgaaggttgcagtgggccaaggtcacaccactgcactccagcctgggcaacagagtgagattctgtctcaaaaaaaaaaaaaaaaaaagtaccatttaaatatatgcaatttgttatgtcaagtaaacgtcaataaagctgtttttaaaaggttgtatataaatattatacaccagaattcctataattagctgaaaattaatcccattcattcctaacaacaaatatttatctagcacctaccatatgccaggcaccctgctagagttctagggatacaatggtaagcaaattgacaatattcctaacctcatgaagtagtctgccaaggaagcagagattaataaaagatttagacagacacatataatacaaactgtaacaagtgctataaaggaaaggtacacaaggagaagacagactataataggaaaatcaaatctactctgacagaaaaatcagtgatggataccccaaggaagtcacatgtaaactgaggaagaaaaggataataaattgaagatggaactttgctcagaagagacactaatagaagcaaaggctctgaggaaggagaaagcataaatctttgaaggaaagggaagaaatccaaaaggccaaaatcaaaacaatgtagacaacactattggtcacctatttaataactgttccacttctctttcctcattcctaccagaagttcaatcatctaccctcctctatatagtcacatgctttagaggaagctgactccatccttgattttaggaaatggatactctttgttttgacttaagtcaatcatggcaacctctgtctccctgccagtgactggtaaaggaatagcatgagtaaaagcagaaaaaagcgttattggatgctctaaacaagaaatggtagtttggactatggaggtaccaatggatatggagatgtacatgaagaaataagaaagagcaaagtgtccaggatgttccctaggtttcttaggtaaataatagtctatcctttctatacagccaaaagcaggtgaaatattttttaaatatattattaataatcttatgttactcttaatgtatttctctaaaattagtaaaaattatttaaagatcacacataaacatacctgatacttatgtaggatactggcctcagaaggccaggcaaacttatcaagaatgaatcaatgaggctaattagatttcgtcttaggatacaaacattgttttatagaattctaacatataattaacaataatggctacaaataacatcaattttggaaaatcatttattttgccttgccttccttaaaaaattatttaggacaacaaaatgtctcaataaattaaaagtcaaaccaagaatttaaaaacgtctatggatttttgaccactctgtgctattttaaaatcattccagagaaactagatagagatatataatcagttatgctaaccaaacttatataaattagggcttataacagtaataattaagactcttattacagatatcaactgacccaggcaaaatataaattaccttgacaagttgatgaagtgccatttctttcagaaggtggtgtgcttggatagttgaaatgacgtgaagttccttggttcatgtcattgtttgtaaaatcctttgaatggcatgcacaacaacatgacaattgtacttcagctttttcacttacgccctcatctgctggtttccctaaaaatgaaagaacatctatttataatatatctaattaaataaacatcaatcattctctacagcccagttcacccaggattgggaggtttcctaagataaaagatttttagggctaacaccaggaaggtacctggcaaacctggacaagctggtcactttatctgcagcatcaaattaggacaggagagaaataagccaaaaacatttggttggacacagtggctcacgcctgttatcctagcactttgggaggccgaggcaggcacatcaattgaagtcaggagtttgagaccagcctggccaatatggtaaaaccctgtctctactaaaaatacaaaaattagccaggcgtggtagtgcacgcctgtaataccacctactcaggaggctgaggcaggagaatcgcttgaacctgggaggcagaggtagcaatgagcagaggtcatgccactgcactccagcctaggcgacagagcaagactctgtttcaaaaaccaaaacaaaaacatttatagggaaattatcacccatttatgtaatctgtaaaaatctaataatctaagattaataatgtaataaaagtacaactactcaagtattcatttatattaagatttatctaaaattcaagaatggattgaaacaaggacataaagtatatccttaattactactctttccctaaatgaagaatttgaaagttcaatatattagataataatctaacacataagggttaaatcattcccccaattatacttctatttcaaatagaaacatgttttttgatgtattaattctgatacttgcatcaaaatacattttaaggtcaatattcaaatcaataaaataggtaattttagtgctaaatataataccaaattgtaataagtttacattgtgtgcctacattgtatacaaatgtacaattcatatacccccaaaaaagctaaataatttatttttaaaaataatcagtattgacattgttttcccatttgtaccatataaatattcctaattctattttccagacatctgccattaagacctgaagttagtaccgataaactcatttttcccaaaattaacgtcagtgagaatcaaaatgaacaaccaaaagcttatgaagtatttactgtttttttttttttttgagacagagtttcgctcctgttgcccaggctgaagtgcaatggcgcgatctcggctcactgcaacctccacctcccaggttcaagtgattctcctgtctcagcctctcgagtagctgggattacaggcacacgccacctcaccaggctaattttgtatttttagtagagacggggtttctccacgttggtctggctggtctcaaacttccaacctcaggtgatccacctgccttggcctcccaaagtgctgggattacaggcgtgcgccactgtgcccagccttaatatactcttattagtaacaaacatttcattttaaaaatgtatctatatacctatatacacatacatacatacatacacataccatgtttaacctcaattctcctaatactttaggttaaagccatctaatgtaactaaaatgtatccaatattatatttctgattcattatgtagtttatatagtctttcatagaagtaaatattaataggcatttgttagcaaacaaagtaaaaatagtttggcctggtggctcatgcctataatcccaactatttaagaggctgaagcaggagaatcacttgagcctgggagtttgaggttacagtgagctatgatcacatcactatgctccatcccaggtgacgccactcaaaaaaaaaaaaaaaaaaaggaaaagtagtgggaagtaatataaattcatacaaacaatattatactaaatttctgtttatctggcatttaaacaagtagaaagatcaactagaatgtattcttttttatatattccatgtgctgatgttatgagaaaggaacaggtaagcaaatatattaaaggatttccccccccaaaaaaagcaattagtatgtgttacagcatcatgacagcccatctcttgcatcttttacatacaaattatcatctaattatttttataataactctaagcatccaaaactttgtttgtttgttttctttcacccaggctggagtgcacaatcacagctctctgcaacctcgacctcctgggctcaagcgatcctaccacctcagtctcccaagtagctgggactacaagtgtacatcaccatgcctggctaattgattgtcaatttttgtagagatggggtatcaccatgctgcccaggctgccaagtctttatgtactttccgactcatcaaaagactaaattatgttcaatactattttagcattaattaaacatattttgctatattgaaactcttttggaagatttataacttatttcaaagaaacatctttaaacaaatatttaagttagcgacagcatggctgaaccagtctggataaagaatactgtattatattttctcagatcccagtaagtaacctgaagatatcaagcaactacttaccactaagagattgttgccatgctaaagcagaacaaagtaaggctaagctttttccacttcctgtgggactctccaacaaacaatgttgcttgctgtttaatcctctgagaatctatgaacacagaaaccaatgaaaataataaacatattaactttataaaggtctctctccatctgaagtttaaatagaacagcaaggaaaagtgagattgcactccagggaacacaacaatagcagaaggaactcatatttagttaaatccaaactgtattcctttacacctctccctaagattatcttactaaaatatcaagcacaagagaagaaatgaaaagaggaatctgtgccagaattatgcaagcatcaatgatttctgtaaaaccagaatacattactacctatcaatatatttgatgaaaacttaatgaaggatactgctagtgagagtacaaattgttacaactacttggtagagaaataaacaatttcttataaagtgaagatgcccagctccagcaagtccagttctatgtgtacacccaaaacctttcccacatgaacacaaggaaacatgcataagaaagttcacagcagcagccacgcttgtaatcccatcactttgggaggccaaggcaggtggatcatttgaggtcaggagttcgagaccagcctggccaacacggtgaaaacccgtctctactaaaaatccaaaaattagctgggcgtggtggcgcatccctgtaatcccagctactggggaggctgaggcaggagaatagcttcagcctgggaggcaggggttgcagtgagccaagatcgcgccactgcactccaatctgggcgacagagtgagacgctgtctcaaaaacaaaaagaaaaaaggaagttcacagcagcattgtttgtaactgcaaaagaatgtaaacaacctaaatgtctaccaataagaaaatggacagataaattctggtatattcatccaatggaatttatacaacaattaaaatggattaagaatcaacagggctatacattaaaaataatgctaagttaaaaaactcaagttctgcataaaaagtaaggtaccgtatcacatcaatgcacagtttcaaatatgtaaaataatattctatattgtttatggatacatatatatgtaataaaagtataaaaacatggctaggaaaggaaaagaacaaattcaggatactaataatcttgttaccagtaaataagatatgaagtacatatggcaaaatagctgtaacaaaactggaaagctggtttactcaaatttgtatattaattttctacatgatccaaacaaaacaatggtgagaaaaatagtattctgtgtaaaagatagtaaatactctgtttttacttaaaagtatagcagtttttcattaaaagtatagaaattccaaatgagctgagaacatttttcagaatgttcttctttgccttaaccacagatatatacagtttagctgcaatcagtagtttcccagaggttagatattcttccaagtgaacccagaaaatattctccatttactgagaatatgaatgcagtcaaatactcaatgtactttatgggtcataagtatctatatcttaataaaaacttaactgctgaaaaatacttacagaattcatcatagcaagctgtgacgggtaagctttataaggaaagtaaatcttcaccccaccaattgtatattcagaccacattgaagacatagtgctttcctgtttatttcagattcctaactacaacagaaatgaaaatgtcaaatattgagacacgccttacaaagaaaaccagagaaccaaaactaagggagaaatctggaaacagaaactggaattaataaaagtataaacaaaacaaatggaaacaaaataatttcctagtcttataaatcacagcggtcaagagcatgtcttagagtctaacaaatctagatttgtatccttcactctgccaggtattagttgtgtgacttcgggaaagttagttaccttctctaagccacagtgactgcatgtaccccataggattgtggtaatgattaagtgaaataatgtgcaaatatgataaagtacataccagattaaacatgagctcttgtaacagttagatcagatgtaatcaacctacagaaacaaataagttcctgaggcctctctctctacaactccgtagcatcactctttctccagttcttcccctttgtgttacccctttctgcagtcctccagttagaagaaaaaagctgttgaccagaagtcaggaagcataggttctataccaggttttggtatactgcctggtatgttatctttgactcaggtatgttatctttgacatatctttttacttctctagtctaagatccttacctgtgataacccctaatttaatttaaaggctgttataagaaagagaatgccttggcacatagtaggcactcaaaaatgttatttgaagatcagtagagtattatagaaacaggatgggctttggagtaaggcaatcctaagtttgaattccactgtgtgaatcacttaacttctcagagcttgtttctccatatatggaattgaaatactactacttacctgacagggtttttatgaaaattagccagggcagaatgtacctaacccaggatctgatacagatgaggatcactttaatcctcacctcttctaaaccacttttcttaacctaggatccacggatgtaattcagagacaatgacctcttgaaattgcaagcataagtttctgtacatgattgcatttttctgaagaaaaagtccatagatttctcttcccgtaaatatatctcaaggaaatcaaaatagcgggggaaattacatgcactgttattaaataaggaattatttgcagtcgtgggaaaaaattgtaaattcaacctggggacgaagagatggttaaaatagcaattcaatagattatgaagccaataataacattcaccactgcttgtgtgctccaaaagaagaaccattattctagggtattttccagctctaacattttgcgattctatctgtaaagctctgagaatgaggtatggtggaatctaacctcttaatatgaatcaacaactcaacgctggcctagttactcagatataatctagaaatcagcccgtcctgtaccccggctgtggcaacaatagtgtcaaaatggggtagcttcccccaaacattgcataaaggataaacacacccctctgcctcccgccaagaattaacgcagcgcccgggctaagaacaggcctgcgctcaaaggaggtaaggataggctccctcctcaggttttctgccccgtatctccctcctcccccagccagttgagatccccgagaccctggtgatccagtcaccacccatgggcccgcaggctgtgcagaagaactcaagccctttccgtggacttccctccgacttgcctgtctggagggaaaccgaagcaacgctctgagctccgattcactgaagaaaaggaaaggaaaccgacttctgagagcaaataaagcggagccctggaagagaagaaagggcacgagcccttcctcctccctcttcctgggggtgctgctacttccccggccttgtgtgcaggactggggccgccgttacctttcctcgacccaccagacccctcaaccacacaacccgagacgaattcccgcctcccgccccctcgactcccagcgcccaatagcccagcgagctcgaccaatcacccgccaaggccaggaatcaaccagtcccggtgcgaggtgagggggcgagaacttcggcatccaatggaagtctccgaaaaaaaaaaaaaaaaacaagggctcaaggtacgcggcggaaggttgtcgccactccagcaaatccctagtgtttgtttaaagtagaaagtcgtctcagtttgacttttgaaaccggcgatagagtctatgcagcaaattccttggaaactggaagtctcattcttcacgtccgtagaccagcggaaaggaagtatctttaagctttgccaagttctttgagcatttatcagtgccactcaatttaaattacccagctttgcagtagccaggatagcgaagactcaggcgggaattgccagactgctgagccaaacttaagtaagaaggctctgtgacatcgggctacaatctatctttcataatattcaaggctttcccggatgctaaaataagccagagacaagctctcagggtctccagtatcagatcacttctacttccattaaggcttatccgccttccatccctcctaagaccagtttattatttgccagtagtactttaatttactcaaagaaaaggaggttcatcatgatcttgccaaatcatgttatttttttaaaaaatctgtataacctaaacttggctcaaataataatgtctccaaaaggcaaaacctcatactgtttctcaaattcaagtaactagtttggggaaggagtttgctgggaagaaagagcaggagggggctagttgatttgtttgcctcattgtcaattgtgatatatcttaccaa

SEQ ID NO: 16 - protein, 1249 aa, Pan troglodytes
MSSMWSEYTIGGVKIYFPYKAYPSQLAMMNSILRGLNSKQHCLLESPTGSGKSLALLCSALAWQQSLSGKPADEGVSEKAEVQLSCCCACHSKDFTNNDMNQGTSRHFNYPSTPPSERNGTSSTCQDSPEKTTLAAKLSAKKQASIYRDENDDFQVEKKRIRPLETTQQIRKRHCFGTEVHNLDAKVDSGKTVKLNSPLEKINSFSPQKPPGHCSRCCCSTKQGNSQESSNTIKKDHTGKSKIPKIYFGTRTHKQIAQITRELRRTAYSGVPMTILSSRDHTCVHPEVVGNFNRNEKCMELLDGKNGKSCYFYHGVHKISDQHTLQTFQGMCKAWDIEELVSLGKKLKSCPYYTARELIQDADIIFCPYNYLLDAQIRESMDLNLKEQVVILDEAHNIEDCARESASYSVTEVQLRFARDELDSMVNNNIRKKDHEPLRAVCCSLINWLEANAEYLVERDYESACKIWSGNEMLLTLHKMGITTATFPILQGHFSAVLQKEEKISPIYGKEEAREVPVISASTQIMLKGLFMVLDYLFRQNSRFADDYKIAIQQTYSWTNQIDISDKNGLLVLPKNKKRSRQKTAVHVLNFWCLNPAVAFSDINGKVQTIVLTSGTLSPMKSFSSELGVTFTIQLEANHIIKNSQVWVGTIGSGPKGRNLCATFQNTETFEFQDEVGALLLSVCQTVSQGILCFLPSYKLLEKLKERWLSTGLWHNLELVKTVIVEPQGGEKTNFDELLQVYYDAIKYKGEKDGALLVAVCRGKVSEGLDFSDDNARAVITIGIPFPNVKDLQVELKRQYNDHHSKLRGLLPGRQWYEIQAYRALNQALGRCIRHRNDWGALILVDDRFRNNPSRYISGLSKWVRQQIQHHSTFESALESLAEFSKKHQKVLNVSIKDRTNIQDNESTLEMTSLKYSTPPYLLEAASHLSPENFVEDEAKICVQELQCPKIITKNSPLPSSIISRKEKNDPVFLEEAGKAEKIVISRSTSPTFNKQTKRVSWSSFNSLGQYFTGKIPKATPELRSSENSASSPPRFKTEKMESKTVLPFTDKCESSNLTVNTSFGSCPQSETIISSLKIDATLTRKNHSEHPLCSEEALDPDIELSLVSEEDKQSTSNRDFETEAEDESIYFTPELYDPEDTDEEKNDLAETDRGNRLANNSDCILAKDLFEIRTIKEVDSAREVKAEDCIDTKLNGILHIEESKIDDIDGNVKTTWIKELELGKTHEIEIKNFKPSPSKNKGMFPGFK

SEQ ID NO: 17 - protein, 1214 aa, Canis familiaris
MSSLWSEYTIGGVKITFPYKAYPSQLAMMNSIVRGLNSKQHCLLESPTGSGKSLALLCSTLAWQQSVSGKLVDESLSKTEVSSSCCCACHSKNFTNIDLNQGTSHHFNSPSTPPSERYDTSSTCQDSPEKTTLAAKLSAKKQASIKRDENDDFQVEKKRIRPLETTQQIRKRHCFEKKVHHVDAKVASGKTTKLNSPLEKINSFSPQNPPGHCSRCCCSTKQGSNQDSSNTTKKDHGGKSKIPKIYFGTRTHKQIAQITRELRRTAYSGVPMTILSSRDHTCVHPEVVGNFNRNEKCMELLDGKNGKSCYFYHGVHKISNQHTLQTLQGMCKAWDIEELVSLGKKLKACPYYTARELIEDAHIIFCPYNYLLDAQIRESMDINLKEQVVILDEAHNIEDCARESASYSVTEVQLRFARDELDSMVNNNIRKKNHEPLRAVCYSLINWLEANSEHLVERDYESSCKIWSGSEMVLNLHKMGITTATFPILQGHFSAVLQKEEKVLPIHGKEETREVPIISASTQIMLKGLFMVFDYLFRQNSRFADDYKVAIQQTYSWINQTDTSDKNGFFAPAKNKKHLRQKTAVHVLNFWCLNPAVAFSDINGKVLTIVLTSGTLSPMKSFSSELGVTFTIQLEANHVINNSQVWVGTIGSGPKGRNLCATFQHTETFEFQDEVGALLLSVCQTVNQGILCFLPSYKLLEKLKERWLYTGLWHNLELVKTVIVEPQGGEKTDFNELLQVYYDAIKYKGEKDGALLVAVCRGKVSEGLDFSDDNARAVITIGIPFPNVKDLQVELKRQYNDQHSKLRGLLPGRQWYEIQAYRALNQALGRCIRHKNDWGALILVDDRFRSNPSRYISGLSKWIRQQIQHHSTFESALESLSDFSRKHQKVINVSKEDRKFTQDSESILEVTCLKDNTLTYLEAASPLIPENPRKGEAKIRVQEQQCLTITESPPLPRGIISKKEKDDPVLSEEFAQAVKAEKNVISRSTSPIFNKQTKPVSWSNFNSLERYFTGEILTAGPKFRSPEDCASSISTLETEEGYKTVLPLTDKCESSSPMLNASCSQSEVISSVKIDSTLLKRNCSKQLFCCEEAVDPDIELSLLGEEAKSSTSHRASETEAEDESIYFSPELYDPADTNEEKNELVESDRDNRFVDHSNCILVEDLFEIRTIKGVDSVQEMKAEDCTVTTLNRLMHIKESKIDNIDSNMKKNSYK

SEQ ID NO: 18 - protein, 1174 aa, Mus musculus
MSSVLSDYTIGGVKIHFPCRAYPAQLAMMNSIVRGLNSSQHCLLESPTGSGKSLALLCSALAWQQSLSEKPVDEGLNKKPEAPPSCSCACHSKNFTYSDTNLDTSPHFNSPSKPSSGRNGVSTPCQDSPEKNTLAAKLSAKKQASIHRDEDDDFQVEKKRIRPLETTQQIRKRHCLEKDVHHVDARLASEKRVKPESPIGKSFSDRKDSFQNVDGLCSRCCCSAKQGNNQEPANTVKKDHGGQCKRPKIYFGTRTHKQIAQITRELRKTAYSGVPMTILSSRDHSCVHPEVVGNFNRKEKCMELLDGKHGKSCYFYHGVHKISNQQTLQHLQGMSRAWDIEELVSLGRKLKACPYYTARELIEDADIVFCPYNYLLDSQIRETMDIKLKGQVVILDEAHNIEDCARESASYSVTEVQLRFARDELDSLINGNIRKKSHEPLRDVCYNLINWLETNSKHLVERGYESSCKIWSGNEMLLNLYRMGITTATFPVLQRHLSAVLQKEEKVTPIHGKEEAIQIPIISASTQVVLKGLFMVLDYLFRENSRFADDYKVAIQQTYSWTNQIAIFDKTGVLAVPKNKKHSRQKIGVNALNFWCLNPAVAFSDINDKVRTIVLTSGTLSPLKSFSSELGVTFSIQLEANHVISNSQVWVGTVGSGPKGRNLCATFQHTETFEFQDEVGMLLLSVCQTVSQGILCFLPSYKLLEKLRERWIFTGLWHSLESVKTVIAEPQGGEKTDFDELLQVYYDAIKFKGEKDGALLIAVCRGKVSEGLDFSDDNARAVITVGIPFPNVKDLQVELKRQYNDHHSKSRGLLPGRQWYEIQAYRALNQALGRCIRHKNDWGALILVDDRFNNNPNRYISGLSKWVRQQIQHHSSFASALESLTEFSRRHQKVTNRSKKDEKCTKDNEPTLEVACLEDSTFTSVSESSHQSPENSTEEAEVCVQELQCPQVATKSPSVASHGVSRRKKSDPGLRGESLQTMKTEKNEISRSSSPTFGKQTEPVNWPIFNSLRRHFNSKVKNCTPVLKSSKNRAPGSSTFNKTALPLTGNCVPSNETADTSLGPCLQSEVIISPVKIEATPATNYSKQVFCCEKDLLPDTELSPGTEEAKCPSSNKAAETEVDDDSECFTPELFDPVDTNEENGELVETDRSSHSSDCFSAEELFETATGFGQK

SEQ ID NO: 19 - protein, 1166 aa, Rattus norvegicus
MSSVLSEYTIGGVKIHFPCRAYPAQLAMMNSIVRGLNSSQHCLLESPTGSGKSLALLCSALAWQQSLTGKPVDEGLNKKPEAPSSCSCSCHSKNFTYSDTNLDTSPHFSSPSKPSSERNAVSSPCRDSPERNSLAAKLSAKKHASIHEDDDFQVEKKRIRPLETTQQIRKRHCLEKDVHHLDARLASEKRVKPESPIRKTSSSFQNPDGLCSRCCCSANQGINKESANTVKKDNGDQSKRPKIYFGTRTHKQIAQITRELRKTAYSGVPMTILSSRDHTCVHPEVVGNFNRNEKCMELLDGKHGKSCYFYHGVHRISNQQTLQFLHGISKAWDIEELVSLGRKLKACPYYTARELIDSADIIFCPYNYLLDSQIRESMDIKLKEQVVILDEAHNIEECARETASYSVTEVQLRFARDELDSLINSNIRKKSHEPLRDVCYNLINWLETNSKNLVERDYESSCKIWSGNEMLLNLHRMGITAASFPVLQKHLSAVLQKEEKVTSTHGKEEAIQIPIISASTQIMLKGLFMVLDYLFRKNSRFADDYKVAIQQTYSWTNQIAIFDKNALLPVPKNKKHSRQKIGVNVLNFWCLNPAVAFSDINDKVRTIVLTSGTLSPLKSFSSELGVTFNIQLEANHVISNSQVWVGTVGSGPKGRNLCATFQHTETFEFQDEVGMLLLSVCQTVSQGILCFLPSYKLLEKLRERWIFTGLWHSLESVKTVIAEPQGGEKTDFDELLQVYYDAIKFKGEKDGALLIAVCRGKVSEGLDFSDDNARAVITIGIPFPNVKDLQVELKRQYNDHHSKLRGLLPGRQWYEIQAYRALNQALGRCIRHKNDWGALILVDDRFNNNPDRYISGLSKWVRQQIQHHSTFASALESLTEFSKRHQKVTNRSKKEEKCTKENGSTLDVACLEGSTLTSVSEASHQTPENSLEEEAKVCVQEQRYPQMAAENPSGPSHGVSRRKESDPGLREKSVQTMKTEKSEISRSSSPTFGKQTEPVNWPIFNSLKRHFNSKVKNRTPVLKSSKNHASASSAFNKTALPLTGKCVSSSAPANTPLAPCPQSEVIIPSVKADTTPAKTHCKQNEKDPSPDAELSPVTEEAKGSSSNPAVGTEVDDDSVCFTPELFDPVSTDEENSELVETDRSSNNSDCLSAEELFETVTGFGQK

SEQ ID NO: 20 - protein, 1252 aa, Gallus gallus
MSSDVSQYTIGGVKIMFPCKAYPSQLAMMNAIVKGLNNRQHCLLESPTGSGKSLALLCSALSWQQSLYEKSLLKSSCEKEDREPAASLPCRCVCHSRSESSEATAGASHGAACSNNYETGGSVKHGDQLSDTECKENNTLASKLSAKKRASACGNECDDFQVERKRIRPLETEQQVRKRHCFSKEVQLVDALEVYNQRKNGELIVHSEKSVKNTSPQTLFSSCTECSCSSGKETRKDSGNTKKKANGDQTFIPKIFFGTRTHKQIAQITRELKRTAYSGVPMTILSSRDYTCIHPVVSSSNSNRNELCVELLEGKHGKSCLYYHGVHKLSEHYALQSAHNTYQAWDIEDLVSLGKKLRACPYFAARELMVGADIVFCPYNYLLDPQIRESMEINLKGQVVILDEAHNIEDSAREAVSYSVTESQLNAAREELDFMVNNNIRQKDHEQLRAMCCSLTNWLRESSSQLVETGYETSCKVWSGKEMLNHFHDMGITNISFPILQKHLSAVLEKEEKISMFGKEELVEIPIVSSATQIVLKGLFMVLLYLFKDNSRFADDYRVALQQTYAWTNDNQPDVSDTSAFFTKTKHKRNLRHKTVVHMLNFWCLNPAVAFSDLNDVRTVVLTSGTLSPMDSFSSELGVKFSIQLEANHVIRNSQVWVGTIGTGPNGRKLCATFQHTETFEFQDEVGALLLSVCQKVGQGILCFLPSYKLLDKLKDRWIHTGLWRNLELVKTVIAEPQGGAKSDFDELLKIYYDAIKFKGEKDGALLIAVCRGKVSEGLDFCDENARAVITIGIPFPNVKDLQVELKRKYNDQHKTTRGLLPGSQWYEIQAYRALNQALGRCIRHRSDWGALILVDDRFRNNPNKYITGLSKWIRQQVQHHENFGSALESLHAFAERNQKGIDFSSQCSNEVFHVPLNSKEPSSASQQEATIHLSPDVPVKSEEQSFVPETHLTTTINSINPGPSNQPGGQKVDVESCSHNGIQRRKHMDSTPRRPANKTEKKSDRTNSDFMKEHCCFKPLTSTPLPVATNCVSTASSKQRKNVNSASELIGGVNQCQSSFTLEHKPSIPESHLETTNFSVKSTEAPVAEEHLDEQKLQIEPCSELPSVGGRPELSVLEISAEDEDESLYFTPELYDDAESEEQEMRPLDPDENQIECGKPTVADDLFVISTSKTLSEPKEMINDDGRNTSLHGTMLSDISKNSTVNIEKMTNGEEAEQVESQEVDTKKRKISLSRSRNKGVSPFLLDSTST

SEQ ID NO: 21 - protein, 39 aa, Homo sapiens
CATFQNTETFEFQDEVGALLLSVCQTVSQGILCFLPSYK

SEQ ID NO: 22 - protein, 11 aa, Homo sapiens
AKEFCVSCHLT

(19) United States
(12) Patent Application Publication
(10) Pub. No.: US 2012/0225786 A1
(43) Pub. Date: Sep. 6, 2012

(54) RISK VARIANTS FOR CANCER

(76) Inventors: THORUNN RAFNAR, Reykjavik (IS); PATRICK SULEM, Reykjavik (IS)

(21) Appl. No.: 13/410,880

(22) Filed: Mar. 2, 2012

(30) Foreign Application Priority Data
Mar. 2, 2011 (IS) 050009
Jul. 22, 2011 (IS) 050017

(51) Int. Cl. C40B 20/00; C12Q 1/34; C40B 40/06; G01N 33/574

(52) U.S. Cl. 506/2; 435/7.4; 435/18; 506/16

(57) ABSTRACT

It has been found that variants on chromosome 17q23.2 in the BRIP1 gene are associated with risk of cancer in humans. The invention provides diagnostic applications using such variants, including methods of determining susceptibility of cancer.

### INTRODUCTION

Malignant cancer is characterized by uncontrolled growth within specific cell groups, invasion that intrudes upon and destroys adjacent tissues. Cancer often metastasizes, wherein tumor cells spread to other locations in the body via the lymphatic system or through the bloodstream.

Ovarian cancer is the fifth most common cause of cancer death in women in the US. Five-year relative survival rate is less than 45% with the stage at diagnosis being the major prognostic factor. Only 19% of ovarian cancer cases are diagnosed while the cancer is still localized and chances of cure are over 90%. A striking 68% are diagnosed after the cancer has already metastasized.

Ovarian cancer causes non-specific symptoms. Most women with ovarian cancer report one or more symptoms such as abdominal pain or discomfort, an abdominal mass, bloating, back pain, urinary urgency, constipation, tiredness and a range of other non-specific symptoms, as well as more specific symptoms such as pelvic pain, abnormal vaginal bleeding or involuntary weight loss.

In the absence of effective treatment for advanced ovarian cancer, the major emphasis is on developing screening programs that will detect the disease at an early stage. Ovarian cancer screening with transvaginal ultrasound (TVU) and CA-125 was evaluated in the Prostate, Lung, Colorectal and Ovarian (PLCO) Trial, including almost 40,000 women. Screening identified both early- and late-stage neoplasms; however, the predictive value of both tests was relatively low and the effect of screening on ovarian cancer mortality still needs a longer follow-up. This trial, along with other studies, has led the U.S. Preventive Services Task Force to conclude that despite evidence that screening with serum CA-125 level or TVU can detect ovarian cancer at an earlier stage than it can be detected in the absence of screening, earlier detection would likely have a small effect on mortality from ovarian cancer.

Given that approximately 1 in 72 women will be diagnosed with cancer of the ovary during their lifetime, repeated screening of the whole population with costly procedures like ultrasound is not a feasible strategy. This is particularly true considering the large number of false positive cases that need follow-up by surgical procedures with the associated risks of side effects. Management strategies that aim to identify those individuals at highest risk of the disease could be used to focus screening efforts on women who will benefit the most from them while minimizing unnecessary interventions and anxiety amongst those at lower risk. Clearly, the implementation of such strategies depends on the development of a risk model that includes all known risk factors.

The strongest factors affecting ovarian cancer risk are family history (genetic factors), age, race, the number of children and endocrine history. Presently, all these factors can be evaluated except for the genetic factors. The role of inherited factors in ovarian cancer has been firmly established in epidemiological and family studies. Studies on 44,788 pairs of twins in the Swedish, Danish, and Finnish twin registries estimated that genetic factors can explain about 22% of ovarian cancer. Furthermore, a meta-analysis of data from 15 observational studies estimated that the relative risk of developing ovarian cancer for a woman with a single first-degree relative affected with ovarian cancer is 3.1. Two cancer syndromes include ovarian cancer as a part of their phenotype; the hereditary breast/ovarian cancer syndrome and Lynch syndrome. The majority of families with extensive family history of breast and ovarian cancer harbour mutations in the breast cancer genes, BRCA1 and BRCA2, while Lynch syndrome is caused by mutations in DNA mismatch repair genes. However, these two cancer syndromes constitute a small fraction (5-15%) of ovarian cancer cases. It has been found that the Icelandic founder BRCA2 999del5 mutation is present in 6% of ovarian cancer cases in Iceland and is associated with a 20-fold increase in the risk of the disease. The remaining genetic risk of ovarian cancer is likely due to the combined effects of multiple variants yet to be identified.

### SUMMARY OF THE INVENTION

The present inventors have discovered that variants on chromosome 17q23.2 in the human BRIP1 gene are associated with increased risk of cancer, including ovarian cancer, pancreatic cancer, breast cancer and rectal cancer. The present invention relates to the utilization of such variants in the risk management of cancer. For simplicity, many details of the invention, including details related to BRIP1 or techniques or materials for practicing the invention are described in the context of predicting susceptibility to ovarian cancer. It should be understood that such details are also are applicable to predicting susceptibility for other cancers identified herein.

In one aspect, the invention provides a method of determining a susceptibility to cancer, the method comprising analyzing sequence data from a human individual for at least one polymorphic marker in the human BRIP1 gene, or an encoded BRIP1 protein, wherein different alleles of the at least one polymorphic marker are associated with different susceptibilities to cancer in humans, and determining a susceptibility to cancer from the sequence data. In certain embodiments, the sequence data is nucleic acid sequence data. In a preferred embodiment, the at least one polymorphic marker is an −/AA insertion/deletion polymorphism at position 57,208,601 in NCBI Build 36 (SEQ ID NO:12) (chr17:57208601 ins+AA) or a TT deletion at position 57213073 in NCBI Build 36 (chr17: 57213073 delTT).

Another aspect relates to a method of method of determining a susceptibility to a cancer, the method comprising analyzing data representative of at least one allele of a BRIP1 gene in a human subject, wherein different alleles of the human BRIP1 gene are associated with different susceptibilities to at least one cancer in humans, and determining a susceptibility to a cancer for the human subject from the data.

The invention also provides a method of determining a susceptibility to Ovarian Cancer, the method comprising analyzing sequence data from a human subject for at least one variant in the human BRIP1 gene, or in an encoded human BRIP1 protein, wherein different alleles of the at least one variant are associated with different susceptibilities to Ovarian Cancer in humans, and determining a susceptibility to Ovarian Cancer for the human subject from the sequence data. Further provided is a method of analyzing nucleic acid sequence data from a human individual for at least one polymorphic marker selected from the group consisting of: an −/AA insertion/deletion polymorphism between position 57,208,601 and 57,208,602 in NCBI Build 36 (SEQ ID NO:12); rs34289250 (SEQ ID NO:1); rs12938171 (SEQ ID NO:2); an A/T polymorphism at position 55,422,245 in NCBI Build 36 (SEQ ID NO:3); a C/T polymorphism at position 55,217,320 in NCBI Build 36 (SEQ ID NO:4); rs12451939 (SEQ ID NO:5); a G/T polymorphism at position 56,567,990 in NCBI Build 36 (SEQ ID NO:6); an A/C polymorphism at position 56,478,611 in NCBI Build 36 (SEQ ID NO:7); a C/T polymorphism at position 56,505,864 in NCBI Build 36 (SEQ ID NO:8); and rs12937080 (SEQ ID NO:9), and markers in linkage disequilibrium therewith, wherein different alleles of the at least one polymorphic marker are associated with different susceptibilities to Ovarian Cancer in humans, and determining a susceptibility to Ovarian Cancer from the nucleic acid sequence data.

A further aspect provides a method of determining whether a human subject is at increased risk of developing cancer, such as ovarian cancer, pancreatic cancer, breast cancer and colorectal cancer, the method comprising analyzing amino acid sequence data about a BRIP1 polypeptide from the subject, wherein a determination of the presence of a truncated BRIP1 polypeptide compared with a wild-type BRIP1 polypeptide with sequence as set forth in SEQ ID NO:13 is indicative that the subject is at increased risk of developing the cancer.

Also provided is a method of determining whether an individual is at increased risk of developing ovarian cancer, the method comprising steps of obtaining a biological sample containing nucleic acid from the individual; determining, in the biological sample, nucleic acid sequence about the BRIP1 gene; and comparing the sequence information to the wild-type sequence of BRIP1; wherein an identification of a mutation in BRIP1 in the individual is indicative that the individual is at increased risk of developing ovarian cancer.

The invention further provides a method of identification of a marker for use in assessing susceptibility to Ovarian cancer in human individuals, the method comprising (a) identifying at least one polymorphic marker in the human BRIP1 gene; (b) obtaining sequence information about the at least one polymorphic marker in a group of individuals diagnosed with ovarian cancer; and (c) obtaining sequence information about the at least one polymorphic marker in a group of control individuals; wherein determination of a significant difference in frequency of at least one allele in the at least one polymorphism in individuals diagnosed with ovarian cancer as compared with the frequency of the at least one allele in the control group is indicative of the at least one polymorphism being useful for assessing susceptibility to ovarian cancer. Further provided are prognostic methods and methods of assessing probability of response to treatment. Thus, the invention provides a method of predicting prognosis of an individual diagnosed with Ovarian Cancer, the method comprising obtaining sequence data about a human individual about at least one variant in the human BRIP1 gene, wherein different alleles of the at least one variant are associated with different susceptibilities to Ovarian Cancer in humans, and predicting prognosis of Ovarian Cancer from the sequence data. A further aspect of the invention relates to a method of predicting prognosis of an individual diagnosed with ovarian cancer, the method comprising obtaining sequence data about a human individual about at least one polymorphic marker in the human BRIP1 gene, wherein different alleles of the at least one polymorphic marker are associated with different susceptibilities to ovarian cancer in humans, and predicting prognosis of ovarian cancer from the sequence data. Also provided is a method of assessing probability of response of a human individual to a therapeutic agent for preventing, treating and/or ameliorating symptoms associated with ovarian cancer, comprising obtaining sequence data about a human individual identifying at least one allele of at least one polymorphic marker in the human BRIP1 gene, wherein different alleles of the at least one polymorphic marker are associated with different probabilities of response to the therapeutic agent in humans, and determining the probability of a positive response to the therapeutic agent from the sequence data.

Another aspect of the invention relates to methods of selecting treatment regimens. Thus, one aspect of the invention provides a method of selecting a treatment regimen for a human subject with ovarian cancer, the method comprising analyzing data representative of at least one allele of a BRIP1 gene in a human subject with ovarian cancer to identify the presence or absence of a loss-of-function BRIP1 mutant allele, and selecting a therapeutic regimen of a therapeutic agent for treating ovarian cancer for a subject identified from the data as having the loss-of-function BRIP1 mutant allele. Another such aspect related to a method comprising determining the presence or absence of at least one allele of at least one polymorphic marker in a nucleic acid sample obtained from the individual, wherein the at least one allele causes a loss of function or loss of expression of a BRIP1, and selecting for treatment with a therapeutic agent for ovarian cancer a subject identified as having the at least one allele in the nucleic acid sample.

The invention also provides kits. In one such aspect, the invention relates to a kit for assessing susceptibility to ovarian cancer in human individuals, the kit comprising reagents for selectively detecting at least one at-risk variant for ovarian cancer in the individual, wherein the at least one at-risk variant is a marker in the human BRIP1 gene or an amino acid substitution in an encoded BRIP1 protein, and a collection of data comprising correlation data between the at least one at-risk variant and susceptibility to ovarian cancer.

Further provided is the use of an oligonucleotide probe in the manufacture of a diagnostic reagent for diagnosing and/or assessing a susceptibility to ovarian cancer, wherein the probe is capable of hybridizing to a segment of the human BRIP1 gene with sequence as given by SEQ ID NO:10, and wherein the segment is 15-400 nucleotides in length.

The invention also provides computer-implemented methods and applications. In one such application, the invention relates to a system comprising a computer implemented method for identifying susceptibility to a cancer in a human subject, the system comprising at least one processor, at least one computer-readable medium, a susceptibility database operatively coupled to a computer-readable medium of the system and containing population information correlating the presence or absence of one or more alleles of the human BRIP1 gene and susceptibility to a cancer in a population of humans, a measurement tool that receives an input about the human subject and generates information from the input about the presence or absence of at least one mutant BRIP1 allele indicative of a BRIP1 defect in the human subject; and an analysis tool that is operatively coupled to the susceptibility database and the measurement tool, is stored on a computer-readable medium of the system, and is adapted to be executed on a processor of the system, to compare the information about the human subject with the population information in the susceptibility database and generate a conclusion with respect to susceptibility to the cancer for the human subject.

Another system is provided for assessing or selecting a treatment protocol for a subject diagnosed with a cancer, comprising at least one processor, at least one computer-readable medium, a medical treatment database operatively connected to a computer-readable medium of the system and containing information correlating the presence or absence of at least one mutant BRIP1 allele and efficacy of treatment regimens for the cancer, a measurement tool to receive an input about the human subject and generate information from the input about the presence or absence of the at least one mutant BRIP1 allele indicative of a BRIP1 defect in a human subject diagnosed with the cancer; and a medical protocol tool operatively coupled to the medical treatment database and the measurement tool, stored on a computer-readable medium of the system, and adapted to be executed on a processor of the system, to compare the information with respect to presence or absence of the at least one mutant BRIP1 allele for the subject and the medical treatment database, and generate a conclusion with respect to at least one of (1) the probability that one or more medical treatments will be efficacious for treatment of the cancer for the patient, and (2) which of two or more medical treatments for the cancer will be more efficacious for the patient.

Also provided is a computer-readable medium having computer executable instructions for determining susceptibility to Ovarian Cancer in a human individual, the computer readable medium comprising sequence data identifying at least one allele of at least one polymorphic marker in the individual; a routine stored on the computer readable medium and adapted to be executed by a processor to determine risk of developing Ovarian Cancer for the at least one polymorphic marker; wherein the at least one polymorphic marker is a marker in the human BRIP1 gene, or an encoded BRIP1 protein, that is associated with susceptibility of Ovarian Cancer in humans.

A further aspect relates to an apparatus for determining a susceptibility to Ovarian Cancer in a human individual, comprising a processor; a computer readable memory having computer executable instructions adapted to be executed on the processor to analyze sequence information about at least one human individual with respect to at least one marker in the human BRIP1 gene or an encoded human BRIP1 protein that is associated with susceptibility of Ovarian Cancer in humans, and generate an output based on the marker sequence information, wherein the output comprises at least one measure of susceptibility to Ovarian Cancer for the human individual.

Further details of these and other aspects of the inventions are described in the following detailed description of the invention.

It should be understood that all combinations of features described herein are contemplated, even if the combination of features is not specifically found in the same sentence or paragraph as set forth in the following detailed description.

### BRIEF DESCRIPTION OF THE DRAWINGS

The foregoing and other objects, features and advantages of the invention will be apparent from the following more particular description of preferred embodiments of the invention.

FIG. 1 provides a diagram illustrating a system comprising computer implemented methods utilizing risk variants as described herein.

FIG. 2 shows an exemplary system for determining risk of cancer as described further herein.

FIG. 3 shows a system for selecting a treatment protocol for a subject diagnosed with a cancer.

FIG. 4 shows a schematic representation of the location and consequences of the two indels found in exon 12 (Spain) and exon 14 (Iceland) of BRIP1. The mRNA is transcribed of the minus strand.

FIG. 5 shows loss-of-heterozygosity (LOH) in tumor samples from carriers of Chr17:57208601 ins+AA. Sequence of the region around Chr17:57208601 ins+AA in 10 tumor samples from heterozygous carriers of the insert. The tumors are ordered with the tumor showing no LOH first, followed by one tumor with partial LOH and 8 tumors with significant or complete loss of the wild-type allele.

FIG. 6 shows sequence traces of the region over Chr17:57208601 ins+AA in a heterozygous carrier of the insert showing loss of the wild-type allele and mRNA expression in the tumor. Top panel: Sequence of germline DNA from blood, Center panel: Sequence of tumor DNA, Bottom panel: Sequence of cDNA from tumor.

FIG. 7 depicts a multi-species alignment of BRIP1 amino acid sequences from H. sapiens, P. troglodytes, C. familiaris, M. musculus, R. norvegicus, and G. gallus (SEQ ID NOs:13 (H. sapiens) and 16-20, respectively). Symbols below the sequence alignment highlight residues that are fully (*) or partially (: or .) conserved between the species.

### DETAILED DESCRIPTION

### Definitions

Unless otherwise indicated, nucleic acid sequences are written left to right in a 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by the ordinary person skilled in the art to which the invention pertains.

The following terms shall, in the present context, have the meaning as indicated:

A “polymorphic marker”, sometime referred to as a “marker”, as described herein, refers to a genomic polymorphic site. Each polymorphic marker has at least two sequence variations characteristic of particular alleles at the polymorphic site. Thus, genetic association to a polymorphic marker implies that there is association to at least one specific allele of that particular polymorphic marker. The marker can comprise any allele of any variant type found in the genome, including SNPs, mini- or microsatellites, insertion-deletions, translocations and copy number variations (insertions, deletions, duplications). Polymorphic markers can be of any measurable frequency in the population. For mapping of disease genes, polymorphic markers with population frequency higher than 5-10% are in general most useful. However, polymorphic markers may also have lower population frequencies, such as 1-5% frequency, or even lower frequency. The term shall, in the present context, be taken to include polymorphic markers with any population frequency.

An “allele” refers to the nucleotide sequence of a given locus (position) on a chromosome. A polymorphic marker allele thus refers to the composition (i.e., sequence) of the marker on a chromosome. Genomic DNA from an individual contains two alleles (e.g., allele-specific sequences) for any given polymorphic marker, representative of each copy of the marker on each chromosome. Sequence codes for nucleotides used herein are: A=1, C=2, G=3, T=4.

Sequence conucleotide ambiguity as described herein is according to WIPO ST.25:

| IUB code | Meaning |
|---|---|
| A | Adenosine |
| C | Cytidine |
| G | Guanine |
| T | Thymidine |
| R | G or A |
| Y | T or C |
| K | G or T |
| M | A or C |
| S | G or C |
| W | A or T |
| B | C, G or T |
| D | A, G or T |
| H | A, C or T |
| V | A, C or G |
| N | A or G or C or T, unknown or other |

A nucleotide position at which more than one sequence is possible in a population (either a natural population or a synthetic population, e.g., a library of synthetic molecules) is referred to herein as a “polymorphic site”.

A “Single Nucleotide Polymorphism” or “SNP” is a DNA sequence variation occurring when a single nucleotide at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the National Center for Biotechnological Information (NCBI).

A “variant”, as described herein, refers to a segment of DNA that comprises a polymorphic site. A “marker” or a “polymorphic marker”, as defined herein, is a variant.

A “microsatellite” is a polymorphic marker that has multiple small repeats of bases that are 2-8 nucleotides in length (such as CA repeats) at a particular site, in which the number of repeat lengths varies in the general population.

An “indel”, or an “insertion-deletion” is a common form of polymorphism comprising a small insertion or deletion that is typically only a few nucleotides long. One example of an indel is the −/AA polymorphism in the BRIP1 gene described herein (chr17:57208601 ins+AA). This indel introduces two A nucleotides (AA) between position 57,208,601 and 57,208,602 in NCBI Build 36 of the human genome assembly, as further shown in SEQ ID NO:12 herein. At this position, there are thus two possible alleles: (a) no insertion, thus the sequence is the wild-type sequence (SEQ ID NO:10 and SEQ ID NO:11); and (b) an insertion of AA between position 57,208,601 and 57,208,602 in NCBI Build 36, corresponding to a TT insertion between position 2345 and 2346 in SEQ ID NO:10 (complementary sequence, since gene is transcribed from the minus strand).

A “haplotype,” as described herein, refers to a segment of genomic DNA that is characterized by a specific combination of alleles arranged along the segment. For diploid organisms such as humans, a haplotype comprises one member of the pair of alleles for two or more polymorphic markers or loci along the segment. In a certain embodiment, the haplotype can comprise two or more alleles, three or more alleles, four or more alleles, or five or more alleles.

Allelic identities are described herein in the context of the marker name and the particular allele of the marker, e.g., “2 rs34289250” refers to the 2 allele of marker rs34289250, and is equivalent to “rs34289250 allele 2”. Furthermore, allelic codes are as for individual markers, i.e. 1=A, 2=C, 3=G and 4=T.

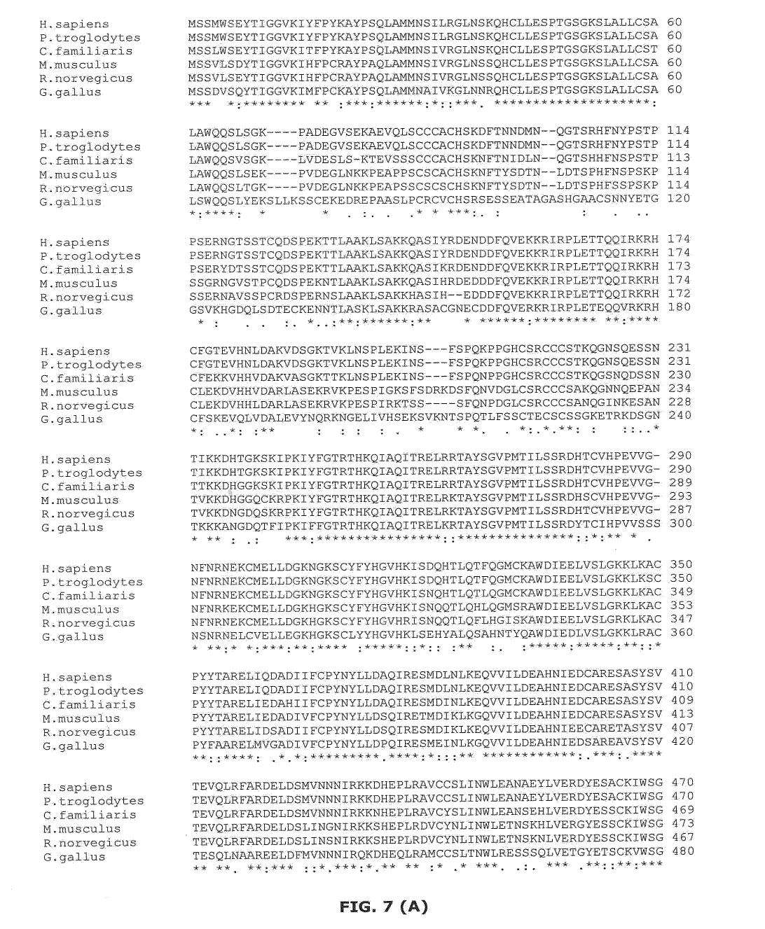

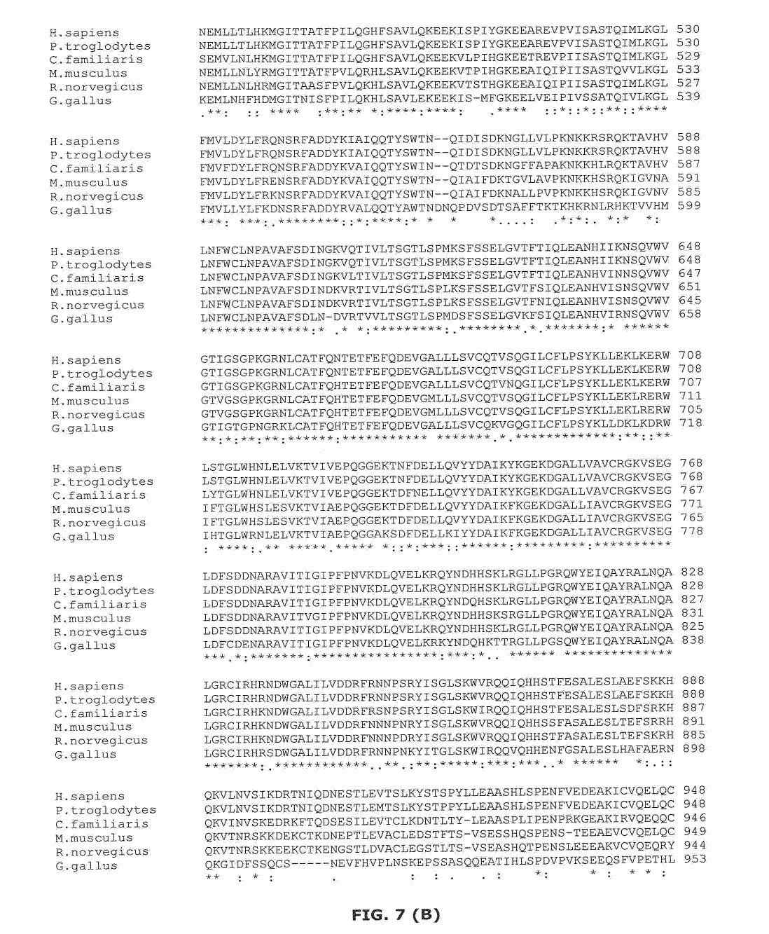

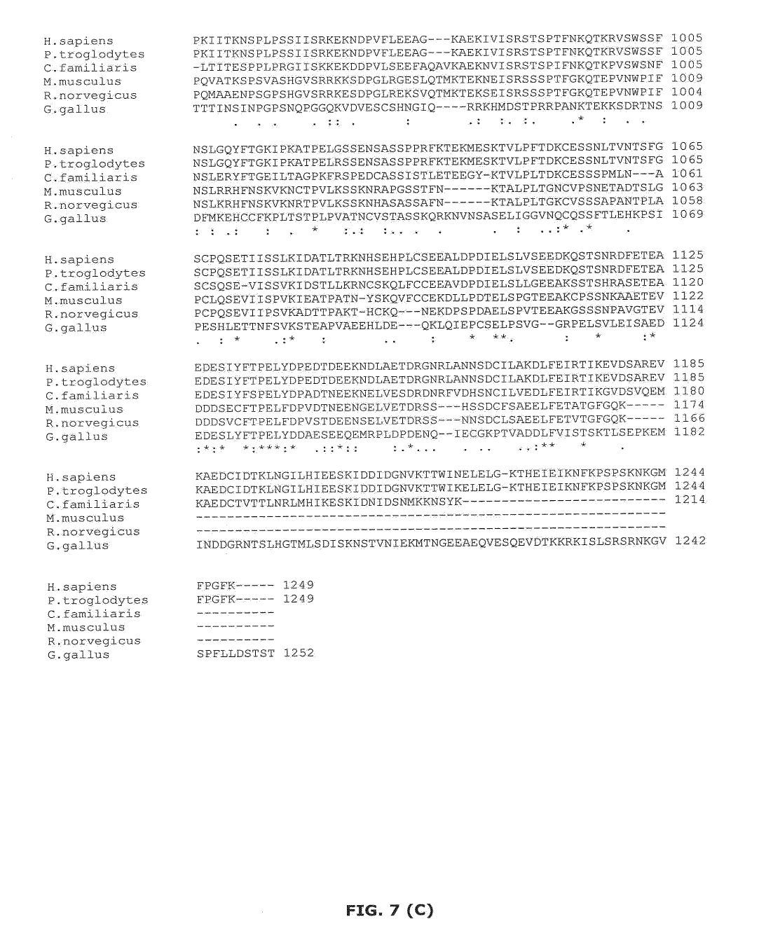

The term “BRIP1”, as described herein, refers to the BRCA1-interacting protein 1 gene on chromosome 17q22. This gene is sometimes also referred to as BRCA1-associated C-terminal helicase 1 (BACH1). The nucleotide sequence of the gene is shown in SEQ ID NO:10 herein (corresponding to accession number NM_{—}032043.2). The −/AA insertion/deletion polymorphism (chr17:57208601 ins+AA) corresponds to a −/TT indel at position 2345 (i.e., between position 2345 and 2346) in the sequence of a BRIP1 transcript (cDNA) as set forth in SEQ ID NO:10. Species homologs for human BRIP1 have been identified and characterized. Deduced amino acid sequences for human and exemplary other vertebrate species homologs are shown in FIG. 7.

The term “susceptibility”, as described herein, refers to the proneness of an individual towards the development of a certain state (e.g., a certain trait, phenotype or disease), or towards being less able to resist a particular state than the average individual. The term encompasses both increased susceptibility and decreased susceptibility. Thus, particular alleles at polymorphic markers may be characteristic of increased susceptibility (i.e., increased risk) of Ovarian cancer, as characterized by a relative risk (RR) or odds ratio (OR) of greater than one for the particular allele. Alternatively, the markers and/or haplotypes of the invention are characteristic of decreased susceptibility (i.e., decreased risk) of Ovarian cancer, as characterized by a relative risk of less than one.

The term “and/or” shall in the present context be understood to indicate that either or both of the items connected by it are involved. In other words, the term herein shall be taken to mean “one or the other or both”.

The term “look-up table”, as described herein, is a table that correlates one form of data to another form, or one or more forms of data to a predicted outcome to which the data is relevant, such as phenotype or trait. For example, a look-up table can comprise a correlation between allelic data for at least one polymorphic marker and a particular trait or phenotype, such as a particular disease diagnosis, that an individual who comprises the particular allelic data is likely to display, or is more likely to display than individuals who do not comprise the particular allelic data. Look-up tables can be multidimensional, i.e. they can contain information about multiple alleles for single markers simultaneously, or they can contain information about multiple markers, and they may also comprise other factors, such as particulars about diseases diagnoses, racial information, biomarkers, biochemical measurements, therapeutic methods or drugs, etc.

The term “database” refers to a collection of data organized for one or more purposes. In the context of the invention, databases may be organized in a digital format for access, analysis, or processing by a computer. The data are typically organized to model features relevant to the invention. For instance, one component of data in a database may be information about variations in a population, such as genetic variation with respect to BRIP1, but also variation with respect to other medically informative parameters, including other genetic loci, race, ethnicity, sex, age, behaviors and lifestyle (tobacco consumption (smoking), alcohol consumption (drinking), exercise, body mass indices), glucose tolerance/diabetes, and any other factors that medical personnel may measure in the context of standard medical care or specific diagnoses. Other components of the database may include one or more sets of data relating to susceptibility to a disease in a population, and/or suitability or success of a disease treatment, and/or suitability or success of a protocol for screening for or presenting a disease. Preferably the data is organized to permit analysis of how the biological variation in the population correlates with the susceptibility to disease and/or the suitability or success of the treatment, protocol etc. A look-up datable (or the information in a look-up table) may be stored in a database to facilitate aspects of the invention.

A “computer-readable medium”, is an information storage medium that can be accessed by a computer using a commercially available or custom-made interface. Exemplary computer-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, etc.

The term “biological sample” refers to a sample obtained from an individual that contains nucleic acid and/or protein and/or fluid containing organic and/or inorganic metabolites and substances. In many variations of the invention, the biological sample comprises nucleic acid suitable for genetic analysis.

A “nucleic acid sample” as described herein, refers to a sample obtained from an individual that contains nucleic acid (DNA or RNA). In certain embodiments, i.e. the detection of specific polymorphic markers and/or haplotypes, the nucleic acid sample comprises genomic DNA. Such a nucleic acid sample can be obtained from any source that contains genomic DNA, including a blood sample, sample of amniotic fluid, sample of cerebrospinal fluid, or tissue sample from skin, muscle, buccal or conjunctival mucosa, placenta, gastrointestinal tract or other organs.

The term “antisense agent” or “antisense oligonucleotide” refers, as described herein, to molecules, or compositions comprising molecules, which include a sequence of purine an pyrimidine heterocyclic bases, supported by a backbone, which are effective to hydrogen bond to corresponding contiguous bases in a target nucleic acid sequence. The backbone is composed of subunit backbone moieties supporting the purine and pyrimidine heterocyclic bases at positions which allow such hydrogen bonding. These backbone moieties are cyclic moieties of 5 to 7 atoms in size, linked together by phosphorous-containing linkage units of one to three atoms in length. In certain preferred embodiments, the antisense agent comprises an oligonucleotide molecule.

### Variants on Chromosome 17q23.2 Associate with Cancer

It has been discovered that variants on chromosome 17q23.2 are associated with risk of cancer. In particular, it has been discovered that mutations in the BRIP1 gene have a large effect on the risk of cancer.

The inventors identified a large number of SNP variants in this chromosomal region that confer significant risk of ovarian cancer. Further sequencing analysis revealed a frame-shift two basepair insertion (−/AA; chr17: 57208601 ins+AA) in exon 14 of the gene encoding BRCA1-interacting protein 1 (BRIP1). This two-basepair insertion confers a risk of over 7, and has a carrier frequency of about 0.7% in the population, but about 5% in individuals with ovarian cancer. Further analysis of this variant revealed its association with other cancers, including Pancreatic cancer, Colorectal Cancer, Upper Airways Cancer and Rectal cancer.

A second frameshift mutation, a TT insertion/deletion polymorphism (−/TT) at position 57,213,073 in NCBI Build 36 (chr17: 57213073 delTT; corresponds to AA deletion in position 2008-2009 in the cDNA sequence set forth in SEQ ID NO:10) was further identified. Subsequent analysis showed that this variant confers risk of both ovarian and breast cancer.

Ovarian tumors from BRIP1 mutation carriers show loss of the wild type allele suggesting that the BRIP1 gene behaves like a classical tumor suppressor gene in ovarian cancer; one copy of the gene is lost or defective due a heterozygous germline mutation, and the second wild-type (normal) copy is lost in the tumor.

Due to the relatively low prevalence of ovarian cancer, apparent sporadic cases of ovarian cancer may thus actually be due to rare mutations with large effects. These findings, which are described in more detail in the following, show that BRIP1 variants are predictive of risk of cancer.

### Variants in BRIP1 are Predictive of Cancer Risk

The BRIP1 gene (BRCA1 interacting protein 1; also called BACH1 and FANCJ) was identified by screening for proteins that interact with the C-terminal BRCT domain in BRCA1 (reviewed in Cantor & Suillemette, Future Oncol 7:253-261 (2011)).

BRIP1 interacts with the BRCT domain of BRCA1 and has several BRCA1-dependent, as well as independent, functions in preserving the integrity of the genome. It is required for homologous recombination (HR)-mediated double strand break repair (Litman, R., et al. Cancer Cell 8:255 (2005)), the execution of a G2/M cell-cycle checkpoint (Yu,X. et al., Science 302:639 (2003)) and for normal progression through S-phase by assisting in the resolution of stalled replication forks (Kumaraswamy, E., et al. Mol Cell Bio/27:6733 (2007)). Furthermore, mutations in BRIP1 that impair its helicase function render cells highly sensitive to crosslinking agents such as cisplatin (Bridge, W. L., et al. Nat Genet. 37:953 (2005)). BRIP1 is located about 20 Mb telomeric to BRCA1, in a region that is frequently lost in ovarian tumors and previous studies have suggested that a tumor suppressor, distinct from BRCA1, may reside in this region (Godwin, A. K., et al. Am J Hum Genet. 55:666 (1994)).

BRIP1 contains two major structural domains, a helicase domain spanning residues 1-888 (Cantor, et al. Cell 105:149-160 (2001)), and a BRCA1 binding domain that spans residues 979-1063 (Cantor, et al. PNAS 101:2357-2362 (2004)). The helicase domain further contains a nuclear localization signal (residues 158-175). Loss of function of BRIP1 may thus occur through mutations that affect the helicase domain, the BRCA1 binding domain, or both. Loss of function may also be a result of reduced or complete obliteration of expression of BRIP1, or loss of BRIP1 transcript.

The −/AA insertion/deletion polymorphism (chr17: 57208601 ins+AA) introduces two nucleotides in exon 14 of the human BRIP1 gene, between position 57,208,601 and 57,208,602, as set forth in SEQ ID NO:12 (between position 59 and 60), corresponding to position 94835 and 94836 in the genomic sequence of BRIP1 as set forth in SEQ ID NO:15 herein. The result of this insertion is that a stretch of four A residues is increased in size to six consecutive A residues. The chr17: 57213073 delTT mutation leads to a two basepair deletion at position 57213073 (position 99307 in SEQ ID NO:15; i.e. position 99308-99309 is deleted), which results in a frameshift and premature termination of protein translation at codon 576.

The present inventors have further shown that the wild-type allele of BRIP1 is lost in tumors of heterozygous carriers of frameshift mutations, thus showing that BRIP1 behaves like a tumor suppressor gene (Example 5). This has important clinical implications, as it shows a direct functional relationship between mutations in the gene and loss of function in tumors.

The BRIP1 gene interacts with the breast cancer BRCA1 gene and functions in regulating DNA double strand break repair pathways. Variants in the BRCA1 and BRCA2 genes are known to confer significant increased risk of breast and ovarian cancer. However, variants in the BRIP1 gene have to date not been significantly implicated in risk of other cancers, in particular have no high risk variants in this gene been associated with ovarian cancer.

The loss-of-function effect of BRIP1 mutants in tumors shows that an underlying biological may be the loss of activity of one copy BRIP1 in germline DNA. Therefore, it is likely that other germline variants in the gene also lead to ovarian cancer, in particular loss-of-function and loss of expression variants. In other words, other variants in the human BRIP1 gene that lead to loss of function of one copy of the gene, (e.g. nonsense and frameshift variants), or variants that lead to reduced or no expression of the gene, are also predictive of risk of ovarian cancer. Such variants are thus also within scope of the invention as described further herein.

A number of factors permit prediction of which BRIP1 mutations result in loss of function. For example, nonsense mutations that introduce a stop codon are expected to cause loss of function, with earlier introduction of the stop codon (closer to start codon, resulting in elimination of more of the protein) being more likely to cause loss of function. Similarly, frameshift mutations usually cause drastic changes to the amino acid sequence of the encoded protein, and often further result in introduction of a premature stop codon, and therefore are expected to cause loss of function.

In addition, many missense mutations are predicted to cause loss of function, and the character of the missense mutation can be analyzed to improve the prediction. For example, missense mutations that occur in highly conserved regions of BRIP1, as assessed by inter-species alignments such as shown in FIG. 7, are expected to be more likely to cause loss of function than mutations in highly variable regions. Missense mutations that occur in residues recognized as important for the structure or activity of a functional domain of BRIP1 are more likely to cause loss of function

The wild-type BRIP1 polypeptide sequence is set forth in SEQ ID NO: 13. The BRIP1 gene is conserved over a range of species, and a multiple species alignment is depicted in FIG. 7. The alignment shows that the helicase domain is highly conserved across species ranging from humans to chicken. Accordingly, a mutation in the helicase domain is contemplated to affect the translation of the polypeptide or the activity of the translated polypeptide. In addition, mutations in the iron-sulfur (Fe—S) domain, the Nuclear Localization Signal (NLS) domain, the ATP-binding domain and serine 990, which is reported to be required for binding to BRCA1 (Cantor et al., Future Oncol. 7: 253-261 (2011)), are also contemplated to affect the activity of the BRIP1 polypeptide. Various domains are identified in the SwissProt database, and are listed in the table below. Mutations in any of these domains, or outside of these domains, are contemplated to affect the activity of the BRIP1 polypeptide. In various embodiments of the methods of the disclosure, the residues of particular interest are those that are conserved across the species identified in FIG. 7. This is because the conservation of one or more amino acids suggests an evolutionary significance, and loss or mutation of the one or more amino acids can lead to a loss in overall BRIP1 activity.

Table of BRIP1 domains.

| Domain Type | Start | End | Description |
|---|---|---|---|
| Superfamily | 5 | 408 | |
| Superfamily | 656 | 869 | |
| Smart | 17 | 441 | DEAD-like_helicase |
| Pfam | 248 | 415 | DEAD_2 |
| TIGRfam | 149 | 884 | DNA_helicase_DNA-repair_Rad3 |
| Prosite_profiles | 11 | 442 | Helic_SF1/SF2_ATP-bd_DinG/Rad3 |
| Smart | 13 | 437 | Helicase-like_DEXD_c2 |
| Smart | 698 | 851 | Helicase_ATP-dep_c2 |

Missense mutations that result in non-conservative substitutions that introduce new amino acids with different side chain characteristics are more likely to cause loss of function than conservative mutations. Mutations that alter a promoter region or splice site are more likely to affect transcription and expression levels than mutations in other noncoding regions of the gene.

For any mutation that is identified, the mutation's effect on various BRIP1 functions can be confirmed with in vitro experiments as described in Examples below pertaining to BRIP1 functional assays.

### Methods of Determining Susceptibility to Cancer

Accordingly, in one aspect, the invention provides a method of analyzing data representative of at least one allele of a BRIP1 gene (SEQ ID NO:15) in a human subject, wherein different alleles of the human BRIP1 gene are associated with different susceptibilities to at least one cancer in humans, and determining a susceptibility to a cancer for the human subject from the data. In certain embodiments, the method is predictive of susceptibility of a cancer selected from ovarian cancer, pancreatic cancer, colorectal cancer, upper airways cancer and breast cancer. In certain preferred embodiments, the cancer is ovarian cancer.

The data can be any type of data that is representative of polymorphic alleles in the BRIP1 gene. In certain embodiments, the data is nucleic acid sequence data. The sequence data is data that is sufficient to provide information about particular alleles. In certain embodiments, the nucleic acid sequence data is obtained from a biological sample comprising or containing nucleic acid from the human individual. The nucleic acids sequence may suitably be obtained using a method that comprises at least one procedure selected from (i) amplification of nucleic acid from the biological sample; (ii) hybridization assay using a nucleic acid probe and nucleic acid from the biological sample; (iii) hybridization assay using a nucleic acid probe and nucleic acid obtained by amplification of the biological sample, and (iv) sequencing, in particular high-throughput sequencing. The nucleic acid sequence data may also be obtained from a preexisting record. For example, the preexisting record may comprise a genotype dataset for at least one polymorphic marker. In certain embodiments, the determining comprises comparing the sequence data to a database containing correlation data between the at least one polymorphic marker and susceptibility to ovarian cancer. In certain embodiments, the sequence data is provided as genotype data, identifying the presence or absence of particular alleles at polymorphic locations.

In some embodiments, the analyzing comprises analyzing the data for the presence or absence of at least one mutant allele indicative of a BRIP1 defect. The BRIP1 defect may for example be a premature truncation or frameshift of an encoded BRIP1 protein, relative to a wild-type amino acid sequence, such as the wild-type amino acid sequence presented in SEQ ID NO:13 herein. The BRIP1 defect may also be expression of a BRIP1 protein with reduced activity compared to a wild-type BRIP1 protein. The activity can for example be BRIP1 binding to the C-terminal BRCT domain of BRCA1 (e.g., the BRCT domain of wild-type BRCA1). The activity can also be DNA-dependent ATPase activity, and the activity may also be DNA helicase activity. In one embodiment, the BRIP1 defect is selected from defects that impair any of these activities.

Determination of BRIP1 binding to BRCT domain of BRCA1, ATPase activity and DNA helicase activity can be performed using standard assays well known to the skilled person, some of which are described herein. As noted above, such assays can be used to confirm that a particular BRIP1 mutation impairs or eliminates a BRIP1 activity and therefor would be expected to carry an increased susceptibility for cancers as described herein.

The data to be analyzed by the method of the invention is suitably obtained by analysis of a biological sample from a human subject to obtain information about particular alleles in the genome of the individual. In certain embodiments, the information is nucleic acid information which comprises sufficient sequence to identify the presence or absence of at least one allele in the subject (e.g. a mutant allele). The information can also be nucleic acid information that identifies at least one allele of a polymorphic marker that is in linkage disequilibrium with a mutant allele. Linkage disequilibrium may suitably be determined by the correlation coefficient between polymorphic sites. In one embodiment, the sites are correlated by values of the correlation coefficient r² of greater than 0.5. Other suitable values of r² that are also appropriate to characterize polymorphic sites in LD are however also contemplated, as discussed further herein. The information may also be information about measurement of quantity of length of BRIP1 mRNA, wherein the measurement is indicative of the presence or absence of the mutant allele. For example, mutant alleles may result in premature truncation of transcribed mRNA which can be detected by measuring the length of mRNA. The information may further be measurement of quantity of BRIP1 protein, wherein the measurement of protein is indicative of the presence or absence of a mutant allele. Truncated transcripts will result in truncated forms of translated polypeptides, which can be measured using standard methods known in the art. For example, truncated proteins or proteins arising from a frameshift may have fewer or different epitopes from wildtype protein and can be distinguished with immunoassays. Truncated proteins or proteins altered in other ways may migrate differently and be distinguished with electrophoresis. The information obtained may also be measurement of BRIP1 activity, wherein the measurement is indicative of the mutant allele. The activity is suitably selected from DNA helicase activity, ATPase activity and ability to bind to the BRCT domain of BRCA1. In one embodiment, the information is selected from any one of the above mentioned types of information.

In a further embodiment of the invention, a biological sample is obtained from the human subject prior to the analyzing steps. The analyzing may also suitably be performed by analyzing data from a preexisting record about the human subject. The preexisting record may for example include sequence information or genotype information about the individual, which can identify the presence or absence of mutant alleles.

In certain embodiments, information about risk for the human subject can be determined using methods known in the art. Some of these methods are described herein. For example, information about odds ratio (OR), relative risk (RR) or lifetime risk (LR) can be determined from information about the presence or absence of particular mutant alleles of BRIP1.

In certain embodiments, the mutant allele of BRIP is a frameshift mutation or a nonsense mutation. In one preferred embodiment, the mutant allele is a frameshift mutation. In certain embodiments, the frameshift mutation is selected from the group consisting of chr17:57208601 ins+AA and chr17: 57213073 delTT. In another embodiment, the mutant allele is a missense mutation in BRIP1 that results in expression of a BRIP1 protein with reduced or no activity compared to a wild-type BRIP1 protein. The mutant allele may also be a promoter polymorphism that leads to decreased expression of BRIP1.

In certain embodiments, the mutant allele in BRIP1 is not one, or a combination of, the following mutant alleles: P47A, G69fs8x, R173c, V193I, R251c, Q255H, M299I, A349P, H291D, R543fs 12x, W647C, R707c, K752fs 11x, R798x, Y800X, R831fs3x, G859fs3x, Q944E, K998fs59x and P1034L.

In certain embodiments, the mutant allele is not one of, or a combination of, the following mutant allele: P47A, R173c, V193I, M299I, R798x, Q944E, K998fs59x and P1034L.

In this context, frameshift mutations are indicated by “fs”, and the following number indicates the position of the first stop codon created by the new reading frame. Thus, “G69fs8x” indicates that a frameshift occurs starting at codon 69, and that a stop codon is introduced 8 codons downstream, counting from codon 69.

It should be apparent from the foregoing that another aspect of the invention may relate to a method of determining whether an individual is at increased risk of developing ovarian cancer, the method comprising steps of (a) obtaining a biological sample containing nucleic acid from the individual; (b) determining, in the biological sample, nucleic acid sequence about the BRIP1 gene, and (c) comparing the sequence information to the wild-type sequence of BRIP1, as set forth in SEQ ID NO:10 herein, wherein the identification of a mutation in BRIP1 in the individual is indicative that the individual is at increased risk of developing ovarian cancer.

Alternatively, the invention provides a method of determining whether an individual is at increased risk of developing ovarian cancer, the method comprising steps of determining, in a biological sample from the individual, nucleic acid sequence about the BRIP1 gene, and comparing the sequence information to the wild-type sequence of BRIP1, as set forth in SEQ ID NO:10 herein, wherein the identification of a mutation in BRIP1 in the individual is indicative that the individual is at increased risk of developing ovarian cancer.

The mutation may be a missense mutation, a promoter mutation, a nonsense mutation or a frameshift mutation in BRIP1. The mutation may further result in a BRIP1 defect as described in the above.

In any of the methods described herein, the human subject or human individual whose susceptibility of cancer is being assessed may be a male or a female. It will be readily apparent that risk for, e.g., ovarian cancer will be assessed in females, although assays of the invention, when practiced on males, may have informative value for female relatives in the context of ovarian cancer risk.

In another aspect, the invention provides a method of determining a susceptibility to Ovarian Cancer, the method comprising analyzing sequence data from a human subject for at least one variant in the human BRIP1 gene, or in an encoded human BRIP1 protein, wherein different alleles of the at least one variant are associated with different susceptibilities to Ovarian Cancer in humans, and determining a susceptibility to Ovarian Cancer for the human subject from the sequence data. In a preferred embodiment, the variant is the −/AA insertion/deletion polymorphism between position 57,208,601 and 57,208,602 in NCBI Build 36 (SEQ ID NO:12). In another embodiment, the variant is a variant in linkage disequilibrium with the −/AA insertion/deletion polymorphism.

The −/AA insertion/deletion results in an increase in length of a stretch of A residues in the human BRIP1 gene. Thus, the wild-type sequence has a stretch of AAAA beginning at position 57,208,602 in NCBI Build 36, and the insertion of two A residues results in a stretch of six consecutive A nucleotides (AAAAAA). The skilled person will thus appreciate that in principle the location of the indel may be anywhere within the stretch of four A residues; the resulting stretch of nucleotides would always be that of six consecutive A residues. The present inventors have for the sake of convenience, placed the indel at the first position in the stretch, i.e. between position 57,208,601 and 57,208,602.

In certain embodiments, the data that is obtained is nucleic acid sequence data. In certain embodiments, the nucleic acid sequence data is obtained from a biological sample comprising or containing nucleic acid from the human individual. The nucleic acids sequence may suitably be obtained using a method that comprises at least one procedure selected from (i) amplification of nucleic acid from the biological sample; (ii) hybridization assay using a nucleic acid probe and nucleic acid from the biological sample; (iii) hybridization assay using a nucleic acid probe and nucleic acid obtained by amplification of the biological sample, and (iv) sequencing, in particular high-throughput sequencing. The nucleic acid sequence data may also be obtained from a preexisting record. For example, the preexisting record may comprise a genotype dataset for at least one polymorphic marker. In certain embodiments, the determining comprises comparing the sequence data to a database containing correlation data between the at least one polymorphic marker and susceptibility to ovarian cancer.

Certain risk alleles have been found to be predictive of increased risk of ovarian cancer. Thus, in certain embodiments, determination of the presence of at least one allele selected from the group consisting of an AA insertion between position 57,208,601 and 57,208,602 in NCBI Build 36 (SEQ ID NO:12); a C allele of rs34289250 (SEQ ID NO:1); an A allele of rs12938171(SEQ ID NO:2); an A allele of an A/T polymorphism at position 55,422,245 in NCBI Build 36 (SEQ ID NO:3); a C allele of an C/T polymorphism at position 55,217,320 in NCBI Build 36 (SEQ ID NO:4); a G allele of rs12451939 (SEQ ID NO:5); a G allele of a G/T polymorphism at position 56,567,990 in NCBI Build 36 (SEQ ID NO:6); an A allele of an A/C polymorphism at position 56,478,611 in NCBI Build 36 (SEQ ID NO:7); a C allele of a C/T polymorphism at position 56,505,864 in NCBI Build 36 (SEQ ID NO:8); and an G allele of rs12937080 (SEQ ID NO:9) is indicative of an increased susceptibility of Ovarian Cancer for the human subject

The AA insertion is indicative of increased risk of ovarian cancer. Thus, in certain embodiment, determination of the presence of the AA insertion is indicative of increased risk of ovarian cancer for the individual. Determination of the absence of the AA insertion, or another variant allele conferring increased risk of ovarian cancer is indicative that the individual does not have the increased risk conferred by the allele.

Alternatively, the allele that is detected can be the allele of the complementary strand of DNA, such that the nucleic acid sequence data identifies at least one allele which is complementary to any of the alleles of the polymorphic markers referenced above. For example, the allele that is detected may be the complementary TT allele of the at-risk AA allele of the −/AA insertion/deletion polymorphism.

It is contemplated that in certain embodiments of the invention, it may be convenient to prepare a report of results of risk assessment. Thus, certain embodiments of the methods of the invention comprise a further step of preparing a report containing results from the determination of risk, wherein said report is written in a computer readable medium, printed on paper, or displayed on a visual display. In certain embodiments, it may be convenient to report results of susceptibility to at least one entity selected from the group consisting of the individual, a guardian of the individual, a genetic service provider, a physician, a medical organization, and a medical insurer.

### Obtaining Nucleic Acid Sequence Data

Sequence data can be nucleic acid sequence data, which may be obtained by means known in the art. Sequence data is suitably obtained from a biological sample of genomic DNA, RNA, or cDNA (a “test sample”) from an individual (“test subject). For example, nucleic acid sequence data may be obtained through direct analysis of the sequence of the polymorphic position (allele) of a polymorphic marker. Suitable methods, some of which are described herein, include, for instance, whole genome sequencing methods, whole genome analysis using SNP chips (e.g., Infinium HD BeadChip), cloning for polymorphisms, non-radioactive PCR-single strand conformation polymorphism analysis, denaturing high pressure liquid chromatography (DHPLC), DNA hybridization, computational analysis, single-stranded conformational polymorphism (SSCP), restriction fragment length polymorphism (RFLP), automated fluorescent sequencing; clamped denaturing gel electrophoresis (CDGE); denaturing gradient gel electrophoresis (DGGE), mobility shift analysis, restriction enzyme analysis; heteroduplex analysis, chemical mismatch cleavage (CMC), RNase protection assays, use of polypeptides that recognize nucleotide mismatches, such as E. coli mutS protein, allele-specific PCR, and direct manual and automated sequencing. These and other methods are described in the art (see, for instance, Li et al., Nucleic Acids Research, 28(2): e1 (i-v) (2000); Liu et al., Biochem Cell Bio 80:17-22 (2000); and Burczak et al., Polymorphism Detection and Analysis, Eaton Publishing, 2000; Sheffield et al., Proc. Natl. Acad. Sci. USA, 86:232-236 (1989); Orita et al., Proc. Natl. Acad. Sci. USA, 86:2766-2770 (1989); Flavell et al., Cell, 15:25-41 (1978); Geever et al., Proc. Natl. Acad. Sci. USA, 78:5081-5085 (1981); Cotton et al., Proc. Natl. Acad. Sci. USA, 85:4397-4401 (1985); Myers et al., Science 230:1242-1246 (1985); Church and Gilbert, Proc. Natl. Acad. Sci. USA, 81:1991-1995 (1988); Sanger et al., Proc. Natl. Acad. Sci. USA, 74:5463-5467 (1977); and Beavis et al., U.S. Pat. No. 5,288,644).

Recent technological advances have resulted in technologies that allow massive parallel sequencing to be performed in relatively condensed format. These technologies share sequencing-by-synthesis principle for generating sequence information, with different technological solutions implemented for extending, tagging and detecting sequences. Exemplary technologies include 454 pyrosequencing technology (Nyren, P. et al. Anal Biochem 208:171-75 (1993); http://www.454.com), Illumina Solexa sequencing technology (Bentley, D. R. Curr Opin Genet Dev 16:545-52 (2006); http://www.illumina.com), and the SOLID technology developed by Applied Biosystems (ABI) (http://www.appliedbiosystems.com; see also Strausberg, R. L., et al. Drug Disc Today 13:569-77 (2008)). Other sequencing technologies include those developed by Pacific Biosciences (http://www.pacificbiosciences.com), Complete Genomics (http://www.completegenomics.com), Intelligen Bio-Systems (http://www.intelligentbiosystems.com), Genome Corp (http://www.genomecorp.com), ION Torrent Systems (http://www.iontorrent.com) and Helicos Biosciences (http://www.helicosbio.som). It is contemplated that sequence data useful for performing the present invention may be obtained by any such sequencing method, or other sequencing methods that are developed or made available. Thus, any sequence method that provides the allelic identity at particular polymorphic sites (e.g., the absence or presence of particular alleles at particular polymorphic sites) is useful in the methods described and claimed herein.

Alternatively, hybridization methods may be used (see Current Protocols in Molecular Biology, Ausubel et al., eds., John Wiley & Sons, including all supplements). For example, a biological sample of genomic DNA, RNA, or cDNA (a “test sample”) may be obtained from a test subject. The subject can be an adult, child, or fetus. The DNA, RNA, or cDNA sample is then examined. The presence of a specific marker allele can be indicated by sequence-specific hybridization of a nucleic acid probe specific for the particular allele. The presence of more than one specific marker allele or a specific haplotype can be indicated by using several sequence-specific nucleic acid probes, each being specific for a particular allele. A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample.

In certain embodiments, determination of a susceptibility to ovarian cancer comprises forming a hybridization sample by contacting a test sample, such as a genomic DNA sample, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 10, 15, 30, 50, 100, 250 or 500 nucleotides in length that is sufficient to specifically hybridize under stringent conditions to appropriate mRNA or genomic DNA. For example, the nucleic acid probe can comprise all or a portion of the nucleotide sequence of the BRIP1 gene, or the probe can be the complementary sequence of such a sequence. Hybridization can be performed by methods well known to the person skilled in the art (see, e.g., Current Protocols in Molecular Biology, Ausubel et al., eds., John Wiley & Sons, including all supplements). In one embodiment, hybridization refers to specific hybridization, i.e., hybridization with no mismatches (exact hybridization). In one embodiment, the hybridization conditions for specific hybridization are high stringency.

Specific hybridization, if present, is detected using standard methods. If specific hybridization occurs between the nucleic acid probe and the nucleic acid in the test sample, then the sample contains the allele that is complementary to the nucleotide that is present in the nucleic acid probe.

Additionally, or alternatively, a peptide nucleic acid (PNA) probe can be used in addition to, or instead of, a nucleic acid probe in the hybridization methods described herein. A PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen et al., Bioconjug. Chem. 5:3-7 (1994)). The PNA probe can be designed to specifically hybridize to a molecule in a sample suspected of containing one or more of the marker alleles shown herein to be associated with risk of ovarian cancer.

In one embodiment of the invention, a test sample containing genomic DNA obtained from the subject is collected and the polymerase chain reaction (PCR) is used to amplify a fragment comprising one or more polymorphic marker. As described herein, identification of particular marker alleles can be accomplished using a variety of methods. In another embodiment, determination of susceptibility is accomplished by expression analysis, for example using quantitative PCR (kinetic thermal cycling). This technique can, for example, utilize commercially available technologies, such as TaqMan® (Applied Biosystems, Foster City, Calif.). The technique can for example assess the presence of an alteration in the expression or composition of a polypeptide or splicing variant(s) that is encoded by an associated nucleic acid described herein. Alternatively, this technique may assess expression levels of genes or particular splice variants of genes, that are affected by one or more of the variants described herein. Further, the expression of the variant(s) can be quantified as physically or functionally different.

Allele-specific oligonucleotides can also be used to detect the presence of a particular allele in a nucleic acid. An “allele-specific oligonucleotide” (also referred to herein as an “allele-specific oligonucleotide probe”) is an oligonucleotide of any suitable size, for example an oligonucleotide of approximately 10-50 base pairs or approximately 15-30 base pairs, that specifically hybridizes to a nucleic acid which contains a specific allele at a polymorphic site (e.g., a polymorphic marker). An allele-specific oligonucleotide probe that is specific for one or more particular alleles at polymorphic markers can be prepared using standard methods (see, e.g., Current Protocols in Molecular Biology, supra). PCR can be used to amplify the desired region. Specific hybridization of an allele-specific oligonucleotide probe to DNA from a subject is indicative of the presence of a specific allele at a polymorphic site (see, e.g., Gibbs et al., Nucleic Acids Res. 17:2437-2448 (1989) and WO 93/22456).

With the addition of analogs such as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures (Tm) of 64° C. and 74° C. when in complex with complementary DNA or RNA, respectively, as opposed to 28° C. for both DNA and RNA for the corresponding DNA nonamer. Substantial increases in Tm are also obtained when LNA monomers are used in combination with standard DNA or RNA monomers. For primers and probes, depending on where the LNA monomers are included (e.g., the 3′ end, the 5′ end, or in the middle), the Tm could be increased considerably. It is therefore contemplated that in certain embodiments, LNAs are used to detect particular alleles at polymorphic sites associated with particular vascular conditions, as described herein.

In certain embodiments, arrays of oligonucleotide probes that are complementary to target nucleic acid sequence segments from a subject can be used to identify polymorphisms in a nucleic acid. For example, an oligonucleotide array can be used. Oligonucleotide arrays typically comprise a plurality of different oligonucleotide probes that are coupled to a surface of a substrate in different known locations. These arrays can generally be produced using mechanical synthesis methods or light directed synthesis methods that incorporate a combination of photolithographic methods and solid phase oligonucleotide synthesis methods, or by other methods known to the person skilled in the art (see, e.g., Bier et al., Adv Biochem Eng Biotechnol 109:433-53 (2008); Hoheisel, Nat Rev Genet. 7:200-10 (2006); Fan et al., Methods Enzymol 410:57-73 (2006); Raqoussis & Elvidge, Expert Rev Mol Diagn 6:145-52 (2006); Mockler et al., Genomics 85:1-15 (2005), and references cited therein, the entire teachings of each of which are incorporated by reference herein). Many additional descriptions of the preparation and use of oligonucleotide arrays for detection of polymorphisms can be found, for example, in U.S. Pat. No. 6,858,394, U.S. Pat. No. 6,429,027, U.S. Pat. No. 5,445,934, U.S. Pat. No. 5,700,637, U.S. Pat. No. 5,744,305, U.S. Pat. No. 5,945,334, U.S. Pat. No. 6,054,270, U.S. Pat. No. 6,300,063, U.S. Pat. No. 6,733,977, U.S. Pat. No. 7,364,858, EP 619 321, and EP 373 203, the entire teachings of which are incorporated by reference herein.

Also, standard techniques for genotyping can be used to detect particular marker alleles, such as fluorescence-based techniques (e.g., Chen et al., Genome Res. 9(5): 492-98 (1999); Kutyavin et al., Nucleic Acid Res. 34:e128 (2006)), utilizing PCR, LCR, Nested PCR and other techniques for nucleic acid amplification. Specific commercial methodologies available for SNP genotyping include, but are not limited to, TaqMan genotyping assays and SNPlex platforms (Applied Biosystems), gel electrophoresis (Applied Biosystems), mass spectrometry (e.g., MassARRAY system from Sequenom), minisequencing methods, real-time PCR, Bio-Plex system (BioRad), CEQ and SNPstream systems (Beckman), array hybridization technology (e.g., Affymetrix GeneChip; Perlegen), BeadArray Technologies (e.g., Illumine GoldenGate and Infinium assays), array tag technology (e.g., Parallele), and endonuclease-based fluorescence hybridization technology (Invader; Third Wave).

Suitable biological sample in the methods described herein can be any sample containing nucleic acid (e.g., genomic DNA) and/or protein from the human individual. For example, the biological sample can be a blood sample, a serum sample, a leukapheresis sample, an amniotic fluid sample, a cerbrospinal fluid sample, a hair sample, a tissue sample from skin, muscle, buccal, or conjuctival mucosa, placenta, gastrointestinal tract, or other organs, a semen sample, a urine sample, a saliva sample, a nail sample, a tooth sample, and the like. Preferably, the sample is a blood sample, a saliva sample or a buccal swab.

### Protein Analysis

Missense, nonsense and frameshift nucleic acid variations may lead to an altered amino acid sequence, as compared to the non-variant (e.g., wild-type) protein, due to amino acid substitutions, deletions, or insertions, or truncations. Variations at splice sites may also lead to splice variation. In such instances, detection of an amino acid substitution or a truncated amino acid sequence of the variant protein may be useful. Thus, nucleic acid sequence data may be obtained through indirect analysis of the nucleic acid sequence of the allele of the polymorphic marker, i.e. by detecting a protein variation.

The variants described herein result in altered BRIP1 protein. Accordingly, one aspect of the invention relates to a method of determining whether a human subject is at increased risk of developing cancer, the method comprising analyzing amino acid sequence data about a BRIP1 polypeptide from the subject, wherein a determination of the presence of an altered BRIP1 polypeptide compared with a wild-type BRIP1 polypeptide with sequence as set forth in SEQ ID NO:13 is indicative that the subject is at increased risk of developing cancer. In certain embodiments, the cancer is selected from ovarian cancer, pancreatic cancer, upper airways cancer and breast cancer. In one embodiment, the cancer is ovarian cancer.

In certain embodiment, the altered BRIP1 polypeptide is a truncated BRIP1 polypeptide compared with wild-type BRIP1. In certain embodiments, the altered BRIP1 polypeptide has a reduced activity compared with wild-type BRIP1, wherein the activity is selected from (1) BRIP1 binding to C-terminal BRCT domain of BRCA1, (2) DNA-dependent ATPase activity and (3) DNA helicase activity.

Methods of detecting variant proteins are known in the art. For example, direct amino acid sequencing of the variant protein followed by comparison to a reference amino acid sequence can be used. Alternatively, SDS-PAGE followed by gel staining can be used to detect variant proteins of different molecular weights. Also, Immunoassays, e.g., antibody assays, e.g., immunofluorescent immunoassays, immunoprecipitations, radioimmunoasays, ELISA, and Western blotting, in which an antibody specific for an epitope comprising the variant sequence among the variant protein and non-variant or wild-type protein can be used. In certain embodiments, the amino acid sequence data about BRIP1 protein is obtained or deduced from a preexisting record.

In certain embodiments of the present invention, an amino acid substitution in the human BRIP1 protein is detected. In another embodiment, a truncated polypeptide encoded by an altered BRIP1 gene sequence is detected. In one embodiment, the truncated polypeptide is encoded by the −/AA insertion deletion polymorphism between position 57,208,601 and 57,208,602 in NCBI Build 36 (SEQ ID NO:12) (chr17:57208601 ins+AA). In another embodiment, the truncated polypeptide is encoded by the 57213073 delTT polymorphism. In one embodiment, the truncated polypeptide is a BRIP1 polypeptide that is truncated at codon 687, with an alternate sequence starting at codon 680, as shown in SEQ ID NO:14 herein. In one embodiment, the truncated polypeptide is a BRIP1 polypeptide that is truncated at codon 576. The detection may be suitably performed, for example using any of the methods described in the above, or any other suitable method known to the skilled artisan.

Methods of detecting expression levels are known in the art. For example, ELISA, radioimmunoassays, immunofluorescence, and Western blotting can be used to compare the expression of protein levels. Alternatively, Northern blotting can be used to compare the levels of mRNA. These processes are described in Sambrook et al., Molecular Cloning: A Laboratory Manual, 3^{rd} ed. Cold Spring Harbor Laboratory Press, Cold Spring Harbor, N.Y. (2001).

Any of these methods may be performed using a nucleic acid (e.g., DNA, mRNA) or protein of a biological sample obtained from the human individual for whom a susceptibility is being determined. The biological sample can be any nucleic acid or protein containing sample obtained from the human individual. For example, the biological sample can be any of the biological samples described herein.

### Number of Polymorphic Markers/Genes Analyzed

With regard to the methods of determining a susceptibility described herein, the methods can comprise obtaining sequence data about any number of polymorphic markers and/or about any number of genes. For example, the method can comprise obtaining sequence data for about at least 1, 2, 3, 4, 5, 6, 7, 8, 9, 10, 15, 20, 25, 30, 40, 50, 100, 500, 1000, 10,000 or more polymorphic markers. In certain embodiments, the sequence data is obtained from a microarray comprising probes for detecting a plurality of markers. The polymorphic markers can be the ones of the group specified herein or they can be different polymorphic markers that are not specified herein. In a specific embodiment, the method comprises obtaining sequence data about at least two polymorphic markers. In certain embodiments, each of the markers may be associated with a different gene. For example, in some instances, if the method comprises obtaining nucleic acid data about a human individual identifying at least one allele of a polymorphic marker, then the method comprises identifying at least one allele of at least one polymorphic marker. Also, for example, the method can comprise obtaining sequence data about a human individual identifying alleles of multiple, independent markers, which are not in linkage disequilibrium.

### Linkage Disequilibrium

Linkage Disequilibrium (LD) refers to a non-random assortment of two genetic elements. For example, if a particular genetic element (e.g., an allele of a polymorphic marker, or a haplotype) occurs in a population at a frequency of 0.50 (50%) and another element occurs at a frequency of 0.50 (50%), then the predicted occurrence of a person's having both elements is 0.25 (25%), assuming a random distribution of the elements. However, if it is discovered that the two elements occur together at a frequency higher than 0.25, then the elements are said to be in linkage disequilibrium, since they tend to be inherited together at a higher rate than what their independent frequencies of occurrence (e.g., allele or haplotype frequencies) would predict. Roughly speaking, LD is generally correlated with the frequency of recombination events between the two elements. Allele or haplotype frequencies can be determined in a population by genotyping individuals in a population and determining the frequency of the occurrence of each allele or haplotype in the population. For populations of diploids, e.g., human populations, individuals will typically have two alleles for each genetic element (e.g., a marker, haplotype or gene).

Many different measures have been proposed for assessing the strength of linkage disequilibrium (LD; reviewed in Devlin, B. & Risch, N., Genomics 29:311-22 (1995)). Most capture the strength of association between pairs of biallelic sites. Two important pairwise measures of LD are r² (sometimes denoted Δ²) and |D′| (Lewontin, R., Genetics 49:49-67 (1964); Hill, W. G. & Robertson, A. Theor. Appl. Genet. 22:226-231 (1968)). Both measures range from 0 (no disequilibrium) to 1 (‘complete’ disequilibrium), but their interpretation is slightly different. |D′| is defined in such a way that it is equal to 1 if just two or three of the possible haplotypes are present, and it is <1 if all four possible haplotypes are present. Therefore, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 1 if only two haplotypes are present. Markers which are correlated by an r² value of 1 are said to be perfectly correlated. In such an instance, the genotype of one marker perfectly predicts the genotype of the other.

The r² measure is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots.

A significant r² indicative of markers being in linkage disequilibrium may be at least 0.1, such as at least 0.15, 0.2, 0.25, 0.3, 0.35, 0.4, 0.45, 0.5, 0.55, 0.6, 0.65, 0.7, 0.75, 0.8, 0.85, 0.9, 0.91, 0.92, 0.93, 0.94, 0.95, 0.96, 0.97, 0.98, 0.99 or 1.0. A significant r² indicates that the markers are highly correlated, and therefore in linkage disequilibrium. Highly correlated markers must, be definition, show highly comparable results in association mapping, since the genotypes for one marker predicts the genotype for another, correlated, marker. In one specific embodiment of invention, the significant r² value can be at least 0.2. In another specific embodiment of invention, the significant r² value can be at least 0.5. In one specific embodiment of invention, the significant r² value can be at least 0.8. Alternatively, linkage disequilibrium as described herein, refers to linkage disequilibrium characterized by values of r² of at least 0.2, such as 0.3, 0.4, 0.5, 0.6, 0.7, 0.8, 0.85, 0.9, 0.95, 0.96, 0.97, 0.98, 0.99. Thus, linkage disequilibrium represents a correlation between alleles of distinct markers. It is measured by correlation coefficient or |D′| (r² up to 1.0 and |D′| up to 1.0). Linkage disequilibrium can be determined in a single human population, as defined herein, or it can be determined in a collection of samples comprising individuals from more than one human population. In one embodiment of the invention, LD is determined in a sample from one or more of the HapMap populations. These include samples from the Yoruba people of Ibadan, Nigeria (YRI), samples from individuals from the Tokyo area in Japan (JPT), samples from individuals Beijing, China (CHB), and samples from U.S. residents with northern and western European ancestry (CEU), as described (The International HapMap Consortium, Nature 426:789-796 (2003)). In one such embodiment, LD is determined in the Caucasian CEU population of the HapMap samples. In another embodiment, LD is determined in the African YRI population. In yet another embodiment, LD is determined in samples from the Icelandic population. In certain embodiments, LD is determined in a white population.

If all polymorphisms in the genome were independent at the population level (i.e., no LD between polymorphisms), then every single one of them would need to be investigated in association studies, to assess all different polymorphic states. However, due to linkage disequilibrium between polymorphisms, tightly linked polymorphisms are strongly correlated, which reduces the number of polymorphisms that need to be investigated in an association study to observe a significant association. Another consequence of LD is that many polymorphisms may give an association signal due to the fact that these polymorphisms are strongly correlated.

Genomic LD maps have been generated across the genome, and such LD maps have been proposed to serve as framework for mapping disease-genes (Risch, N. & Merkiangas, K, Science 273:1516-1517 (1996); Maniatis, N., et al., Proc Natl Acad Sci USA 99:2228-2233 (2002); Reich, D E et al, Nature 411:199-204 (2001)).

It is now established that many portions of the human genome can be broken into series of discrete haplotype blocks containing a few common haplotypes; for these blocks, linkage disequilibrium data provides little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33:382-387 (2003)).

Haplotype blocks (LD blocks) can be used to map associations between phenotype and haplotype status, using single markers or haplotypes comprising a plurality of markers. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

It has thus become apparent that for any given observed association to a polymorphic marker in the genome, it is likely that additional markers in the genome also show association. This is a natural consequence of the uneven distribution of LD across the genome, as observed by the large variation in recombination rates. The markers used to detect association thus in a sense represent “tags” for a genomic region (i.e., a haplotype block or LD block) that is associating with a given disease or trait, and as such are useful for use in the methods and kits of the invention.

By way of example, the −/AA indel, encoding the truncated form of BRIP1 shown in SEQ ID NO:14 herein, may be detected directly to determine risk of ovarian cancer. Alternatively, any marker in linkage disequilibrium with the −/AA indel may be detected to determine risk. In other embodiments, markers in linkage disequilibrium with any one of the markers rs34289250 (SEQ ID NO:1); rs12938171 (SEQ ID NO:2); an A/T polymorphism at position 55,422,245 in NCBI Build 36 (SEQ ID NO:3); a C/T polymorphism at position 55,217,320 in NCBI Build 36 (SEQ ID NO:4); rs12451939 (SEQ ID NO:5); a G/T polymorphism at position 56,567,990 in NCBI Build 36 (SEQ ID NO:6); an A/C polymorphism at position 56,478,611 in NCBI Build 36 (SEQ ID NO:7); a C/T polymorphism at position 56,505,864 in NCBI Build 36 (SEQ ID NO:8); and rs12937080 (SEQ ID NO:9), may be used to determine risk.

Suitable surrogate markers may be selected using public information, such as from the International HapMap Consortium (http://www.hapmap.org) and the International 1000genomes Consortium (http://www.1000genomes.org). The markers may also be suitably selected from results of whole-genome sequencing. The stronger the linkage disequilibrium (i.e., the higher the correlation) to the anchor marker, the better the surrogate, and thus the mores similar the association detected by the surrogate is expected to be to the association detected by the anchor marker. Markers with values of r² equal to 1 are perfect surrogates for the at-risk variants, i.e. genotypes for one marker perfectly predicts genotypes for the other. In other words, the surrogate will, by necessity, give exactly the same association data to any particular disease as the anchor marker. Markers with smaller values of r² than 1 can also be surrogates for the at-risk anchor variant.

The present invention encompasses the assessment of such surrogate markers for the markers as disclosed herein. Such markers are annotated, mapped and listed in public databases, as well known to the skilled person, or can alternatively be readily identified by sequencing the region or a part of the region identified by the markers of the present invention in a group of individuals, and identify polymorphisms in the resulting group of sequences. As a consequence, the person skilled in the art can readily and without undue experimentation identify and select appropriate surrogate markers.

One consequence of LD is that causative variants are not necessarily the variants first used for detecting an association signal. It is for example contemplated that a variant that is in linkage disequilibrium with the −/AA indel may be a functionally relevant variant. Alternatively, one or more variants in linkage disequilibrium with one or more of the markers rs34289250 (SEQ ID NO:1); rs12938171 (SEQ ID NO:2); an A/T polymorphism at position 55,422,245 in NCBI Build 36 (SEQ ID NO:3); a C/T polymorphism at position 55,217,320 in NCBI Build 36 (SEQ ID NO:4); rs12451939 (SEQ ID NO:5); a G/T polymorphism at position 56,567,990 in NCBI Build 36 (SEQ ID NO:6); an A/C polymorphism at position 56,478,611 in NCBI Build 36 (SEQ ID NO:7); a C/T polymorphism at position 56,505,864 in NCBI Build 36 (SEQ ID NO:8); and rs12937080 (SEQ ID NO:9) may be a functionally relevant variant predictive of risk of ovarian cancer.

### Association Analysis

For single marker association to a disease, the Fisher exact test can be used to calculate two-sided p-values for each individual allele. Correcting for relatedness among patients can be done by extending a variance adjustment procedure previously described (Risch, N. & Teng, J. Genome Res., 8:1273-1288 (1998)) for sibships so that it can be applied to general familial relationships. The method of genomic controls (Devlin, B. & Roeder, K. Biometrics 55:997 (1999)) can also be used to adjust for the relatedness of the individuals and possible stratification.

For both single-marker and haplotype analyses, relative risk (RR) and the population attributable risk (PAR) can be calculated assuming a multiplicative model (haplotype relative risk model) (Terwilliger, J. D. & Ott, J., Hum. Hered. 42:337-46 (1992) and Falk, C. T. & Rubinstein, P, Ann. Hum. Genet. 51 (Pt 3):227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes, hᵢ and hⱼ, risk(hᵢ)/risk(hⱼ)=(fᵢ/pᵢ)/(fⱼ/pⱼ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

### Risk Assessment and Diagnostics

Within any given population, there is an absolute risk of developing a disease or trait, defined as the chance of a person developing the specific disease or trait over a specified time-period. For example, a woman's lifetime absolute risk of breast cancer is one in nine. That is to say, one woman in every nine will develop breast cancer at some point in their lives. Risk is typically measured by looking at very large numbers of people, rather than at a particular individual. Risk is often presented in terms of Absolute Risk (AR) and Relative Risk (RR). Relative Risk is used to compare risks associating with two variants or the risks of two different groups of people. For example, it can be used to compare a group of people with a certain genotype with another group having a different genotype. For a disease, a relative risk of 2 means that one group has twice the chance of developing a disease as the other group. The risk presented is usually the relative risk for a person, or a specific genotype of a person, compared to the population with matched gender and ethnicity. Risks of two individuals of the same gender and ethnicity could be compared in a simple manner. For example, if, compared to the population, the first individual has relative risk 1.5 and the second has relative risk 0.5, then the risk of the first individual compared to the second individual is 1.5/0.5=3.

### Risk Calculations

The creation of a model to calculate the overall genetic risk involves two steps: i) conversion of odds-ratios for a single genetic variant into relative risk and ii) combination of risk from multiple variants in different genetic loci into a single relative risk value.

### Deriving Risk from Odds-Ratios

Most gene discovery studies for complex diseases that have been published to date in authoritative journals have employed a case-control design because of their retrospective setup. These studies sample and genotype a selected set of cases (people who have the specified disease condition) and control individuals. The interest is in genetic variants (alleles) which frequency in cases and controls differ significantly.

The results are typically reported in odds ratios, that is the ratio between the fraction (probability) with the risk variant (carriers) versus the non-risk variant (non-carriers) in the groups of affected versus the controls, i.e. expressed in terms of probabilities conditional on the affection status:

OR=(Pr(c|A)/Pr(nc|A))/(Pr(c|C)/Pr(nc|C))

Sometimes it is however the absolute risk for the disease that we are interested in, i.e. the fraction of those individuals carrying the risk variant who get the disease or in other words the probability of getting the disease. This number is typically not directly measured in case-control studies, in part, because the ratio of cases versus controls is typically not the same as that in the general population. However, under certain assumption, we can estimate the risk from the odds ratio.

It is well known that under the rare disease assumption, the relative risk of a disease can be approximated by the odds ratio. This assumption may however not hold for many common diseases. Still, it turns out that the risk of one genotype variant relative to another can be estimated from the odds ratio expressed above. The calculation is particularly simple under the assumption of random population controls where the controls are random samples from the same population as the cases, including affected people rather than being strictly unaffected individuals. To increase sample size and power, many of the large genome-wide association and replication studies use controls that were neither age-matched with the cases, nor were they carefully scrutinized to ensure that they did not have the disease at the time of the study. Hence, they often approximate a random sample from the general population. It is noted that this assumption is rarely expected to be satisfied exactly, but the risk estimates are usually robust to moderate deviations from this assumption.

Calculations show that for the dominant and the recessive models, where we have a risk variant carrier, “c”, and a non-carrier, “nc”, the odds ratio of individuals is the same as the risk ratio between these variants:

OR=Pr(A|c)/Pr(A|nc)=r

And likewise for the multiplicative model, where the risk is the product of the risk associated with the two allele copies, the allelic odds ratio equals the risk factor:

OR=Pr(A|aa)/Pr(A|ab)=Pr(A|ab)/Pr(A|bb)=r

Here “a” denotes the risk allele and “b” the non-risk allele. The factor “r” is therefore the relative risk between the allele types.

For many of the studies published in the last few years, reporting common variants associated with complex diseases, the multiplicative model has been found to summarize the effect adequately and most often provide a fit to the data superior to alternative models such as the dominant and recessive models.

### Determining Risk

In the present context, an individual who is at an increased susceptibility (i.e., increased risk) for ovarian cancer is an individual who is carrying at least one at-risk variant as described herein. In certain embodiments, the variant is within the human BRIP1 gene, or a variant encoded by a variation in the human BRIP1 gene. In one embodiment, significance associated with a marker is measured by a relative risk (RR). In another embodiment, significance associated with a marker or haplotye is measured by an odds ratio (OR). In a further embodiment, the significance is measured by a percentage. In one embodiment, a significant increased risk is measured as a risk (relative risk and/or odds ratio) of at least 2.0, including but not limited to at least 3.0, at least 3.5, at least 4.0, at least 5.0, at least 6.0, at least 7.0, at least 8.0, at least 9.0, at least 10.0, at least 11.0, at least 12.0, at least 13.0, at least 14.0, at least 15.0, at least 16.0, at least 18.0, at least 20.0, at least 22.0, or at least 24.0. In a particular embodiment, a risk (relative risk and/or odds ratio) of at least 5.0 is significant. In another particular embodiment, a risk of at least 7.0 is significant.

An at-risk variant as described herein is one where at least one allele of at least one marker is more frequently present in an individual at risk for ovarian cancer (affected), or diagnosed with ovarian cancer, compared to the frequency in a comparison group (control), such that the presence of the marker allele is indicative of susceptibility to ovarian cancer. The control group may in one embodiment be a population sample, i.e. a random sample from the general population. In another embodiment, the control group is represented by a group of individuals who are disease-free, i.e. individuals who have not been diagnosed with Ovarian cancer.

The person skilled in the art will appreciate that for markers with two alleles present in the population being studied (such as SNPs), and wherein one allele is found in increased frequency in a group of individuals with a trait or disease in the population, compared with controls, the other allele of the marker will be found in decreased frequency in the group of individuals with the trait or disease, compared with controls. In such a case, one allele of the marker (the one found in increased frequency in individuals with the trait or disease) will be the at-risk allele, while the other allele will be a protective allele.

### Database

Determining susceptibility can alternatively or additionally comprise comparing nucleic acid sequence data and/or protein sequence data (e.g., genotype data) to a database containing correlation data between polymorphic markers and susceptibility to ovarian cancer. The database can be part of a computer-readable medium described herein.

In a specific aspect of the invention, the database comprises at least one measure of susceptibility to ovarian cancer for the polymorphic markers. For example, the database may comprise risk values associated with particular genotypes at such markers. The database may also comprise risk values associated with particular genotype combinations for multiple such markers.

In another specific aspect of the invention, the database comprises a look-up table containing at least one measure of susceptibility to ovarian cancer for the polymorphic markers.

### Further Steps

The methods disclosed herein can comprise additional steps which may occur before, after, or simultaneously with one of the aforementioned steps of the method of the invention. In a specific embodiment of the invention, the method of determining a susceptibility to cancer further comprises reporting the susceptibility to at least one entity selected from the group consisting of the individual, a guardian of the individual, a genetic service provider, a physician, a medical organization, and a medical insurer. The reporting may be accomplished by any of several means. For example, the reporting can comprise sending a written report on physical media or electronically or providing an oral report to at least one entity of the group, which written or oral report comprises the susceptibility. Alternatively, the reporting can comprise providing the at least one entity of the group with a login and password, which provides access to a report comprising the susceptibility posted on a password-protected computer system.

### Study Population

In a general sense, the methods and kits described herein can be utilized from samples containing nucleic acid material (DNA or RNA) or protein material from any source and from any individual, or from genotype or sequence data derived from such samples. In preferred embodiments, the individual is a human individual. The individual can be an adult, child, or fetus. In some embodiments, the individual is a female individual. The nucleic acid or protein source may be any sample comprising nucleic acid or protein material, including biological samples, or a sample comprising nucleic acid or protein material derived therefrom. The present invention also provides for assessing markers in individuals who are members of a target population. Such a target population is in one embodiment a population or group of individuals at risk of developing ovarian cancer, based on other genetic factors, biomarkers, biophysical parameters, or lifestyle factors.

The Icelandic population is a Caucasian population of Northern European ancestry. A large number of studies reporting results of genetic linkage and association in the Icelandic population have been published in the last few years. Many of those studies show replication of variants, originally identified in the Icelandic population as being associating with a particular disease, in other populations (Sulem, P., et al. Nat Genet May 17, 2009 (Epub ahead of print); Rafnar, T., et al. Nat Genet. 41:221-7 (2009); Gretarsdottir, S., et al. Ann Neurol 64:402-9 (2008); Stacey, S. N., et al. Nat Genet. 40:1313-18 (2008); Gudbjartsson, D. F., et al. Nat Genet. 40:886-91 (2008); Styrkarsdottir, U., et al. N Engl J Med 358:2355-65 (2008); Thorgeirsson, T., et al. Nature 452:638-42 (2008); Gudmundsson, J., et al. Nat. Genet. 40:281-3 (2008); Stacey, S. N., et al., Nat. Genet. 39:865-69 (2007); Helgadottir, A., et al., Science 316:1491-93 (2007); Steinthorsdottir, V., et al., Nat. Genet. 39:770-75 (2007); Gudmundsson, J., et al., Nat. Genet. 39:631-37 (2007); Frayling, T M, Nature Reviews Genet 8:657-662 (2007); Amundadottir, L. T., et al., Nat. Genet. 38:652-58 (2006); Grant, S. F., et al., Nat. Genet. 38:320-23 (2006)). Thus, genetic findings in the Icelandic population have in general been replicated in other populations, including populations from Africa and Asia.

It is thus believed that the markers described herein to be associated with risk of ovarian cancer will show similar association in other human populations. It is further contemplated that additional variants in the human BRIP1 gene may be conferring risk of ovarian cancer in other populations. Particular embodiments comprising individual human populations are thus also contemplated and within the scope of the invention. Such embodiments relate to human subjects that are from one or more human population including, but not limited to, white populations, Caucasian populations, European populations, American populations, Eurasian populations, Asian populations, Central/South Asian populations, East Asian populations, and African populations. In certain embodiments, the invention pertains to individuals from Caucasian populations. In certain embodiments, the invention pertains to Icelandic individuals. In certain embodiments, the invention pertains to white individuals.

The population origin of individuals can be determined using methods known in the art. In certain embodiments, the origin of individuals is determined through self-reporting. In such embodiments, individuals describe their population origin themselves. For example, individuals may characterize themselves as belonging to any of the above mentioned populations. This method is routinely used in the art, for example in clinical studies.

Alternatively, the population origin of individuals may be determined at the nucleic acid level using genetic markers, which is a method well known to the skilled person. Using groups of individuals from specific populations/ethnic groups as a reference, it is possible to assign genomic material of unknown origin to particular populations. This may be routinely performed by the skilled person, using genetic markers that are population specific, and thus appropriate for determining genetic origin of nucleic acid samples.

In certain embodiments, the invention relates to markers identified in specific populations, as described in the above. The person skilled in the art will appreciate that measures of linkage disequilibrium (LD) may give different results when applied to different populations. This is due to different population history of different human populations as well as differential selective pressures that may have led to differences in LD in specific genomic regions. It is also well known to the person skilled in the art that certain markers, e.g. SNP markers, have different population frequency in different populations, or are polymorphic in one population but not in another. The person skilled in the art will however apply the methods available and as taught herein to practice the present invention in any given human population. This may include assessment of polymorphic markers in the LD region of the present invention, so as to identify those markers that give strongest association within the specific population. Thus, the at-risk variants of the present invention may reside on different haplotype background and in different frequencies in various human populations. However, utilizing methods known in the art and the markers of the present invention, the invention can be practiced in any given human population.

### Diagnostic Methods

Polymorphic markers associated with increased susceptibility of cancer, e.g. ovarian cancer, are useful in diagnostic methods. While methods of diagnosing cancer are known in the art, the detection risk markers for ovarian cancer advantageously may be useful for detection of cancer at its early stages and may also reduce the occurrence of misdiagnosis. In this regard, the invention further provides methods of diagnosing cancer comprising obtaining sequence data identifying at least one risk allele as described herein, in conjunction with carrying out one or more clinical diagnostic steps for the identification of cancer. Such diagnostic steps may include transvaginal ultrasound (TVU) and determination of CA-125 levels in the blood. The diagnostic steps may further include assessment of symptoms selected from abdominal pain or discomfort, an abdominal mass, bloating, back pain, urinary urgency, constipation, tiredness, pelvic pain, abnormal vaginal bleeding and involuntary weight loss.

The present invention pertains in some embodiments to methods of clinical applications of diagnosis, e.g., diagnosis performed by a medical professional. In other embodiments, the invention pertains to methods of diagnosis or methods of determination of a susceptibility performed by a layman. The layman can be the customer of a sequencing or genotyping service. The layman may also be a genotype or sequencing service provider, who performs analysis on a DNA sample from an individual, in order to provide service related to genetic risk factors for particular traits or diseases, based on the genotype status of the individual (i.e., the customer). Sequencing methods include for example those discussed in the above, but in general any suitable sequencing method may be used in the methods described and claimed herein. Recent technological advances in genotyping technologies, including high-throughput genotyping of SNP markers, such as Molecular Inversion Probe array technology (e.g., Affymetrix GeneChip), and BeadArray Technologies (e.g., Illumina GoldenGate and Infinium assays) have made it possible for individuals to have their own genome assessed for up to one million SNPs simultaneously, at relatively little cost. The resulting genotype information, which can be made available to the individual, can be compared to information about disease or trait risk associated with various SNPs, including information from public literature and scientific publications.

The application of disease-associated alleles as described herein, can thus for example be performed by the individual, through analysis of his/her genotype data, by a health professional based on results of a clinical test, or by a third party, including the genotype or sequencing service provider. The third party may also be service provider who interprets genotype or sequence information from the customer to provide service related to specific genetic risk factors, including the genetic markers described herein. In other words, the diagnosis or determination of a susceptibility of genetic risk can be made by health professionals, genetic counselors, third parties providing genotyping and/or sequencing service, third parties providing risk assessment service or by the layman (e.g., the individual), based on information about the genotype status of an individual and knowledge about the risk conferred by particular genetic risk factors (e.g., particular SNPs). In the present context, the term “diagnosing”, “diagnose a susceptibility” and “determine a susceptibility” is meant to refer to any available method for determining a susceptibility or risk of disease, including those mentioned above.

In certain embodiments, a sample containing genomic DNA or protein from an individual is collected. Such sample can for example be a buccal swab, a saliva sample, a blood sample, or other suitable samples containing genomic DNA or protein, as described further herein. In certain embodiments, the sample is obtained by non-invasive means (e.g., for obtaining a buccal sample, saliva sample, hair sample or skin sample). In certain embodiments, the sample is obtained by non-surgical means, i.e. in the absence of a surgical intervention on the individual that puts the individual at substantial health risk. Such embodiments may, in addition to non-invasive means also include obtaining sample by extracting a blood sample (e.g., a venous blood sample). The genomic DNA or protein obtained from the individual is then analyzed using any common technique available to the skilled person, such as high-throughput technologies for genotyping and/or sequencing. Results from such methods are stored in a convenient data storage unit, such as a data carrier, including computer databases, data storage disks, or by other convenient data storage means. In certain embodiments, the computer database is an object database, a relational database or a post-relational database. The genotype data is subsequently analyzed for the presence of certain variants known to be susceptibility variants for a particular human condition, such as the genetic variants described herein associated with risk of ovarian cancer. Genotype and/or sequencing data can be retrieved from the data storage unit using any convenient data query method. Calculating risk conferred by a particular genotype for the individual can be based on comparing the genotype of the individual to previously determined risk (expressed as a relative risk (RR) or and odds ratio (OR), for example) for the genotype, for example for an heterozygous carrier of an at-risk variant. The calculated risk for the individual can be the relative risk for a person, or for a specific genotype of a person, compared to the average population with matched gender and ethnicity. The average population risk can be expressed as a weighted average of the risks of different genotypes, using results from a reference population, and the appropriate calculations to calculate the risk of a genotype group relative to the population can then be performed. Alternatively, the risk for an individual is based on a comparison of particular genotypes, for example heterozygous carriers of an at-risk allele of a marker compared with non-carriers of the at-risk allele. The calculated risk estimated can be made available to the customer via a website, preferably a secure website.

### Methods of Selecting Individuals for Therapy

Most currently-used cancer treatments aim to introduce damaging lesions into DNA of replicating cells to the extent that the cell cannot repair the damage and will die. The two most common form of damage are DNA double stranded breaks (DSB) and DNA single stranded breaks (SSB) (reviewed in Yap et al, CA Cancer 61, 31 (2011)). Several anticancer drugs, as well as ionizing radiation, cause toxic double stranded breaks in DNA while other anticancer drugs produce different kinds of primary DNA lesions, including single stranded breaks.

Cells can employ a diverse range of DNA repair pathways to counteract the damage, depending on the types of lesions and repair required (Yap et al, CA Cancer 61, 31 (2011)). For example, the homologous recombination (HR) and non-homologous end joining pathways may be utilized for the repair of double stranded breaks in DNA. The HR system is highly conserved and error free, and is therefore the favored form of double stranded break repair. Unlike homologous recombination, the non-homologous end joining pathway is error prone and may lead to genomic instability. The major pathways that are used to repair single-stranded breaks in DNA are the nucleotide excision repair, base excision repair, or mismatch repair. Of these, base excision repair, is a key pathway for the repair of single-stranded breaks and encompasses the sensing of the DNA lesion, followed by recruitment of several other repair effectors through the action of Poly(ADP-ribose) polymerase (PARP).

Many tumor cells have specific genetic lesions in pathways that are important for DNA repair. This can be exploited by targeting genetically defective tumor cells with a specific molecular therapy that inhibits the remaining repair machinery, resulting in selective tumor cell killing (Helleday, T., Carcinogenesis 31, 955 (2010)). For example, tumor cells that have mutations in the breast cancer genes BRCA1 or BRCA2 have a defective homologous recombination repair pathway, making these cells dependent on the error-prone non-homologous end joining mechanism for repairing double-stranded breaks. When these cells are treated with PARP inhibitors, the PARP enzymes can no longer perform repair of single-stranded breaks. Unrepaired single-stranded breaks in PARP inhibited cells may be converted into toxic double-stranded breaks during replication, which will not be repaired efficiently in the absence of homologous recombination-mediated repair, and results in cell death. The demonstration of single-agent antitumor activity and the wide therapeutic index of PARP inhibitors in BRCA1 and BRCA2 mutation carriers with advanced breast or ovarian cancers provide strong evidence for the clinical application of this approach (Fong et al N Engl J Med 361, 123 (2009), Fong et al J Clin Oncol 28, 2512 (2010)).

BRIP1 interacts with the BRCT domain of BRCA1 and has several BRCA1-dependent, as well as independent, functions in preserving the structural and genetic integrity of the genome (reviewed by (Cantor, S. B. and Guillemette, G., FANCJ/BACH1/BRIP1. Future Oncol 7, 253)). It has been shown that BRIP1 is required for homologous recombination-mediated repair of double-stranded breaks, suggesting that BRIP1-deficient tumor cells will have the same sensitivity to PARP inhibitors as has been shown for BRCA1 and BRCA2 deficient tumors.

In addition to its role in homologous recombination-mediated DNA repair, BRIP1 is required for normal progression through S-phase by assisting in the resolution of stalled replication forks. Mutations in BRIP1 that impair its helicase function, render cells highly sensitive to crosslinking agents such as cisplatin, suggesting that BRIP1-mutant ovarian cancer cells may be good candidates for platinum drugs.

Given that individuals who carry truncating germline mutations in BRIP1 also have LOH over BRIP1, suggests that measurement of BRIP1 in the germline of ovarian cancer patients may help to identify patients who are likely to be responsive to drugs that target DNA repair pathways including PARP inhibitors, and cisplatin-class drugs.

It is therefore contemplated that individuals with ovarian cancer that carry loss-of-function mutations in BRIP1 are more likely to show a positive response to treatment for ovarian cancer, such as treatment by PARP inhibitors and/or a DNA crosslinking agents, than individuals that do not carry such mutations.

Accordingly, in one aspect of the invention, a method of assessing the responsiveness of a human individual to a therapeutic agent for ovarian cancer is provided, the method comprising determining the presence or absence of a loss-of-function mutation in the human BRIP1 gene in the genome of the individual, wherein a determination of the presence of the mutation is indicative that the individual is responsive to the therapeutic agent.

Another aspect provides a method of selecting a human subject with ovarian cancer for treatment with an ovarian cancer therapeutic agent, the method comprising determining the presence or absence of at least one allele of at least one polymorphic marker in a nucleic acid sample obtained from the individual, wherein the at least one allele causes a loss of function or loss of expression of BRIP1, and selecting for treatment with the therapeutic agent a subject identified as having the at least one allele in the nucleic acid sample. The nucleic acid sample may in certain embodiments be from an ovarian cancer tumor. In certain embodiments, the selecting step comprises selecting for treatment with the therapeutic agent a subject identified as having loss of heterozygosity of BRIP1, indicative of a loss-of-function of BRIP1 in the nucleic acid sample.

A further aspect provides a method of selecting a therapeutic regimen for a human subject with ovarian cancer, the method comprising analyzing data representative of at least one allele of a BRIP1 gene in a human subject with ovarian cancer to identify the presence or absence of a loss-of-function BRIP1 mutant allele, and selecting a therapeutic regimen of a therapeutic agent for treating ovarian cancer for a subject identified from the data as having the loss-of-function BRIP1 mutant allele. The BRIP1 gene is preferably a gene with sequence as set forth in SEQ ID NO:15 herein.

The analyzing suitably includes screening for presence or absence of mutant alleles in BRIP1 that are predictive of risk of ovarian cancer. In certain embodiments, such screening comprises screening for (a) a premature truncation or frameshift of an encoded BRIP1 protein, relative to the BRIP1 amino acid sequence set forth in SEQ ID NO: 13, (b) expression of a BRIP1 protein with reduced activity compared to a wild-type BRIP1 protein (SEQ ID NO: 13), or (c) reduced expression of BRIP1 protein, compared to another allele of the variant. The reduced activity is in certain embodiments an activity selected from BRIP1 binding to C-terminal BRCT motifs of wild-type BRCA1 protein, DNA-dependent ATPase activity, and DNA helicase activity. Such reduced activity can be determined using methods known in the art, including those described herein.

In certain embodiments, a tumor sample from an individual with ovarian cancer is analyzed for BRIP1 expression. Determination of the loss of wild-type BRIP1 protein in a tumor sample is in certain embodiments indicative of loss of heterozygosity (LOH). The selecting can therefore in certain embodiments comprise identifying an individual who also lacks wildtype BRIP1 in an ovarian cancer tumor sample.

The chemotherapy agent is preferably a PARP inhibitor or a DNA crosslinking agent. In preferred embodiments, the PARP inhibitor is selected from the group consisting of iniparib (4-iodo-3-nitrobenzamide), Olaparib (AZD-2281; 4-[(3-[(4-cyclopropylcarbonyl)piperazin-4-yl]carbonyl)-4-fluorophenyl]methyl(2H)phthalazin-1-one); Veliparib (ABT-888; 2-((R)-2-Methylpyrrolidin-2-yl)-1H-benzimidazole-4-carboxamide); Rucaparib (AG 014699; 8-Fluoro-2-{4-[(methylamino)methyl]phenyl}-1,3,4,5-tetrahydro-6H-azepino[5,4,3-cd]indol-6-one); 3-aminobenzamide; CEP 9722 (Cephalon); MK 4827 (Merck); KU-0059436 (AZD2281).

The DNA crosslinking agent is preferably selected from the group consisting of alkylating agents, such as Carmustine (1,3-bis(2-chloroethyl)-1-nitrosourea (BCNU)) and Nitrogen mustard (e.g., bis(2-chloroethyl)ethylamine, bis(2-chloroethyl)methylamine and tris(2-chloroethyl)amine), cisplatin ((SP-4-2)-diamminedichloridoplatinum) and cisplatin derivatives. In more preferred embodiments, the DNA crosslinking agent is cisplatin or a cisplatin derivative.

In certain embodiments, cisplatin derivatives are selected from the group consisting of Carboplatin (cis-diammine(cyclobutane-1,1-dicarboxylate-O,O′)platinum(II)) and Oxaliplatin ([(1R,2R)-cyclohexane-1,2-diamine](ethanedioato-O,O′)platinum(II)).

Methods of the invention relating to selecting patients may further include a step of administering the therapeutic to the human individual selected for the therapy. Methods of the invention relating to selecting patients may further include a step of prescribing the therapeutic for the human for self-administration, or administration by a medical professional other than the professional that selects the patient.

### Prognostic Methods

In addition to the utilities described above, the polymorphic markers of the invention are useful in determining a prognosis of a human individual with cancer. The variants described herein are indicative of risk of cancer, including ovarian cancer. Individuals carrying mutant alleles that predispose to cancer are at increased risk of the cancer. Such mutant alleles are predicted to be indicative of prognosis of the cancer.

The prognosis predicted can be any type of prognosis relating to the progression of the cancer, including ovarian cancer, and/or relating to the chance of recovering from the cancer. The prognosis can, for instance, relate to the severity of the cancer, or how the cancer will respond to therapeutic treatment.

Accordingly, the invention provides a method of predicting prognosis of an individual experiencing symptoms associated with, or an individual diagnosed with, ovarian cancer. The method comprises analyzing data representative of at least one allele of a BRIP1 gene in a human subject, wherein different alleles of the human BRIP1 gene are associated with different susceptibilities to at least one cancer in humans, and determining a prognosis of the human subject from the data. In certain embodiments, the cancer is ovarian cancer. The analyzing may comprise analysis for a mutation in BRIP1 that leads to loss of function or loss of expression of BRIP1. In certain embodiments, the analyzing comprises analyzing for the presence or absence of at least one mutant allele indicative of a BRIP1 defect selected from the group consisting of premature truncation or frameshift of an encoded BRIP1 protein, relative to the BRIP1 amino acid sequence set forth in SEQ ID NO:13, expression of a BRIP1 protein with reduced activity compared to a wild-type BRIP1 protein (SEQ ID NO:13), and reduced expression of BRIP1 protein, compared to wild-type BRIP1.

With regard to the prognostic methods described herein, the sequence data can be nucleic acid sequence data or amino acid sequence data. For example, in one embodiment, determination of the presence of a frameshift mutation or a nonsense mutation in BRIP1 is indicative of prognosis of ovarian cancer. The determination of the presence of a mutation in BRIP1 that leads to loss of function or loss of expression of BRIP1 is in certain embodiments indicative of a worsened prognosis of ovarian cancer. In other words, the presence of such mutations is in certain embodiments indicative that the individual has a worse prognosis of the cancer than do individuals with ovarian cancer who do not carry such mutations.

In some variations, the prognostic method further includes one or more additional steps, such as a step relating to generating the data by analyzing a biological sample; and/or a step involving selecting or administering a medial protocol to the subject, as described elsewhere herein.

### Methods of Treatment

It may be useful to select individuals for treatment based on the presence of altered forms of BRIP1, including mutations in BRIP1 that cause premature stop codons, or otherwise result in protein with reduced or no activity. As discussed in the above, it is contemplated that loss-of-function mutations in BRIP1 result in tumors that are particularly susceptible to therapy using PARP inhibitors or crosslinking agents. Therefore, it is contemplated that it may be beneficial to select individuals for therapy based on whether the individuals are carriers of such mutations.

Accordingly, the invention provides in one aspect a method of treatment of ovarian cancer, the method comprising steps of (a) determining the presence or absence of a mutation that causes a loss of function or loss of expression of BRIP1 in a nucleic acid sample from the human individual; (b) selecting for treatment an individual determined to have such a mutation; and (c) administering to the selected individual a pharmaceutically acceptable amount of a therapeutic agent for ovarian cancer selected from a PARP inhibitor and a DNA crosslinking agent.

In certain embodiments, the therapeutic agent is a PARP inhibitor or a DNA crosslinking agent. The PARP inhibitor may suitably be selected from the group consisting of iniparib (4-iodo-3-nitrobenzamide), Olaparib (AZD-2281; 4-[(3-[(4-cyclopropylcarbonyl)piperazin-4-yl]carbonyl)-4-fluorophenyl]methyl(2H)phthalazin-1-one); Veliparib (ABT-888; 2-((R)-2-Methylpyrrolidin-2-yl)-1H-benzimidazole-4-carboxamide); Rucaparib (AG 014699; 8-Fluoro-2-{4-[(methylamino)methyl]phenyl}-1,3,4,5-tetrahydro-6H-azepino[5,4,3-cd]indol-6-one); 3-aminobenzamide; CEP 9722 (Cephalon); MK 4827 (Merck); KU-0059436 (AZD2281).

The DNA crosslinking agent is preferably selected from the group consisting of alkylating agents, such as Carmustine (1,3-bis(2-chloroethyl)-1-nitrosourea (BCNU)) and Nitrogen mustard (e.g., bis(2-chloroethyl)ethylamine, bis(2-chloroethyl)methylamine and tris(2-chloroethyl)amine), cisplatin ((SP-4-2)-diamminedichloridoplatinum) and cisplatin derivatives. In certain more preferred embodiments, the DNA crosslinking agent is cisplatin or a cisplatin derivative.

In certain embodiments, cisplatin derivatives are selected from the group consisting of Carboplatin (cis-diammine(cyclobutane-1,1-dicarboxylate-O,O′)platinum(II)) and Oxaliplatin ([(1R,2R)-cyclohexane-1,2-diamine](ethanedioato-O,O′)platinum(II)).

### Kits

Kits useful in the methods of the invention comprise components useful in any of the methods described herein, including for example, primers for nucleic acid amplification, hybridization probes (e.g. probes for detecting particular mutant alleles), restriction enzymes (e.g., for RFLP analysis), allele-specific oligonucleotides, antibodies, e.g., antibodies that bind to an altered BRIP1 polypeptide (e.g. a missense variant in BRIP1 or a truncated BRIP1 polypeptide) or to a non-altered (native) BRIP1 polypeptide, means for amplification of nucleic acids, means for analyzing the nucleic acid sequence of nucleic acids, means for analyzing the amino acid sequence of polynucleotides, etc. The kits can for example include necessary buffers, nucleic acid primers for amplifying nucleic acids (e.g., a nucleic acid segment comprising one or more of the polymorphic markers as described herein), and reagents for allele-specific detection of the fragments amplified using such primers and necessary enzymes (e.g., DNA polymerase). Additionally, kits can provide reagents for assays to be used in combination with the methods of the present invention, e.g., reagents for use with other diagnostic assays for ovarian cancer or related conditions.

In one embodiment, the invention pertains to a kit for assaying a sample from a subject to detect a susceptibility to cancer (e.g., ovarian cancer) in the subject, wherein the kit comprises reagents necessary for selectively detecting at least one at-risk variant for cancer in the individual, wherein the at least one at-risk variant is a polymorphic marker in the human BRIP1 gene or an amino acid substitution in an encoded BRIP1 protein. In certain embodiments, the markers encodes a BRIP1 protein with a defect selected from (a) premature truncation or frameshift of BRIP1 polypeptide, relative to wild-type BRIP1; (b) expression of BRIP1 protein with reduced activity compared with wild-type BRIP1, wherein the activity is selected from (1) BRIP1 binding to BRCT motif in BRCA1, (2) DNA-dependent ATPase activity, and (3) DNA helicase activity, and (c) reduced expression of BRIP1 protein compared with wild-type BRIP1. In a particular embodiment, the reagents comprise at least one contiguous oligonucleotide that hybridizes to a fragment of the genome of the individual comprising at least one polymorphism of the present invention. In another embodiment, the reagents comprise at least one pair of oligonucleotides that hybridize to opposite strands of a genomic segment obtained from a subject, wherein each oligonucleotide primer pair is designed to selectively amplify a fragment of the genome of the individual that includes at least one polymorphism associated with the condition risk. In one such embodiment, the polymorphism is selected from chr17:57208601 ins+AA and chr17: 57213073 delTT. In yet another embodiment the fragment is at least 20 base pairs in size. Such oligonucleotides or nucleic acids (e.g., oligonucleotide primers) can be designed using portions of the nucleic acid sequence flanking the polymorphism. In another embodiment, the kit comprises one or more labeled nucleic acids capable of allele-specific detection of one or more specific polymorphic markers or haplotypes, and reagents for detection of the label. Suitable labels include, e.g., a radioisotope, a fluorescent label, an enzyme label, an enzyme co-factor label, a magnetic label, a spin label, an epitope label.

In certain embodiments, determination of the presence of a particular marker allele is indicative of a increased susceptibility of cancer. In another embodiment, determination of the presence of a particular marker allele is indicative of prognosis of ovarian cancer, or selection of appropriate therapy for ovarian cancer. In another embodiment, the presence of the marker allele or haplotype is indicative of response to therapy for ovarian cancer. In yet another embodiment, the presence of the marker allele is indicative of progress of treatment of ovarian cancer.

In certain embodiments, the kit comprises reagents for detecting no more than 100 alleles in the genome of the individual. In certain other embodiments, the kit comprises reagents for detecting no more than 20 alleles in the genome of the individual.

In a further aspect of the present invention, a pharmaceutical pack (kit) is provided, the pack comprising a therapeutic agent and a set of instructions for administration of the therapeutic agent to humans diagnostically tested for an at-risk variant for ovarian cancer. The therapeutic agent can be a small molecule drug, an antibody, a peptide, an antisense or RNAi molecule, or other therapeutic molecules. In one embodiment, an individual identified as a carrier of at least one variant of the present invention is instructed to take a prescribed dose of the therapeutic agent. In one such embodiment, an individual identified as a homozygous carrier of at least one variant of the present invention (e.g., an at-risk variant) is instructed to take a prescribed dose of the therapeutic agent. In another embodiment, an individual identified as a non-carrier of at least one variant of the present invention (e.g., an at-risk variant) is instructed to take a prescribed dose of the therapeutic agent.

The kit may additionally or alternatively comprise reagents for detecting an amino acid variation in a human BRIP1 protein (e.g., an amino acid substitution, or a truncated or otherwise altered amino acid sequence of an encoded BRIP1 protein). In one embodiment, the kit comprises at least one antibody for selectively detecting a truncated BRIP1 polypeptide compared with wild-type BRIP1 (SEQ ID NO:13). Other reagents useful for detecting amino acid variations are known to the skilled person and are also contemplated.

In certain embodiments, the kit further comprises a set of instructions for using the reagents comprising the kit. In certain embodiments, the kit further comprises a collection of data comprising correlation data between the at least one at-risk variant and susceptibility to Ovarian cancer.

### Antisense Agents

The nucleic acids and/or variants described herein, or nucleic acids comprising their complementary sequence, may be used as antisense constructs to control gene expression in cells, tissues or organs. The methodology associated with antisense techniques is well known to the skilled artisan, and is for example described and reviewed in AntisenseDrug Technology: Principles, Strategies, and Applications, Crooke, ed., Marcel Dekker Inc., New York (2001). In general, antisense agents (antisense oligonucleotides) are comprised of single stranded oligonucleotides (RNA or DNA) that are capable of binding to a complimentary nucleotide segment. By binding the appropriate target sequence, an RNA-RNA, DNA-DNA or RNA-DNA duplex is formed. The antisense oligonucleotides are complementary to the sense or coding strand of a gene. It is also possible to form a triple helix, where the antisense oligonucleotide binds to duplex DNA.

Several classes of antisense oligonucleotide are known to those skilled in the art, including cleavers and blockers. The former bind to target RNA sites, activate intracellular nucleases (e.g., RnaseH or Rnase L), that cleave the target RNA. Blockers bind to target RNA, inhibit protein translation by steric hindrance of the ribosomes. Examples of blockers include nucleic acids, morpholino compounds, locked nucleic acids and methylphosphonates (Thompson, Drug Discovery Today, 7:912-917 (2002)). Antisense oligonucleotides are useful directly as therapeutic agents, and are also useful for determining and validating gene function, for example by gene knock-out or gene knock-down experiments. Antisense technology is further described in Layery et al., Curr. Opin. Drug Discov. Devel. 6:561-569 (2003), Stephens et al., Curr. Opin. Mol. Ther. 5:118-122 (2003), Kurreck, Eur. J. Biochem. 270:1628-44 (2003), Dias et al., Mol. Cancer Ter. 1:347-55 (2002), Chen, Methods Mol. Med. 75:621-636 (2003), Wang et al., Curr. Cancer Drug Targets 1:177-96 (2001), and Bennett, Antisense Nucleic Acid Drug. Dev. 12:215-24 (2002).

In certain embodiments, the antisense agent is an oligonucleotide that is capable of binding to a particular nucleotide segment. In certain embodiments, the nucleotide segment comprises the human BRIP1 gene. In certain other embodiments, the antisense nucleotide is capable of binding to a nucleotide segment of a human BRIP1 transcript, as set forth in SEQ ID NO:10. In one embodiment, the antisense nucleotide is capable of binding the a nucleotide segment of a human BRIP1 transcript with sequence as set forth in SEQ ID NO:10 that has a TT insertion between position 2345 and 2346. In another embodiment, the antisense nucleotide is capable of binding the a nucleotide segment of the human BRIP1 gene that has an AA insertion between position 57,208,601 and 57,208,602 in NCBI Build 36 (SEQ ID NO:12). Antisense nucleotides can be from 5-400 nucleotides in length, including 5-200 nucleotides, 5-100 nucleotides, 10-50 nucleotides, and 10-30 nucleotides. In certain preferred embodiments, the antisense nucleotides is from 14-50 nucleotides in length, including 14-40 nucleotides and 14-30 nucleotides.

The variants described herein can also be used for the selection and design of antisense reagents that are specific for particular variants. Using information about the variants described herein, antisense oligonucleotides or other antisense molecules that specifically target mRNA molecules that contain one or more variants of the invention can be designed. In this manner, expression of mRNA molecules that contain one or more variant of the present invention can be inhibited or blocked. In one embodiment, the antisense molecules are designed to specifically bind a particular allelic form of the target nucleic acid, thereby inhibiting translation of a product originating from this specific allele, but which do not bind other or alternate variants at the specific polymorphic sites of the target nucleic acid molecule. In one embodiment, the antisense molecule is designed to specifically bind to nucleic acids comprising the AA insertion in BRIP1. As antisense molecules can be used to inactivate mRNA so as to inhibit gene expression, and thus protein expression, the molecules can be used for disease treatment. The methodology can involve cleavage by means of ribozymes containing nucleotide sequences complementary to one or more regions in the mRNA that attenuate the ability of the mRNA to be translated. Such mRNA regions include, for example, protein-coding regions, in particular protein-coding regions corresponding to catalytic activity, substrate and/or ligand binding sites, or other functional domains of a protein.

The phenomenon of RNA interference (RNAi) has been actively studied for the last decade, since its original discovery in C. elegans (Fire et al., Nature 391:806-11 (1998)), and in recent years its potential use in treatment of human disease has been actively pursued (reviewed in Kim & Rossi, Nature Rev. Genet. 8:173-204 (2007)). RNA interference (RNAi), also called gene silencing, is based on using double-stranded RNA molecules (dsRNA) to turn off specific genes. In the cell, cytoplasmic double-stranded RNA molecules (dsRNA) are processed by cellular complexes into small interfering RNA (siRNA). The siRNA guide the targeting of a protein-RNA complex to specific sites on a target mRNA, leading to cleavage of the mRNA (Thompson, Drug Discovery Today, 7:912-917 (2002)). The siRNA molecules are typically about 20, 21, 22 or 23 nucleotides in length. Thus, one aspect of the invention relates to isolated nucleic acid molecules, and the use of those molecules for RNA interference, i.e. as small interfering RNA molecules (siRNA). In one embodiment, the isolated nucleic acid molecules are 18-26 nucleotides in length, preferably 19-25 nucleotides in length, more preferably 20-24 nucleotides in length, and more preferably 21, 22 or 23 nucleotides in length.

Another pathway for RNAi-mediated gene silencing originates in endogenously encoded primary microRNA (pri-miRNA) transcripts, which are processed in the cell to generate precursor miRNA (pre-miRNA). These miRNA molecules are exported from the nucleus to the cytoplasm, where they undergo processing to generate mature miRNA molecules (miRNA), which direct translational inhibition by recognizing target sites in the 3′ untranslated regions of mRNAs, and subsequent mRNA degradation by processing P-bodies (reviewed in Kim & Rossi, Nature Rev. Genet. 8:173-204 (2007)).

Clinical applications of RNAi include the incorporation of synthetic siRNA duplexes, which preferably are approximately 20-23 nucleotides in size, and preferably have 3′ overlaps of 2 nucleotides. Knockdown of gene expression is established by sequence-specific design for the target mRNA. Several commercial sites for optimal design and synthesis of such molecules are known to those skilled in the art.

Other applications provide longer siRNA molecules (typically 25-30 nucleotides in length, preferably about 27 nucleotides), as well as small hairpin RNAs (shRNAs; typically about 29 nucleotides in length). The latter are naturally expressed, as described in Amarzguioui et al. (FEBS Lett. 579:5974-81 (2005)). Chemically synthetic siRNAs and shRNAs are substrates for in vivo processing, and in some cases provide more potent gene-silencing than shorter designs (Kim et al., Nature Biotechnol. 23:222-226 (2005); Siolas et al., Nature Biotechnol. 23:227-231 (2005)). In general siRNAs provide for transient silencing of gene expression, because their intracellular concentration is diluted by subsequent cell divisions. By contrast, expressed shRNAs mediate long-term, stable knockdown of target transcripts, for as long as transcription of the shRNA takes place (Marques et al., Nature Biotechnol. 23:559-565 (2006); Brummelkamp et al., Science 296: 550-553 (2002)).

Since RNAi molecules, including siRNA, miRNA and shRNA, act in a sequence-dependent manner, the variants presented herein can be used to design RNAi reagents that recognize specific nucleic acid molecules comprising specific alleles and/or haplotypes (e.g., the alleles and/or haplotypes of the present invention), while not recognizing nucleic acid molecules comprising other alleles or haplotypes. These RNAi reagents can thus recognize and destroy the target nucleic acid molecules. As with antisense reagents, RNAi reagents can be useful as therapeutic agents (i.e., for turning off disease-associated genes or disease-associated gene variants), but may also be useful for characterizing and validating gene function (e.g., by gene knock-out or gene knock-down experiments).

Delivery of RNAi may be performed by a range of methodologies known to those skilled in the art. Methods utilizing non-viral delivery include cholesterol, stable nucleic acid-lipid particle (SNALP), heavy-chain antibody fragment (Fab), aptamers and nanoparticles. Viral delivery methods include use of lentivirus, adenovirus and adeno-associated virus. The siRNA molecules are in some embodiments chemically modified to increase their stability. This can include modifications at the 2′ position of the ribose, including 2′-O-methylpurines and 2′-fluoropyrimidines, which provide resistance to Rnase activity. Other chemical modifications are possible and known to those skilled in the art.

The following references provide a further summary of RNAi, and possibilities for targeting specific genes using RNAi: Kim & Rossi, Nat. Rev. Genet. 8:173-184 (2007), Chen & Rajewsky, Nat. Rev. Genet. 8: 93-103 (2007), Reynolds, et al., Nat. Biotechnol. 22:326-330 (2004), Chi et al., Proc. Natl. Acad. Sci. USA 100:6343-6346 (2003), Vickers et al., J. Biol. Chem. 278:7108-7118 (2003), Agami, Curr. Opin. Chem. Biol. 6:829-834 (2002), Layery, et al., Curr. Opin. Drug Discov. Devel. 6:561-569 (2003), Shi, Trends Genet. 19:9-12 (2003), Shuey et al., Drug Discov. Today 7:1040-46 (2002), McManus et al., Nat. Rev. Genet. 3:737-747 (2002), Xia et al., Nat. Biotechnol. 20:1006-10 (2002), Plasterk et al., curr. Opin. Genet. Dev. 10:562-7 (2000), Bosher et al., Nat. Cell Biol. 2:E31-6 (2000), and Hunter, Curr. Biol. 9:R440-442 (1999).

### Nucleic Acids and Polypeptides

The nucleic acids and polypeptides described herein can be used in methods and kits of the present invention. An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography (e.g., HPLC). An isolated nucleic acid molecule of the invention can comprise at least about 50%, at least about 80% or at least about 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 250 kb, 200 kb, 150 kb, 100 kb, 75 kb, 50 kb, 25 kb, 10 kb, 5 kb, 4 kb, 3 kb, 2 kb, 1 kb, 0.5 kb or 0.1 kb of the nucleotides that flank the nucleic acid molecule in the genomic DNA of the cell from which the nucleic acid molecule is derived.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence containing a polymorphic site associated with a marker or haplotype described herein). Such nucleic acid molecules can be detected and/or isolated by allele- or sequence-specific hybridization (e.g., under high stringency conditions). Stringency conditions and methods for nucleic acid hybridizations are well known to the skilled person (see, e.g., Current Protocols in Molecular Biology, Ausubel, F. et al, John Wiley & Sons, (1998), and Kraus, M. and Aaronson, S., Methods Enzymol., 200:546-556 (1991), the entire teachings of which are incorporated by reference herein.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, at least 40%, at least 50%, at least 60%, at least 70%, at least 80%, at least 90%, or at least 95%, of the length of the reference sequence. The actual comparison of the two sequences can be accomplished by methods well known to the skilled person, for example, using the NBLAST and XBLAST programs, as described in Altschul, S. et al., Nucleic Acids Res., 25:3389-3402 (1997). Another example of an algorithm is BLAT (Kent, W. J. Genome Res. 12:656-64 (2002)).

The present invention also provides isolated nucleic acid molecules that contain a fragment or portion that hybridizes under highly stringent conditions to a nucleic acid that comprises, or consists of, the nucleotide sequence of the human BRIP1 gene as set forth in SEQ ID NO:15, or a nucleotide sequence comprising, or consisting of, the complement of the nucleotide sequence of SEQ ID NO:15. In certain embodiments, the nucleotide sequence comprises at least one polymorphic allele as described herein (e.g., chr17:57208601 ins+AA or chr17: 57213073 delTT). The nucleic acid fragments of the invention may suitably be at least about 15, at least about 18, 20, 23 or 25 nucleotides, and can be up to 30, 40, 50, 100, 200, 300 or 400 nucleotides in length.

The nucleic acid fragments of the invention are used as probes or primers in assays such as those described herein. “Probes” or “primers” are oligonucleotides that hybridize in a base-specific manner to a complementary strand of a nucleic acid molecule. In addition to DNA and RNA, such probes and primers include polypeptide nucleic acids (PNA), as described in Nielsen, P. et al., Science 254:1497-1500 (1991). A probe or primer comprises a region of nucleotide sequence that hybridizes to at least about 15, typically about 20-25, and in certain embodiments about 40, 50 or 75, consecutive nucleotides of a nucleic acid molecule. In one embodiment, the probe or primer comprises at least one allele of at least one polymorphic marker or at least one haplotype described herein, or the complement thereof. In particular embodiments, a probe or primer can comprise 100 or fewer nucleotides; for example, in certain embodiments from 6 to 50 nucleotides, or, for example, from 12 to 30 nucleotides. In other embodiments, the probe or primer is at least 70% identical, at least 80% identical, at least 85% identical, at least 90% identical, or at least 95% identical, to the contiguous nucleotide sequence or to the complement of the contiguous nucleotide sequence. In another embodiment, the probe or primer is capable of selectively hybridizing to the contiguous nucleotide sequence or to the complement of the contiguous nucleotide sequence. Often, the probe or primer further comprises a label, e.g., a radioisotope, a fluorescent label, an enzyme label, an enzyme co-factor label, a magnetic label, a spin label, an epitope label.

### Antibodies

The invention also provides antibodies which bind to an epitope comprising either a BRIP1 variant amino acid sequence (e.g., a polypeptide comprising an amino acid substitution or a truncated polypeptide) encoded by a variant allele or the reference amino acid sequence encoded by the corresponding non-variant or wild-type allele of BRIP1. The term “antibody” as used herein refers to immunoglobulin molecules and immunologically active portions of immunoglobulin molecules, i.e., molecules that contain antigen-binding sites that specifically bind an antigen. A molecule that specifically binds to a polypeptide of the invention is a molecule that binds to that polypeptide or a fragment thereof (e.g., a BRIP1 polypeptide with sequence as set forth in SEQ ID NO:13, or a fragment thereof), but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or a fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4: 72 (1983)), the EBV-hybridoma technique (Cole et al., Monoclonal Antibodies and Cancer Therapy, Alan R. Liss,1985, Inc., pp. 77-96) or trioma techniques. The technology for producing hybridomas is well known (see generally Current Protocols in Immunology (1994) Coligan et al., (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature 266:55052 (1977); R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981)). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurtZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9: 1370-1372 (1991); Hay et al., Hum. Antibod. Hybridomas 3:81-85 (1992); Huse et al., Science 246: 1275-1281 (1989); and Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide as described herein by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. The antibody can be coupled to a detectable substance to facilitate its detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies can furthermore be useful for assessing expression of proteins, e.g. BRIP1 expression. Antibodies specific BRIP1, or variants or truncated forms of BRIP1, may be used to determine the expression levels of BRIP1 in a sample from an individual.

Antibodies can be used in other methods. Thus, antibodies are useful as diagnostic tools for evaluating proteins, such as variant proteins of the invention, in conjunction with analysis by electrophoretic mobility, isoelectric point, tryptic or other protease digest, or for use in other physical assays known to those skilled in the art. Antibodies may also be used in tissue typing. In one such embodiment, a specific variant protein has been correlated with expression in a specific tissue type, and antibodies specific for the variant protein can then be used to identify the specific tissue type. For example, BRIP1 antibodies may be used to determine the expression levels of BRIP1 in ovarian cancer tumors.

Subcellular localization of proteins, including variant proteins, can also be determined using antibodies, and can be applied to assess aberrant subcellular localization of the protein in cells in various tissues. Such use can be applied in genetic testing, but also in monitoring a particular treatment modality. For example, it may be useful to determine the expression levels of BRIP1 in tumor samples from an individual. In certain embodiments, expression levels of BRIP1 in ovarian tumor samples are determined.

Antibodies are further useful for inhibiting variant protein function, for example by blocking the binding of a variant protein to a binding molecule or partner. Such uses can also be applied in a therapeutic context in which treatment involves inhibiting a variant protein's function. An antibody can be for example be used to block or competitively inhibit binding, thereby modulating (i.e., agonizing or antagonizing) the activity of the protein. Antibodies can be prepared against specific protein fragments containing sites required for specific function or against an intact protein that is associated with a cell or cell membrane. For administration in vivo, an antibody may be linked with an additional therapeutic payload, such as radionuclide, an enzyme, an immunogenic epitope, or a cytotoxic agent, including bacterial toxins (diphtheria or plant toxins, such as ricin). The in vivo half-life of an antibody or a fragment thereof may be increased by pegylation through conjugation to polyethylene glycol.

The present invention further relates to kits for using antibodies in the methods described herein. This includes, but is not limited to, kits for detecting the presence or absence of a protein (e.g., BRIP1, or variants or truncated forms thereof) in a test sample. One preferred embodiment comprises antibodies such as a labelled or labelable antibody and a compound or agent for detecting proteins in a biological sample, means for determining the amount or the presence and/or absence of protein (e.g., BRIP1, or variants or truncated forms thereof) in the sample, and means for comparing the amount of variant protein in the sample with a standard, as well as instructions for use of the kit.

### Computer-Implemented Aspects

As understood by those of ordinary skill in the art, the methods and information described herein may be implemented, in all or in part, as computer executable instructions on known computer readable media. For example, the methods described herein may be implemented in hardware. Alternatively, the method may be implemented in software stored in, for example, one or more memories or other computer readable medium and implemented on one or more processors. As is known, the processors may be associated with one or more controllers, calculation units and/or other units of a computer system, or implanted in firmware as desired. If implemented in software, the routines may be stored in any computer readable memory such as in RAM, ROM, flash memory, a magnetic disk, a laser disk, or other storage medium, as is also known. Likewise, this software may be delivered to a computing device via any known delivery method including, for example, over a communication channel such as a telephone line, the Internet, a wireless connection, etc., or via a transportable medium, such as a computer readable disk, flash drive, etc.

More generally, and as understood by those of ordinary skill in the art, the various steps described above may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

When implemented in software, the software may be stored in any known computer readable medium such as on a magnetic disk, an optical disk, or other storage medium, in a RAM or ROM or flash memory of a computer, processor, hard disk drive, optical disk drive, tape drive, etc. Likewise, the software may be delivered to a user or a computing system via any known delivery method including, for example, on a computer readable disk or other transportable computer storage mechanism.

Thus, another aspect of the invention is a system that is capable of carrying out a part or all of a method of the invention, or carrying out a variation of a method of the invention as described herein in greater detail. Exemplary systems include, as one or more components, computing systems, environments, and/or configurations that may be suitable for use with the methods and include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like. In some variations, a system of the invention includes one or more machines used for analysis of biological material (e.g., genetic material), as described herein. In some variations, this analysis of the biological material involves a chemical analysis and/or a nucleic acid amplification.

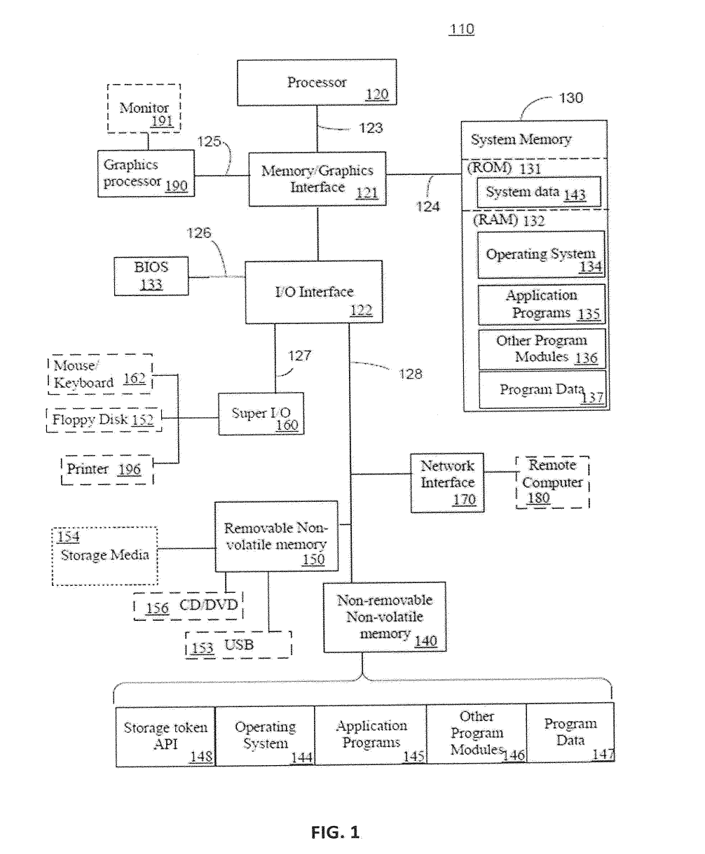

With reference to FIG. 1, an exemplary system of the invention, which may be used to implement one or more steps of methods of the invention, includes a computing device in the form of a computer 110. Components shown in dashed outline are not technically part of the computer 110, but are used to illustrate the exemplary embodiment of FIG. 1. Components of computer 110 may include, but are not limited to, a processor 120, a system memory 130, a memory/graphics interface 121, also known as a Northbridge chip, and an I/O interface 122, also known as a Southbridge chip. The system memory 130 and a graphics processor 190 may be coupled to the memory/graphics interface 121. A monitor 191 or other graphic output device may be coupled to the graphics processor 190.

A series of system busses may couple various system components including a high speed system bus 123 between the processor 120, the memory/graphics interface 121 and the I/O interface 122, a front-side bus 124 between the memory/graphics interface 121 and the system memory 130, and an advanced graphics processing (AGP) bus 125 between the memory/graphics interface 121 and the graphics processor 190. The system bus 123 may be any of several types of bus structures including, by way of example, and not limitation, such architectures include Industry Standard Architecture (ISA) bus, Micro Channel Architecture (MCA) bus and Enhanced ISA (EISA) bus. As system architectures evolve, other bus architectures and chip sets may be used but often generally follow this pattern. For example, companies such as Intel and AMD support the Intel Hub Architecture (IHA) and the Hypertransport™ architecture, respectively.

The computer 110 typically includes a variety of computer-readable media. Computer-readable media can be any available media that can be accessed by computer 110 and includes both volatile and nonvolatile media, removable and non-removable media. By way of example, and not limitation, computer readable media may comprise computer storage media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other physical medium which can be used to store the desired information and which can accessed by computer 110.

The system memory 130 includes computer storage media in the form of volatile and/or nonvolatile memory such as read only memory (ROM) 131 and random access memory (RAM) 132. The system ROM 131 may contain permanent system data 143, such as identifying and manufacturing information. In some embodiments, a basic input/output system (BIOS) may also be stored in system ROM 131. RAM 132 typically contains data and/or program modules that are immediately accessible to and/or presently being operated on by processor 120. By way of example, and not limitation, FIG. 1 illustrates operating system 134, application programs 135, other program modules 136, and program data 137.

The I/O interface 122 may couple the system bus 123 with a number of other busses 126, 127 and 128 that couple a variety of internal and external devices to the computer 110. A serial peripheral interface (SPI) bus 126 may connect to a basic input/output system (BIOS) memory 133 containing the basic routines that help to transfer information between elements within computer 110, such as during start-up.

A super input/output chip 160 may be used to connect to a number of ‘legacy’ peripherals, such as floppy disk 152, keyboard/mouse 162, and printer 196, as examples. The super I/O chip 160 may be connected to the I/O interface 122 with a bus 127, such as a low pin count (LPC) bus, in some embodiments. Various embodiments of the super I/O chip 160 are widely available in the commercial marketplace.

In one embodiment, bus 128 may be a Peripheral Component Interconnect (PCI) bus, or a variation thereof, may be used to connect higher speed peripherals to the I/O interface 122. A PCI bus may also be known as a Mezzanine bus. Variations of the PCI bus include the Peripheral Component Interconnect-Express (PCI-E) and the Peripheral Component Interconnect-Extended (PCI-X) busses, the former having a serial interface and the latter being a backward compatible parallel interface. In other embodiments, bus 128 may be an advanced technology attachment (ATA) bus, in the form of a serial ATA bus (SATA) or parallel ATA (PATA).

The computer 110 may also include other removable/non-removable, volatile/nonvolatile computer storage media. By way of example only, FIG. 1 illustrates a hard disk drive 140 that reads from or writes to non-removable, nonvolatile magnetic media. The hard disk drive 140 may be a conventional hard disk drive.

Removable media, such as a universal serial bus (USB) memory 153, firewire (IEEE 1394), or CD/DVD drive 156 may be connected to the PCI bus 128 directly or through an interface 150. A storage media 154 may couple through interface 150. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like.

The drives and their associated computer storage media discussed above and illustrated in FIG. 1, provide storage of computer readable instructions, data structures, program modules and other data for the computer 110. In FIG. 1, for example, hard disk drive 140 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a mouse/keyboard 162 or other input device combination. Other input devices (not shown) may include a microphone, joystick, game pad, satellite dish, scanner, or the like. These and other input devices are often connected to the processor 120 through one of the I/O interface busses, such as the SPI 126, the LPC 127, or the PCI 128, but other busses may be used. In some embodiments, other devices may be coupled to parallel ports, infrared interfaces, game ports, and the like (not depicted), via the super I/O chip 160.

The computer 110 may operate in a networked environment using logical connections to one or more remote computers, such as a remote computer 180 via a network interface controller (NIC) 170. The remote computer 180 may be a personal computer, a server, a router, a network PC, a peer device or other common network node, and typically includes many or all of the elements described above relative to the computer 110. The logical connection between the NIC 170 and the remote computer 180 depicted in FIG. 1 may include a local area network (LAN), a wide area network (WAN), or both, but may also include other networks. Such networking environments are commonplace in offices, enterprise-wide computer networks, intranets, and the Internet. The remote computer 180 may also represent a web server supporting interactive sessions with the computer 110, or in the specific case of location-based applications may be a location server or an application server.

In some embodiments, the network interface may use a modem (not depicted) when a broadband connection is not available or is not used. It will be appreciated that the network connection shown is exemplary and other means of establishing a communications link between the computers may be used.

In some variations, the invention is a system for identifying susceptibility to a cancer in a human subject. For example, in one variation, the system includes tools for performing at least one step, preferably two or more steps, and in some aspects all steps of a method of the invention, where the tools are operably linked to each other. Operable linkage describes a linkage through which components can function with each other to perform their purpose.
In some variations, a system of the invention is a system for identifying susceptibility to a cancer in a human subject, and comprises:
(a) at least one processor;
(b) at least one computer-readable medium;
(c) a susceptibility database operatively coupled to a computer-readable medium of the system and containing population information correlating the presence or absence of one or more alleles of the human BRIP1 gene and susceptibility to a cancer in a population of humans;
(d) a measurement tool that receives an input about the human subject and generates information from the input about the presence or absence of at least one mutant BRIP1 allele indicative of a BRIP1 defect in the human subject; and
(e) an analysis tool or routine that:
   (i) is operatively coupled to the susceptibility database and the information generated by the measurement tool,
   (ii) is stored on a computer-readable medium of the system,
   (iii) is adapted to be executed on a processor of the system, to compare the information about the human subject with the population information in the susceptibility database and generate a conclusion with respect to susceptibility to the cancer for the human subject.

Exemplary processors (processing units) include all variety of microprocessors and other processing units used in computing devices. Exemplary computer-readable media are described above. When two or more components of the system involve a processor or a computer-readable medium, the system generally can be created where a single processor and/or computer readable medium is dedicated to a single component of the system; or where two or more functions share a single processor and/or share a single computer readable medium, such that the system contains as few as one processor and/or one computer readable medium. In some variations, it is advantageous to use multiple processors or media, for example, where it is convenient to have components of the system at different locations. For instance, some components of a system may be located at a testing laboratory dedicated to laboratory or data analysis, whereas other components, including components (optional) for supplying input information or obtaining an output communication, may be located at a medical treatment or counseling facility (e.g., doctor's office, health clinic, HMO, pharmacist, geneticist, hospital) and/or at the home or business of the human subject (patient) for whom the testing service is performed.

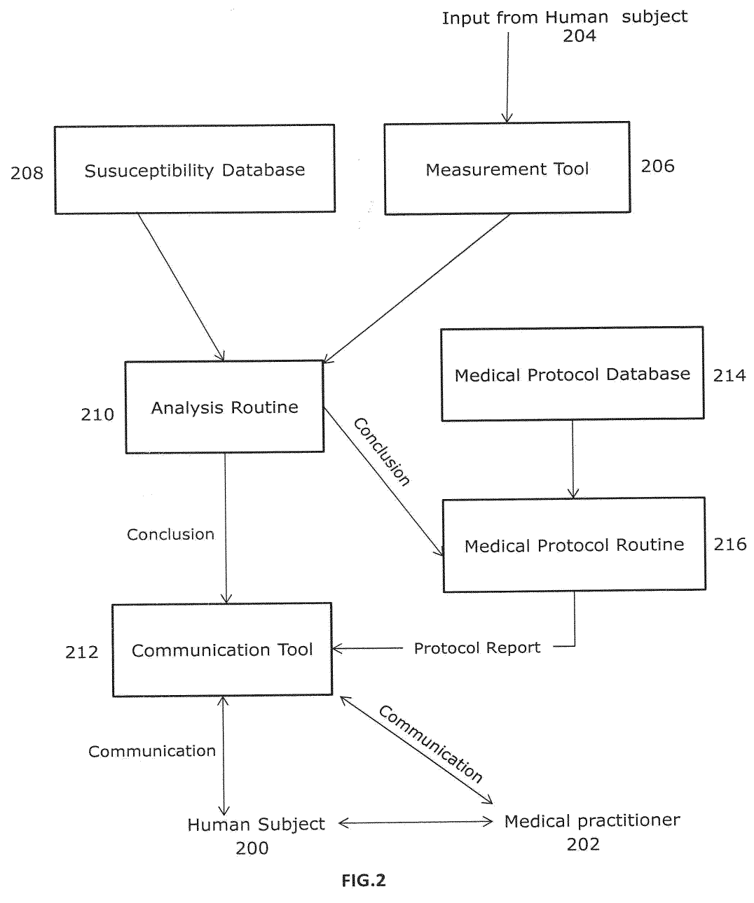

Referring to FIG. 2, an exemplary system includes a susceptibility database 208 that is operatively coupled to a computer-readable medium of the system and that contains population information correlating the presence or absence of one or more alleles of the human BRIP1 gene and susceptibility to a cancer in a population of humans. For example, the one or more alleles of the BRIP1 gene include mutant alleles that cause, or are indicative of, a BRIP1 defect such as reduced or lost function, as described elsewhere herein.

In a simple variation, the susceptibility database contains 208 data relating to the frequency that a particular allele of BRIP1 has been observed in a population of humans with the cancer and a population of humans free of the cancer. Such data provides an indication as to the relative risk or odds ratio of developing the cancer for a human subject that is identified as having the allele in question. In another variation, the susceptibility database includes similar data with respect to two or more alleles of BRIP1, thereby providing a useful reference if the human subject has any of the two or more alleles. In still another variation, the susceptibility database includes additional quantitative personal, medical, or genetic information about the individuals in the database diagnosed with the cancer or free of the cancer. Such information includes, but is not limited to, information about parameters such as age, sex, ethnicity, race, medical history, weight, diabetes status, blood pressure, family history of the cancer, smoking history, and alcohol use in humans and impact of the at least one parameter on susceptibility to the cancer. The information also can include information about other genetic risk factors for the cancer besides BRIP1. These more robust susceptibility databases can be used by an analysis routine 210 to calculate a combined score with respect to susceptibility or risk for developing the cancer.

In addition to the susceptibility database 208, the system further includes a measurement tool 206 programmed to receive an input 204 from or about the human subject and generate an output that contains information about the presence or absence of the at least one BRIP1 allele of interest. (The input 204 is not part of the system per se but is illustrated in the schematic FIG. 2.) Thus, the input 204 will contain a specimen or contain data from which the presence or absence of the at least one BRIP1 allele can be directly read, or analytically determined. In a simple variation, the input contains annotated information about genotypes or allele counts for BRIP1 in the genome of the human subject, in which case no further processing by the measurement tool 206 is required, except possibly transformation of the relevant information about the presence/absence of the BRIP1 allele into a format compatible for use by the analysis routine 210 of the system.

In another variation, the input 204 from the human subject contains data that is unannotated or insufficiently annotated with respect to BRIP1, requiring analysis by the measurement tool 206. For example, the input can be genetic sequence of a chromosomal region or chromosome on which BRIP1 resides, or whole genome sequence information, or unannotated information from a gene chip analysis of a variable loci in the human subject's genome. In such variations of the invention, the measurement tool 206 comprises a tool, preferably stored on a computer-readable medium of the system and adapted to be executed on a processor of the system, to receive a data input about a subject and determine information about the presence or absence of the at least one mutant BRIP1 allele in a human subject from the data. For example, the measurement tool 206 contains instructions, preferably executable on a processor of the system, for analyzing the unannotated input data and determining the presence or absence of the BRIP1 allele of interest in the human subject. Where the input data is genomic sequence information, and the measurement tool optionally comprises a sequence analysis tool stored on a computer readable medium of the system and executable by a processor of the system with instructions for determining the presence or absence of the at least one mutant BRIP1 allele from the genomic sequence information.

In yet another variation, the input 204 from the human subject comprises a biological sample, such as a fluid (e.g., blood) or tissue sample, that contains genetic material that can be analyzed to determine the presence or absence of the BRIP1 allele of interest. In this variation, an exemplary measurement tool 206 includes laboratory equipment for processing and analyzing the sample to determine the presence or absence (or identity) of the BRIP1 allele(s) in the human subject. For instance, in one variation, the measurement tool includes: an oligonucleotide microarray (e.g., “gene chip”) containing a plurality of oligonucleotide probes attached to a solid support; a detector for measuring interaction between nucleic acid obtained from or amplified from the biological sample and one or more oligonucleotides on the oligonucleotide microarray to generate detection data; and an analysis tool stored on a computer-readable medium of the system and adapted to be executed on a processor of the system, to determine the presence or absence of the at least one BRIP1 allele of interest based on the detection data.

To provide another example, in some variations the measurement tool 206 includes: a nucleotide sequencer (e.g., an automated DNA sequencer) that is capable of determining nucleotide sequence information from nucleic acid obtained from or amplified from the biological sample; and an analysis tool stored on a computer-readable medium of the system and adapted to be executed on a processor of the system, to determine the presence or absence of the at least one mutant BRIP1 allele based on the nucleotide sequence information.

In some variations, the measurement tool 206 further includes additional equipment and/or chemical reagents for processing the biological sample to purify and/or amplify nucleic acid of the human subject for further analysis using a sequencer, gene chip, or other analytical equipment.

The exemplary system further includes an analysis tool or routine 210 that: is operatively coupled to the susceptibility database 208 and operatively coupled to the measurement tool 206, is stored on a computer-readable medium of the system, is adapted to be executed on a processor of the system to compare the information about the human subject with the population information in the susceptibility database 208 and generate a conclusion with respect to susceptibility to the cancer for the human subject. In simple terms, the analysis tool 210 looks at the BRIP1 alleles identified by the measurement tool 206 for the human subject, and compares this information to the susceptibility database 208, to determine a susceptibility to the cancer for the subject. The susceptibility can be based on the single parameter (the identity of one or more BRIP1 alleles), or can involve a calculation based on other genetic and non-genetic data, as described above, that is collected and included as part of the input 204 from the human subject, and that also is stored in the susceptibility database 208 with respect to a population of other humans. Generally speaking, each parameter of interest is weighted to provide a conclusion with respect to susceptibility to the cancer. Such a conclusion is expressed in the conclusion in any statistically useful form, for example, as an odds ratio, a relative risk, or a lifetime risk for subject developing the cancer.

In some variations of the invention, the system as just described further includes a communication tool 212. For example, the communication tool is operatively connected to the analysis routine 210 and comprises a routine stored on a computer-readable medium of the system and adapted to be executed on a processor of the system, to: generate a communication containing the conclusion; and to transmit the communication to the human subject 200 or the medical practitioner 202, and/or enable the subject or medical practitioner to access the communication. (The subject and medical practitioner are depicted in the schematic FIG. 2, but are not part of the system per se, though they may be considered users of the system. The communication tool 212 provides an interface for communicating to the subject, or to a medical practitioner for the subject (e.g., doctor, nurse, genetic counselor), the conclusion generated by the analysis tool 210 with respect to susceptibility to the cancer for the subject. Usually, if the communication is obtained by or delivered to the medical practitioner 202, the medical practitioner will share the communication with the human subject 200 and/or counsel the human subject about the medical significance of the communication. In some variations, the communication is provided in a tangible form, such as a printed report or report stored on a computer readable medium such as a flash drive or optical disk. In some variations, the communication is provided electronically with an output that is visible on a video display or audio output (e.g., speaker). In some variations, the communication is transmitted to the subject or the medical practitioner, e.g., electronically or through the mail. In some variations, the system is designed to permit the subject or medical practitioner to access the communication, e.g., by telephone or computer. For instance, the system may include software residing on a memory and executed by a processor of a computer used by the human subject or the medical practitioner, with which the subject or practitioner can access the communication, preferably securely, over the internet or other network connection. In some variations of the system, this computer will be located remotely from other components of the system, e.g., at a location of the human subject's or medical practitioner's choosing.

In some variations of the invention, the system as described (including embodiments with or without the communication tool) further includes components that add a treatment or prophylaxis utility to the system. For instance, value is added to a determination of susceptibility to a cancer when a medical practitioner can prescribe or administer a standard of care that can reduce susceptibility to the cancer; and/or delay onset of the cancer; and/or increase the likelihood of detecting the cancer at an early stage, to facilitate early treatment when the cancer has not spread and is most curable. Exemplary lifestyle change protocols include loss of weight, increase in exercise, cessation of unhealthy behaviors such as smoking, and change of diet. Exemplary medicinal and surgical intervention protocols include administration of pharmaceutical agents for prophylaxis; and surgery, including in extreme cases surgery to remove a tissue or organ before it has become cancerous. Exemplary diagnostic protocols include non-invasive and invasive imaging; monitoring metabolic biomarkers; and biopsy screening.

For example, in some variations, the system further includes a medical protocol database 214 operatively connected to a computer-readable medium of the system and containing information correlating the presence or absence of the at least one BRIP1 allele of interest and medical protocols for human subjects at risk for the cancer. Such medical protocols include any variety of medicines, lifestyle changes, diagnostic tests, increased frequencies of diagnostic tests, and the like that are designed to achieve one of the aforementioned goals. The information correlating a BRIP1 allele with protocols could include, for example, information about the success with which the cancer is avoided or delayed, or success with which the cancer is detected early and treated, if a subject has a BRIP1 susceptibility allele and follows a protocol.

The system of this embodiment further includes a medical protocol tool or routine 216, operatively connected to the medical protocol database 214 and to the analysis tool or routine 210. The medical protocol tool or routine 216 preferably is stored on a computer-readable medium of the system, and adapted to be executed on a processor of the system, to: (i) compare (or correlate) the conclusion that is obtained from the analysis routine 210 (with respect to susceptibility to cancer for the subject) and the medical protocol database 214, and (ii) generate a protocol report with respect to the probability that one or more medical protocols in the medical protocol database will achieve one or more of the goals of reducing susceptibility to the cancer; delaying onset of the cancer; and increasing the likelihood of detecting the cancer at an early stage to facilitate early treatment. The probability can be based on empirical evidence collected from a population of humans and expressed either in absolute terms (e.g., compared to making no intervention), or expressed in relative terms, to highlight the comparative or additive benefits of two or more protocols.

Some variations of the system just described include the communication tool 212. In some examples, the communication tool generates a communication that includes the protocol report in addition to, or instead of, the conclusion with respect to susceptibility.

Information about BRIP1 allele status not only can provide useful information about identifying or quantifying susceptibility to cancers; it can also provide useful information about possible causative factors for a human subject identified with a cancer, and useful information about therapies for the cancer patient. In some variations, systems of the invention are useful for these purposes.

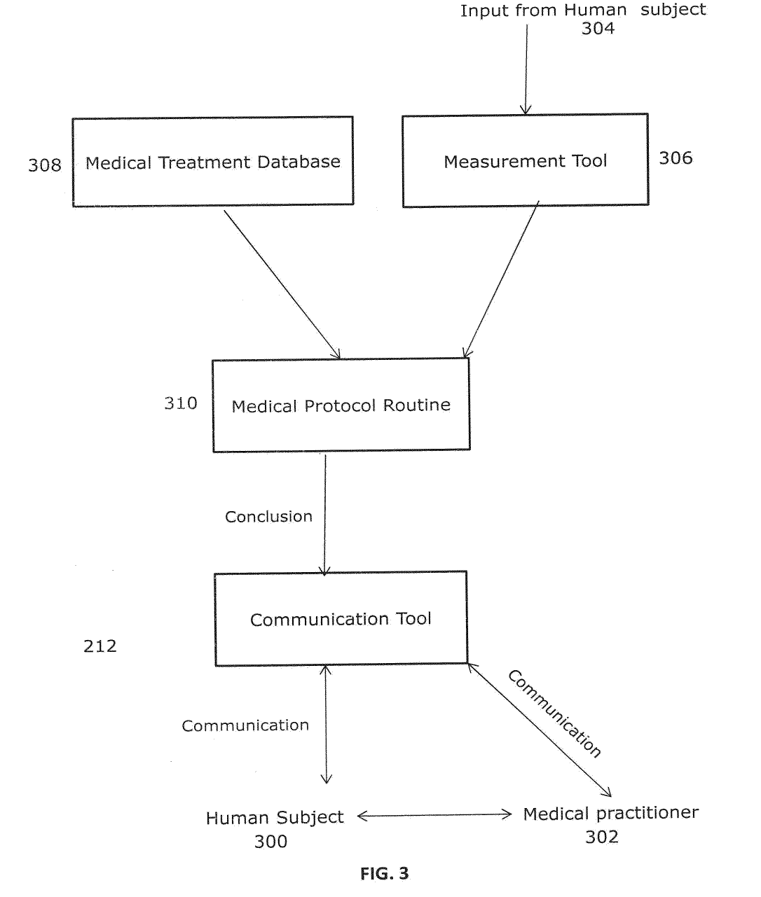

For instance, in some variations the invention is a system for assessing or selecting a treatment protocol for a subject diagnosed with a cancer. An exemplary system, schematically depicted in FIG. 3, comprises:
(a) at least one processor;
(b) at least one computer-readable medium;
(c) a medical treatment database 308 operatively connected to a computer-readable medium of the system and containing information correlating the presence or absence of at least one BRIP1 allele and efficacy of treatment regimens for the cancer;
(d) a measurement tool 306 to receive an input (304, depicted in FIG. 3 but not part of the system per se) about the human subject and generate information from the input 304 about the presence or absence of the at least one BRIP1 allele indicative of a BRIP1 defect in a human subject diagnosed with the cancer; and
(e) a medical protocol routine or tool 310 operatively coupled to the medical treatment database 308 and the measurement tool 306, stored on a computer-readable medium of the system, and adapted to be executed on a processor of the system, to compare the information with respect to presence or absence of the at least one BRIP1 allele for the subject and the medical treatment database, and generate a conclusion with respect to at least one of:
   (i) the probability that one or more medical treatments will be efficacious for treatment of the cancer for the patient; and
   (ii) which of two or more medical treatments for the cancer will be more efficacious for the patient.

Preferably, such a system further includes a communication tool 312 operatively connected to the medical protocol tool or routine 310 for communicating the conclusion to the subject 300, or to a medical practitioner for the subject 302 (both depicted in the schematic of FIG. 3, but not part of the system per se). An exemplary communication tool comprises a routine stored on a computer-readable medium of the system and adapted to be executed on a processor of the system, to generate a communication containing the conclusion; and transmit the communication to the subject or the medical practitioner, or enable the subject or medical practitioner to access the communication.

The present invention will now be exemplified by the following non-limiting examples.

### Example 1

An analysis of sequence variants in the entire genome was performed, the aim being to identify sequence variants that predispose to ovarian cancer. With this purpose, a study of 873 Icelandic women diagnosed with ovarian cancer and over 37,000 Icelandic population controls was performed. All cases had been diagnosed with Invasive Ovarian Cancer, excluding individuals with borderline tumors. The analysis included data for 15,957,390 autosomal SNPs that include those identified through whole genome sequencing of 457 Icelanders and imputed into Icelandic cancer cases and controls.

Genotype data used for analysis contained genotype chip data for genotyped individuals supplemented with genotype information generated by imputation into ungenotyped relatives of genotyped subjects. A total of 68 Icelandic ovarian cancer cases had been chip typed with the Illumina HumanHap300 or CNV370 chips and an additional 572 ovarian cancer cases without chip genotype information were assigned in silico genotypes. The genotypes of the cases were compared to the genotypes of controls matched on genotype informativeness, including 41,607 chip typed controls. Logistic regression was used to test for association between SNPs and disease, treating disease status as the response and expected genotype counts from imputation or allele counts from direct genotyping as covariates. Testing was performed using the likelihood ratio statistic.

The association analysis yielded a number of genome-wide significant association (P<5×10⁻⁸) signals on chromosome 17q23.2 (Table 1). The most significant SNP was found to be rs34289250, which is located in the BRIP1 gene (OR=7.95, p-value 5.6×10⁻¹³). The markers identified are all located in a segment that spans about 2 Mb, and contains a number of genes, including TMEM49, TUBD1, RNFT1, DHX40P1, HEATR6, CA4, USP32, C17orf64, APPBP2, PPM1D, BCAS3, TBX2, C17orf82, TBX4, NACA2, BRIP1, INTS2, and MED13.

TABLE 1
Association results for markers in chromosome 17q23.2 that are associated with ovarian
cancer. Shown are: marker identity, p-value of association with ovarian cancer, odds ratio (OR),
number of individuals whose genotype was imputed, frequency of effect allele in population,
information content of imputation, location of marker on chromosome 17 in NCBI Build 36, identity of
effect and other allele, and reference to SEQ ID showing flanking sequence and location of that
marker.

| | | | # | | | | Effect | Other | SEQ ID |
|---|---|---|---|---|---|---|---|---|---|
| SNP | P adj | OR | indiv | Freq | Info | Location | allele | allele | NO: |
| rs34289250 | 5.65E−13 | 7.95 | 37538 | 0.008944 | 0.91 | 57,235,428 | C | T | 1 |
| rs12938171 | 2.05E−12 | 6.59 | 37540 | 0.011346 | 0.94 | 57,335,137 | A | G | 2 |
| chr17: 55422245 | 5.62E−09 | 10.6 | 37602 | 0.003988 | 0.88 | 55,422,245 | A | T | 3 |
| chr17: 55217320 | 5.71E−09 | 10.61 | 37598 | 0.00404 | 0.87 | 55,217,320 | C | T | 4 |
| rs12451939 | 7.13E−09 | 4.22 | 37540 | 0.983001 | 0.95 | 57,402,424 | G | A | 5 |
| chr17: 56567990 | 7.20E−09 | 10.23 | 37535 | 0.004065 | 0.90 | 56,567,990 | G | T | 6 |
| chr17: 56478611 | 7.66E−09 | 10.19 | 37536 | 0.004077 | 0.90 | 56,478,611 | A | C | 7 |
| chr17: 56505864 | 7.66E−09 | 10.19 | 37536 | 0.004077 | 0.90 | 56,505,864 | C | T | 8 |
| rs12937080 | 8.26E−09 | 4.19 | 37540 | 0.983041 | 0.96 | 57,284,519 | G | A | 9 |

### Example 2

Inspection of the genomic sequence over the BRIP1 gene in carriers of the at-risk C allele of rs34289250 revealed a two nucleotide insertion in exon 14 of BRIP1. This insertion introduces two A nucleotides (AA) between position 57,208,601 and 57,208,602 (chr17:57208601 ins+AA). Thus, a stretch of AAAA at position 57,208,601-57,208,604 increases in size to a stretch of AAAAAA. This mutation inserts TT in the mRNA sequence at position 2345, causing a frameshift and a premature termination of protein translation. As shown in Table 2, the insertion leads to a frameshift in the transcribed BRIP1 sequence, starting at codon 680, resulting in a premature STOP codon at codon 688 (last translated amino acid at codon 687).

TABLE 2
Results of sequencing in the BRIP1 gene. The upper part (reference) shows the
reference wild-type cDNA sequence (middle line) and its translated polynucleotide product (top
line; SEQ ID NO: 21). The lower part (mutation) shows the mutated sequence. The two-
nucleotide TT insertion in the trancribed sequence is shown in bold (position 2345-2346 in the
altered transcript). The altered peptide starts with codon 680, and the predicted STOP codon is
in codon 688 (SEQ ID NO: 14). The bottom line in each case shows the corresponding wild-type
sequence in NCBI Build 36 (inverse orientation (3′ to 5′) compared with the transcript since gene
is transcribed on opposite strand). The * in the translated protein sequence of the mutated form
indicates a STOP codon, which is predicted to terminate translation of the encoded protein.
reference:

| | |
|---|---|
| 661 | C A T F Q N T E T F E F Q D E V G A L L |
| 2287 | TGTGCTACCTTCCAGAATACTGAAACATTTGAGTTCCAAGATGAAGTGGGAGCACTTTTG |
| 57208660 | ACACGATGGAAGGTCTTATGACTTTGTAAACTCAAGGTTCTACTTCACCCTCGTGAAAAC |
| 681 | L S V C Q T V S Q G I L C F L P S Y K |
| 2347 | TTATCTGTGTGCCAGACTGTGAGCCAAGGAATTTTGTGTTTCTTGCCATCTTACAAG |
| 57208600 | AATAGACACACGGTCTGACACTCGGTTCCTTAAAACACAAAGAACGGTAGAATGTTC |

mutation +AA (+TT in transcribed sequence), first stop codon in position 688:

| | |
|---|---|
| 661 | C A T F Q N T E T F E F Q D E V G A L F |
| 2287 | TGTGCTACCTTCCAGAATACTGAAACATTTGAGTTCCAAGATGAAGTGGGAGCACTTTTT |
| 57208660 | ACACGATGGAAGGTCTTATGACTTTGTAAACTCAAGGTTCTACTTCACCCTCGTGAAAA- |
| 681 | C Y L C A R L * A K E F C V S C H L T |
| | TGTTATCTGTGTGCCAGACTGTGAGCCAAGGAATTTTGTGTTTCTTGCCATCTTACAAG |
| 57208600 | -CAATAGACACACGGTCTGACACTCGGTTCCTTAAAACACAAAGAACGGTAGAATGTTC |

TABLE 3
Results of association analysis of the —/AA insertion/deletion
polymorphism in BRIP1 in Iceland. Shown are p-values of association,
relative risk, number of affected individuals assessed, allelic
frequency of insertion, number of controls assessed and the frequency
of the insertion polymorphism in controls.

| p-val | RR | #aff | aff. freq | #con | con. freq |
|---|---|---|---|---|---|
| 8.11E−06 | 7.12 | 291 | 0.0241 | 1159 | 0.00345 |

To investigate this mutation further, direct Sanger sequencing was performed on 291 individuals with Ovarian cancer and 1159 controls over the −/AA insertion in exon 14 of BRIP1.

As can be seen in Table 3, the insertion is significantly associated with risk of ovarian cancer. The insertion has an allelic frequency of 0.35% in controls and about 2.4% in cases, which means that the carrier frequency is about 0.7% in controls and about 5% in cases. Given the functional consequence of the AA insertion, it is likely a causative variant for the association to ovarian cancer observed in Icelandic samples.

Further analysis included Sanger sequencing and microsatellite genotyping; in total, we genotyped the insert directly in 11,741 Icelandic cancer cases and 3,913 controls, including 318 ovarian cancer cases. Using these data, the insert was imputed into all available cancer cases and controls, both those who had been chip genotyped and un-genotyped relatives of chip-typed individuals. Combining results from directly genotyped and imputed cancer cases, the association between ovarian cancer and the insert became even more significant (OR=8.13 (95% CI 4.74, 13.95), P=2.8×10⁻¹⁴), (Table 4).

TABLE 4
Association of the BRIP mutation, chr17: 57208601 ins + AA, with cancer in the Icelandic population.
For the individual cancers, directly typed cases are compared to 3,913 directly typed controls,
in each case excluding the known cases of the cancer being tested from the controls. The
remaining cancer cases and controls that have been chip typed or in silico genotyped are
compared to over 40,000 controls and then combined using the Mantel-Haenszel model
(OR with 95% CI and P value given).

| | | | | | | Removing ovarian | |
|---|---|---|---|---|---|---|---|
| | N genotyped | | | | | cancer cases | |
| Phenotype | Directly | Chipped | In silico | OR (95% CI) | P | OR (95% CI) | P |
| Ovarian Cancer | 318 | 2 | 548 | 8.13 (4.74, 13.95) | 2.8 × 10⁻¹⁴ | — | — |
| Pancreatic Cancer | 158 | 6 | 1,074 | 2.71 (1.31, 5.58) | 0.0069 | 2.58 (1.22, 5.46) | 0.013 |
| Breast Cancer | 2,740 | 173 | 2,543 | 1.28 (0.84, 1.96) | 0.25 | 1.28 (0.84, 1.96) | 0.26 |
| All Cancers | 4,457 | 9,874 | 25,280 | 1.50 (1.25, 1.79) | 8.9 × 10⁻⁶ | 1.36 (1.14, 1.64) | 0.00092 |

### Example 3

Mutations in the genes previously shown to increase risk of ovarian cancer, i.e. BRCA1, BRCA2 and the MMR genes, also carry an increased risk of other cancer types, mainly breast and pancreatic cancers in the case of BRCA1 and BRCA2 and colorectal and endometrial cancers in the case of the MMR genes. Experimentation was performed to determine whether the chr17:57208601 ins+AA insert is associated with an increase in the risk of other cancer types, using information from the nation-wide Icelandic Cancer Registry and direct genotype information from 14,331 directly genotyped and 16,873 in silico genotyped cancer cases (representing 23 different cancer types). Analysis of the risk of individual cancer types showed an excess of pancreatic cancer in carriers (OR=2.71, P=0.0069).

In contrast to the individual cancers, when all the cancers were analyzed together, carriers of the insert had an increased risk of being diagnosed with cancer in general (OR=1.50, P=1.5×10⁻⁶) (Table 5). Given the large effect of the insert on ovarian cancer and the fact that ovarian cancer can co-occur with other cancer types, the analysis was repeated excluding the ovarian cancer cases (Table 6). Even without the ovarian cancer cases, the risk of any cancer remained significant (OR=1.36, P=9.2×10⁻⁴). Notably, although no cancer type was individually significantly associated with the insert after correcting for the number of tests, three cancer types had P values <0.05, i.e. cancers of the pancreas, rectum and upper airways. In total, 16 of the 22 cancer types showed an effect in the same direction as ovarian cancer. The lifespan of 8,342 deceased Icelanders who had been directly typed or imputed for the chr17:57208601 ins+AA insert was determined along with 7,604 deceased Icelanders with high quality in silico genotypes who were born after 1900 and lived to be at least 50 years old. It was found that the insert reduces lifespan in the Icelandic population by 3.6 years (95% CI 1.5, 5.7 years, P=7.2×10⁻⁴).

TABLE 5
Association of the BRIP mutation, chr17: 57208601 ins + AA, with 23 cancers or having any cancer in the Icelandic population.

| | N | | | Directly typed | | Imputed | | Combined | | |
|---|---|---|---|---|---|---|---|---|---|---|
| Phenotype | Direct | Chipped | In silico | OR | P | OR | P | OR (95% CI) | P | Pₕₑₜ |
| Bladder Cancer | 754 | 42 | 1,049 | 0.32 | 0.079 | 0.98 | 0.97 | 0.66 (0.31, 1.40) | 0.28 | 0.17 |
| Brain Cancer Glioma | 121 | 4 | 298 | 0.00 | 0.18 | 1.03 | 0.98 | 0.63 (0.07, 5.62) | 0.68 | 0.20 |
| Brain Cancer Meningioma | 139 | 2 | 79 | 1.76 | 0.48 | 1.02 | 0.99 | 1.57 (0.39, 6.37) | 0.52 | 0.76 |
| Breast Cancer | 2,740 | 173 | 2,543 | 1.60 | 0.097 | 0.94 | 0.86 | 1.28 (0.84, 1.96) | 0.25 | 0.23 |
| Cervix Uteri Cancer | 232 | 19 | 584 | 1.08 | 0.92 | 0.23 | 0.14 | 0.61 (0.18, 2.01) | 0.42 | 0.22 |
| Chronic Lymphocytic Leukemia | 113 | 7 | 283 | 1.08 | 0.94 | 1.48 | 0.71 | 1.28 (0.29, 5.66) | 0.75 | 0.84 |
| Colon Cancer | 291 | 601 | 1,944 | 3.03 | 0.031 | 0.85 | 0.63 | 1.25 (0.72, 2.16) | 0.43 | 0.038 |
| Endometrial Cancer | 428 | 16 | 507 | 0.56 | 0.41 | 2.40 | 0.13 | 1.33 (0.55, 3.19) | 0.53 | 0.11 |
| Esophagus Cancer | 103 | 2 | 576 | 2.38 | 0.32 | 1.12 | 0.88 | 1.57 (0.50, 4.91) | 0.43 | 0.52 |
| Gastric Cancer | 354 | 13 | 2,907 | 2.43 | 0.083 | 1.24 | 0.54 | 1.54 (0.87, 2.70) | 0.14 | 0.28 |
| Kidney Cancer | 519 | 18 | 1,036 | 1.88 | 0.16 | 0.44 | 0.23 | 1.20 (0.57, 2.49) | 0.63 | 0.074 |
| Liver Cancer | 70 | 5 | 374 | 0.00 | 0.31 | 1.37 | 0.72 | 1.15 (0.21, 6.22) | 0.87 | 0.29 |
| Lung Cancer | 273 | 631 | 3,160 | 1.37 | 0.65 | 1.40 | 0.19 | 1.39 (0.87, 2.23) | 0.16 | 0.98 |
| Lymphoma Hodgkin | 79 | 2 | 215 | 4.71 | 0.041 | 1.19 | 0.89 | 3.30 (0.92, 11.85) | 0.067 | 0.36 |
| Lymphoma Non Hodgkin | 259 | 22 | 604 | 2.85 | 0.046 | 0.54 | 0.45 | 1.74 (0.74, 4.13) | 0.21 | 0.086 |
| Multiple Myeloma | 115 | 3 | 365 | 1.06 | 0.96 | 2.40 | 0.21 | 1.89 (0.60, 5.98) | 0.28 | 0.53 |
| Ovarian Cancer | 318 | 2 | 548 | 5.86 | 3.7E−06 | 11.60 | 7.10E−10 | 8.13 (4.74, 13.95) | 2.8E−14 | 0.22 |
| Pancreatic Cancer | 161 | 6 | 1,077 | 2.29 | 0.26 | 2.87 | 0.014 | 2.71 (1.31, 5.58) | 0.0069 | 0.79 |
| Prostate Cancer | 2,244 | 235 | 2,567 | 1.12 | 0.73 | 0.78 | 0.46 | 0.94 (0.59, 1.49) | 0.80 | 0.44 |
| Rectal Cancer | 77 | 230 | 781 | 3.29 | 0.18 | 2.09 | 0.062 | 2.25 (1.11, 4.58) | 0.025 | 0.64 |
| Testicular Cancer | 196 | 1 | 108 | 1.25 | 0.77 | 1.24 | 0.9 | 1.25 (0.31, 4.93) | 0.75 | 1.00 |
| Thyroid Cancer | 550 | 12 | 595 | 0.88 | 0.82 | 0.22 | 0.16 | 0.66 (0.25, 1.74) | 0.40 | 0.26 |
| Upper Airway Cancer | 35 | 35 | 381 | 3.52 | 0.33 | 0.02 | 0.044 | 0.76 (0.09, 6.40) | 0.80 | 0.026 |
| All Cancers* | 4,457 | 9,874 | 25,280 | 1.78 | 0.048 | 1.47 | 5.60E−05 | 1.50 (1.25, 1.79) | 8.90E−06 | 0.53 |

For the individual cancers, directly typed cases are compared to 3,913 directly typed controls, in each case excluding the known cases of the cancer being tested from the controls (OR and P value given). The remaining cancer cases and controls that have been chip typed or in silico genotyped are also compared (OR and P value given) and then combined using the Mantel-Haenszel model (OR with 95% CI and P value given). A P-value for testing for the difference between the ORs in the two groups is given in the Phet column.
*Due to the relatively small number of directly genotyped controls we removed the chip typed cases from the list of directly genotyped cases when testing for association with any cancer type, thus including all chip typed cases in the second group. The P-values and 95% CIs have been adjusted using a genomic control correction factor for testing each phenotype based on the chip typed and in silico genotype data.

TABLE 6
Association of the BRIP mutation, chr17: 57208601 ins + AA, with 22 cancers or having any cancer in the Icelandic population
after removal of all ovarian cancer cases.

| | | | | Directly | | | | | | |
|---|---|---|---|---|---|---|---|---|---|---|
| | N | | | typed | | Imputed | | Combined | | |
| Phenotype | Direct | Chipped | In silico | OR | P | OR | P | OR (95% CI) | P | Pₕₑₜ |
| Bladder Cancer | 752 | 42 | 1,048 | 0.32 | 0.079 | 1.00 | 1.0 | 0.66 (0.31, 1.42) | 0.29 | 0.16 |
| Brain Cancer Glioma | 121 | 4 | 298 | 0.00 | 0.18 | 1.05 | 0.96 | 0.74 (0.12, 4.65) | 0.75 | 0.19 |
| Brain Cancer Meningioma | 136 | 2 | 79 | 1.80 | 0.47 | 1.04 | 0.98 | 1.61 (0.40, 6.53) | 0.51 | 0.76 |
| Breast Cancer | 2,712 | 173 | 2,516 | 1.57 | 0.12 | 0.97 | 0.93 | 1.28 (0.84, 1.96) | 0.26 | 0.28 |
| Cervix Uteri Cancer | 230 | 19 | 579 | 1.09 | 0.91 | 0.24 | 0.15 | 0.62 (0.19, 2.06) | 0.44 | 0.23 |
| Chronic Lymphocytic Leukemia | 112 | 7 | 282 | 1.09 | 0.94 | 1.54 | 0.68 | 1.30 (0.29, 5.79) | 0.73 | 0.82 |
| Colon Cancer | 286 | 601 | 1,937 | 3.07 | 0.029 | 0.87 | 0.68 | 1.27 (0.73, 2.22) | 0.39 | 0.04 |
| Endometrial Cancer | 421 | 16 | 499 | 0.57 | 0.43 | 2.23 | 0.19 | 1.24 (0.50, 3.05) | 0.64 | 0.14 |
| Esophagus Cancer | 103 | 2 | 576 | 2.38 | 0.32 | 1.15 | 0.86 | 1.59 (0.51, 4.98) | 0.42 | 0.53 |
| Gastric Cancer | 352 | 13 | 2,900 | 2.43 | 0.082 | 1.28 | 0.48 | 1.58 (0.89, 2.78) | 0.12 | 0.30 |
| Kidney Cancer | 518 | 18 | 1,034 | 1.87 | 0.16 | 0.45 | 0.24 | 1.21 (0.58, 2.52) | 0.61 | 0.081 |
| Liver Cancer | 69 | 5 | 374 | 0.00 | 0.32 | 1.39 | 0.70 | 1.18 (0.23, 6.14) | 0.84 | 0.29 |
| Lung Cancer | 267 | 631 | 3,151 | 1.40 | 0.63 | 1.36 | 0.24 | 1.36 (0.85, 2.19) | 0.20 | 0.97 |
| Lymphoma Hodgkin | 79 | 2 | 215 | 4.69 | 0.041 | 1.21 | 0.88 | 3.32 (0.92, 11.94) | 0.066 | 0.37 |
| Lymphoma Non Hodgkin | 258 | 21 | 603 | 2.85 | 0.046 | 0.56 | 0.48 | 1.77 (0.74, 4.19) | 0.20 | 0.093 |
| Multiple Myeloma | 113 | 3 | 364 | 1.08 | 0.95 | 2.46 | 0.20 | 1.93 (0.61, 6.12) | 0.26 | 0.52 |
| Pancreatic Cancer | 158 | 6 | 1,074 | 1.54 | 0.61 | 2.95 | 0.012 | 2.58 (1.22, 5.46) | 0.013 | 0.49 |
| Prostate Cancer | 2,244 | 235 | 2,567 | 1.12 | 0.73 | 0.80 | 0.51 | 0.95 (0.60, 1.51) | 0.83 | 0.48 |
| Rectal Cancer | 75 | 230 | 779 | 3.38 | 0.17 | 2.15 | 0.053 | 2.32 (1.14, 4.73) | 0.020 | 0.65 |
| Testicular Cancer | 196 | 1 | 108 | 1.24 | 0.78 | 1.26 | 0.90 | 1.25 (0.32, 4.93) | 0.75 | 0.99 |
| Thyroid Cancer | 544 | 12 | 589 | 0.89 | 0.83 | 0.23 | 0.17 | 0.67 (0.25, 1.77) | 0.42 | 0.27 |
| Upper Airway Cancer | 33 | 35 | 380 | 0.00 | 0.49 | 0.02 | 0.046 | 0.02 (0.00, 0.76) | 0.035 | 0.92 |
| All Cancers | 4,205 | 9,806 | 24,732 | 1.51 | 0.18 | 1.35 | 0.0023 | 1.36 (1.14, 1.64) | 0.00092 | 0.73 |

Table legend is the same as for Table 5 except ovarian cancer cases have been removed from both cases and controls.

### Example 4

To screen for additional mutations in BRIP1 that might contribute to ovarian cancer risk in Iceland or other populations, the whole gene (exons, introns and upstream regulatory region) was sequenced in ovarian cancer cases and controls from Iceland, the Netherlands and Spain, using a pooling strategy where samples from ovarian cancer cases and controls were pooled separately, amplified by long-range PCR and sequenced using Solexa technology. Further details of the protocols used for this purpose are provided below under Example 6.

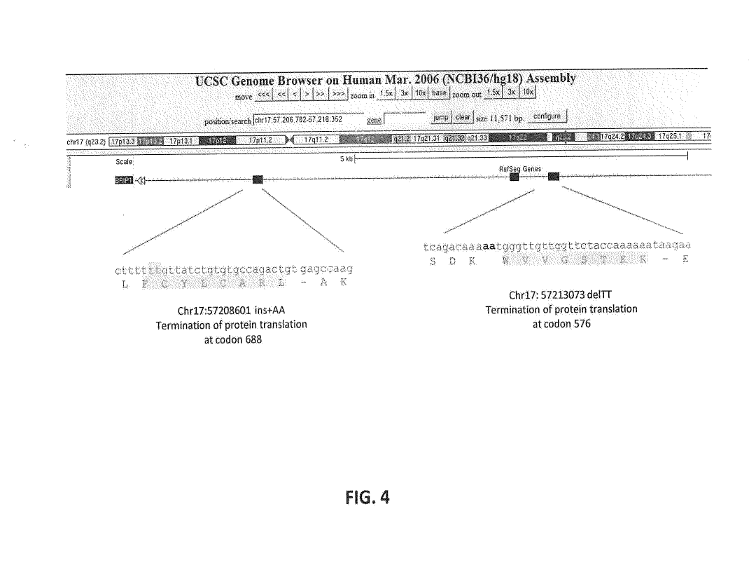

One deletion was detected in the Spanish case pools but not in control pools, chr17: 57213073 delTT (i.e., a two basepair deletion following position 57213073) corresponding to deletion of TT in position 2008-2009 in SEQ ID NO:10). This out-of-frame mutation is located in exon 12 and leads to a termination of protein translation at amino acid 576 (FIG. 4). Genotyping of the deletion in cancer cases and controls from Spain showed that the deletion is very rare (allelic frequency 0.03% in controls, N=1,780) but associates with a greatly increased risk of ovarian cancer (OR=24, P=0.017) and a significant risk of breast cancer (OR=11, P=0.009) (Table 7). The deletion was not found in any of the 2,758 Spanish cases with other cancer types, except for a single case of lung cancer. In addition to the Spanish deletion, 11 coding variants identified through the pool sequencing were genotyped (10 missense variants and the known FA variant R798X), as well as three missense variants that have previously been found in breast cancer cases (M299I, Q944E and P1034L), in the three study populations (Table 8). In short, these variants were either found in similar frequencies in cases and controls or they were too rare to be conclusively associated with ovarian cancer. It is therefore contemplated that association with ovarian cancer may be confined to mutations leading to truncated transcripts.

TABLE 7
Association of the BRIP mutation, chr17: 57213073 delTT, with
cancer in a Spanish population.

| | N individuals | | | | | |
|---|---|---|---|---|---|---|
| | | Non- | N alleles | Allele | | |
| Phenotype | Carriers | carriers | Mut/Wt | freq. | OR | P |
| Controls | 1 | 1779 | 1/3559 | 0.03% | 1.00 | |
| Ovarian cancer | 2 | 142 | 2/286 | 0.70% | 25.00 | 0.016 |
| Breast cancer | 6 | 921 | 6/1848 | 0.32% | 11.17 | 0.0079 |
| Lung cancer | 1 | 514 | 1/1029 | 0.10% | 3.34 | |
| Colorectal | 0 | 497 | 0/994 | 0.00% | 0.00 | |
| cancer | | | | | | |
| Endometrial | 0 | 130 | 0/260 | 0.00% | 0.00 | |
| cancer | | | | | | |
| Bladder cancer | 0 | 238 | 0/476 | 0.00% | 0.00 | |
| Prostate cancer | 0 | 699 | 0/1398 | 0.00% | 0.00 | |
| Basal cell | 0 | 222 | 0/444 | 0.00% | 0.00 | |
| carcinoma | | | | | | |
| Cutaneous | 0 | 278 | 0/556 | 0.00% | 0.00 | |
| melanoma | | | | | | |
| Thyroid cancer | 0 | 90 | 0/180 | 0.00% | 0.00 | |
| Kidney cancer | 0 | 89 | 0/178 | 0.00% | 0.00 | |

TABLE 8
Allele counts of coding variants in BRIP1 in ovarian cancer cases and controls from Iceland, The Netherlands and Spain

| | Iceland # alleles | | | | The Netherlands # alleles | | | | Spain # alleles | | | |
|---|---|---|---|---|---|---|---|---|---|---|---|---|
| | Cases | | Controls | | Cases | | Controls | | Cases | | Controls | |
| Variant | Variant | Wild type | Variant | Wild type | Variant | Wild type | Variant | Wild type | Variant | Wild type | Variant | Wild type |
| P47A | 0 | 370 | 0 | 482 | 1 | 533 | 2 | 1188 | 0 | 284 | * | * |
| R173C | 2 | 434 | 23 | 2713 | 1 | 549 | 7 | 1439 | 0 | 288 | * | * |
| V193I | 4 | 524 | 13 | 2721 | 4 | 546 | 7 | 1437 | 0 | 288 | * | * |
| L195P | 0 | 436 | 2 | 2554 | 1 | 549 | 1 | 1445 | 0 | 286 | * | * |
| K209R | 0 | 436 | 0 | 2554 | 1 | 549 | 0 | 1446 | 0 | 284 | * | * |
| C214S | 0 | 436 | 0 | 2538 | 1 | 549 | 0 | 1366 | 0 | 274 | 0 | 848 |
| E262G | 0 | 438 | 0 | 2542 | 0 | 552 | 0 | 1394 | 0 | 272 | 0 | 858 |
| M299I | 0 | 438 | 0 | 2544 | 0 | 552 | 0 | 1376 | 0 | 274 | 0 | 856 |
| K297R | 1 | 437 | 0 | 2550 | 1 | 551 | 3 | 1373 | 0 | 274 | 0 | 856 |
| R419Q | 0 | 378 | 0 | 496 | 1 | 547 | 0 | 1280 | 0 | 288 | * | * |
| Q741H | 1 | 435 | 3 | 2181 | 0 | 540 | * | * | 0 | 286 | 5 | 813 |
| D745D | 0 | 436 | 1 | 2177 | 0 | 540 | * | * | 1 | 285 | 1 | 821 |
| R798X | 1 | 435 | 7 | 3051 | 0 | 544 | * | * | 0 | 284 | * | * |
| Q944E | 0 | 378 | 0 | 2854 | 0 | 538 | * | * | 0 | 282 | * | * |
| P1034L | 0 | 436 | 0 | 898 | 0 | 540 | * | * | 0 | 282 | * | * |

Variants were genotyped by Sanger sequencing of the relevant exons.
Shown are the number of mutant and wild type alleles among cases and controls in each population.
*Indicates that genotyping was not done in control groups since no variant alleles were found in the cases.

### Example 5

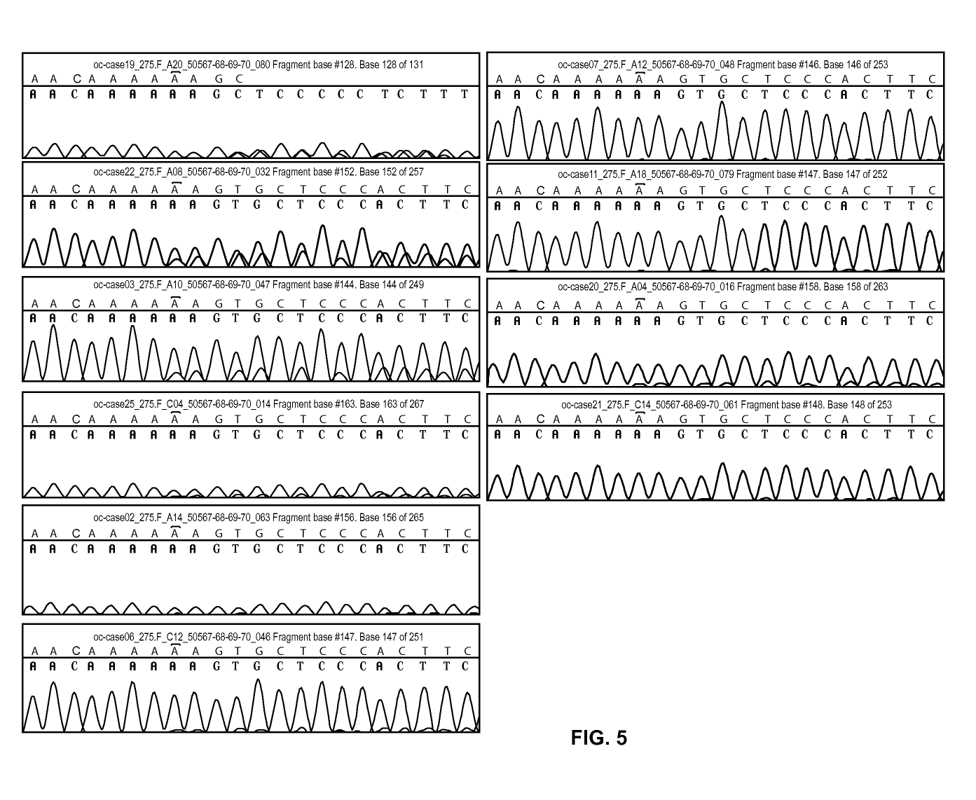

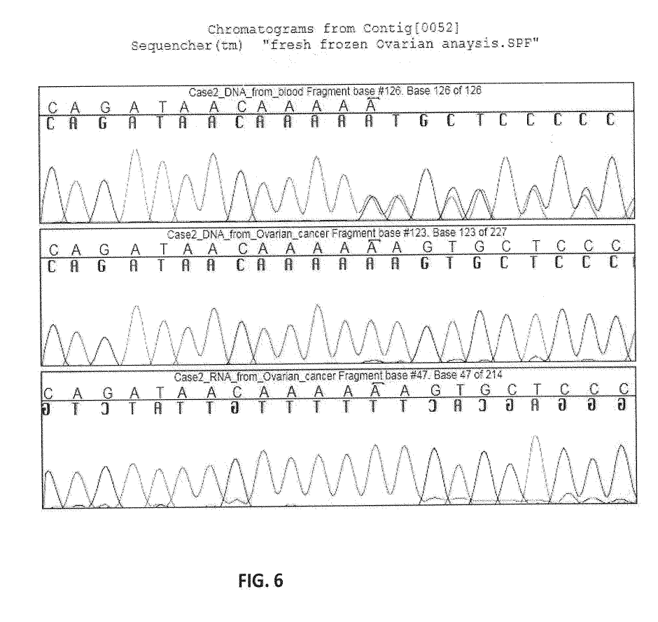

It was tested whether BRIP1 conforms to the classical paradigm of a tumor suppressor gene where the wild-type allele is lost in tumors of heterozygous carriers. Ovarian tumor samples from 10 carriers of the Icelandic mutation were obtained (2 fresh-frozen and 8 paraffin-embedded) and PCR and Sanger sequencing of genomic DNA was used to test for LOH. Eight of the 10 tumors showed loss of the wild-type allele (FIG. 5). Whole-genome sequencing of tumor DNA from the two fresh-frozen tumors further confirmed the LOH at the BRIP1 locus (Table 9). Finally, the ratio of wild-type to mutant mRNA was assessed using Sanger sequencing of cDNA. Both tumors showed a greatly reduced mRNA expression from the wild-type allele compared to the mutant allele supporting loss of the wild type allele in most of the cells within the tumor sample (FIG. 6).

TABLE 9
Whole-genome sequencing. Number of mutant and wild-type sequence
reads in the area of chr17: 57208601 ins + AA

| | Germ-line DNA | | | Tumor DNA | | |
|---|---|---|---|---|---|---|
| | | # WT | # Mut | | # WT | # Mut |
| | Coverage | alleles | alleles | Coverage | alleles | alleles |
| Case 6 | 44 | 28 | 29 | 23 | 5 | 23 |
| Case 11 | 15 | 16 | 14 | 27 | 7 | 37 |

Results from whole genome sequencing of germ-line DNA and tumor DNA from two heterozygous carriers of chr17: 57208601 ins + AA.
Shown is the sequence coverage for each of the samples, the number of sequence reads for each allele, wild-type (WT)and mutant (Mut). Pathology review of the tumor samples estimated the tumor cell percentage to be 50-60% for case 6 and 60-70% for case 11

### Example 6

### Methods

### Study Populations:

### Iceland.

Cancer cases were identified in the nation-wide Icelandic Cancer Registry (ICR; www.krabbameinsskra.is) which includes information on the age, month and year of diagnosis, month and year of death, SNOMED code and ICD-10 classification. A total of 868 cases of invasive ovarian cancer were diagnosed in Iceland from 1955-2009. Blood samples were collected from all prevalent cases available at the start of 2001, and all incident cases since then or a total of 224 cases. In addition, paraffin-embedded tissues samples from additional 116 ovarian cancer cases was obtained from the Biobank of Landspitali hospital. Thus, DNA from a total of 340 cases was available for genotyping. Written informed consent was obtained from all live cases. All projects at deCODE Genetics have been approved by the National Bioethics Committee and the Data Protection Authority of Iceland. The Icelandic controls used in this study consisted of individuals from other ongoing genome-wide association studies at deCODE. In addition to the directly genotyped cases, the ovarian cancer case group was augmented by in-silico genotyping of un-genotyped cases by imputation as described below.

### Spain.

Ovarian cancer cases were recruited from the Oncology Department of Zaragoza Hospital between September 2007 and February 2008. The Spanish control samples were from individuals who attended the University Hospital in Zaragoza for diseases other than cancer. Controls were questioned to rule out prior cancers before the blood sample was collected. Study protocols were approved by the Institutional Review Board of Zaragoza University Hospital and all subjects gave written informed consent.

The Netherlands.

Ovarian cancer cases, diagnosed between 1989 and 2006 and still alive in 2008, were identified from the population-based cancer registry of the Comprehensive Cancer Center The Netherlands, Location Nijmegen, in the Mid-Eastern part of the Netherlands (8). Dutch control individuals were recruited within the Nijmegen Biomedical Study (NBS) (25).

### Finland.

Paraffin-embedded samples from Finnish ovarian cancer cases were used in the study. Informed consent was obtained from consecutive cases of epithelial ovarian cancer patients during 2008-2009 while they were visiting the Tampere University Hospital gynecological oncology clinic for adjuvant chemotherapy or follow-up, during 2008-2009. The 55 cases used in the study were patients who also had a matching blood sample. The 1,000 Finnish control samples were blood derived genomic DNA from anonymous Finnish blood donors.

### Illumina Genome-Wide Genotyping:

The Icelandic chip-typed samples were assayed with the Illumina Human Hap300, Hap CNV370, Hap 610, 1M or Omni-1 Quad bead chips at deCODE genetics. Only the 317,503 SNPs from the Human Hap300 chip were used in the long range phasing and the subsequent SNP imputations. SNPs were excluded if they had (i) yield lower than 95%, (ii) minor allele frequency less than 1% in the population or (iii) significant deviation from Hardy-Weinberg equilibrium in the controls (P <0.001), (iv) if they produced an excessive inheritance error rate (over 0.001), (v) if there was substantial difference in allele frequency between chip types (from just a single chip if that resolved all differences, but from all chips otherwise). All samples with a call rate below 97% were excluded from the analysis. The final set of SNPs used for long range phasing was composed of 297,835 autosomal SNPs.

### Whole-Genome Sequencing and SNP Imputations:

SNPs were imputed based on unpublished data from the Icelandic whole genomic sequencing project (457 Icelandic individuals) selected for various neoplastic, cardiovascular and psychiatric conditions. All of the individuals were sequenced to a depth of at least 10×. Sixteen million SNPs were imputed based on this set of individuals.

1. Sample Preparation.
Paired-end libraries for sequencing were prepared according to the manufacturer's instructions (Illumina). In short, approximately 5 μg of genomic DNA, isolated from frozen blood samples, were fragmented to a mean target size of 300 bp using a Covaris E210 instrument. The resulting fragmented DNA was end repaired using T4 and Klenow polymerases and T4 polynucleotide kinase with 10 mM dNTP followed by addition of an ‘A’ base at the ends using Klenow exo fragment (3′ to 5′-exo minus) and dATP (1 mM). Sequencing adaptors containing ‘T’ overhangs were ligated to the DNA products followed by agarose (2%) gel electrophoresis. Fragments of about 400 bp were isolated from the gels (QIAGEN Gel Extraction Kit), and the adaptor-modified DNA fragments were PCR enriched for ten cycles using Phusion DNA polymerase (Finnzymes Oy) and PCR primers PE 1.0 and PE 2.0 (Illumina). Enriched libraries were further purified using agarose (2%) gel electrophoresis as described above. The quality and concentration of the libraries were assessed with the Agilent 2100 Bioanalyzer using the DNA 1000 LabChip (Agilent). Barcoded libraries were stored at −20° C. All steps in the workflow were monitored using an in-house laboratory information management system with barcode tracking of all samples and reagents.

2. DNA Sequencing.
Template DNA fragments were hybridized to the surface of flow cells (Illumina PE flowcell, v4) and amplified to form clusters using the Illumina cBot. In brief, DNA (3-10 μM) was denatured, followed by hybridization to grafted adaptors on the flowcell. Isothermal bridge amplification using Phusion polymerase was then followed by linearization of the bridged DNA, denaturation, blocking of 3′ ends and hybridization of the sequencing primer. Sequencing-by-synthesis was performed on Illumina GAIIx instruments equipped with paired-end modules. Paired-end libraries for whole genome sequencing were sequenced using either 2×101 or 2×120 cycles of incorporation and imaging with Illumina sequencing kits, v4 or v5 (TruSeq). Each library or sample was initially run on a single lane for validation followed by further sequencing of lanes with targeted raw cluster densities of 500-700 k/mm2, depending on the version of the data imaging and analysis packages. Imaging and analysis of the data was performed using SCS2.6/RTA1.6, SCS2.8/RTA1.8 or SCS2.9&RTA1.9 software packages from Illumina, respectively. Real-time analysis involved conversion of image data to base-calling in real-time.

3. Alignment.
For each lane in the DNA sequencing output, the resulting qseq files were converted into fastq files using an in-house script. All output from sequencing was converted, and the Illumina quality filtering flag was retained in the output. The fastq files were then aligned against Build 36 of the human reference sequence using bwa version 0.5.7 (26).

4. BAM File Generation.
SAM file output from the alignment was converted into BAM format using samtools version 0.1.8 (27), and an in-house script was used to carry the Illumina quality filter flag over to the BAM file. The BAM files for each sample were then merged into a single BAM file using samtools. Finally, Picard version 1.17 (see http://picard.sourceforge.net/) was used to mark duplicates in the resulting sample BAM files.

5. SNP Calling and Genotyping in Whole-Genome Sequencing.
A two-step approach was applied. The first step was to detect SNPs by identifying sequence positions where at least one individual could be determined to be different from the reference sequence with confidence (quality threshold of 20) based on the SNP calling feature of the pileup tool samtools (27). SNPs that always differed heterozygous or homozygous from the reference were removed. The second step was to use the pileup tool to genotype the SNPs at the positions that were flagged as polymorphic. Because sequencing depth varies and hence the certainty of genotype calls also varies, genotype likelihoods rather than deterministic calls were calculated (see below). Of the 2.5 million SNPs reported in the HapMap2 CEU samples, 96.3% were observed in the whole-genome sequencing data. Of the 6.9 million SNPs reported in the 1000 Genomes Project data, 89.4% were observed in the whole-genome sequencing data.

Long Range Phasing:
Long range phasing of all chip-genotyped individuals was performed with methods described previously (10, 28). In brief, phasing was achieved using an iterative algorithm which phases a single proband at a time given the available phasing information about everyone else that shares a long haplotype identically by state with the proband. Given the large fraction of the Icelandic population that has been chip-typed, accurate long range phasing is available genome-wide for all chip-typed Icelanders.

### Genotype Imputation:

The SNPs identified and genotyped through sequencing were imputed into all Icelanders who had been phased with long range phasing using the same model as used by IMPUTE (29). The genotype data from sequencing can be ambiguous due to low sequencing coverage. In order to phase the sequencing genotypes, an iterative algorithm was applied for each SNP with alleles 0 and 1. With H representing the long range phased haplotypes of the sequenced individuals, the following algorithm was applied:
1. For each haplotype h in H, use the Hidden Markov Model of IMPUTE to calculate for every other k in H, the likelihood, denoted γ_{h,k}, of h having the same ancestral source as k at the SNP.
2. For every h in H, initialize the parameter θₕ, which specifies how likely the one allele of the SNP is to occur on the background of h from the genotype likelihoods obtained from sequencing. The genotype likelihood L_{g} is the probability of the observed sequencing data at the SNP for a given individual assuming g is the true genotype at the SNP. If L₀, L₁ and L₂ are the likelihoods of the genotypes 0, 1 and 2 in the individual who carries h, then set

$${θ}_{h} = \frac{{L}_{2} + \frac{1}{2} {L}_{1}}{{L}_{2} + {L}_{1} + {L}_{0}} .$$

3. For every pair of haplotypes h and k in H that are carried by the same individual, use the other haplotypes in H to predict the genotype of the SNP on the backgrounds of h and k: τₕ=Σ_{lεH\{h}}γ_{h,l}θₗ and τₖ=Σ_{lεH\{k}}γ_{k,l}θₗ. Combining these predictions with the genotype likelihoods from sequencing gives un-normalized updated phased genotype probabilities: P₀₀=(1−τₕ)(1−τₖ)L₀, P₁₀=τₕ(1−τₖ)½L₁, P₀₁=(1−τₕ)τₖ½L₁ and P₁₁=τₕτₖL₂. Now use these values to update θₕ and θₖ to

$${θ}_{h} = \frac{{P}_{10} + {P}_{11}}{{P}_{00} + {P}_{01} + {P}_{10} + {P}_{11}} and {θ}_{k} = \frac{{P}_{01} + {P}_{11}}{{p}_{00} + {P}_{01} + {P}_{10} + {P}_{11}} .$$

4. Repeat step 3 when the maximum difference between iterations is greater than a convergence threshold ε. We used ε=10⁻⁷.

Given the long range phased haplotypes and θ, the allele of the SNP on a new haplotype h not in H, is imputed as Σ_{lεH}γ_{h,l}θₗ.

The above algorithm can easily be extended to handle simple family structures such as parent-offspring pairs and triads by letting the P distribution run over all founder haplotypes in the family structure. The algorithm also extends easily to the X-chromosome. If source genotype data are only ambiguous in phase, such as chip genotype data, then the algorithm is still applied, but all but one of the Ls will be 0. In some instances, the reference set was intentionally enriched for carriers of the minor allele of a rare SNP in order to improve imputation accuracy. In this case, expected allele counts is biased toward the minor allele of the SNP. Call the enrichment of the minor allele E and let θ′ be the expected minor allele count calculated from the naïve imputation method, and let θ be the unbiased expected allele count, then

$${θ}^{′} = \frac{E θ}{1 - θ + E θ}$$

and hence

$$θ = \frac{{θ}^{′}}{E + ( 1 - E ) {θ}^{′}} .$$

This adjustment was applied to all imputations based on enriched imputations sets. We note that if θ′ is 0 or 1, then θ will also be 0 or 1, respectively.

### In Silico (Genealogy-Based) Genotyping:

In addition to imputing sequence variants from the whole genome sequencing effort into chip genotyped individuals, a second imputation step where genotypes were imputed into relatives of chip genotyped individuals was also performed, creating in silico genotypes. The inputs into the second imputation step are the fully phased (in particular every allele has been assigned a parent of origin) imputed and chip type genotypes of the available chip typed individuals. The algorithm used to perform the second imputation step consists of:
1. For each ungenotyped individual (the proband), find all chip genotyped individuals within two meiosis of the individual. The six possible types of two meiosis relatives of the proband are (ignoring more complicated relationships due to pedigree loops): Parents, full and half siblings, grandparents, children and grandchildren. If all pedigree paths from the proband to a genotyped relative go through other genotyped relatives, then that relative is excluded. E.g. if a parent of the proband is genotyped, then the proband's grandparents through that parent are excluded. If the number of meiosis in the pedigree around the proband exceeds a threshold (we used 12), then relatives are removed from the pedigree until the number of meiosis falls below 12, in order to reduce computational complexity.
2. At every point in the genome, calculate the probability for each genotyped relative sharing with the proband based on the autosomal SNPs used for phasing. A multipoint algorithm based on the hidden Markov model Lander-Green multipoint linkage algorithm using fast Fourier transforms is used to calculate these sharing probabilities (30, 31). First single point sharing probabilities are calculated by dividing the genome into 0.5cM bins and using the haplotypes over these bins as alleles. Haplotypes that are the same, except at most at a single SNP, are treated as identical. When the haplotypes in the pedigree are incompatible over a bin, then a uniform probability distribution was used for that bin. The most common causes for such incompatibilities are recombinations within the pedigree, phasing errors and genotyping errors. Note that since the input genotypes are fully phased, the single point information is substantially more informative than for unphased genotyped, in particular one haplotype of the parent of a genotyped child is always known. The single point distributions are then convolved using the multipoint algorithm to obtain multipoint sharing probabilities at the center of each bin. Genetic distances were obtained from the most recent version of the deCODE genetic map (32).
3. Based on the sharing probabilities at the center of each bin, all the SNPs from the whole genome sequencing are imputed into the proband. To impute the genotype of the paternal allele of a SNP located at x, flanked by bins with centers at x_{left} and x_{right}. Starting with the left bin, going through all possible sharing patterns ν, let I_{ν} be the set of haplotypes of genotyped individuals that share identically by descent within the pedigree with the proband's paternal haplotype given the sharing pattern ν and P(ν) be the probability of ν at the left bin—this is the output from step 2 above—and let eᵢ be the expected allele count of the SNP for haplotype i. Then

$${e}_{v} = \frac{{\sum }_{i ∈ {I}_{v}} {e}_{i}}{{\sum }_{i ∈ {I}_{v}} 1}$$

is the expected allele count of the paternal haplotype of the proband given ν and an overall estimate of the allele count given the sharing distribution at the left bin is obtained from e_{left}=Σ_{ν}P(ν)e_{ν}. If I_{ν} is empty then no relative shares with the proband's paternal haplotype given ν and thus there is no information about the allele count. The probability that some genotyped relative shared the proband's paternal haplotype is therefore stored, O_{left}=Σ_{ν,I}_{_{ν}}=P(V) and an expected allele count, conditional on the proband's paternal haplotype being shared by at least one genotyped relative:

$${c}_{left} = \frac{{\sum }_{v , {I}_{v \neq ∅}} P ( v ) {e}_{v}}{{\sum }_{v , {I}_{v \neq ∅}} P ( v )} .$$

In the same way calculate O_{right} and c_{right}. Linear interpolation is then used to get an estimates at the SNP from the two flanking bins:

$$O = {O}_{left} + \frac{x - {x}_{left}}{{x}_{right} - {x}_{left}} ( {O}_{right} - {O}_{left} ) , \\ c = {c}_{left} + \frac{x - {x}_{left}}{{x}_{right} - {x}_{left}} ( {c}_{right} - {c}_{left} ) .$$

If θ is an estimate of the population frequency of the SNP then Oc+(1−O)θ is an estimate of the allele count for the proband's paternal haplotype. Similarly, an expected allele count can be obtained for the proband's maternal haplotype.

### Case-Control Association Testing:

Logistic regression was used to test for association between SNPs and disease, treating disease status as the response and expected genotype counts from imputation or allele counts from direct genotyping as covariates. Testing was performed using the likelihood ratio statistic. When testing for association based on the in silico genotypes, controls were matched to cases based on the informativeness of the imputed genotypes, such that for each case C controls of matching informativeness where chosen. Failing to match cases and controls will lead to a highly inflated genomic control factor, and in some cases may lead to spurious false positive findings. The informativeness of each of the imputation of each one of an individual's haplotypes was estimated by taking the average of

$$a ( e , θ ) = \{ \begin{matrix}\frac{e - θ}{1 - θ} , & e \geq θ \\ \frac{θ - e}{θ} , & e < θ\end{matrix}$$

over all SNPs imputed for the individual, where e is the expected allele count for the haplotype at the SNP and θ is the population frequency of the SNP. Note that α(θ,θ)=0 and α(0,θ)=α(1,θ)=1. The mean informativeness values cluster into groups corresponding to the most common pedigree configurations used in the imputation, such as imputing from parent into child or from child into parent. Based on this clustering of imputation informativeness, the haplotypes of individuals were divided into seven groups of varying informativeness which created 27 groups of individuals of similar imputation informativeness; 7 groups of individuals with both haplotypes having similar informativeness, 21 groups of individuals with the two haplotypes having different informativeness, minus the one group of individuals with neither haplotype being imputed well. Within each group the ratio of the number of controls and the number of cases is calculated, and the largest integer C that was less than this ratio in all the groups is chosen. For example, if in one group there are 10.3 times as many controls as cases and if in all other groups this ratio was greater, then C=10 would be set and within each group there would be a random selection of ten times as many controls as there are cases.

### Inflation Factor Adjustment:

In order to account for the relatedness and stratification within our case and control sample sets the method of genomic control is applied based on chip markers. For the ovarian cancer genome-wide association study the correction factors based on the genomic control was 1.12.

### Sequencing of BRIP1 in Pooled DNA:

1. Target Enrichment of BRIP1 in Pooled DNA Samples.
Pooled DNA samples were enriched for BRIP1 (chr17:57,110,449-57,302,085, Build36/Hg18) using long range PCR (L-PCR). Primers were designed for overlapping (300-500 bp) fragments of approximately 6 kb in size. Primer design was done using the following restrictions: Tm, 62° C.; length 23 bp; GC content, 40-60% and by avoiding repeats and known SNP's or indels. Primer pairs which initially failed to generate products were re-designed to generate fragments of 3-3.5 kb in size. The sequences of the L-PCR primers are shown in Supplementary table 7. The Expand Long-Range dNTP pack from Roche Applied Science was used for L-PCR following the manufacturer's instructions. Each PCR reaction was done in a volume of 50 μl with an input of 80 ng of DNA per reaction. All PCR reactions were performed on MJR PTC-225 thermocyclers with 96-well blocks. PCR products were seperated using agarose (1.2%) gel electrophoresis and detected with BlueView (Sigma) staining. Bands of correct size were cut out and the DNA was isolated using Ultrafree DA DNA spin columns (Millipore). The concentration of each L-PCR product was measured using PicoGreen fluorescence measurements and the products (50 ng each) were combined into a single enriched sample for each pool. Final concentration and size distribution of the combined samples were assessed using the Agilent Bioanalyzer 2100 with a DNA 12000 LabChip kit.

2. Preparation of Target Enriched Samples for Indexed Sequencing.
Indexed paired-end libraries for sequencing were prepared using the TruSeq™ sample preparation kit according to the manufacturer's instructions (Illumina) and as described for the tumor samples above. Appropriate TruSeq sequencing adaptors for multiplexing (index #1-3) were employed

3. DNA Sequencing of Target Enriched Samples:
Libraries with index #1, #2 and #3 were mixed together in equal quantities to generate indexed stocks of 2 nM. Samples were clustered as described above for whole genome sequencing using a template concentration of 3 pm. Sequencing was performed on Illumina GAIIx instruments equipped with paired-end modules. Paired-end samples (three indexed samples per lane) were sequenced using 2×50 cycles of incorporation and imaging with TruSeq Illumina sequencing kits, v5. Imaging and analysis of the data was performed using the SCS2.9/RTA1.9 software packages from Illumina, respectively.

### Analysis of Pool Sequencing Data:

A simple likelihood ratio test was used to test for the presence of a SNP at every exonic position. Since the number of individuals in each pool is less than 50, implying a minimal pool frequency of 1/100, only SNPs reaching an estimated pool frequency of at least 0.005 were considered. Indel calling within exons was done manually. Potential inserts and deletions falling within exons were identified using the samtools alignment results and all the resulting candidates were then inspected by eye. Only the Icelandic two-base insert and the Spanish two-base deletion were deemed to be likely to be real polymorphisms.

### Microsatellite Genotyping:

The PCR amplifications were set up and pooled using Zymark SciClone ALH 500 robots. The sequences of the primers used for genotyping are listed in Supplementary table 8. The reaction volume was 5 μl, and, for each PCR, 20 ng of genomic DNA was amplified in the presence of 2 μmol of each primer, 0.14 U AmpliTaq Gold, 0.33 mmol/liter dNTPs, and 3.3 mmol/liter MgCl₂. The PCR conditions were 95° C. for 10 minutes, then stepdown 4 cycles of 15 s at 94° C., 30 s at 63° C.-2.5° C. per cycle, and 30 s at 72° C., 11 cycles of 15 s at 94° C., 30 s at 55° C., and 1 min at 72° C., at last 22 cycles of 15 s at 89° C., 30 s at 55° C., and 1 min at 72° C. The PCR products were supplemented with an internal size standard, and the fragments were separated and detected on an Applied Biosystems model 3730 sequencer, using Genescan (v. 3.0) peak-calling software. Alleles were called using an internal allele-calling program (33).

### Association Analysis

For association analysis of the replication datasets, a standard likelihood ratio statistic was used, implemented in the NEMO software (34) to calculate two-sided P-values for each individual allele, assuming a multiplicative model for ovarian cancer risk, i.e. the ovarian cancer risk multiplies by the number of risk alleles a person carries. Results from multiple case-control groups were combined using a Mantel-Haenszel model in which the groups were allowed to have different population frequencies for alleles and genotypes but were assumed to have common odds ratios.

Isolation of Nucleic Acids from Fresh-Frozen and Paraffin-Embedded Tissue Samples:

DNA was extracted from sectioned frozen and paraffin-embedded tissue using MasterPure reagents (Epicentre Biotechnologies). For paraffin-embedded tissue, paraffin was removed prior to extraction by heating to 95° C. in Tissue and Cell Lysis buffer (TCLS), cooling on ice and transferring the lysate to clean tubes. Reagent volumes were adjusted according to the amount of tissue. Tissue sections were lysed at room temperature whilst rotating until visibly digested. The manufacturer's protocol was followed for the remainder of the protocol. RNA was extracted from sectioned frozen tissue using RNAzol® RT (Molecular Research Centre). Tissue sections were homogenized using a rotor-stator homogenizer. The manufacturer's protocol for isolation of RNA containing fraction >200 bp was followed.

### Test for BRIP1 Expression and LOH in Ovarian Tumor Samples:

RNA samples from fresh-frozen ovarian tumor samples were converted to cDNA using the High Capacity cDNA Reverse Transcriptase kit (Applied Biosystems Inc.). The region around the 2 bp deletion in exon 12 was amplified from cDNA from frozen tumors and genomic DNA from frozen and paraffin-embedded tumors using conventional PCR (primer sequences are listed in Supplementary table 9). The PCR fragments were sequenced using the same primers and the Big-Dye R terminator v3.1 chemistry, followed by loading onto a 3730 DNA Analyzer (Applied Biosystems Inc.).

### Whole-Genome Sequencing of Tumor DNA:

The TruSeq™ sample preparation kit (Illumine) was employed for the preparation of sequencing libraries for genomic DNA from tumor samples and matched germline/blood samples. The method was the essentially the same as described above for germ-line DNA above, except the DNA input was 1 μg and the supplied TruSeq adaptors and PCR primer cocktail were used. Purification of samples following PCR amplification was also done using AMPure XP beads instead of gel electrophoresis.

### Example 7

### BRIP1 Binding to C-Terminal BRCT Motifs of Wild-Type Human BRCA1 Protein

The BRCA1-interacting fraction of BRIP1 extends from amino acid residues 979-1006 and phosphorylation of Ser990 is important for the binding. Missense mutations within this region may affect the binding of BRIP1 to BRCA1. In addition, mutations in BRIP1 that are located outside of the BRCA1-binding region might also affect the interaction between the two proteins if the mutation affect the three-dimensional structure of the protein.

In order to test if a particular mutation affects the binding of BRIP1 to BRCA, the following assay could be applied as described by Cantor et al Cell 105:149-160 (2001).

A recombinant protein “bait”, composed of the BRCT region of BRCA1 (amino acids 1529-1863) fused to glutathione S-transferase (GST), is immobilized on glutathione-coated sepharose beads. The mutant BRIP1 cDNA is cloned into an appropriate plasmid vector, labeled (³⁵S) BRIP1 protein is produced by in vitro translation and incubated with the BRCT-GST fusion protein. After washing of the beads, binding between BRCT-GST and BRIP1 is assessed by SDS gel electrophoresis and autoradiography. Wild-type BRIP1 protein, cloned and produced in the same manner is used as positive control and empty vector as a negative control.

### Example 8

### Assay for Determining DNA-Dependent ATPase Activity

BRIP1 contains both DNA-dependent ATPase and helicase activities and the helicase activity is dependent on the ATPase activity. To test the ATPase activity of BRIP1, the mutant BRIP1 cDNA to be tested can be cloned into a FLAG-epitope containing baculovirus vector, transfected into insect cells and the recombinant protein purified using an anti-FLAG antibody. Wild-type BRIP1 cDNA is also expressed in the same manner for use as positive control. The ATPase activity of the BRIP1 proteins is then assessed by measuring the release of free phosphate during ATP hydrolysis in the presence of different types of DNA (calf thymus DNA, circular single-stranded M13 DNA, and supercoiled pcDNA3.0) as cofactors, as described (Cantor et al. PNAS 101:2357-2362 (2004)).

### Example 9

### Determination of DNA Helicase Activity

To test for the helicase activity of mutant BRIP1 protein, the helicase assays described by Cantor et al. PNAS 101:2357-2362 (2004) may be used. Briefly, the mutant BRIP1 cDNA is cloned into a FLAG-epitope containing baculovirus vector, transfected into insect cells and the recombinant protein is purified using anti-FLAG antibody. Wild-type BRIP1 cDNA is also expressed in the same manner and used as positive control.

Various types of DNA and RNA oligonucleotides are used to construct the substrates for helicase assays and all DNA oligonucleotides are designed to be complementary to a segment of M13 mp 18 single-stranded DNA (M13). Oligonucleotides are used to generate partially double-stranded DNA and DNA:RNA duplexes by annealing to M13 DNA. After annealing, the annealed primer is extended by one nucleotide with DNA polymerase I (Klenow fragment) by using [α-32P]GTP. After a purification step, helicase activity is measured by detecting the displacement of labeled DNA or RNA oligonucleotide from the partially duplexed substrate. The reaction is initiated by addition of immunoprecipitated, recombinant BRIP1 and incubated at 30° C. for 30 min. The reaction is stopped and the reaction products are resolved by electrophoresis in an 8% native TBE polyacrylamide gel containing 15% glycerol.

### Example 10

### Determination of Expression Levels of BRIP1 Protein

Missense mutations, although they may not alter the function of the protein, may result in a less stable protein product. Insufficient levels of the BRIP1 protein will have adverse effect on genomic integrity and increase the risk of tumor formation.

In order to test the stability of mutant BRIP1 proteins, a cycloheximide-chase analysis can be applied as described in Cantor et al Cell 105:149-160 (2001). Mutant and wild-type BRIP1 cDNAs are cloned into an expression vector containing an appropriate tag, such as the Myc epitope. A eukaryotic cell line is transfected with the vectors, lysates are collected at fixed time points and the quantity of recombinant protein is assessed by western blotting using an anti-Myc antibody. To assess the half-life of the recombinant proteins, cycloheximide (an inhibitor of protein biosynthesis) is added to the cultures at a set time after transfection and the level of wild-type and mutant BRIP1 protein is assessed by western blotting at serial timepoints.

1. A method of determining a susceptibility to a cancer, the method comprising:
analyzing a biological sample from a human subject to obtain data representative of at least one allele of a BRIP1 gene (SEQ ID NO:15) in a human subject, wherein different alleles of the human BRIP1 gene are associated with different susceptibilities to at least one cancer in humans, and
determining a susceptibility to a cancer for the human subject from the data, wherein the data is analyzed for the presence or absence of at least one mutant allele indicative of a BRIP1 defect selected from the group consisting of:
(a) premature truncation or frameshift of an encoded BRIP1 protein, relative to the BRIP1 amino acid sequence set forth in SEQ ID NO:13;
(b) expression of a BRIP1 protein with reduced activity compared to a wild-type BRIP1 protein (SEQ ID NO:13), wherein the activity is at least one BRIP1 activity selected from:
(i) BRIP1 binding to C-terminal BRCT motifs of wildtype human BRCA1 protein;
(ii) DNA-dependent ATPase activity; and
(iii) DNA helicase activity;
(c) reduced expression of BRIP1 protein, compared to wild-type BRIP1,
and wherein mutant alleles indicative of the defect are associated with increased susceptibility to the cancer.

2. The method according to claim 1, wherein the cancer is selected from the group consisting of ovarian cancer, pancreatic cancer, colorectal cancer, upper airways cancer, and breast cancer.

3. The method according to claim 1, wherein the cancer is ovarian cancer.

4. (canceled)

5. The method of claim 1, comprising analyzing the data for the presence or absence of at least one mutant allele that results in elimination of the at least one activity.

6. The method of claim 1, wherein the analyzing data comprises analyzing the biological sample from the human subject to obtain information selected from the group consisting of:
(a) nucleic acid sequence information, wherein the nucleic acid sequence information comprises sequence sufficient to identify the presence or absence of the mutant allele in the subject;
(b) nucleic acid sequence information, wherein the nucleic acid sequence information identifies at least one allele of a polymorphic marker in linkage disequilibrium (LD) with the mutant allele, wherein the LD is characterized by a value for r² of at least 0.5;
(c) measurement of the quantity or length of BRIP1 mRNA, wherein the measurement is indicative of the presence or absence of the mutant allele;
(d) measurement of the quantity of BRIP1 protein, wherein the measurement is indicative of the presence or absence of the mutant allele; and
(e) measurement of BRIP1 activity, wherein the measurement is indicative of the presence or absence of the mutant allele.

7. The method of claim 6, comprising analyzing the biological sample to obtain the nucleic acid sequence information.

8. (canceled)

9. (canceled)

10. The method of claim 1, wherein the presence of the mutant allele is indicative of increased susceptibility to the cancer with a relative risk (RR) or odds ratio (OR) of at least 2.0.

11. The method of claim 1, wherein the mutant allele comprises a BRIP1 frameshift mutation.

12. The method of claim 11, wherein the mutation is selected from the group consisting of chr17:57208601 ins+AA and chr17: 57213073 delTT.

13. The method of claim 1, wherein the mutant allele is a BRIP1 nonsense mutation.

14. The method of claim 12, wherein the mutant allele is a BRIP1 missense mutation which results in expression of a BRIP1 protein with reduced activity compared to a wild-type BRIP1 protein.

15. The method of claim 14, wherein the missense mutation results in elimination of BRIP1 activity.

16. A method of determining whether an individual is at increased risk of developing ovarian cancer, the method comprising steps of
obtaining a biological sample containing nucleic acid from the individual;
determining, in the biological sample, nucleic acid sequence data about BRIP1 gene; and
comparing the sequence information to wild-type BRIP1 (SEQ ID NO: 10) sequence;
wherein an identification of a mutation in BRIP1 in the individual is indicative of the individual being at increased risk of developing ovarian cancer.

17. The method of claim 16, wherein the mutation is a missense mutation, a nonsense mutation or a frameshift mutation in BRIP1.

18. The method of claim 16, wherein the mutation results in a BRIP1 defect selected from the group consisting of:
(a) premature truncation or frameshift of an encoded BRIP1 protein, relative to the BRIP1 amino acid sequence set forth in SEQ ID NO:13;
(b) expression of a BRIP1 protein with reduced activity compared to a wild-type BRIP1 protein (SEQ ID NO:13), wherein the activity is at least one BRIP1 activity selected from:
(i) BRIP1 binding to C-terminal BRCT motifs of wildtype human BRCA1 protein;
(ii) DNA-dependent ATPase activity; and
(iii) DNA helicase activity;
(c) reduced expression of BRIP1 protein, compared to wild-type BRIP1,
wherein mutant alleles indicative of the defect are associated with increased susceptibility to the cancer.

19. A method of determining whether a human subject is at increased risk of developing ovarian cancer, the method comprising analyzing a biological sample from the human subject to obtain amino acid sequence data about a BRIP1 polypeptide from the subject, and
determining whether the subject is at increased risk of developing ovarian cancer from the amino acid sequence data, wherein a determination of the presence of a truncated BRIP1 polypeptide compared with a wild-type BRIP1 polypeptide with sequence as set forth in SEQ ID NO:13 is indicative that the subject is at increased risk of developing ovarian cancer.

20. The method of claim 19, wherein the amino acid sequence data is obtained from the biological sample using a method that comprises at least one procedure selected from:
(i) an antibody assay; and
(ii) protein sequencing.

21. (canceled)

22. A method for determining a susceptibility to a cancer in a human individual, comprising
determining the presence or absence of at least one allele of at least one polymorphic marker in a nucleic acid sample obtained from the individual, wherein the at least one allele causes a loss of function or loss of expression of BRIP1, and
determining a susceptibility to the cancer from the presence or absence of the at least one allele, wherein the presence of the at least one allele is indicative of a susceptibility to the cancer.

23. The method of claim 22, wherein the at least one polymorphic marker is selected from the group consisting of polymorphic markers that cause a loss of function of a BRIP1.

24. The method of claim 22, wherein the at least one polymorphic marker is selected from the group consisting of polymorphic markers that cause a frameshift mutation or nonsense mutation in BRIP1 with sequence as set forth in SEQ ID NO:10.

25. The method according to claim 22, wherein the cancer is selected from the group consisting of ovarian cancer, pancreatic cancer, colorectal cancer, upper airways cancer, and breast cancer.

26. The method of claim 22, wherein the cancer is ovarian cancer.

27. A method of determining a susceptibility to ovarian cancer, the method comprising:
analyzing a biological sample from a human subject for evidence of an allele of BRIP1 (SEQ ID NO: 15) that results in impaired BRIP1 function, wherein the presence of an allele of BRIP1 with impaired function is associated with elevated susceptibility to ovarian cancer in humans, and
determining a susceptibility to ovarian cancer for the human subject from the presence or absence of evidence of the allele of BRIP1 that results in the impaired BRIP1 function.

28. The method according to claim 27, comprising analyzing for the presence of a BRIP1 allele with impaired function selected from the group consisting of:
(a) alleles resulting in premature truncation or frameshift of an encoded BRIP1 protein, relative to the BRIP1 amino acid sequence set forth in SEQ ID NO:13;
(b) alleles encoding a BRIP1 protein with reduced activity compared to a wild-type BRIP1 protein (SEQ ID NO:13), wherein the activity is at least one BRIP1 activity selected from:
(i) BRIP1 binding to C-terminal BRCT motifs of wildtype BRCA1 protein;
(ii) DNA-dependent ATPase activity; and
(iii) DNA helicase activity;
(c) alleles resulting in reduced expression of BRIP1 protein.

29-49. (canceled)

50. A system for identifying susceptibility to a cancer in a human subject, the system comprising:
at least one processor;
at least one computer-readable medium;
a susceptibility database operatively coupled to a computer-readable medium of the system and containing population information correlating the presence or absence of one or more alleles of the human BRIP1 gene and susceptibility to a cancer in a population of humans;
a measurement tool that receives an input about the human subject and generates information from the input about the presence or absence of at least one mutant BRIP1 allele indicative of a BRIP1 defect in the human subject, wherein the BRIP1 defect is selected from the group consisting of:
(a) premature truncation of an encoded BRIP1 protein, relative to the BRIP1 amino acid sequence set forth in SEQ ID NO: 13;
(b) expression of a BRIP1 protein with reduced activity compared to a wild-type BRIP1 protein (SEQ ID NO: 13), wherein the activity is at least one BRIP1 activity selected from:
(i) BRIP1 binding to C-terminal BRCT motifs of wildtype human BRCA1 protein;
(ii) DNA-dependent ATPase activity; and
(iii) DNA helicase activity; and
(c) reduced expression of BRIP1 protein, compared to wildtype expression,
wherein mutant alleles indicative of the defect are associated with increased susceptibility to the cancer; and
an analysis tool that:
is operatively coupled to the susceptibility database and the measurement tool,
is stored on a computer-readable medium of the system,
is adapted to be executed on a processor of the system, to compare the information about the human subject with the population information in the susceptibility database and generate a conclusion with respect to susceptibility to the cancer for the human subject.

51. The system according to claim 50, further including:
a communication tool operatively coupled to the analysis tool, stored on a computer-readable medium of the system and adapted to be executed on a processor of the system to communicate to the subject, or to a medical practitioner for the subject, the conclusion with respect to susceptibility to the cancer for the subject.

52. The system according to claim 50, wherein the cancer is selected from the group consisting of ovarian cancer, pancreatic cancer, colorectal cancer, upper airways cancer, and breast cancer.

53. The system according to claim 50, wherein the cancer is ovarian cancer.

54. (canceled)

55. The system according to claim 50, wherein the measurement tool comprises a tool stored on a computer-readable medium of the system and adapted to be executed by a processor of the system to receive a data input about a subject and determine information about the presence or absence of the at least one mutant BRIP1 allele in a human subject from the data.

56. The system according to claim 55, wherein the data is genomic sequence information, and the measurement tool comprises a sequence analysis tool stored on a computer readable medium of the system and adapted to be executed by a processor of the system to determine the presence or absence of the at least one mutant BRIP1 allele from the genomic sequence information.

57. The system according to claim 50, wherein the input about the human subject is a biological sample from the human subject, and wherein the measurement tool comprises a tool to identify the presence or absence of the at least one mutant BRIP1 allele in the biological sample, thereby generating information about the presence or absence of the at least one mutant BRIP1 allele in a human subject.

58. The system according to claim 57, wherein the measurement tool includes:
an oligonucleotide microarray containing a plurality of oligonucleotide probes attached to a solid support;
a detector for measuring interaction between nucleic acid obtained from or amplified from the biological sample and one or more oligonucleotides on the oligonucleotide microarray to generate detection data; and
an analysis tool stored on a computer-readable medium of the system and adapted to be executed on a processor of the system, to determine the presence or absence of the at least one mutant BRIP1 allele based on the detection data.

59. The system according to claim 57, wherein the measurement tool includes:
a nucleotide sequencer capable of determining nucleotide sequence information from nucleic acid obtained from or amplified from the biological sample; and
an analysis tool stored on a computer-readable medium of the system and adapted to be executed on a processor of the system, to determine the presence or absence of the at least one mutant BRIP1 allele based on the nucleotide sequence information.

60. (canceled)

61. The system according to claim 51, wherein the communication tool is operatively connected to the analysis routine and comprises a routine stored on a computer-readable medium of the system and adapted to be executed on a processor of the system, to:
generate a communication containing the conclusion; and
transmit the communication to the subject or the medical practitioner, or enable the subject or medical practitioner to access the communication.

62. The system according to claim 61, wherein the communication expresses the susceptibility to the cancer in terms of odds ratio or relative risk or lifetime risk.

63. (canceled)

64. The system according to claim 50, wherein the susceptibility database further includes information about at least one parameter selected from the group consisting of age, sex, ethnicity, race, medical history, weight, diabetes status, blood pressure, family history of the cancer, and smoking history in humans and impact of the at least one parameter on susceptibility to the cancer.

65. A system for assessing or selecting a treatment protocol for a subject diagnosed with a cancer, comprising:
at least one processor;
at least one computer-readable medium;
a medical treatment database operatively connected to a computer-readable medium of the system and containing information correlating the presence or absence of at least one mutant BRIP1 allele and efficacy of treatment regimens for the cancer;
a measurement tool to receive an input about the human subject and generate information from the input about the presence or absence of the at least one mutant BRIP1 allele indicative of a BRIP1 defect in a human subject diagnosed with the cancer; and
a medical protocol tool operatively coupled to the medical treatment database and the measurement tool, stored on a computer-readable medium of the system, and adapted to be executed on a processor of the system, to compare the information with respect to presence or absence of the at least one mutant BRIP1 allele for the subject and the medical treatment database, and generate a conclusion with respect to at least one of:
the probability that one or more medical treatments will be efficacious for treatment of the cancer for the patient; and
which of two or more medical treatments for the cancer will be more efficacious for the patient.

66. The system according to claim 65, wherein the measurement tool comprises a tool stored on a computer-readable medium of the system and adapted to be executed by a processor of the system to receive a data input about a subject and determine information about the presence or absence of the at least one mutant BRIP1 allele in a human subject from the data.

67. The system according to claim 66, wherein the data is genomic sequence information, and the measurement tool comprises a sequence analysis tool stored on a computer readable medium of the system and adapted to be executed by a processor of the system to determine the presence or absence of the at least one mutant BRIP1 allele from the genomic sequence information.

68. The system according to claim 65, wherein the input about the human subject is a biological sample from the human subject, and wherein the measurement tool comprises a tool to identify the presence or absence of the at least one mutant BRIP1 allele in the biological sample, thereby generating information about the presence or absence of the at least one mutant BRIP1 allele in a human subject.

69. The system according to claim 65, further comprising a communication tool operatively connected to the medical protocol routine for communicating the conclusion to the subject, or to a medical practitioner for the subject.

70. The system according to claim 69, wherein the communication tool comprises a routine stored on a computer-readable medium of the system and adapted to be executed on a processor of the system, to:
generate a communication containing the conclusion; and
transmit the communication to the subject or the medical practitioner, or enable the subject or medical practitioner to access the communication.
